(19) United States
(12) Patent Application Publication
(10) Pub. No.: US 2025/0051437 A1
(43) Pub. Date: Feb. 13, 2025

(54) CDH17 ANTIBODIES AND METHODS OF TREATING CANCER

(71) Applicant: The Regents of the University of California, Oakland, CA (US)

(72) Inventors: Dennis Slamon, Los Angeles, CA (US); Martina S. McDermott, Los Angeles, CA (US); Neil A. O'Brien, Los Angeles, CA (US)

(21) Appl. No.: 18/717,817

(22) PCT Filed: Dec. 7, 2022
(86) PCT No.: PCT/US22/52132

§ 371 Date: Jun. 7, 2024

Related U.S. Application Data
(60) Provisional application No. 63/349,258, filed on Jun. 6, 2022.
(60) Provisional application No. 63/286,894, filed on Dec. 7, 2021.

(51) Int. Cl. C07K 16/28; A61K 47/68; A61P 35/00; A61P 37/04; G01N 33/53; G01N 33/68

(52) U.S. Cl. CPC C07K 16/28; A61K 47/68031; A61K 47/6849; A61P 35/00; A61P 37/04; G01N 33/53; G01N 33/68; C07K 2317/24; C07K 2317/33; C07K 2317/565; C07K 2317/622; C07K 2317/734; C07K 2317/92; G01N 2333/705

(57) ABSTRACT

The present disclosure provides antigen-binding proteins which bind to CDH17; bispecific antigen-binding proteins which bind to CDH17 and a second antigen; and conjugates thereof. Related polypeptides, nucleic acids, vectors, host cells, and conjugates are further provided herein. Kits and pharmaceutical compositions comprising such entities are moreover provided. Also provided are methods of making an antigen-binding protein and methods of treating a subject having cancer.

### CROSS-REFERENCE TO RELATED APPLICATIONS

This application claims the benefit of U.S. Provisional Application No. 63/286,894, filed Dec. 7, 2021; and U.S. Provisional Application No. 63/349,258, filed Jun. 6, 2022; the entire contents of each of said applications are incorporated herein in their entirety by this reference.

### FIELD OF THE DISCLOSURE

The present disclosure relates, in general, to antibodies specific for Cadherin-17 (CDH17) and uses thereof to treat cancer.

### BACKGROUND

Antibodies constitute powerful therapeutic agents characterized by limited side effects due to their ability to specifically target a distinct antigen on a cell, bacteria, virus, or toxin. There is a clinical need to provide new antibodies, such as the antibodies described herein to address the medical needs of patients relating to CDH17-associated disease.

### SUMMARY

Provided herein are antigen-binding proteins which bind to CDH17. In various aspects, the antigen-binding protein of the present disclosure binds to a human CDH17 and optionally binds to a mouse CDH17. In various aspects, the antigen-binding protein binds to the extracellular domain (ECD) of CDH17. In various embodiments, the disclosure provides an antigen binding protein against CDH17.

In various instances, the antigen binding protein binds to CDH17 and does not bind to any other member of the CDH family. In various aspects, the antigen binding protein binds to CDH17 endogenously expressed by human cancer cells, e.g., HPAF-II pancreatic cells. In various instances, the antigen-binding proteins of the present disclosure inhibit tumor growth in a subject, e.g., a human, without any other moiety attached to the antigen-binding protein. In various instances, the antigen-binding proteins unconjugated to a heterologous moiety (e.g., unconjugated to any chemotherapeutic agent, drug or toxic moiety) inhibit tumor growth in a subject, e.g., a human.

In various aspects, the antigen-binding protein binds to CDH17 expressed by human cancer cells. In various aspects, the antigen-binding protein inhibits a binding interaction between human CDH17 and a reference anti-CDH17 antibody. Without being bound to a particular theory, the inhibiting action of the antigen-binding proteins provided herein allow such entities to be useful in methods of reducing tumor growth and treating a subject with a tumor or cancer. As further discussed herein, in various aspects, the antigen-binding protein is an antibody, antigen-binding antibody fragment thereof, or antibody protein product.

The present disclosure also provides antigen-binding proteins comprising at least 3, 4, 5, or all amino acid sequences of a specified group of amino acid sequences. In various aspects, the antigen-binding proteins comprise at least 3, 4, 5, or 6 complementary determining region (CDR) amino acid sequences of CDH17 antibodies disclosed herein.

The present disclosure further provides antigen-binding proteins comprising amino acid sequences as detailed herein.

The present disclosure provides a bispecific antigen-binding protein that binds to CDH17 and a second antigen. The bispecific antigen-binding protein may comprise any one of the antigen-binding protein described here. The second antigen may be a cell surface protein, optionally a protein whose binding modulates immune response. The bispecific antigen-binding protein may take any structure, e.g., diabody, TandAb (tandem diabody), BiTE (bispecific T cell engager), etc.

The present disclosure also provides a conjugate that comprises an antigen-binding protein or a bispecific antigen-binding protein and a heterologous moiety (e.g., a cytotoxic drug). The conjugate may comprise a cleavable linker or a noncleavable linker. The conjugate may have a various number of heterologous moiety (an agent) conjugated to the antigen-binding protein or a bispecific antigen-binding protein described herein, preferably 1-8 agents per protein or 3-8 agents per protein. The conjugate may be a site-specific conjugate. The conjugate may be a homogenous conjugate or a heterogeneous conjugate.

Related polypeptides, nucleic acids, vectors, host cells, and conjugates are further provided herein. Kits and pharmaceutical compositions comprising such entities are moreover contemplated.

Also provided are methods of making an antigen-binding protein. In various embodiments, the method comprises culturing a host cell comprising a nucleic acid encoding an antigen-binding protein or a polypeptide as described herein so as to express the antigen-binding protein or polypeptide.

Methods of treating a subject having cancer are additionally provided herein. In various embodiments, the method comprises administering to the subject the pharmaceutical composition of the present disclosure in an amount effective for treating the cancer in the subject.

Also provided are methods of treating a subject with a CDH17-expressing cancer comprising administering to the subject a pharmaceutical composition described herein. In various embodiments, the CDH17-expressing cancer expresses CDH17. Further contemplated is a method of inhibiting tumor growth in a subject, comprising administering to the subject a pharmaceutical composition described herein.

A method of reducing tumor size in a subject, or preventing the recurrence of cancer in a subject comprising administering to the subject a pharmaceutical composition described herein.

Also provided herein is a method of treating cancer in a subject diagnosed to be a over-expresser of CDH17, comprising administering to the subject a pharmaceutical composition described herein.

In various embodiments, the administering induces apoptosis in tumor cells, for example in cells expressing CDH17, resulting in tumor regression or slowing of tumor growth. In various embodiments, the administration induces antibody-dependent cell-mediated cytotoxicity (ADCC) or Complement-dependent cytotoxicity (CDC), tumor necrosis and death or depletion of cells, and/or disruption of tumor cell adherence, each of which result in tumor regression or slowing of tumor growth.

### BRIEF DESCRIPTION OF THE DRAWINGS

FIG. 1 represents a graph of CDH17 expression in human cancers.

FIG. 2A-FIG. 2B show results of sequence comparison of human CDH17 (UniProtKB accession number Q12864) against crab-eating macaque CDH17 (UniProtKB accession number A0A2K5X818), mouse CDH17 (UniProtKB accession number Q9R100) and rat CDH17 (UniProtKB accession number P55281) as percent identity and phylogram and topology of CDH17 transmembrane protein (A) and aligned sequences with yellow highlight indicating extracellular domain of human CDH17 (B).

FIG. 3 represents graphs of tumor volume (mm³) as a function of time (days) (A), mean change in tumor volume (mm³) at Day 32 (B) of tumors, and percent change in body weight (C) in mice bearing human CDH17-positive colorectal tumors (SNU-C1; ATCC CRL-5972) after treatment weekly at 10 mg/kg with control IgG antibody (human IgG1) or 6 different chimeric monoclonal antibodies directed against CDH17 protein. The chimeric monoclonal antibodies comprise mouse VH and VL sequences, human IgG1 constant region, and human Ig kappa constant region.

FIG. 4 represents graphs of tumor volume (mm³) as a function of time (days) (A), mean change in tumor volume (mm³) at Day 28 (B) of tumors, and percent change in body weight (C) in mice bearing human CDH17-positive colorectal tumors (SNU-C1; ATCC CRL-5972) after treatment weekly at 10 mg/kg with control human IgG1 antibody, two different chimeric monoclonal anti-CDH17 antibodies (CDH17-653-m or CDH17-657-m) or eight different humanized anti-CDH17 antibodies (CDH17-646-h7, CDH17-653-h42, CDH17-653-h43, CDH17-657-h16, CDH17-663-h7, CDH17-670-h12, CDH17-675-h11 or CDH17-683-h6); note that “CDH17-” may be replaced with “07-0”, which refers to the same antibody (for examples, an antibody designated as CDH17-646-h7 is the same antibody as one designated as 07-0646-h7; CDH17-663-h7 is also known as 07-0663-h7 and CDH17 hAb 0663-h7).

FIG. 5 represents graphs of tumor volume (mm³) as a function of time (days) (A) and mean change in tumor volume (mm³) at Day 25 (B) of tumors in mice bearing human CDH17-positive pancreatic tumors (HPAF-II) after treatment weekly at 10 mg/kg with control human IgG1 antibody or eight different humanized anti-CDH17 antibodies (CDH17-646-h7, CDH17-653-h42, CDH17-653-h43, CDH17-657-h16, CDH17-663-h7, CDH17-670-h12, CDH17-675-h11 or CDH17-683-h6).

FIG. 6 represents graphs of tumor volume (mm³) as a function of time (days) (A) and mean change in tumor volume (mm³) at Day 24 (B) of tumors in mice bearing a human CDH17-negative melanoma cell line (M202) after treatment weekly at 10 mg/kg with control human IgG1 antibody or six different humanized anti-CDH17 antibodies (CDH17-646-h7, CDH17-653-h42, CDH17-653-h43, CDH17-663-h7, CDH17-670-h12 or CDH17-683-h6).

FIG. 7 shows selective tumor growth inhibition induced by antibody-drug conjugate (ADC; VC-PAB-MMAE) of chimeric monoclonal anti-CDH17 antibody, CDH17-653, over unconjugated antibody or control IgG antibody in mice bearing human CDH17-positive tumors (A-C) and over human CDH17-negative tumors (D-F). Xenografts were obtained from a human colorectal cancer cell line, SNU-C1 (A), a human pancreatic cancer cell line, PaTu8988s (B), a human colorectal cancer cell line, LS513 (C), human small cell lung carcinoma cell lines, H524 (D) and COR-L279 (E) and a human melanoma cell line, M202 (F). Mice were treated with 5 mg/kg ADC or 10 mg/kg unconjugated parental CDH17-653 or IgG control by IV tail vein injection for 3 weekly repeat doses.

FIG. 8 shows tumor growth inhibition in tumor volume (mm³) over time (A), mean change in tumor volume (mm³) at Day 42 (B), and percent change in body weight (C), induced by three different humanized anti-CDH17 antibody-drug conjugates (ADCs; VC-PAB-MMAE) in a CDH17-positive human colorectal cancer xenograft (SNU-C1) following weekly treatment with 5 mg/kg ADC for 3 repeat doses. Antibody-drug conjugates: CDH17-ADC-646-h7, CDH17-ADC-653-h43 and CDH17-ADC-663-h7. IgG is non-targeting control antibody.

FIG. 9 shows tumor growth inhibition in tumor volume (mm³) over time (A) or mean change in tumor volume (mm³) at Day 27 (B) induced by three different humanized anti-CDH17 antibody-drug conjugates (ADCs; VC-PAB-MMAE) in a CDH17-positive human pancreatic cancer xenograft (HPAF-II) following weekly treatment with 5 mg/kg ADC for 3 repeat doses. Antibody-drug conjugates: CDH17-ADC-646-h7, CDH17-ADC-653-h43 and CDH17-ADC-663-h7. IgG is non-targeting control antibody.

FIG. 10 represents a graph of tumor volume (mm³) as a function of time (days) (A) or mean change in tumor volume (mm³) at Day 31 (B) of tumors in mice bearing human CDH17-negative melanoma (M202) after treatment weekly at 5 mg/kg for 3 repeat doses with control IgG antibody or humanized anti-CDH17 antibody-drug conjugate (VC-PAB-MMAE) (CDH17-ADC-646-h7, CDH17-ADC-653-h43 or CDH17-ADC-663-h7), showing loss of anti-tumor activity in CDH17-negative xenografts by the ADCs.

FIG. 11 shows time course of internalization of humanized anti-CDH17 antibodies, 07-0646-h7 (top panels) and 07-0663-h7 (bottom panels), in a CDH17-positive human colorectal cancer cell line, LS513.

FIG. 12 shows time course of internalization of humanized anti-CDH17 antibodies, 07-0646-h7 (top panels), 07-0653-h33 (middle panels) and 07-0663-h7 (bottom panels), in a CDH17-positive human pancreatic cancer cell line, HPAF-II.

FIG. 13 shows time course of internalization of humanized anti-CDH17 antibody-drug conjugates (ADCs), 07-0646-h7-VC-PAB-MMAE (top panels) and 07-0653-h43-VC-PAB-MMAE (bottom panels), in a CDH17-positive human colorectal cancer cell line, LS513.

FIG. 14 shows time course of internalization of a humanized anti-CDH17 antibody (07-0663-h7) conjugated to monomethyl auristatin E, MMAE (07-0663-h7-VC-PAB-MMAE; top panels), or Alexa Fluor 647, AF647 (07-0663-h7-AF647; bottom panels), in a CDH17-positive human colorectal cancer cell line, LS513.

FIG. 15 shows flow cytometry assessment of the binding activity of three humanized anti-CDH17 antibodies to HEK293 cells overexpressing human, monkey, mouse or rat CDH17 as fusion proteins to mGFP (human or mouse CDH17) or moxGFP (monkey or rat CDH17) fluorescent protein. Top panel-anti-CDH17 signal following detection of anti-CDH17 antibody with a fluorescently labeled secondary antibody; bottom panel-GFP signal.

FIG. 16 summarizes dissociation constant, K_{D}, determined for three humanized anti-CDH17 antibodies binding to HEK293T cells overexpressing a fusion protein comprising a human CDH17-mGFP, monkey CDH17-moxGFP, mouse CDH17-mGFP, or rat CDH17-moxGFP protein.

FIG. 17 summarizes biochemical, biophysical and cell biological characteristics of three lead humanized anti-CDH17 antibodies along with properties of some commercial antibodies.

FIG. 18 shows SDS-PAGE analysis on stability of three humanized anti-CDH7 antibodies stored for 5 weeks at −80° C., 4° C., RT or 37° C. with left side of each gel showing stored antibodies subjected to reducing condition and right side showing stored antibodies subjected to non-reducing condition just prior to electrophoresis.

FIG. 19 shows size exclusion chromatogram and SDS-PAGE analysis of humanized anti-CDH7 antibody, 07-0646-h7, before and after treatment with Peptide-N-Glycosidase F (PNGase F).

FIG. 20 shows the results of epitope mapping for CDH17 hAb 07-0663-h7 binding to peptides spanning the ECD of CDH17. CDH17 hAb 07-0663-h7 bound 3 peptides with an overlapping 7 amino acid region, LDANGII. Mutations were introduced into the 7 amino acid core region to further refine the CDH17 hAb 07-0663-h7 binding region.

FIG. 21 shows epitope mapping data for CDH17 hAb 07-0663-h7 indicating a sequence of 5 amino acids, DANGI, is the critical epitope core region for CDH17 hAb 07-0663-h7 binding.

FIG. 22 shows two graphs showing (A) 07-0646-h7 ADCC potency in LS513, SNUC1, and M202 cells, and (B) ADCC potency of multiple CDH17 antibodies in LS513 cells. NFAT activation, which indicates the induced ADCC response, was assessed by determining Lucia luciferase activity in the supernatant. The data show that CDH17 antibodies, in particular 07-646-h7 and 07-663-h7, induce ADCC in CDH17-positive cell lines.

FIG. 23 shows CDH17-CD3 bispecific antibodies and their effect on T-Cell activation. Treatment of CDH17-positive cell lines (HPAF2 and SNUC1) with the 07-0653-h43Bs bispecific antibody resulted in T-cell activation compared to no target cell control. By contrast, there was no difference in the T-cell activation level between CDH17-positive cells and a no-target cell control for the 07-0646-h7Bs and 07-0663-h7Bs bispecific antibodies.

### DETAILED DESCRIPTION

The present disclosure describes an antigen binding protein against CDH17, e.g., specific for CDH17, to treat CDH17-expressing cancers.

### Antigen Binding Proteins

Provided herein are antigen-binding proteins that bind to CDH17. In various embodiments, the antigen binding proteins bind to isoform CDH17. The antigen-binding proteins of the present disclosure can take any one of many forms of antigen-binding proteins known in the art. In various embodiments, the antigen-binding proteins of the present disclosure take the form of an antibody, or antigen-binding antibody fragment, or an antibody protein product.

In various embodiments of the present disclosure, the antigen-binding protein comprises, consists essentially of, or consists of an antibody. As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342:877-883), within a framework region (designated framework regions 1-4, FR1, FR2, FR3, and FR4, by Kabat et al., 1991; see also Chothia and Lesk, 1987, supra). CDRs can be annotated in various ways including the method according to Kabat, AbM, or IMGT. Accordingly, the CDRs of the same antibody can comprise different sequences, depending on which method was used to annotate the CDR sequences. Such is exemplified in the Tables presented herein.

In related embodiments, the residues of the framework are altered. The heavy chain framework regions which can be altered lie within regions designated H-FR1, H-FR2, H-FR3 and H-FR4, which surround the heavy chain CDR residues, and the residues of the light chain framework regions which can be altered lie within the regions designated L-FR1, L-FR2, L-FR3 and L-FR4, which surround the light chain CDR residues. An amino acid within the framework region may be replaced, for example, with any suitable amino acid identified in a human framework or human consensus framework.

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa-or lambda-type light chain constant region, e.g., a human kappa-or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in various embodiments, the antibody is an antibody of isotype IgA, IgD, IgE, IgG, or IgM, including any one of IgG1, IgG2, IgG3 or IgG4. In various aspects, the antibody comprises a constant region comprising one or more amino acid modifications, relative to the naturally-occurring counterpart, in order to improve half-life/stability or to render the antibody more suitable for expression/manufacturability. In various instances, the antibody comprises a constant region wherein the C-terminal Lys residue that is present in the naturally-occurring counterpart is removed or clipped.

The antibody can be a monoclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antigen-binding protein is an antibody, such as a human antibody. In certain aspects, the antigen-binding protein is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence. Information, including sequence information for human antibody heavy and light chain constant regions is publicly available through the Uniprot database as well as other databases well-known to those in the field of antibody engineering and production. For example, the IgG1 constant region is available from the Uniprot database as described below, incorporated herein by reference. Additionally, in another example, the IgG2 constant region is available from the Uniprot database as Uniprot number P01859, incorporated herein by reference.

Merely by way of example, the sequence for a murine immunoglobulin kappa light chain constant region or an immunoglobulin gamma-2A heavy chain constant region includes the following.

| Name: | Sequence: |
|---|---|
| Immunoglobulin | RADAAPTVSIFPPSSEQLTSGGASVVCFLNNFYPKDINVKWKI |
| kappa constant, | DGSERQNGVLNSWTDQDSKDSTYSMSSTLTLTKDEYERHNSY |
| mouse | TCEATHKTSTSPIVKSFNRNEC |
| (IGKC_MOUSE; | |
| UniProt ID: | |
| P01837) | |
| Ig gamma-2A | AKTTAPSVYPLAPVCGDTTGSSVTLGCLVKGYFPEPVTLTWN |
| chain C region, A | SGSLSSGVHTFPAVLQSDLYTLSSSVTVTSSTWPSQSITCNVAH |
| allele, mouse | PASSTKVDKKIEPRGPTIKPCPPCKCPAPNLLGGPSVFIFPPKIK |
| (GCAA_MOUSE; | DVLMISLSPIVTCVVVDVSEDDPDVQISWFVNNVEVHTAQTQ |
| UniProt ID: | THREDYNSTLRVVSALPIQHQDWMSGKEFKCKVNNKDLPAPI |
| P01863) | ERTISKPKGSVRAPQVYVLPPPEEEMTKKQVTLTCMVTDFMP |
| | EDIYVEWTNNGKTELNYKNTEPVLDSDGSYFMYSKLRVEKK |
| | NWVERNSYSCSVVHEGLHNHHTTKSFSRTPGK |

Merely by way of example, the sequences for the human immunoglobulin kappa light chain constant region, human immunoglobulin lambda constant 2 light chain region, human IgG1 heavy chain constant region, and human IgG2 heavy chain constant region include the following.

| Name: | Sequence: |
|---|---|
| Immunoglobulin | RTVAAPSVFIFPPSDEQLKSGTASVVCLLNNFYPRE |
| kappa constant, | AKVQWKVDNALQSGNSQESVTEQDSKDSTYSLSS |
| human | TLTLSKADYEKHKVYACEVTHQGLSSPVTKSFNRGEC |
| (IGKC_HUMAN; | |
| UniProt ID: | |
| 01834) | |
| Immunoglobulin | GQPKAAPSVTLFPPSSEELQANKATLVCLISDFYPGAV |
| lambda constant 2, | TVAWKADSSPVKAGVETTTPSKQSNNKYAASSYLSLT |
| human | PEQWKSHRSYSCQVTHEGSTVEKTVAPTECS |
| (IGLC2_HUMAN; | |
| UniProt ID: | |
| P0DOY2) | |
| Immunoglobulin | ASTKGPSVFPLAPSSKSTSGGTAALGCLVKDYFPEPVTVSWNS |
| heavy constant | GALTSGVHTFPAVLQSSGLYSLSSVVTVPSSSLGTQTYICNVN |
| gamma 1, human | HKPSNTKVDKKVEPKSCDKTHTCPPCPAPELLGGPSVFLFPPK |
| (IGHG1_HUMAN; | PKDTLMISRTPEVTCVVVDVSHEDPEVKFNWYVDGVEVHNA |
| UniProt ID: | KTKPREEQYNSTYRVVSVLTVLHQDWLNGKEYKCKVSNKAL |
| P01857) | PAPIEKTISKAKGQPREPQVYTLPPSRDELTKNQVSLTCLVKGF |
| | YPSDIAVEWESNGQPENNYKTTPPVLDSDGSFFLYSKLTVDKS |
| | RWQQGNVFSCSVMHEALHNHYTQKSLSLSPGK |
| Immunoglobulin | ASTKGPSVFPLAPCSRSTSESTAALGCLVKDYFPEPVTVSWNSG |
| heavy constant | ALTSGVHTFPAVLQSSGLYSLSSVVTVPSSNFGTQTYTCNVDHK |
| gamma 2, human | PSNTKVDKTVERKCCVECPPCPAPPVAGPSVFLFPPKPKDTLMIS |
| (IGHG2_HUMAN; | RTPEVTCVVVDVSHEDPEVQFNWYVDGVEVHNAKTKPREEQF |
| Uniprot ID: | NSTFRVVSVLTVVHQDWLNGKEYKCKVSNKGLPAPIEKTISKT |
| P01859) | KGQPREPQVYTLPPSREEMTKNQVSLTCLVKGFYPSDISVEWESNGQ |
| | PENNYKTTPPMLDSDGSFFLYSKLTVDKSRWQQGNVFSCSVMHEAL |
| | HNHYTQKSLSLSPGK |

In some embodiments, in identifying the light chain variable region (VL sequence) and light chain framework 4 (LFR4) by the Kabat and AbM methods, the VL sequence and LFR4 may be identified as ending with a tripeptide, RTV, at their C-terminus (for example, see amino acid sequences presented for 07-0646-h7 (Tables 2B and 2C), 07-0653-h43 (Tables 3B and 3C), and 07-0663-h7 (Tables 4B and 4C)). In cases where the VL sequence and LFR4 end with a RTV tripeptide, the light chain constant region that joins the VL sequence to make a complete immunoglobulin kappa light chain (without its signal sequence) is an immunoglobulin kappa light chain constant region with the sequence as given in UniProt ID: P01834 but lacking RTV at the amino terminus. This avoids duplicating the RTV tripeptide sequence in the fully reassembled immunoglobulin kappa light chain. In some embodiments, an alternate human immunoglobulin kappa constant region light chain comprises the amino acid sequence of:

AAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKS
FNRGEC.

In other embodiments, where the amino acid sequences of the immunoglobulin light chain variable region region (VL sequence) and light chain framework 4 (LFR4) were defined using IMGT method, these sequences do not end with the RTV tripeptide sequence but rather end with a sequence, LEIK, at their C-terminus (for example, see amino acid sequences presented for 07-0646-h7 (Table 2A), 07-0653-h43 (Table 3A), and 07-0663-h7 (Table 4A)). In some such embodiments, the immunoglobulin kappa light chain constant region that joins the VL to produce a complete human kappa light chain (without the signal sequence) is the amino acid sequence as provided by UniProt ID: P01834 in its entirety.

In some embodiments, nucleic acid sequences encoding the complete immunoglobulin kappa light chain (without its signal sequence) avoids duplication of the RTV tripeptide sequence found at the border of the light chain variable region (as in Tables 2B, 2C, 3B, 3C, 4B and 4C) and constant region (UniProt ID: P01834).

An antibody can be cleaved into fragments by enzymes, such as, e.g., papain and pepsin. Papain cleaves an antibody to produce two Fab fragments and a single Fc fragment. Pepsin cleaves an antibody to produce a F(ab′) 2 fragment and a pFc′ fragment. In various aspects of the present disclosure, the antigen-binding protein of the present disclosure is an antigen-binding fragment of an antibody (a.k.a., antigen-binding antibody fragment, antigen-binding fragment, antigen-binding portion). In various instances, the antigen-binding antibody fragment is a Fab fragment or a F(ab′) 2 fragment.

The architecture of antibodies has been exploited to create a growing range of alternative antibody formats that spans a molecular-weight range of at least about 12-150 kDa and has a valency (n) range from monomeric (n=1), to dimeric (n=2), to trimeric (n=3), to tetrameric (n=4), and potentially higher; such alternative antibody formats are referred to herein as “antibody protein products”. Antibody protein products include those based on the full antibody structure and those that mimic antibody fragments which retain full antigen-binding capacity, e.g., scFvs, Fabs and VHH/VH (discussed below). The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. In some embodiments, the scFv can be an anti-CDH17 scFv, comprising the light chain and heavy chain variable regions of any of the antibodies presently described herein. In some embodiments, the scFv can be an anti-CDH17 scFv comprising the light chain and heavy chain variable regions of the 07-0646-h7, 07-0653-h43, or 07-0663-h7 anti-CDH17 antibody. In some embodiments, the scFv comprises the sequence of 07-0646-7scFv (SEQ ID NO: 96), 07-0653-h43scfv (SEQ ID NO: 97), or 07-0663-h7scfv (SEQ ID NO: 98). In some embodiments, the 07-0646-7scFv (SEQ ID NO: 96), 07-0653-h43scfv (SEQ ID NO: 97), or 07-0663-h7scfv (SEQ ID NO: 98) scFv can be encoded by a nucleic acid sequence of SEQ ID NO: 102, 103, or 104, respectively. In some embodiments, any antigen-binding protein of the present disclosure may further comprise a detectable marker that may facilitate detection and/or purification of the antigen-binding protein. Accordingly, in some embodiments, the 07-0646-7scFv (SEQ ID NO: 96), 07-0653-h43scfv (SEQ ID NO: 97), or 07-0663-h7scfv (SEQ ID NO: 98) scFv may further comprise a detectable marker. In some such embodiments, the detectable marker may be an epitope tag. As used herein, in some embodiments, the epitope tag can be a C-terminal (HIS) 6 epitope tag. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4 (5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, bispecific antibody (BsAb) fragments, bispecific fusion proteins, and BsAb conjugates. See, e.g., Spiess et al., Molecular Immunology 67 (2) Part A: 97-106 (2015).

In various aspects, the antigen-binding protein of the present disclosure comprises, consists essentially of, or consists of any one of these antibody protein products. In various aspects, the antigen-binding protein of the present disclosure comprises, consists essentially of, or consists of any one of an scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody, BsIgG, appended IgG, BsAb fragment, bispecific fusion protein, and BsAb conjugate.

In various instances, the antigen-binding protein of the present disclosure is an antibody protein product in monomeric form, or polymeric, oligomeric, or multimeric form. In certain embodiments in which the antibody comprises two or more distinct antigen binding regions fragments, the antibody is considered bispecific, trispecific, or multi-specific, or bivalent, trivalent, or multivalent, depending on the number of distinct epitopes that are recognized and bound by the antibody.

In various embodiments, an anti-CDH17 antibody or antibody variant thereof is selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody.

In various aspects, the antigen-binding protein of the present disclosure is linked to a therapeutic agent. As described below, the therapeutic agent may be any known in the art, including, but not limited to, chemotherapeutic agents, cytokines and growth factors, cytotoxic agents, and the like. See “Conjugates” below.

In various aspects, any polypeptide of the present disclosure, e.g., an antigen-binding protein, may further comprise a heterologous peptide or polypeptide. In some embodiments, the heterologous peptide or polypeptide may be a detectable marker or a tag that can be detected directly (e.g., GFP) or indirectly (e.g., using a secondary antibody that binds to the tag). Examples of detectable markers include various enzymes, prosthetic groups, and tags (e.g., a histidine tag, myc tag, flag tag, etc.). Examples of suitable enzymes include horseradish peroxidase, alkaline phosphatase, β-galactosidase, or acetylcholinesterase. Examples of suitable prosthetic group complexes include streptavidin/biotin and avidin/biotin. Examples of bioluminescent materials include luciferase, luciferin, fluorescent protein (e.g., GFP, RFP, etc.), and aequorin.

### CDH17 and Epitopes

The antigen-binding proteins of the present disclosure bind to CDH17. In some embodiments, the CDH17 is a human CDH17 comprising the amino acid sequence of:

(SEQ ID NO: 19)
MILQAHLHSLCLLMLYLATGYGQEGKFSGPLKPMTFSIYEGQEPSQIIF
QFKANPPAVTFELTGETDNIFVIEREGLLYYNRALDRETRSTHNLQVAA
LDANGIIVEGPVPITIKVKDINDNRPTFLQSKYEGSVRQNSRPGKPFLY
VNATDLDDPATPNGQLYYQIVIQLPMINNVMYFQINNKTGAISLTREGS
QELNPAKNPSYNLVISVKDMGGQSENSFSDTTSVDIIVTENIWKAPKPV
EMVENSTDPHPIKITQVRWNDPGAQYSLVDKEKLPRFPFSIDQEGDIYV
TQPLDREEKDAYVFYAVAKDEYGKPLSYPLEIHVKVKDINDNPPTCPSP
VTVFEVQENERLGNSIGTLTAHDRDEENTANSFLNYRIVEQTPKLPMDG
LFLIQTYAGMLQLAKQSLKKQDTPQYNLTIEVSDKDFKTLCFVQINVID
INDQIPIFEKSDYGNLTLAEDTNIGSTILTIQATDADEPFTGSSKILYH
IIKGDSEGRLGVDTDPHTNTGYVIIKKPLDFETAAVSNIVFKAENPEPL
VFGVKYNASSFAKFTLIVTDVNEAPQFSQHVFQAKVSEDVAIGTKVGNV
TAKDPEGLDISYSLRGDTRGWLKIDHVTGEIFSVAPLDREAGSPYRVQV
VATEVGGSSLSSVSEFHLILMDVNDNPPRLAKDYTGLFFCHPLSAPGSL
IFEATDDDQHLFRGPHFTFSLGSGSLQNDWEVSKINGTHARLSTRHTEF
EEREYVVLIRINDGGRPPLEGIVSLPVTFCSCVEGSCFRPAGHQTGIPT
VGMAVGILLTTLLVIGIILAVVFIRIKKDKGKDNVESAQASEVKPLRSI.

In various aspects, the antigen-binding proteins of the present disclosure bind to an epitope within an amino acid sequence of CDH17. In various aspects, CDH17 is a human CDH17 and the antigen-binding proteins of the present disclosure bind to an epitope within an amino acid sequence of human CDH17, e.g., SEQ ID NO: 19. By “epitope” is meant the region of or within CDH17 which is bound by the antigen-binding protein. In some embodiments, the epitope is a linear epitope. “Linear epitope” refers to the region of or within the CDH17 which is bound by the antigen-binding protein and which region is composed of contiguous amino acids of the amino acid sequence of the CDH17. The amino acids of a linear epitope are adjacent to each other in the primary structure of the CDH17. Accordingly, a linear epitope is a fragment or portion of the amino acid sequence of the antigen, i.e., CDH17. In other various embodiments, the epitope is a conformational or structural epitope. By “conformational epitope” or “structural epitope” is meant an epitope which is composed of amino acids which are located in close proximity to one another only when the CDH17 is in its properly folded state. Unlike linear epitopes, the amino acids of a conformational or structural epitope are not adjacent to each other in the primary structure (i.e., amino acid sequence) of the CDH17. A conformational or structural epitope is not made of contiguous amino acids of the amino acid sequence of the antigen (CDH17).

In various aspects, the epitope is located within the extracellular domain (ECD) of CDH17, e.g., human CDH17. In various aspects, the epitope to which the antigen-binding protein binds is within SEQ ID NO: 51.

In various aspects, the antigen-binding proteins bind to human CDH17 and/or a non-human CDH17. In various instances, the non-human CDH17 is a CDH17 of chimpanzee, Rhesus monkey, dog, cow, mouse, rat, zebrafish, or frog. In various instances, the antigen-binding proteins bind to human CDH17 and/or mouse CDH17.

### Afucosylated Antibodies

Many secreted proteins undergo post-translational glycosylation, a process by which sugar moieties (e.g., glycans, saccharides) are covalently attached to specific amino acids of a protein. In eukaryotic cells, two types of glycosylation reactions occur: (1)N-linked glycosylation, in which glycans are attached to the asparagine of the recognition sequence Asn-X-Thr/Ser, where “X” is any amino acid except proline, and (2)O-linked glycosylation in which glycans are attached to serine or threonine. Regardless of the glycosylation type (N-linked or O-linked), microheterogeneity of protein glycoforms exists due to the large range of glycan structures associated with each site (O or N).

All N-glycans have a common core sugar sequence: Manα1-6 (Manα1-3) Manβ1-4GlcNAcβ1-4GlcNAcβ1-Asn-X-Ser/Thr (Man₃GlcNAc₂Asn) and are categorized into one of three types: (A) a high mannose (HM) or oligomannose (OM) type, which consists of two N-acetylglucosamine (GalNAc) moieties and a large number (e.g., 5, 6, 7, 8 or 9) of mannose (Man) residues (B) a complex type, which comprises more than two GlcNAc moieties and any number of other sugar types or (C) a hybrid type, which comprises a Man residue on one side of the branch and GlcNAc at the base of a complex branch.

N-linked glycans typically comprise one or more monosaccharides of galactose (Gal), N-acetylgalactosamine (GalNAc), galactosamine (GalN), glucose (GLc), N-acetylglucosamine (ClcNAc), glucoasamine (GlcN), mannose (Man), N-Acetylmannosamine (ManNAc), Mannosamine (ManN), xylose (Xyl), NOAcetylneuraminic acid (Neu5 Ac), N-Glycolylneuraminic acid (Neu5Gc), 2-keto-3-doxynononic acid (Kdn), fucose (Fuc), Glucuronic acid (GLcA), Iduronic acid (IdoA), Galacturonic acid (Gal A), mannuronic acid (Man A).

N-linked glycosylation begins in the endoplasmic reticulum (ER), where a complex set of reactions result in the attachment of a core glycan structure made essentially of two GlcNAc residues and three Man residues. The glycan complex formed in the ER is modified by action of enzymes in the Golgi apparatus. If the saccharide is relatively inaccessible to the enzymes, it typically stays in the original HM form. If enzymes can access the saccharide, then many of the Man residues are cleaved off and the saccharide is further modified, resulting in the complex type N-glycans structure. For example, mannosidase-1 located in the cis-Golgi, can cleave or hydrolyze a HM glycan, while fucosyltransferase FUT-8, located in the medial-Golgi, fucosylates the glycan (Hanrue Imai-Nishiya (2007), BMC Biotechnology, 7:84).

Accordingly, the sugar composition and the structural configuration of a glycan structure varies, depending on the glycosylation machinery in the ER and the Golgi apparatus, the accessibility of the machinery enzymes to the glycan structure, the order of action of each enzyme and the stage at which the protein is released from the glycosylation machinery, among other factors.

In exemplary embodiments of the present disclosure, the antigen-binding proteins comprise an Fc polypeptide. The term “Fc polypeptide” as used herein includes native and mutein forms of polypeptides derived from the Fc region of an antibody. In exemplary aspects, the Fc polypeptide of the presently disclosed antigen-binding protein comprises a glycan. In various instances, the glycan lacks fucose or is afucosylated. In exemplary aspects, the antigen-binding protein comprises an afucosylated glycan. As used herein, the term “afucosylated glycan” or “afuco glycan” or “afucosylated glycoform” or “Afuc” refers to glycoforms which lack a core fucose, e.g., an α1,6-linked fucose on the GlcNAc residue involved in the amide bond with the Asn of the N-glycosylation site. Afucosylated glycoforms include, but are not limited to, A1GO, A2GO, A2Gla, A2Gb, A2G2, and A1G1M5. Additional afucosylated glycans include, e.g., A1Gla, GO [H3N4], GO [H4N4], GO [H5N4], FO-N [H3N3]. See, e.g., Reusch and Tejada, Glycobiology 25 (12): 1325-1334 (2015).

The present disclosure also provides a composition, e.g., a pharmaceutical composition, comprising an antigen binding protein comprising an Fc polypeptide comprising an afucosylated glycan. In exemplary aspects, at least or about 25% of the antigen-binding proteins present in the composition are antigen-binding proteins comprising an Fc polypeptide comprising an afucosylated glycan. In exemplary aspects, at least or about 25% of the antigen-binding proteins present in the composition are afucosylated. Optionally, at least 30%, 40%, 50%, 60%, 70%, 80%, or 90% or more of the antigen-binding proteins present in the composition are afucosylated. Methods of producing compositions comprising antigen-binding proteins of a particular glycoprofile are known in the art. In exemplary embodiments, the antigen binding proteins are recombinant produced in cells that are genetically modified to alter the activity of an enzyme of the de novo pathway or the salvage pathway. These two pathways of fucose metabolism are shown in FIG. 29B. In exemplary embodiments, the cells are genetically modified to alter the activity of any one or more of: a fucosyl-transferase (FUT, e.g., FUT1, FUT2, FUT3, FUT4, FUT5, FUT6, FUT7, FUT8, FUT9), a fucose kinase, a GDP-fucose pyrophosphorylase, GDP-D-mannose-4,6-dehydratase (GMD), and GDP-keto-6-deoxymannose-3,5-epimerase, 4-reductase (FX). In exemplary embodiments, the cells are genetically modified to knock-out a gene encoding FX. See, e.g., International Patent Publication No. WO2017/079165 A1; Kanda et al., J Biotechnol 130, 2007, 300-310, Yamane-Ohunuki et al., Biotechnol Bioeng 87, 2004, 614-622, Malphettes et al., Biotechnol Bioeng 106, 2010, 774-783.

### Bispecific Formats

In exemplary aspects, the antigen-binding protein is bispecific and thus capable of binding two different and distinct antigens. In exemplary embodiments, the antigen binding protein is bispecific and binds to CDH17 and a second antigen.

In exemplary instances, the second antigen is a cell surface protein expressed by a T-cell. In exemplary aspects, the cell surface protein is a component of the T-cell receptor (TCR), for example, CD3. In exemplary instances, the second antigen is a costimulatory molecule which assists in T-cell activation, e.g., CD40 or 4-1BB (CD137). In exemplary aspects, the second antigen is an Fc receptor. In various aspects, the Fc receptor is a Fc gamma receptor, Fc-alpha receptor, Fc-epsilon receptor. In exemplary aspects, the Fc receptor is CD64 (Fc-gamma RI), CD32 (Fc-gamma RIIA), CD16A (Fc-gamma RIIIA), CD16b (Fc-gamma RIIIb), FcERI, CD23 (Fc-epsilon RII), CD89 (Fc-epsilon RI), Fcα/μR, or FcRn. In exemplary aspects, the Fc receptor is CD16A. In exemplary instances, the second antigen is an immune checkpoint molecule, e.g., a protein involved in the immune checkpoint pathway. The immune checkpoint pathway and molecules or proteins that function in it are known in the art. See, e.g., Pardoll, Nat Rev Genet 12 (4): 252-264 (2012). In exemplary instances, the immune checkpoint molecule is A2AR, B7-H3, B7-H4, BTLA, CTLA4, IDO, KIR, LAG3, NOX2, PD-1, TIM3, VISTA, or SIGLEC7. Optionally, the immune checkpoint molecule is PD-1, LAG3, TIM3, or CTLA4.

Over fifty formats of bispecific antigen-binding proteins are known in the art, some of which are described in Kontermann and Brinkmann, Drug Discovery Today 20 (7): 838-847 (2015); Zhang et al., Exp Hematol Oncol 6:12 (2017); Spiess et al., Mol Immunol.; 67 (2 Pt A): 95-106 (2015). In exemplary aspects, the bispecific antigen-binding protein of the present disclosure is made through chemical engineering, genetic engineering, or quadroma technology.

In exemplary aspects, the bispecific antigen-binding protein is constructed with some or all of the constant domains of an antibody. In exemplary aspects, the bispecific antigen-binding protein of the present disclosure comprises an Fc polypeptide and retains Fc-mediated effector functions. In various instances, the bispecific antigen-binding protein is a bispecific monoclonal antibody formed by, e.g., chemical cross-linking of two monoclonal antibodies (mabs), or by knob and hold technology. In exemplary aspects, the bispecific antigen-binding protein is made through “knobs-into-holes” technology in which H chain heterodimerization is forced by introducing different mutations into the two CH3 domains resulting in asymmetric antibodies. A “knob” mutation is made into one HC and a “hole” mutation is created in the other HC to promote heterodimerization. In exemplary aspects, the bispecific antigen-binding protein is a bispecifc antibody produced by quadroma technology which is based on the somatic fusion of two different hybridoma cells producing monoclonal antibodies with the desired specificity. Zhang et al., 2017, supra. In exemplary aspects, the bispecific antigen-binding protein is a crossMab, ortho-Fab IgG, DVD-Ig, two in one IgG, IgG-scFv and scFv2-Fc (Kontermann and Brinkmann, 2015, supra. In various aspects, the bispecific antigen-binding protein is an Ig-scFv fusion wherein a new antigen-binding moiety is added to a full length IgG resulting in a fusion protein with tetravalency for two distinct antigens, e.g., IgG C-terminal scFv fusion and IgG N-terminal scFv fusion. In exemplary instances, the bispecific antigen-binding protein is a dual-variable-domain-IgG (DVD-IgG), wherein the LC and HC variable regions of an IgG specific for one antigen are fused to the N-terminal LC and HC variable regions of an IgG specific for a second antigen through a linker to form a DVD-IgG. In exemplary aspects, the bispecific antigen-binding protein is a diabody-Fc fusion which involves the replacement of a Fab fragment of an IgG with a bispecific diabody

In alternative instances, the bispecific antigen-binding protein of the present disclosure does not comprise an Fc polypeptide. In exemplary aspects, the bispecific antigen-binding protein comprises the variable domains of each parental monoclonal antibody, and linkers are cloned and linked to form a single-chain bispecific antibody. In exemplary aspects, the bispecific antigen-binding protein is a tandem scFvs, diabody format, single-chain diabodies, tandem diabodies (TandAbs), dual-affinity retargeting molecules (DARTs), dock-and-lock (DNL), and nanobodies (Fan et al., J Hematol Oncol. 2015; 8:130). In various aspects, the bispecific antigen-binding protein is a bispecific F (mab1) 2, an scFv, a bispecific diabody (BsDb), single-chain bispecific diabody (scBsDb), single-chain bispecific tandem variable domain (scBsTaFv), dock-and-lock trivalent Fab (DNL-(Fab) 3), single-domain antibody (sdAb), or a bispecific single-domain antibody (BssdAb). In exemplary aspects, the bispecific antigen-binding protein is a tandem scFv comprising two scFv fragments linked by an extra peptide linker such as glycine-serine repeat motifs. Optionally, the tandem scFv comprises the structure: VLA-linker1-VHA-linker2-VHB-linker3-VLB (VL and VH derive from the single chain antibody fragment; A and B represent the parental monoclonal antibody A and B). In exemplary aspects, the bispecific antigen-binding protein is a TandAb which contains two pairs of VL and VH domains connected in a single polypeptide chain (Reusch et al., MAbs. 2015; 7 (3): 584-604). Two polypeptide products dimerize in a head-to-tail fashion, forming homodimers with large molecular weight (˜105 kDa) upon expression. In exemplary aspects, the bispecific antigen-binding protein is one produced using crossMab technology which is described in PNAS 108 (27): 11187-92 (2011). CrossMabs do not have any chemical linkers or connectors and are produced by a method that enforces correct light chain association in bispecific heterodimeric IgG antibodies. In exemplary aspects, the CrossMab is a bi-(1+1), tri-(2+1) and tetra-(2+2) valent bispecific crossMab, or is a non-Fc tandem antigen-binding fragment (Fab)-based crossMab. In exemplary instances, the crossMab is a crossMab^{Fab}, a crossMab^{VH-VL}, or a crossMab^{CHI-CL}.

In exemplary aspects, the bispecific antigen-binding protein comprises a single-domain antibody, or a nanobody, comprising a single monomeric variable antibody domain. Optionally, the variable domain is based on the heavy chain variable domain. In alternative aspects, the variable domain is based on the light chain variable domain.

In exemplary aspects, the bispecific antigen-binding protein is a bispecific T cell engager or BiTE®. BiTEs are bivalent small molecules comprising only the variable regions of antibodies in the form of scFvs which are connected by flexible peptidic linkers. In exemplary aspects, the bispecific antigen-binding protein comprises an scFV comprising the LC and HC variable regions of the presently disclosed CDH17 antibodies and the LC and HC variable regions of a second antibody specific for a second antigen. In some embodiments, the BiTE comprises the LC and HC variable region of a second antibody specific for CD3. In some embodiments, the CD3 is CD3E. In some embodiments, the BiTE is 07-0653-h43Bs, 07-0646-h7Bs, or 07-0663-h7Bs. In some embodiments, the 07-0653-h43Bs, 07-0646-h7Bs, and 07-0663-h7Bs comprise the amino acid as provided in SEQ ID NOs: 100, 99, and 101, respectively. In some embodiment, the 07-0653-h43Bs, 07-0646-h7Bs, and 07-0663-h7Bs may be encoded by exemplary nucleic acid molecules comprising the sequence as provided in SEQ ID NOs: 106, 105 and 107, respectively. In some embodiments, the BiTE may further comprise a detectable marker that may facilitate detection and/or purification of the BiTE. In some such embodiments, the BiTE further comprises a C-terminal (HIS) 6 epitope tag. In exemplary instances, the bispecific antigen-binding protein is a dual affinity retargeting (DART), which unlike BiTEs®, the covalent linkage between the two chains of DARTs limits the freedom of the antigen-binding sites. Therefore, DARTs are structurally compact and can form stable contacts between target and effector cells. The DART comprises two engineered Fv fragments which have their own VH exchanged with the VH of the other one. The inter-exchanged Fv domains advantageously releases variant fragments from the conformational constraint by the short linking peptide.

In exemplary aspects, the bispecific antigen binding protein is an HSABody comprising two scFvs fused to modified HSA. HSABodies are described in McDonagh et al., Mol Cancer Ther. 2012; 11 (3): 582-93.

Accordingly, in exemplary aspects, the bispecific antigen-binding protein comprises an antigen binding fragment of any of the presently disclosed CDH17 antibodies. In exemplary aspects, the antigen binding fragment is a Fab. In exemplary aspects, the bispecific antigen-binding protein comprises an F (ab) 2′ of any of the presently disclosed CDH17 antibodies. In exemplary aspects, the bispecific antigen-binding protein comprises an scFv comprising the LC and HC variable regions of any of the presently disclosed CDH17 antibodies. In various aspects, the antigen binding fragment is based on the heavy chain variable region and in other aspects, the antigen binding fragment is based on the light chain variable region. In exemplary aspects, the antigen binding fragment comprises at least part of both HC variable region and LC variable region. In exemplary aspects, the bispecific antigen-binding protein comprises at least one if not both of the LC or HC variable regions of the presently disclosed CDH17 antibodies and at least one if not both of the LC and HC variable regions of a second antibody specific for a second antigen. In exemplary instances, the bispecific antigen-binding protein comprises an scFV comprising the LC and HC variable regions of the presently disclosed CDH17 antibodies and the LC and HC variable regions of a second antibody specific for a second antigen.

### Nucleic Acids

The present disclosure further provides nucleic acids comprising a nucleotide sequence encoding an antigen-binding protein of the present disclosure. By “nucleic acid” as used herein includes “polynucleotide,” “oligonucleotide,” and “nucleic acid molecule,” and generally means a polymer of DNA or RNA, or modified forms thereof, which can be single-stranded or double-stranded, synthesized or obtained (e.g., isolated and/or purified) from natural sources, which can contain natural, non-natural or altered nucleotides, and which can contain a natural, non-natural or altered inter-nucleotide linkage, such as a phosphoroamidate linkage or a phosphorothioate linkage, instead of the phosphodiester found between the nucleotides of an unmodified oligonucleotide. The nucleic acid can comprise any nucleotide sequence which encodes any of the antigen-binding proteins of the present disclosure.

The invention further provides nucleic acid molecules encoding the amino acid sequence corresponding to the antigen-binding proteins of the invention. In some embodiments, the nucleic acid molecule is a DNA (e.g., cDNA) or a hybrid thereof. Alternatively, the molecules is RNA or a hybrid thereof.

In some aspects, the nucleic acids of the present disclosure are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

Any nucleic acid of the present disclosure may be codon-optimized. Codon usage within a gene is helpful in determining the achievable protein expression levels. Certain sequences can be translated more readily by certain hosts, thus selecting the right codons for a given host may be necessary to maximizing expression. Methods of optimizing the codons are well known in the art. For example, there are many online tools, including World Wide Web at codonstatsdb.unr.edu (see Subramanian et al. (2022) Mol Biol Evol 3;39 (8): msac 157.

The nucleic acids in some aspects are constructed based on chemical synthesis and/or enzymatic ligation reactions using procedures known in the art. See, for example, Sambrook et al., supra; and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridme, 5-carboxymethylaminomethyluracil, dihydrouracil, beta-D-galactosylqueosine, inosine, N°-isopentenyladenine, 1-methylguanine, 1-methylinosine, 2,2-dimethylguanine, 2-methyladenine, 2-methylguanine, 3-methylcytosine, 5-methylcytosine, N-substituted adenine, 7-methylguanine, 5-methylammomethyluracil, 5-methoxyaminomethyl-2-thiouracil, beta-D-mannosylqueosine, 5′-methoxycarboxymethyluracil, 5-methoxyuracil, 2-methylthio-N°-isopentenyladenine, uracil-5-oxyacetic acid (v), wybutoxosine, pseudouratil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the present disclosure can be purchased from companies, such as Macromolecular Resources (Fort Collins, CO) and Synthegen (Houston, TX).

### Vector

The nucleic acids of the present disclosure in some aspects are incorporated into a vector. In this regard, the present disclosure provides vectors comprising any of the presently disclosed nucleic acids. In various aspects, the vector is a recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the present disclosure are not naturally-occurring as a whole. However, parts of the vectors can be naturally-occurring. The presently disclosed vectors can comprise any type of nucleotides, including, but not limited to DNA and RNA, which can be single-stranded or double-stranded, synthesized or obtained in part from natural sources, and which can contain natural, non-natural or altered nucleotides. The vectors can comprise naturally-occurring or non-naturally-occurring internucleotide linkages, or both types of linkages. In some aspects, the altered nucleotides or non-naturally occurring internucleotide linkages do not hinder the transcription or replication of the vector.

The vector of the present disclosure can be any suitable vector, and can be used to transduce, transform or transfect any suitable host. Suitable vectors include those designed for propagation and expansion or for expression or both, such as plasmids and viruses. The vector can be a plasmid based expression vector. In various aspects, the vector is selected from the group consisting of the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGTIO, λGTI 1, λZapII (Stratagene), λEMBL4, and λNMI 149, also can be used. Examples of plant expression vectors include pBIOI, pBI101.2, pBI101.3, pBI121 and pBIN19 (Clontech). Examples of animal expression vectors include pEUK-Cl, pMAM and pMAMneo (Clontech). In some aspects, the vector is a viral vector, e.g., a retroviral vector. In various aspects, the vector is an adenovirus vector, an adeno-associated virus (AAV) vector, a Herpes Simplex Virus (HSV) vector, a Vesicular stomatitis virus (VSV) vector, vaccinia virus vector, or lentivirus vector. See, e.g., Howarth et al., Cell Biol. Toxicol. 26 (1): 1-20 (2010). In various aspects, the vector is a baculovirus vector which infects arthropods, e.g., insects. In various aspects, the baculovirus vector is an Autographacalifornica multiple nuclear virus (AcMNPV) or a Bombyxmorinuclear polyhedrosis (BmNPV). See, e.g., Khan, Adv Pharm Bull 3 (2): 257-263 (2013); Miller, Bioessays 11 (4): 91-96 (1989); Atkinson et al., Pestic Sci 28:215-224 (1990).

The vectors of the present disclosure can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from CoIEl, 2 μ plasmid, λ, SV40, bovine papilloma virus, and the like.

In some aspects, the vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA-or RNA-based.

The vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the presently disclosed expression vectors include, for instance, neomycin/G418 resistance genes, hygromycin resistance genes, histidinol resistance genes, tetracycline resistance genes, and ampicillin resistance genes.

The vector can comprise a native or normative promoter operably linked to the nucleotide sequence encoding the polypeptide (including functional portions and functional variants thereof), or to the nucleotide sequence which is complementary to or which hybridizes to the nucleotide sequence encoding the polypeptide. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

### Host Cells

Provided herein are host cells comprising a nucleic acid or vector of the present disclosure. As used herein, the term “host cell” refers to any type of cell that can contain the presently disclosed vector and is capable of producing an expression product encoded by the nucleic acid (e.g., mRNA, protein). The host cell in some aspects is an adherent cell or a suspended cell, i.e., a cell that grows in suspension. The host cell in various aspects is a cultured cell or a primary cell, i.e., isolated directly from an organism, e.g., a human. The host cell can be of any cell type, can originate from any type of tissue, and can be of any developmental stage.

In various aspects, the antigen-binding protein is a glycosylated protein and the host cell is a glycosylation-competent cell. In various aspects, the glycosylation-competent cell is an eukaryotic cell, including, but not limited to, a yeast cell, filamentous fungi cell, protozoa cell, algae cell, insect cell, or mammalian cell. Such host cells are described in the art. See, e.g., Frenzel, et al., Front Immunol 4:217 (2013). In various aspects, the eukaryotic cells are mammalian cells. In various aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NS0, GS-NSO, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinoma human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3 (2): 257-263 (2013)).

For purposes of amplifying or replicating the vector, the host cell is in some aspects is a prokaryotic cell, e.g., a bacterial cell.

Also provided by the present disclosure is a population of cells comprising at least one host cell described herein. The population of cells in some aspects is a heterogeneous population comprising the host cell comprising vectors described, in addition to at least one other cell, which does not comprise any of the vectors. Alternatively, in some aspects, the population of cells is a substantially homogeneous population, in which the population comprises mainly host cells (e.g., consisting essentially of) comprising the vector. The population in some aspects is a clonal population of cells, in which all cells of the population are clones of a single host cell comprising a vector, such that all cells of the population comprise the vector. In various embodiments of the present disclosure, the population of cells is a clonal population comprising host cells comprising a vector as described herein.

### Manufacture Methods

Also provided herein are methods of producing an antigen-binding protein which binds to CDH17. In various embodiments, the method comprises culturing a host cell comprising a nucleic acid comprising a nucleotide sequence encoding the antigen-binding protein as described herein in a cell culture medium and harvesting the antigen-binding protein from the cell culture medium. The host cell can be any of the host cells described herein. In various aspects, the host cell is selected from the group consisting of: CHO cells, NSO cells, COS cells, VERO cells, and BHK cells. In various aspects, the step of culturing a host cell comprises culturing the host cell in a growth medium to support the growth and expansion of the host cell. In various aspects, the growth medium increases cell density, culture viability and productivity in a timely manner. In various aspects, the growth medium comprises amino acids, vitamins, inorganic salts, glucose, and serum as a source of growth factors, hormones, and attachment factors. In various aspects, the growth medium is a fully chemically defined media consisting of amino acids, vitamins, trace elements, inorganic salts, lipids and insulin or insulin-like growth factors. In addition to nutrients, the growth medium also helps maintain pH and osmolality. Several growth media are commercially available and are described in the art. See, e.g., Arora, “Cell Culture Media: A Review” MATER METHODS 3:175 (2013).

In various aspects, the method comprises culturing the host cell in a feed medium. In various aspects, the method comprises culturing in a feed medium in a fed-batch mode. Methods of recombinant protein production are known in the art. See, e.g., Li et al., “Cell culture processes for monoclonal antibody production” MAbs 2 (5): 466-477 (2010).

The method making an antigen-binding protein can comprise one or more steps for purifying the protein from a cell culture or the supernatant thereof and preferably recovering the purified protein. In various aspects, the method comprises one or more chromatography steps, e.g., affinity chromatography (e.g., protein A affinity chromatography), ion exchange chromatography, hydrophobic interaction chromatography. In various aspects, the method comprises purifying the protein using a Protein A affinity chromatography resin.

In various embodiments, the method further comprises steps for formulating the purified protein, etc., thereby obtaining a formulation comprising the purified protein. Such steps are described in Formulation and Process Development Strategies for Manufacturing, eds. Jameel and Hershenson, John Wiley & Sons, Inc. (Hoboken, NJ), 2010.

In various aspects, the antigen-binding protein linked to a polypeptide and the antigen-binding protein is part of a fusion protein. Thus, the present disclosure further provides methods of producing a fusion protein comprising an antigen-binding protein which binds to CDH17. In various embodiments, the method comprises culturing a host cell comprising a nucleic acid comprising a nucleotide sequence encoding the fusion protein as described herein in a cell culture medium and harvesting the fusion protein from the cell culture medium.

### Conjugates

The present disclosure also provides antigen-binding proteins attached, linked or conjugated to a second moiety (e.g., a heterologous moiety, a conjugate moiety). Accordingly, the present disclosure provides a conjugate comprising an antigen-binding protein and a heterologous moiety. As used herein, the term “heterologous moiety” is synonymous with “conjugate moiety” and refers to any molecule (chemical or biochemical, naturally-occurring or non-coded) which is different from the antigen-binding proteins of the present disclosure. Various heterologous moieties include, but are not limited to, a polymer, a carbohydrate, a lipid, a nucleic acid, an oligonucleotide, a DNA or RNA, an amino acid, peptide, polypeptide, protein, therapeutic agent, (e.g., a cytotoxic agent, cytokine), or a diagnostic agent.

In some embodiments, the heterologous moiety is a detectable marker. Examples of detectable markers include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include 1251, 1311, 35S, or 3H. As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or indocyanine (Cy5)) to the antibody, as well as indirect labeling of the antibody by reactivity with a detectable substance. For example, an antibody may be labeled with a nucleic acid sequence that may be amplified and detected, or an antisense oligonucleotide to reduce expression of a particular gene, such that expression can then be detected and measured.

In some embodiments, the heterologous moiety is a polymer. The polymer can be branched or unbranched. The polymer can be of any molecular weight. The polymer in some embodiments has an average molecular weight of between about 2 kDa to about 100 kDa (the term “about” indicating that in preparations of a water soluble polymer, some molecules will weigh more, some less, than the stated molecular weight). The average molecular weight of the polymer is in some aspect between about 5 kDa and about 50 kDa, between about 12 kDa to about 40 kDa or between about 20 kDa to about 35 kDa.

In some embodiments, the polymer is modified to have a single reactive group, such as an active ester for acylation or an aldehyde for alkylation, so that the degree of polymerization can be controlled. The polymer in some embodiments is water soluble so that the protein to which it is attached does not precipitate in an aqueous environment, such as a physiological environment. In some embodiments, when, for example, the composition is used for therapeutic use, the polymer is pharmaceutically acceptable. Additionally, in some aspects, the polymer is a mixture of polymers, e.g., a co-polymer, a block co-polymer.

In some embodiments, the polymer is selected from the group consisting of: polyamides, polycarbonates, polyalkylenes and derivatives thereof including, polyalkylene glycols, polyalkylene oxides, polyalkylene terepthalates, polymers of acrylic and methacrylic esters, including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), polyvinyl polymers including polyvinyl alcohols, polyvinyl ethers, polyvinyl esters, polyvinyl halides, poly(vinyl acetate), and polyvinylpyrrolidone, polyglycolides, polysiloxanes, polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, polypropylene, polyethylenes including poly(ethylene glycol), poly(ethylene oxide), and poly(ethylene terephthalate), and polystyrene.

A particularly preferred water-soluble polymer for use herein is polyethylene glycol (PEG). As used herein, polyethylene glycol is meant to encompass any of the forms of PEG that can be used to derivatize other proteins, such as mono-(C1-C10)alkoxy-or aryloxy-polyethylene glycol. PEG is a linear or branched neutral polyether, available in a broad range of molecular weights, and is soluble in water and most organic solvents.

In some embodiments, the heterologous moiety is a carbohydrate. In some embodiments, the carbohydrate is a monosaccharide (e.g., glucose, galactose, fructose), a disaccharide (e.g., sucrose, lactose, maltose), an oligosaccharide (e.g., raffinose, stachyose), a polysaccharide (a starch, amylase, amylopectin, cellulose, chitin, callose, laminarin, xylan, mannan, fucoidan, galactomannan.

In some embodiments, the heterologous moiety is a lipid. The lipid, in some embodiments, is a fatty acid, eicosanoid, prostaglandin, leukotriene, thromboxane, N-acyl ethanolamine), glycerolipid (e.g., mono-, di-, tri-substituted glycerols), glycerophospholipid (e.g., phosphatidylcholine, phosphatidylinositol, phosphatidylethanolamine, phosphatidylserine), sphingolipid (e.g., sphingosine, ceramide), sterol lipid (e.g., steroid, cholesterol), prenol lipid, saccharolipid, or a polyketide, oil, wax, cholesterol, sterol, fat-soluble vitamin, monoglyceride, diglyceride, triglyceride, a phospholipid.

In some embodiments, the heterologous moiety is a therapeutic agent. The therapeutic agent can be any of those known in the art. Examples of therapeutic agents that are contemplated herein include, but are not limited to, natural enzymes, proteins derived from natural sources, recombinant proteins, natural peptides, synthetic peptides, cyclic peptides, antibodies, receptor agonists, cytotoxic agents, immunoglobins, beta-adrenergic blocking agents, calcium channel blockers, coronary vasodilators, cardiac glycosides, antiarrhythmics, cardiac sympathomemetics, angiotensin converting enzyme (ACE) inhibitors, diuretics, inotropes, cholesterol and triglyceride reducers, bile acid sequestrants, fibrates, 3-hydroxy-3-methylgluteryl (HMG)-CoA reductase inhibitors, niacin derivatives, antiadrenergic agents, alpha-adrenergic blocking agents, centrally acting antiadrenergic agents, vasodilators, potassium-sparing agents, thiazides and related agents, angiotensin II receptor antagonists, peripheral vasodilators, antiandrogens, estrogens, antibiotics, retinoids, insulins and analogs, alpha-glucosidase inhibitors, biguanides, meglitinides, sulfonylureas, thizaolidinediones, androgens, progestogens, bone metabolism regulators, anterior pituitary hormones, hypothalamic hormones, posterior pituitary hormones, gonadotropins, gonadotropin-releasing hormone antagonists, ovulation stimulants, selective estrogen receptor modulators, antithyroid agents, thyroid hormones, bulk forming agents, laxatives, antiperistaltics, flora modifiers, intestinal adsorbents, intestinal anti-infectives, antianorexic, anticachexic, antibulimics, appetite suppressants, antiobesity agents, antacids, upper gastrointestinal tract agents, anticholinergic agents, aminosalicylic acid derivatives, biological response modifiers, corticosteroids, antispasmodics, 5-HT₄ partial agonists, antihistamines, cannabinoids, dopamine antagonists, serotonin antagonists, cytoprotectives, histamine H2-receptor antagonists, mucosal protective agent, proton pump inhibitors, H. pylori eradication therapy, erythropoieses stimulants, hematopoietic agents, anemia agents, heparins, antifibrinolytics, hemostatics, blood coagulation factors, adenosine diphosphate inhibitors, glycoprotein receptor inhibitors, fibrinogen-platelet binding inhibitors, thromboxane-A₂ inhibitors, plasminogen activators, antithrombotic agents, glucocorticoids, mineralcorticoids, corticosteroids, selective immunosuppressive agents, antifungals, drugs involved in prophylactic therapy, AIDS-associated infections, cytomegalovirus, non-nucleoside reverse transcriptase inhibitors, nucleoside analog reverse transcriptse inhibitors, protease inhibitors, anemia, Kaposi's sarcoma, aminoglycosides, carbapenems, cephalosporins, glycopoptides, lincosamides, macrolies, oxazolidinones, penicillins, streptogramins, sulfonamides, trimethoprim and derivatives, tetracyclines, anthelmintics, amebicies, biguanides, cinchona alkaloids, folic acid antagonists, quinoline derivatives, Pneumocystis carinii therapy, hydrazides, imidazoles, triazoles, nitroimidzaoles, cyclic amines, neuraminidase inhibitors, nucleosides, phosphate binders, cholinesterase inhibitors, adjunctive therapy, barbiturates and derivatives, benzodiazepines, gamma aminobutyric acid derivatives, hydantoin derivatives, iminostilbene derivatives, succinimide derivatives, anticonvulsants, ergot alkaloids, antimigrane preparations, biological response modifiers, carbamic acid eaters, tricyclic derivatives, depolarizing agents, nondepolarizing agents, neuromuscular paralytic agents, CNS stimulants, dopaminergic reagents, monoamine oxidase inhibitors, COMT inhibitors, alkyl sulphonates, ethylenimines, imidazotetrazines, nitrogen mustard analogs, nitrosoureas, platinum-containing compounds, antimetabolites, purine analogs, pyrimidine analogs, urea derivatives, antracyclines, actinomycinds, camptothecin derivatives, epipodophyllotoxins, taxanes, vinca alkaloids and analogs, antiandrogens, antiestrogens, nonsteroidal aromatase inhibitors, protein kinase inhibitor antineoplastics, azaspirodecanedione derivatives, anxiolytics, stimulants, monoamind reuptake inhibitors, selective serotonin reuptake inhibitors, antidepressants, benzisooxazole derivatives, butyrophenone derivatives, dibenzodiazepine derivatives, dibenzothiazepine derivatives, diphenylbutylpiperidine derivatives, phenothiazines, thienobenzodiazepine derivatives, thioxanthene derivatives, allergenic extracts, nonsteroidal agents, leukotriene receptor antagonists, xanthines, endothelin receptor antagonist, prostaglandins, lung surfactants, mucolytics, antimitotics, uricosurics, xanthine oxidase inhibitors, phosphodiesterase inhibitors, metheamine salts, nitrofuran derivatives, quinolones, smooth muscle relaxants, parasympathomimetic agents, halogenated hydrocarbons, esters of amino benzoic acid, amides (e.g. lidocaine, articaine hydrochloride, bupivacaine hydrochloride), antipyretics, hynotics and sedatives, cyclopyrrolones, pyrazolopyrimidines, nonsteroidal anti-inflammatory drugs, opioids, para-aminophenol derivatives, alcohol dehydrogenase inhibitor, heparin antagonists, adsorbents, emetics, opoid antagonists, cholinesterase reactivators, nicotine replacement therapy, vitamin A analogs and antagonists, vitamin B analogs and antagonists, vitamin C analogs and antagonists, vitamin D analogs and antagonists, vitamin E analogs and antagonists, vitamin K analogs and antagonists.

The antigen-binding proteins of the present disclosure can be conjugated to one or more cytokines and growth factors that are effective in inhibiting tumor metastasis, and wherein the cytokine or growth factor has been shown to have an antiproliferative effect on at least one cell population. Such cytokines, lymphokines, growth factors, or other hematopoietic factors include, but are not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2 a, cytokine-induced neutrophil chemotactic factor 2 β, β endothelial cell growth factor, endothelin 1, epithelial-derived neutrophil attractant, glial cell line-derived neutrophic factor receptor a 1, glial cell line-derived neutrophic factor receptor a 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor a, transforming growth factor β, transforming growth factor B1, transforming growth factor β1.2, transforming growth factor B2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof.

In some embodiments, the conjugate comprises an antigen-binding protein as described herein and a cytotoxic agent. The cytotoxic agent is any molecule (chemical or biochemical) which is toxic to a cell. In some aspects, when a cytotoxic agent is conjugated to an antigen-binding protein of the present disclosure, the results obtained are synergistic. That is to say, the effectiveness of the combination therapy of an antigen-binding protein and the cytotoxic agent is synergistic, i.e., the effectiveness is greater than the effectiveness expected from the additive individual effects of each. Therefore, the dosage of the cytotoxic agent can be reduced and thus, the risk of the toxicity problems and other side effects is concomitantly reduced. In some embodiments, the cytotoxic agent is a chemotherapeutic agent. Chemotherapeutic agents are known in the art and include, but not limited to, platinum coordination compounds, topoisomerase inhibitors, antibiotics, antimitotic alkaloids and difluoronucleosides, as described in U.S. Pat. No. 6,630,124.

In some embodiments, the chemotherapeutic agent is a platinum coordination compound. The term “platinum coordination compound” refers to any tumor cell growth inhibiting platinum coordination compound that provides the platinum in the form of an ion.

In some embodiments, the platinum coordination compound is cis-diamminediaquoplatinum (II)-ion; chloro(diethylenetriamine)-platinum (II) chloride; dichloro(ethylenediamine)-platinum (II), diammine (1,1-cyclobutanedicarboxylato) platinum (II) (carboplatin); spiroplatin; iproplatin; diammine (2-ethylmalonato)-platinum (II); ethylenediaminemalonatoplatinum (II); aqua (1,2-diaminodyclohexane)-sulfatoplatinum (II); (1,2-diaminocyclohexane) malonatoplatinum (II); (4-caroxyphthalato) (1,2-diaminocyclohexane) platinum (II); (1,2-diaminocyclohexane)-(isocitrato) platinum (II); (1,2-diaminocyclohexane) cis (pyruvato) platinum (II); (1,2-diaminocyclohexane) oxalatoplatinum (II); ormaplatin; and tetraplatin.

In some embodiments, cisplatin is the platinum coordination compound employed in the compositions and methods of the present invention. Cisplatin is commercially available under the name PLATINOL™ from Bristol Myers-Squibb Corporation and is available as a powder for constitution with water, sterile saline or other suitable vehicle. Other platinum coordination compounds suitable for use in the present invention are known and are available commercially and/or can be prepared by conventional techniques. Cisplatin, or cis-dichlorodiammineplatinum II, has been used successfully for many years as a chemotherapeutic agent in the treatment of various human solid malignant tumors. More recently, other diamino-platinum complexes have also shown efficacy as chemotherapeutic agents in the treatment of various human solid malignant tumors. Such diamino-platinum complexes include, but are not limited to, spiroplatinum and carboplatinum. Although cisplatin and other diamino-platinum complexes have been widely used as chemotherapeutic agents in humans, they have had to be delivered at high dosage levels that can lead to toxicity problems such as kidney damage.

In some embodiments, the chemotherapeutic agent is a topoisomerase inhibitor. Topoisomerases are enzymes that are capable of altering DNA topology in eukaryotic cells. They are critical for cellular functions and cell proliferation. Generally, there are two classes of topoisomerases in eukaryotic cells, type I and type II. Topoisomerase I is a monomeric enzyme of approximately 100,000 molecular weight. The enzyme binds to DNA and introduces a transient single-strand break, unwinds the double helix (or allows it to unwind), and subsequently reseals the break before dissociating from the DNA strand. Various topoisomerase inhibitors have recently shown clinical efficacy in the treatment of humans afflicted with ovarian, cancer, esophageal cancer or non-small cell lung carcinoma.

In some aspects, the topoisomerase inhibitor is camptothecin or a camptothecin analog. Camptothecin is a water-insoluble, cytotoxic alkaloid produced by Camptotheca accuminata trees indigenous to China and Nothapodytes foetida trees indigenous to India. Camptothecin exhibits tumor cell growth inhibiting activity against a number of tumor cells. Compounds of the camptothecin analog class are typically specific inhibitors of DNA topoisomerase I. By the term “inhibitor of topoisomerase” is meant any tumor cell growth inhibiting compound that is structurally related to camptothecin. Compounds of the camptothecin analog class include, but are not limited to; topotecan, irinotecan and 9-amino-camptothecin.

In additional embodiments, the cytotoxic agent is any tumor cell growth inhibiting camptothecin analog claimed or described in: U.S. Pat. No. 5,004,758, issued on Apr. 2, 1991 and European Patent Application Number 88311366.4, published on Jun. 21, 1989 as 20′ Publication Number EP 0 321 122; U.S. Pat. No. 4,604,463, issued on Aug. 5, 1986 and European Patent Application Publication Number EP 0 137 145, published on Apr. 17, 1985; U.S. Pat. No. 4,473,692, issued on Sep. 25, 1984 and European Patent Application Publication Number EP 0 074 256, published on Mar. 16, 1983; U.S. Pat. No. 4,545,880, issued on Oct. 8, 1985 and European Patent Application Publication Number EP 0 074 256, published on Mar. 16, 1983; European Patent Application Publication Number EP 0 088 642, published on Sep. 14, 1983; Wani et al., J. Med. Chem., 29, 2358-2363 (1986); Nitta et al., Proc. 14th International Congr. Chemotherapy, Kyoto, 1985, Tokyo Press, Anticancer Section 1, p. 28-30, especially a compound called CPT-11. CPT-11 is a camptothecin analog with a 4-(piperidino)-piperidine side chain joined through a carbamate linkage at C-10 of 10-hydroxy-7-ethyl camptothecin. CPT-11 is currently undergoing human clinical trials and is also referred to as irinotecan; Wani et al, J. Med. Chem., 23, 554 (1980); Wani et. al., J. Med. Chem., 30, 1774 (1987); U.S. Pat. No. 4,342,776, issued on Aug. 3, 1982; U.S. patent application Ser. No. 581,916, filed on Sep. 13, 1990 and European Patent Application Publication Number EP 418 099, published on Mar. 20, 1991; U.S. Pat. No. 4,513,138, issued on Apr. 23, 1985 and European Patent Application Publication Number EP 0 074 770, published on Mar. 23, 1983; U.S. Pat. No. 4,399,276, issued on Aug. 16, 1983 and European Patent Application Publication Number 0 056 692, published on Jul. 28, 1982; the entire disclosure of each of which is hereby incorporated by reference. All of the above-listed compounds of the camptothecin analog class are available commercially and/or can be prepared by conventional techniques including those described in the above-listed references. The topoisomerase inhibitor may be selected from the group consisting of topotecan, irinotecan and 9-aminocamptothecin.

In some embodiments, the camptothecin analog is an active metabolite of irinotecan (CPT-11). In some such embodiments, the camptothecin analog is 7-ethyl-10-hydroxycamptothecin (SN-38). As a metabolite, SN-38 is formed by hydrolysis of irinotecan by carboxylesterases. In some embodiments, SN-38 has one of the following structures:

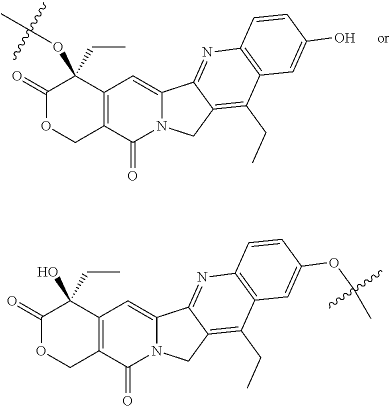 [SMILES: *O[C@]1(CC)C(=O)OCc2c1cc1n(c2=O)Cc2c-1nc1ccc(O)cc1c2CC ; *Oc1ccc2nc3c(c(CC)c2c1)Cn1c-3cc2c(c1=O)COC(=O)[C@]2(O)CC]

SN-38 has been described in U.S. Pat. Nos. 7,999,083; 8,080,250; 8,759,496; 8,999,344; 10,195,288; and 9,808,537.

In some embodiments, the camptothecin analog is exatecan methanesulfonate. Exatecan methanesulfonate is a water-soluble camptothecin (CPT) that exhibits more potent topoisomerase I inhibitory activity and antitumor activity than other CPT analogs. In addition, exatecan is effective against p-glycoprotein (P-gp)-mediated multi-drug resistant cells.

In some embodiments, the camptothecin analog is deruxtecan (Dxd), a potent derivative of exatecan, which has 10-fold higher topoisomerase I inhibitory potency than SN-38. In some embodiments, Dxd has the following structure:

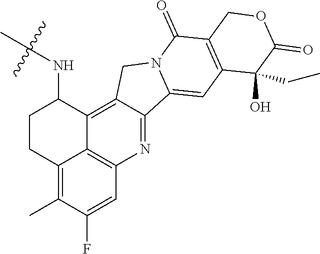 [SMILES: *NC1CCc2c(C)c(F)cc3nc4c(c1c23)Cn1c-4cc2c(c1=O)COC(=O)[C@]2(O)CC]

Dxd has been described in U.S. Pat. Nos. 6,407,115; 10,195,288; 9,808,537; and 6,407,115.

The preparation of numerous compounds of the camptothecin analog class (including pharmaceutically acceptable salts, hydrates and solvates thereof) as well as the preparation of oral and parenteral pharmaceutical compositions comprising such a compounds of the camptothecin analog class and an inert, pharmaceutically acceptable carrier or diluent, is extensively described in U.S. Pat. No. 5,004,758, issued on Apr. 2, 1991 and European Patent Application Number 88311366.4, published on Jun. 21, 1989 as Publication Number EP 0 321 122, the teachings of which are incorporated herein by reference.

In still yet other embodiments of the invention, the chemotherapeutic agent is an antibiotic compound. Suitable antibiotic include, but are not limited to, doxorubicin, mitomycin, bleomycin, daunorubicin and streptozocin.

In some embodiments, the chemotherapeutic agent is an antimitotic alkaloid. In general, antimitotic alkaloids can be extracted from Cantharanthus roseus, and have been shown to be efficacious as anticancer chemotherapy agents. A great number of semi-synthetic derivatives have been studied both chemically and pharmacologically (see, O. Van Tellingen et al, Anticancer Research, 12, 1699-1716 (1992)). The antimitotic alkaloids of the present invention include, but are not limited to, vinblastine, vincristine, vindesine, Taxol and vinorelbine. The latter two antimitotic alkaloids are commercially available from Eli Lilly and Company, and Pierre Fabre Laboratories, respectively (see, U.S. Pat. No. 5,620,985). In some embodiments, the antimitotic alkaloid is vinorelbine.

In other embodiments of the invention, the chemotherapeutic agent is a difluoronucleoside. 2′-deoxy-2′,2′-difluoronucleosides are known in the art as having antiviral activity. Such compounds are disclosed and taught in U.S. Pat. Nos. 4,526,988 and 4,808,614. European Patent Application Publication 184,365 discloses that these same difluoronucleosides have oncolytic activity. In certain aspects, the 2′-deoxy-2′,2′-difluoronucleoside used in the compositions and methods of the present invention is 2′-deoxy-2′,2′-difluorocytidine hydrochloride, also known as gemcitabine hydrochloride. Gemcitabine is commercially available or can be synthesized in a multi-step process as disclosed and taught in U.S. Pat. Nos. 4,526,988, 4,808,614 and 5,223,608, the teachings of which are incorporated herein by reference.

In various aspects, the chemotherapeutic agent is an anti-mitotic agent which inhibits cell division by blocking tubulin polymerization, destabilizing microtubules, or altering microtubule dynamics, e.g., maytansinoid or a derivative thereof (e.g., DM1 or DM4), auristatin or a derivative thereof. In various instances, the chemotherapeutic agent is an auristatin. For instance, the auristatin is in some aspects, dolastatin, Monomethyl auristatin E (MMAE), Monomethyl auristatin E (MMAE), or PF-06380101. Auristatins are described in the art. See, e.g., Maderna, A.; et al., Mol Pharmaceutics 12 (6): 1798-1812 (2015). In various aspects, the conjugate comprises an antibody of the present disclosure in combination with MMAE. Optionally, the conjugate comprises a linker. In some aspects, the linker comprises a cleavable linking moiety. In various instances, the conjugate comprises an antibody of the present disclosure linked to an attachment group which is linked to a cathepsin-cleavable linker, which in turn is linked to a spacer which is linked to MMAE. In aspects, the attachment group is attached to the antibody via a Cys residue of the Fc region of the antibody. In exemplary aspects, the attachment group comprises the structure of Formula I:

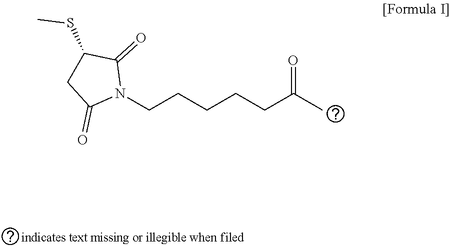 [SMILES: CS[C@H]1CC(=O)N(CCCCCC(C)=O)C1=O]

In exemplary aspects, the cathepsin cleavable linker comprises the structure of Formula II:

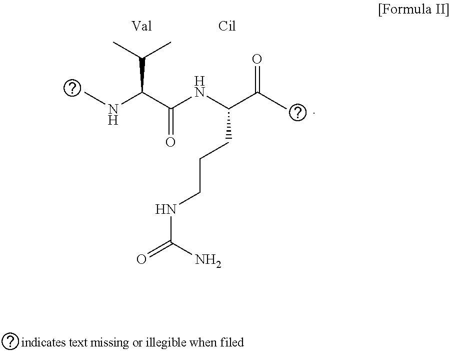 [SMILES: CN[C@H](C(=O)N[C@@H](CCCNC(N)=O)C(C)=O)C(C)C]

In exemplary aspects, the spacer comprises the structure of Formula II:

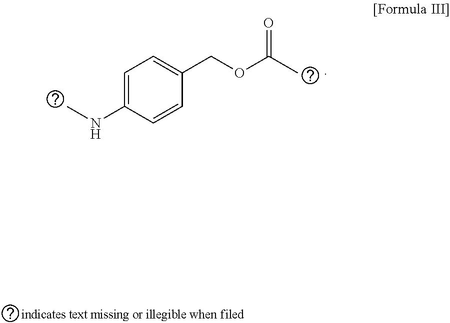

In some embodiments, MMAE has the following structure:

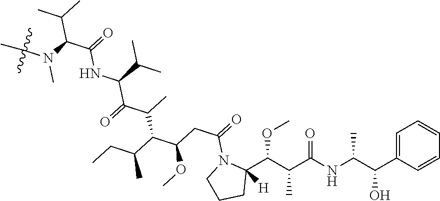 [SMILES: *N(C)[C@H](C(=O)N[C@H](C(=O)C(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

The present disclosure also provides conjugates comprising an antigen-binding protein of the present disclosure linked to a polypeptide, such that the conjugate is a fusion protein. Therefore, the present disclosure provides fusion proteins comprising an antigen-binding protein of the present disclosure linked to a polypeptide. In various embodiments, the polypeptide is a diagnostic label, e.g., a fluorescent protein, such as green fluorescent protein, or other tag, e.g., Myc tag. In various aspects, the polypeptide is one of the cytokines, lymphokines, growth factors, or other hematopoietic factors listed above.

### Linkers

In some embodiments, the conjugate is directly linked to the heterologous moiety. In alternative embodiments, the conjugate comprises a linker that joins the compound of the present disclosure to the heterologous moiety. In some aspects, the linker comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers can be selected according to their expected solubility (hydrophilicity) so as to provide a more soluble conjugate. In some embodiments, the linker provides a functional group that is subject to cleavage by an enzyme or other catalyst or hydrolytic conditions found in the target tissue or organ or cell. In some embodiments, the length of the linker is long enough to reduce the potential for steric hindrance. In some embodiments, the linker is an amino acid or a peptidyl linker. Such peptidyl linkers can be any length. Various linkers are from about 1 to 50 amino acids in length, 5 to 50, 3 to 5, 5 to 10, 5 to 15, or 10 to 30 amino acids in length.

A variety of suitable linkers are known in the art. The linker can be cleavable (a cleavable linker), e.g., under physiological conditions, e.g., under intracellular conditions, such that cleavage of the linker releases the drug in the intracellular environment. Alternatively, the linker can be cleavable under extracellular conditions, e.g., outside the tumor cells or in the vicinity of the tumor mass, such that cleavage of the linker releases the drug that permeates preferentially inside the tumor cells. In other embodiments, the linker is not cleavable (a non-cleavable linker), and the drug is released, for example, by antibody degradation.

The linker can be bonded to a chemically reactive group on the antibody moiety, e.g., to a free amino, imino, hydroxyl, thiol, or carboxyl group (e.g., to the N- or C-terminus, to the epsilon amino group of one or more lysine residues, to the free carboxylic acid group of one or more glutamic acid or aspartic acid residues, to the sulfhydryl group of one or more cysteinyl residues, or to the hydroxyl group of one or more serine or threonine residues). The site to which the linker is bound can be a natural residue in the amino acid sequence of the antibody moiety, or it can be introduced into the antibody moiety, e.g., by DNA recombinant technology (e.g., by introducing a cysteine or protease cleavage site in the amino acid sequence) or by protein biochemistry (e.g., reduction, pH adjustment, or proteolysis). The site to which the linker is bound can also be a non-natural amino acids. The site to which the linker is bound can also be a glycan on the antibody.

Typically, the linker is substantially inert under conditions for which the two groups it is connecting are linked. The term “bifunctional crosslinking agent,” “bifunctional linker” or “crosslinking agent” refers to a modifying agent that possess two reactive groups at each end of the linker, such that one reactive group can be first reacted with the cytotoxic compound to provide a compound bearing the linker moiety and a second reactive group, which can then react with the antibody. Alternatively, one end of the bifunctional crosslinking agent can be first reacted with the antibody to provide an antibody bearing a linker moiety and a second reactive group, which can then react with the cytotoxic compound. The linking moiety may contain a chemical bond that allows for the release of the cytotoxic moiety at a particular site. Suitable chemical bonds are well known in the art and include disulfide bonds, thioether bonds, acid labile bonds, photolabile bonds, protease/peptidase labile bonds, and esterase labile bonds. See, for example, U.S. Pat. Nos. 5,208,020; 5,475,092; 6,441, 163; 6,716,821; 6,913,748; 7,276,497; 7,276,499; 7,368,565; 7,388,026 and 7,414,073, each of which is incorporated herein by reference. In some embodiments, the bonds are disulfide bonds, thioether, and/or protease/peptidase labile bonds. Other linkers that can be used in the present invention include non-cleavable linkers, such as those described in detail in US20050169933, charged linkers, or hydrophilic linkers, such as those described in US 2009/0274713, US 2010/0129314, and WO 2009/134976, each of which is expressly incorporated herein by reference.

In some embodiments, the linker is a hydrophilic linker that confers hydrophilicity to the conjugate. In some embodiments, the hydrophilic linker comprises polyethylene glycol (PEG). In some embodiments, the hydrophilic linker is CLA2. In some embodiments, the CLA2 linker has the following structure:

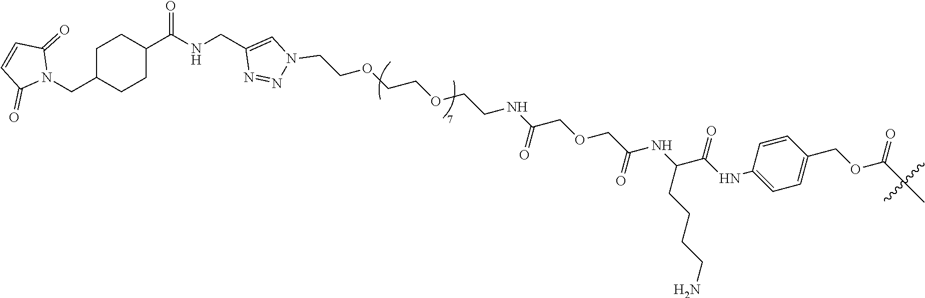

CLA2 has been described in U.S. Pat. Nos. 8,080,250; 8,759,496; and 10,195,288, each of which is incorporated herein by reference.

In some embodiments, the hydrophilic linker is CL2E. In some embodiments, the CL2E has the following structure:

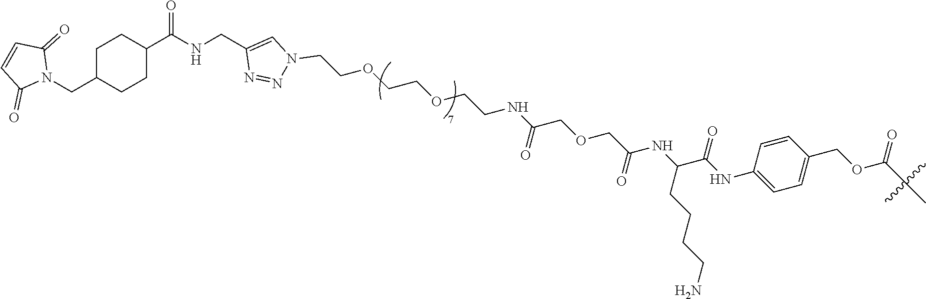

CL2E has been described in U.S. Pat. Nos. 8,080,250; 8,759,496; and 10,195,288, each of which is incorporated herein by reference.

In some embodiments, the linker is cleavable by a cleaving agent that is present in the intracellular environment (e.g., within a lysosome or endosome or caveolea). The linker can be, e.g., a peptide linker that is cleaved by an intracellular or extracellular peptidase or protease enzyme, including, but not limited to, a lysosomal or endosomal protease. In some embodiments, the peptide linker comprises at least two, at least three, at least four, or at least five amino acids long.

In some embodiments, the peptide linker is VC-PAB, comprising valine and citrulline residues. In some such embodiments, the peptide linker is MC-VC-PAB. In some embodiments, the MC-VC-PAB linker has the following structure:

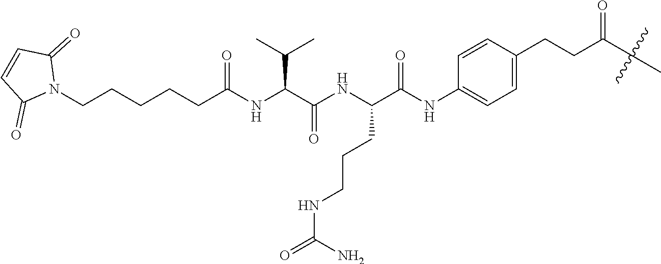 [SMILES: *C(=O)CCc1ccc(NC(=O)[C@H](CCCNC(N)=O)NC(=O)[C@@H](NC(=O)CCCCCN2C(=O)C=CC2=O)C(C)C)cc1]

MC-VC-PAB has been described in U.S. Pat. Nos. 7,659,241; 7,829,531; 6,884,869; 6,214,345; and 6,214,345, each of which is incorporated herein by reference.

In some embodiments, the peptide linker is glycine-glycine-phenylalanine-glycine (GGFG). In some such embodiments, the peptide linker is maleimidocaproyl glycine-glycine-phenylalanine-glycine (MC-GGFG). In some embodiments, the MC-GGFG linker has the following structure:

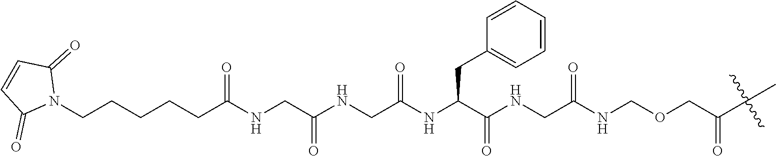 [SMILES: *C(=O)COCNC(=O)CNC(=O)[C@H](Cc1ccccc1)NC(=O)CNC(=O)CNC(=O)CCCCCN1C(=O)C=CC1=O]

MC-GGFG has been described in U.S. Pat. Nos. 9,808,537 and 10,195,288, each of which is incorporated herein by reference.

In other embodiments, the cleavable linker is pH-sensitive, i.e., sensitive to hydrolysis at certain pH values. In some embodiments, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used (see, e.g., U.S. Pat. Nos. 5,122,368; 5,824,805; 5,622,929; Dubowchik and Walker, 1999, Pharm. Therapeutics 83:67-123; Neville et al, 1989, Biol. Chem. 264:14653-14661, each of which is incorporated herein by reference). Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929, which is incorporated herein by reference).

In other embodiments, the linker is cleavable under reducing conditions (e.g., a disulfide linker). Bifunctional crosslinking agents that enable the linkage of an antibody with cytotoxic compounds via disulfide bonds include, but are not limited to, N-succinimidyl-4-(4-nitropyridyl-2-dithio) butanoate, N-succinimidyl-3-(2-pyridyldithio) propionate (SPDP), N-succinimidyl-4-(2-pyridyldithio) pentanoate (SPP), N-succinimidyl-4-(2-pyridyldithio) butanoate (SPDB), N-succinimidyl-4-(2-pyridyldithio)-2-sulfo butanoate (sulfo-SPDB). Sulfo-SPDB is described, e.g., in U.S. Pat. No. 8,236,319, incorporated herein by reference. Alternatively, crosslinking agents that introduce thiol groups such as 2-iminothiolane, homocysteine thiolactone, or S-acetylsuccinic anhydride can be used. In other embodiments, the linker may contain a combination of one or more of the peptide, pH-sensitive, or disulfide linkers described previously.

“Heterobifunctional crosslinking agents” are bifunctional crosslinking agents having two different reactive groups. Heterobifunctional crosslinking agents containing both an amine-reactive N-hydroxysuccinimide group (NHS group) and a carbonyl-reactive hydrazine group can also be used to link cytotoxic compounds with an antibody. Examples of such commercially available heterobifunctional crosslinking agents include succinimidyl 6-hydrazinonicotinamide acetone hydrazone (SANH), succinimidyl 4-hydrazidoterephthalate hydrochloride (SHTH) and succinimidyl hydrazinium nicotinate hydrochloride (SHNH). Conjugates bearing an acid-labile linkage can also be prepared using a hydrazine-bearing benzodiazepine derivative of the present invention. Examples of bifunctional crosslinking agents that can be used include succinimidyl-p-formyl benzoate (SFB) and succinimidyl-p-formylphenoxyacetate (SFPA).

The linkers described herein may be used in any combination with the heterologous moiety described herein. In addition, the linkers described herein can have any chemical reactive moieties (e.g., maleimide, cysteine, etc.) that can react with any part (e.g., an amino acid, disulfide bond, carbohydrate (e.g., those from the post-translational modification), etc.) of the antigen-binding protein of the present disclosure. Often, lysines or cysteines (e.g., cysteines from the reduced disulfide bonds (e.g., from interchain or intrachain disulfide bonds of the antibody or antigen-binding protein) or an engineered unpaired cysteine) on an antibody or an antigen-binding protein have been used as a site for conjuation. All of the above-listed linkers and heterologous moiety described herein are available commercially and/or can be prepared by conventional techniques including those described in the above-listed references.

### Conjugation

The heterologous moiety-to-antigen-binding protein ratio (HAR) represents the number of a heterologous moiety linked per antigen-binding molecule. In some embodiments, the HAR ranges from 1 to 15, 1 to 10, 1 to 9, 1 to 8, 1 to 7, 1 to 6, 1 to 5, 1 to 4, 1 to 3, or 1 to 2. In some embodiments, the HAR ranges from 2 to 10, 2 to 9, 2 to 8, 2 to 7, 2 to 6, 2 to 5, 2 to 4 or 2 to 3. In other embodiments, the HAR is about 2, about 2.5, about 3, about 4, about 5, or about 6. In some embodiments, the HAR ranges from about 2 to about 4. The HAR may be characterized by conventional means such as mass spectrometry, UV/Vis spectroscopy, ELISA assay, and/or HPLC.

In some embodiments, the conjugates are heterogeneous conjugates (also referred to as “conventional”), wherein the antigen-binding proteins are conjugated to a different number of the heterologous moiety. In some embodiments, the heterogeneous conjugates follow a Gaussian distribution or quasi-Gaussian distribution of the conjugates, wherein the distribution centers on the average heterologous moiety loading value with some antigen-binding proteins conjugated with higher than average and some antigen-binding proteins conjugated with lower than the average.

In some embodiments, the conjugates are homogeneous conjugates, wherein the substantial percentage of the antigen-binding proteins are conjugated to a defined number of the heterologous moiety. In some embodiments, the homogeneous conjugates comprise the HAR of 1, 2, 3, 4, 5, 6, 7, 8, 9, or 10. In some embodiments, the homogeneous conjugates comprise the HAR of 2, 4, 6, or 8. In preferred embodiments, the homogeneous conjugates comprise the HAR of 4. In other preferred embodiments, the homogeneous conjugates comprise the HAR of 2. In some embodiments, the homogeneous conjugates comprise greater than or equal to 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent conjugates with the defined HAR. In some embodiments, the homogeneous conjugates comprise about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent conjugates with the defined HAR. In some embodiments, the homogeneous conjugates comprise at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent conjugates with the defined HAR. In some embodiments, the homogeneous conjugates comprise the HAR distribution that is not Gaussian or quasi-Gaussian distribution. In some embodiments, the homogeneity of the homogeneous conjugates is determined by a chromatogram, e.g., HPLC or any suitable chromatography. In some embodiments, the chromatogram is a HIC chromatogram. The homogeneous conjugate may be generated by a site-specific conjugation.

In some embodiments, the heterologous moiety is conjugated to the antigen-binding protein (e.g., antibody) in a site-specific manner. Various site-specific conjugation methods are known in the art, e.g., thiomab or TDC or conjugation at an unpaired cysteine residue (Junutula et al. (2008) Nat. Biotechnol. 26:925-932; Dimasi et al. (2017) Mol. Pharm. 14:1501-1516; Shen et al. (2012) Nat. Biotechnol. 30:184-9); thiol bridge linker (Behrens et al. (2015) Mol. Pharm. 12:3986-98); conjugation at glutamine using a transglutaminase (Dennler et al. (2013) Methods Mol. Bio. 1045:205-15; Dennler et al. (2014) Bioconjug Chem. 25:569-78); conjugation at engineered unnatural amino acid residues (Axup et al. (2012) Proc Natl Acad Sci U.S.A. 104-16101-6; Tian et al. (2014) Proc Natl Acad Sci U.S.A. 111:1766-71; VanBrunt et al. (2015) Bioconjug Chem 26:2249-60; Zimmerman et al. (2014) Bioconjug Chem 25:351-61); selenocysteine conjugation (Li et al. (2017) Cell Chem Biol 24:433-442); glycan-mediated conjugation (Okeley et al. (2013) Bioconjug Chem 24:1650-5); conjugation at galactose or GalNAc analogues (Ramakrishnan and Qasba (2002) J Biol Chem 277:20833-9; van Geel et al. (2015) Bioconjug Chem 26:2233-42); via glycan engineering (Zhou et al. (2014) Bioconjug Chem 25:510-20; Tang et al. (2017) Nat Protoc 12:1702-1721); via a short peptide tag, such as engineering a glutamine tag or sortase A-mediated transpeptidation (Strop et al. (2013) Chem Biol 20:161-7; Beerli et al. (2015) PLOS One 10: e0131177); and via an aldehyde tag (Wu et al. (2009) Proc Natl Acad Sci U.S.A. 106:3000-5), each of which is incorporated herein by reference.

### Unpredictability of Conjugate (e.g., ADC)

It is not possible to predict in advance, simply based on an antibody profile, or a drug payload profile, which antibody-drug conjugates will be sufficiently safe and effective for clinical applications. For example, a particular drug payload may function perfectly well when conjugated to an antibody directed to one target, but it may not work nearly as well when conjugated to an antibody directed to a different target, or even to a different antibody directed to the same target. Why different antibody-drug conjugates display different anti-tumor activity in vivo is not sufficiently well understood to allow accurate predictions in the design of new antibody-drug conjugates. It is speculated that an unpredictable interplay of many factors play a role. These factors may include, for example, the binding affinity of an antibody-drug conjugate to a target antigen, the ability of the conjugate to penetrate solid tumors, as well as the half-life in circulation for proper exposure to tumors without causing toxicity.

The complexity and unpredictability is well demonstrated by antibody affinity alone. Antibodies or antibody-drug conjugates with high affinity track with better cellular uptake, which leads to a higher level of the cytotoxic payloads released inside the cells. Higher affinity is also known to enhance the antibody-dependent cellular cytotoxicity (ADCC). All these attributes favor the cell killing property of antibody-drug conjugates. However, it is also known that high affinity of an antibody or antibody-drug conjugate can prevent efficient tumor penetration via an “antigen barrier effect,” suggesting that in order to achieve a strong anti-tumor activity in vivo, affinity of the antibody-drug conjugate has to be just right: not too high or not too low. To date, it is not known how to predict what will be the most efficient or effective level of affinity for an antibody-drug conjugate.

In addition, in vivo anti-tumor activity cannot be predicted by the mechanism of linkers and payloads alone. For example, O. Ab et al, Mol. Cancer Ther. 14 (&): 1605-1613 (2015), which is incorporated herein by reference, demonstrated that, when tested in pre-clinical cancer models, the same antibody conjugated to the same anti-tubulin toxin via different linkers exhibited dramatically different anti-tumor activity. This example is particularly surprising because the chemical structures of the two linkers are very similar. Moreover, the linker present in the superior conjugate contained a hydrophilic moiety. Hydrophilic metabolites are generally less membrane-permeable, and are thought to be slower in efflux from the lysosomes (the site of conjugate degradation), leading to a delay in the anti-tubulin activity of the released payload. This finding argues for an “ideal” kinetics of payload delivery, but to date, there is no insight into what constitutes such kinetics. Adding to this complexity is the open question of whether ideal kinetics of payload delivery, even if defined for a particular cell type, would apply to all cell types. Thus, it is not possible to predict the most effective in vivo anti-tumor activity merely from the chemical composition of the linker or payload.

### Compositions, Pharmaceutical Compositions and Formulations

Compositions comprising an antigen-binding protein, a nucleic acid, a vector, a host cell, or a conjugate as presently disclosed are provided herein. The compositions in some aspects comprise the antigen-binding proteins in isolated and/or purified form. In some aspects, the composition comprises a single type (e.g., structure) of an antigen-binding protein of the present disclosure or comprises a combination of two or more antigen-binding proteins of the present disclosure, wherein the combination comprises two or more antigen-binding proteins of different types (e.g., structures).

In some aspects, the composition comprises agents which enhance the chemico-physico features of the antigen-binding protein, e.g., via stabilizing the antigen-binding protein at certain temperatures, e.g., room temperature, increasing shelf life, reducing degradation, e.g., oxidation protease mediated degradation, increasing half-life of the antigen-binding protein, etc. In some aspects, the composition comprises any of the agents disclosed herein as a heterologous moiety or conjugate moiety, optionally in admixture with the antigen-binding proteins of the present disclosure or conjugated to the antigen-binding proteins.

In various aspects of the present disclosure, the composition additionally comprises a pharmaceutically acceptable carrier, diluents, or excipient. In some embodiments, the antigen-binding protein, a nucleic acid, a vector, a host cell, or a conjugate as presently disclosed (hereinafter referred to as “active agents”) is formulated into a pharmaceutical composition comprising the active agent, along with a pharmaceutically acceptable carrier, diluent, or excipient. In this regard, the present disclosure further provides pharmaceutical compositions comprising an active agent which is intended for administration to a subject, e.g., a mammal.

In some embodiments, the active agent is present in the pharmaceutical composition at a purity level suitable for administration to a patient. In some embodiments, the active agent has a purity level of at least about 90%, about 91%, about 92%, about 93%, about 94%, about 95%, about 96%, about 97%, about 98% or about 99%, and a pharmaceutically acceptable diluent, carrier or excipient. In some embodiments, the compositions contain an active agent at a concentration of about 0.001 to about 30.0 mg/ml.

In various aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The pharmaceutical composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In various aspects, the pharmaceutical composition comprises formulation materials that are nontoxic to recipients at the dosages and concentrations employed. In specific embodiments, pharmaceutical compositions comprising an active agent and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In various aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbatc, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company, which is incorporated herein by reference.

The pharmaceutical compositions can be formulated to achieve a physiologically compatible pH. In some embodiments, the pH of the pharmaceutical composition can be for example between about 4 or about 5 and about 8.0 or about 4.5 and about 7.5 or about 5.0 to about 7.5. In various embodiments, the pH of the pharmaceutical composition is between 5.5 and 7.5.

The present disclosure provides methods of producing a pharmaceutical composition. In various aspects, the method comprises combining the antigen-binding protein, conjugate, fusion protein, nucleic acid, vector, host cell, or a combination thereof, with a pharmaceutically acceptable carrier, diluent, or excipient.

### Routes of Administration

With regard to the present disclosure, the active agent, or pharmaceutical composition comprising the same, can be administered to the subject via any suitable route of administration. For example, the active agent can be administered to a subject via parenteral, nasal, oral, pulmonary, topical, vaginal, or rectal administration. The following discussion on routes of administration is merely provided to illustrate various embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The active agent of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments contain from about 0.5% to about 25% by weight of the active agent of the present disclosure in solution. Preservatives and buffers can be used. In order to minimize or eliminate irritation at the site of injection, such compositions can contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the present disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Toissel, 4th ed., pages 622-630 (1986), each of which is incorporated herein by reference).

### Dosages

The active agents of the disclosure are believed to be useful in methods of inhibiting tumor growth, as well as other methods, as further described herein, including methods of treating or preventing cancer. For purposes of the disclosure, the amount or dose of the active agent administered should be sufficient to effect, e.g., a therapeutic or prophylactic response, in the subject or animal over a reasonable time frame. For example, the dose of the active agent of the present disclosure should be sufficient to treat cancer as described herein in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the efficacy of the particular active agent and the condition of the animal (e.g., human), as well as the body weight of the animal (e.g., human) to be treated.

Many assays for determining an administered dose are known in the art. For purposes herein, an assay, which comprises comparing the extent to which cancer is treated upon administration of a given dose of the active agent of the present disclosure to a mammal among a set of mammals, each set of which is given a different dose of the active agent, could be used to determine a starting dose to be administered to a mammal. The extent to which cancer is treated upon administration of a certain dose can be represented by, for example, the extent of tumor regression achieved with the active agent in a mouse xenograft model. Methods of assaying tumor regression are known in the art and described herein.

The dose of the active agent of the present disclosure also will be determined by the existence, nature and extent of any adverse side effects that might accompany the administration of a particular active agent of the present disclosure. Typically, the attending physician will decide the dosage of the active agent of the present disclosure with which to treat each individual patient, taking into consideration a variety of factors, such as age, body weight, general health, diet, sex, active agent of the present disclosure to be administered, route of administration, and the severity of the condition being treated. By way of example and not intending to limit the present disclosure, the dose of the active agent of the present disclosure can be about 0.0001 to about 1 g/kg body weight of the subject being treated/day, from about 0.0001 to about 0.001 g/kg body weight/day, or about 0.01 mg to about 1 g/kg body weight/day.

### Controlled Release Formulations

In some embodiments, the active agents described herein can be modified into a depot form, such that the manner in which the active agent of the present disclosure is released into the body to which it is administered is controlled with respect to time and location within the body (see, for example, U.S. Pat. No. 4,450,150). Depot forms of active agents of the present disclosure can be, for example, an implantable composition comprising the active agents and a porous or non-porous material, such as a polymer, wherein the active agent is encapsulated by or diffused throughout the material and/or degradation of the non-porous material. The depot is then implanted into the desired location within the body of the subject and the active agent is released from the implant at a predetermined rate.

The pharmaceutical composition comprising the active agent in certain aspects is modified to have any type of in vivo release profile. In some aspects, the pharmaceutical composition is an immediate release, controlled release, sustained release, extended release, delayed release, or bi-phasic release formulation. Methods of formulating peptides for controlled release are known in the art. See, for example, Qian et al., J Pharm 374:46-52 (2009) and International Patent Application Publication Nos. WO 2008/130158, WO2004/033036; WO2000/032218; and WO 1999/040942, each of which is incorporated herein by reference.

The instant compositions can further comprise, for example, micelles or liposomes, or some other encapsulated form, or can be administered in an extended release form to provide a prolonged storage and/or delivery effect.

### Use

The antigen-binding proteins of the present disclosure are useful for inhibiting tumor growth. Without being bound to a particular theory, the inhibiting action of the antigen-binding proteins provided herein allow such entities to be useful in methods of treating cancer.

Accordingly, provided herein are methods of inhibiting tumor growth in a subject and methods of reducing tumor size in a subject. In various embodiments, the methods comprise administering to the subject the pharmaceutical composition of the present disclosure in an amount effective for inhibiting tumor growth or reducing tumor size in the subject. In various aspects, the growth of an ovarian tumor, melanoma tumor, bladder tumor, or endometrial tumor is inhibited. In various aspects, the size of an ovarian tumor, melanoma tumor, bladder tumor, or endometrial tumor is reduced.

As used herein, the term “inhibit” or “reduce” and words stemming therefrom may not be a 100% or complete inhibition or reduction. Rather, there are varying degrees of inhibition or reduction of which one of ordinary skill in the art recognizes as having a potential benefit or therapeutic effect. In this respect, the antigen-binding proteins of the present disclosure may inhibit tumor growth or reduce tumor size to any amount or level. In various embodiments, the inhibition provided by the methods of the present disclosure is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition). In various embodiments, the reduction provided by the methods of the present disclosure is at least or about a 10% reduction (e.g., at least or about a 20% reduction, at least or about a 30% reduction, at least or about a 40% reduction, at least or about a 50% reduction, at least or about a 60% reduction, at least or about a 70% reduction, at least or about a 80% reduction, at least or about a 90% reduction, at least or about a 95% reduction, at least or about a 98% reduction).

Additionally provided herein are methods of treating a subject with cancer, e.g., CDH17-expressing cancer. In various embodiments, the method comprises administering to the subject the pharmaceutical composition of the present disclosure in an amount effective for treating the cancer in the subject.

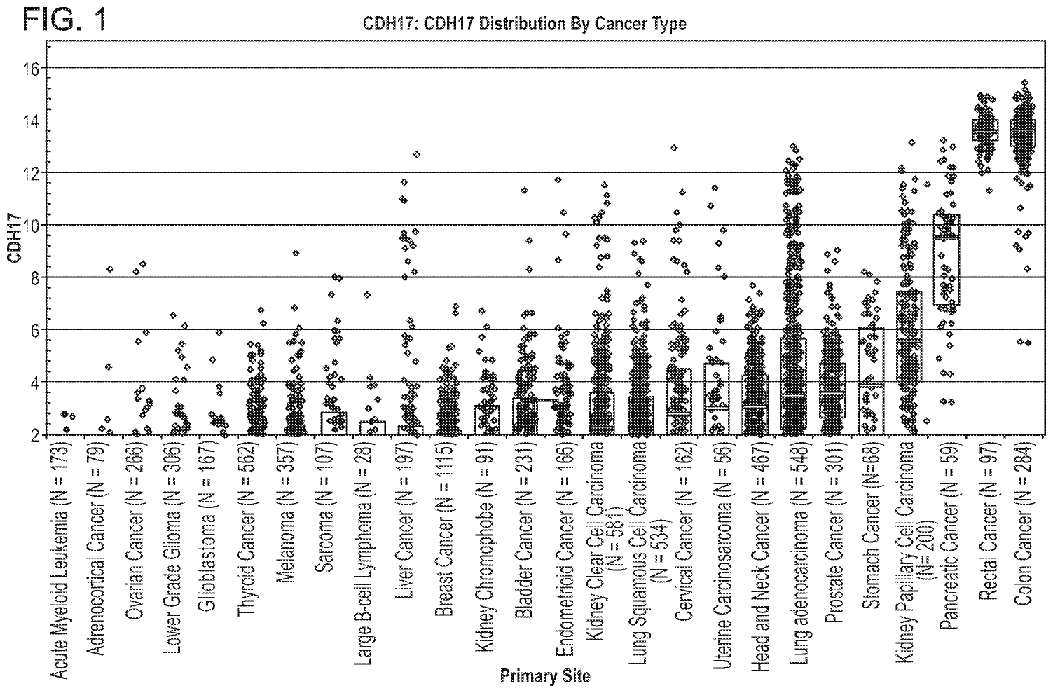

For purposes herein, the cancer of the methods disclosed herein can be any cancer, e.g., any malignant growth or tumor caused by abnormal and uncontrolled cell division that may spread to other parts of the body through the lymphatic system or the blood stream. The cancer in some aspects is one selected from the group consisting of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In various aspects, the cancer is pancreatic cancer, gastrointestinal cancer, bladder cancer, colon cancer, lung cancer, liver cancer, endometrial cancer. In various aspects, the cancer is any cancer characterized by moderate to high expression of CDH17. See, e.g., FIG. 1. In various aspects, the cancer is acute myeloid leukemia, large B-cell lymphoma, stomach cancer, prostate cancer, melanoma, colon cancer, rectal cancer, bladder cancer, cervical cancer, liver cancer, breast cancer, kidney clear cell carcinoma, head and neck cancer, sarcoma, kidney chromophobe cancer, lower grade glioma, adrenocortical cancer, glioblastoma, kidney papillary cell carcinoma, lung squamous cell carcinoma, thyroid cancer, lung adenocarcinoma, pancreatic cancer, endometroid cancer, uterine carcinsarcoma, or ovarian cancer. In various aspects, the cancer is selected from pancreatic cancer, gastrointestinal cancer, bladder cancer, colon cancer, lung cancer, liver cancer, ovarian cancer, endometrioid cancer, uterine cancer, lung cancer, gastric cancer, breast cancer Head and Neck Squamous Cell Carcinoma (HNSCC) cancer, and cervical cancer.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the cancer. For example, the methods can treat cancer by virtue of enhancing the T cell activity or an immune response against the cancer, reducing tumor or cancer growth, reducing metastasis of tumor cells, increasing cell death of tumor or cancer cells, and the like. In various aspects, the methods treat by way of delaying the onset or recurrence of the cancer by at least 1 day, 2 days, 4 days, 6 days, 8 days, 10 days, 15 days, 30 days, two months, 3 months, 4 months, 6 months, 1 year, 2 years, 3 years, 4 years, or more. In various aspects, the methods treat by way increasing the survival of the subject.

The antigen binding proteins of the present disclosure also may be used to detect CDH17 in a sample or diagnose a CDH17-positive cancer. Therefore, the present disclosure provides methods of detecting CDH17 in a sample. In various embodiments, the method comprises contacting the sample with an antigen-binding protein, a conjugate, or a fusion protein, as described herein, and assaying for an immunocomplex comprising the antigen-binding protein, conjugate or fusion protein bound to CDH17. The present disclosure also provides methods of diagnosing a CDH17-positive cancer in a subject. In various embodiments, the method comprises contacting a biological sample comprising cells or tissue obtained from the subject with an antigen-binding protein, a conjugate, or a fusion protein, as described herein, and assaying for an immunocomplex comprising the antigen-binding protein, conjugate or fusion protein bound to CDH17.

### Subjects

In some embodiments of the present disclosure, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human.

### Kits

In some embodiments, the antigen-binding proteins of the present disclosure are provided in a kit. In various aspects, the kit comprises the antigen-binding protein(s) as a unit dose. For purposes herein “unit dose” refers to a discrete amount dispersed in a suitable carrier. In various aspects, the unit dose is the amount sufficient to provide a subject with a desired effect, e.g., inhibition of tumor growth, reduction of tumor size, treatment of cancer. Accordingly, provided herein are kits comprising an antigen-binding protein of the present disclosure optionally provided in unit doses. In various aspects, the kit comprises several unit doses, e.g., a week or month supply of unit doses, optionally, each of which is individually packaged or otherwise separated from other unit doses. In some embodiments, the components of the kit/unit dose are packaged with instructions for administration to a patient. In some embodiments, the kit comprises one or more devices for administration to a patient, e.g., a needle and syringe, and the like. In some aspects, the antigen-binding protein of the present disclosure, a pharmaceutically acceptable salt thereof, a conjugate comprising the antigen-binding protein, or a multimer or dimer comprising the antigen-binding protein, is pre-packaged in a ready to use form, e.g., a syringe, an intravenous bag, etc. In some aspects, the kit further comprises other therapeutic or diagnostic agents or pharmaceutically acceptable carriers (e.g., solvents, buffers, diluents, etc.), including any of those described herein. In particular aspects, the kit comprises an antigen-binding protein of the present disclosure, along with an agent, e.g., a therapeutic agent, used in chemotherapy or radiation therapy.

### VARIOUS EMBODIMENTS

In various embodiments of the present disclosure, the antigen-binding protein binds to a human CDH17 protein. In some embodiments, the antigen-binding protein of the present disclosure binds to the ECD of CDH17. In some embodiments, the antigen-binding protein binds one or more peptide(s) or polypeptide(s) derived from the ECD of CDH17. In some embodiments, the binding of the antigen-binding protein to CDH17 depends on the amino acid sequence of the peptide or polypeptide. In some aspects of the invention, the antigen-binding protein to CDH17 binds the three dimensional conformation (i.e., tertiary structure) of CDH17 or a peptide or polypeptide derived from the ECD of CDH17. In other embodiments, the antigen-binding protein to CDH17 binds a primary structure or conformation of CDH17 or a peptide or polypeptide derived from the ECD of CDH17 (i.e. a linear conformation). In preferred embodiments, the preferred the antigen-binding protein to CDH17 binds LDANGII derived from the ECD of CDH17. In yet other preferred embodiments, the antigen-binding protein binds to DANGI derived from the ECD of CDH17. Specific examples of antigen-binding proteins which binds CDH17 protein include, but are not limited to, those shown in Tables 1-5 and 9.

In some embodiments, the antigen-binding protein of the present disclosure comprises a Fc polypeptide. In some embodiments, the antigen-binding protein of the present disclosure comprises a Fc polypeptide comprising an afucosylated glycan.

In various aspects, the antigen-binding protein of the present disclosure is an antibody, e.g., a monoclonal antibody. In various instances, the antigen-binding protein is an IgG. In various aspects, the antigen-binding protein inhibits at least about 50% colony growth in a soft agar 3D proliferation assays or inhibits tumor growth in xenograft mice injected with human cancer cells. In various aspects, the antigen-binding protein inhibits tumor growth of in xenograft mice injected with ovarian cancer cells, melanoma cancer cells, bladder cancer cells, or endometrial cancer cells. In various instances, the antigen-binding protein inhibits at least 50% tumor growth in xenograft mice injected with ovarian cancer cells, bladder cancer cells, or endometrial cancer cells.

In certain embodiments, the antigen-binding protein comprises: (a) a heavy chain CDR1 comprising the amino acid sequence of: GYTFXDXT (SEQ ID NO: 55) wherein X at position 5 is N, S, R, Q, A or T, and X at position 7 is H, W, Y or F; (b) a heavy chain CDR2 comprising the amino acid sequence of: IFPRDDIV (SEQ ID NO: 14) or a variant sequence thereof which differs by only one or two amino acids or which has at least or about 70% sequence identity; (c) a heavy chain CDR3 comprising the amino acid sequence of: ARPPYYYSRNFYFDY (SEQ ID NO: 15) or a variant sequence thereof which differs by only one or two amino acids or which has at least or about 70% sequence identity; (d) a light chain CDR1 comprising the amino acid sequence of: SIISSSK (SEQ ID NO: 16) or a variant sequence thereof which differs by only one or two amino acids or which has at least or about 70% sequence identity; (e) a light chain CDR2 comprising the amino acid sequence of: GTS (SEQ ID NO: 17) or a variant sequence thereof which differs by only one or two amino acids; (f) a light chain CDR3 comprising the amino acid sequence of: QQWSNYPFT (SEQ ID NO: 18) or a variant sequence thereof which differs by only one or two amino acids or which has at least or about 70% sequence identity; or (g) a combination of any two or moreof (a)-(f). In some embodiments, the antigen-binding protein further binds to an epitope comprising the amino acid sequence of any of THNLQVAALDANGIIVEGPVPIT (SEQ ID NO: 82), THNLQVAALDANGII (SEQ ID NO: 83), QVAALDANGIIVEGP (SEQ ID NO: 84), LDANGIIVEGPVPIT (SEQ ID NO: 85), LDANGII (SEQ ID NO: 53) or DANGI (SEQ ID NO: 54).

In certain embodiments, the antigen-binding protein comprises: (a) a heavy chain CDR1 comprising the amino acid sequence of: GYTFXSXN (SEQ ID NO: 56) wherein X at position 5 is N, S, R, Q, A or T, and X at position 7 is H, W, Y or F; (b) a heavy chain CDR2 comprising the amino acid sequence of: IYPGNGDT (SEQ ID NO: 2) or a variant sequence thereof which differs by only one or two amino acids or which has at least or about 70% sequence identity; (c) a heavy chain CDR3 comprising the amino acid sequence of: ARGRGRYFEY (SEQ ID NO: 3) or a variant sequence thereof which differs by only one or two amino acids or which has at least or about 70% sequence identity; (d) a light chain CDR1 comprising the amino acid sequence of: SSVSSSY (SEQ ID NO: 4) or a variant sequence thereof which differs by only one or two amino acids or which has at least or about 70% sequence identity; (e) a light chain CDR2 comprising the amino acid sequence of: STS (SEQ ID NO: 5) or a variant sequence thereof which differs by only one or two amino acids; (f) a light chain CDR3 comprising the amino acid sequence of: QQYDSSPST (SEQ ID NO: 6) or a variant sequence thereof which differs by only one or two amino acids or which has at least or about 70% sequence identity; or (g) a combination of any two or moreof (a)-(f). In some embodiments, the antigen-binding protein further binds to an epitope comprising the amino acid sequence of any of THNLQVAALDANGIIVEGPVPIT (SEQ ID NO: 82), THNLQVAALDANGII (SEQ ID NO: 83), QVAALDANGIIVEGP (SEQ ID NO: 84), LDANGIIVEGPVPIT (SEQ ID NO: 85), LDANGII (SEQ ID NO: 53) or DANGI (SEQ ID NO: 54).

In certain embodiments, the antigen-binding protein comprises: (a) a heavy chain CDR1 comprising the amino acid sequence of: GYTFXDXY (SEQ ID NO: 57) wherein X at position 5 is N, S, R, Q, A or T, and X at position 7 is H, W, Y or F; (b) a heavy chain CDR2 comprising the amino acid sequence of: IYPYSGGI (SEQ ID NO: 8) or a variant sequence thereof which differs by only one or two amino acids or which has at least or about 70% sequence identity; (c) a heavy chain CDR3 comprising the amino acid sequence of: ARGRGDYFGLFDF (SEQ ID NO: 9) or a variant sequence thereof which differs by only one or two amino acids or which has at least or about 70% sequence identity; (d) a light chain CDR1 comprising the amino acid sequence of: SSLSY (SEQ ID NO: 10) or a variant sequence thereof which differs by only one or two amino acids or which has at least or about 70% sequence identity; (e) a light chain CDR2 comprising the amino acid sequence of: EIS (SEQ ID NO: 11) or a variant sequence thereof which differs by only one or two amino acids; (f) a light chain CDR3 comprising the amino acid sequence of: QQWNYPFT (SEQ ID NO: 12) or a variant sequence thereof which differs by only one or two amino acids or which has at least or about 70% sequence identity; or (g) a combination of any two or moreof (a)-(f). In some embodiments, the antigen-binding protein further binds to an epitope comprising the amino acid sequence of any of THNLQVAALDANGIIVEGPVPIT (SEQ ID NO: 82), THNLQVAALDANGII (SEQ ID NO: 83), QVAALDANGIIVEGP (SEQ ID NO: 84), LDANGIIVEGPVPIT (SEQ ID NO: 85), LDANGII (SEQ ID NO: 53) or DANGI (SEQ ID NO: 54).

In certain embodiments, the antigen-binding protein comprises: (a) a heavy chain CDR1 comprising the amino acid sequence of: GYTFXXXX (SEQ ID NO: 81) wherein X at position 5 is N, S, R, Q, A or T, X at position 6 is D or S, X at position 7 is H, W, Y or F, and X at position 8 is Y, T or N; (b) a heavy chain CDR2 comprising the amino acid sequence of: IFPRDDIV (SEQ ID NO: 14) or a variant sequence thereof which differs by only one or two amino acids or which has at least or about 70% sequence identity; (c) a heavy chain CDR3 comprising the amino acid sequence of: ARPPYYYSRNFYFDY (SEQ ID NO: 15) or a variant sequence thereof which differs by only one or two amino acids or which has at least or about 70% sequence identity; (d) a light chain CDR1 comprising the amino acid sequence of: SIISSSK (SEQ ID NO: 16) or a variant sequence thereof which differs by only one or two amino acids or which has at least or about 70% sequence identity; (e) a light chain CDR2 comprising the amino acid sequence of: GTS (SEQ ID NO: 17) or a variant sequence thereof which differs by only one or two amino acids; (f) a light chain CDR3 comprising the amino acid sequence of: QQWSNYPFT (SEQ ID NO: 18) or a variant sequence thereof which differs by only one or two amino acids or which has at least or about 70% sequence identity; or (g) a combination of any two or moreof (a)-(f). In some embodiments, the antigen-binding protein further binds to an epitope comprising the amino acid sequence of any of THNLQVAALDANGIIVEGPVPIT (SEQ ID NO: 82), THNLQVAALDANGII (SEQ ID NO: 83), QVAALDANGIIVEGP (SEQ ID NO: 84), LDANGIIVEGPVPIT (SEQ ID NO: 85), LDANGII (SEQ ID NO: 53) or DANGI (SEQ ID NO: 54).

In certain embodiments, the antigen-binding protein comprises: (a) a heavy chain CDR1 comprising the amino acid sequence of: GYTFXXXX (SEQ ID NO: 81) wherein X at position 5 is N, S, R, Q, A or T, X at position 6 is D or S, X at position 7 is H, W, Y or F, and X at position 8 is Y, T or N; (b) a heavy chain CDR2 comprising the amino acid sequence of: IYPGNGDT (SEQ ID NO: 2) or a variant sequence thereof which differs by only one or two amino acids or which has at least or about 70% sequence identity; (c) a heavy chain CDR3 comprising the amino acid sequence of: ARGRGRYFEY (SEQ ID NO: 3) or a variant sequence thereof which differs by only one or two amino acids or which has at least or about 70% sequence identity; (d) a light chain CDR1 comprising the amino acid sequence of: SSVSSSY (SEQ ID NO: 4) or a variant sequence thereof which differs by only one or two amino acids or which has at least or about 70% sequence identity; (e) a light chain CDR2 comprising the amino acid sequence of: STS (SEQ ID NO: 5) or a variant sequence thereof which differs by only one or two amino acids; (f) a light chain CDR3 comprising the amino acid sequence of: QQYDSSPST (SEQ ID NO: 6) or a variant sequence thereof which differs by only one or two amino acids or which has at least or about 70% sequence identity; or (g) a combination of any two or moreof (a)-(f). In some embodiments, the antigen-binding protein further binds to an epitope comprising the amino acid sequence of any of THNLQVAALDANGIIVEGPVPIT (SEQ ID NO: 82), THNLQVAALDANGII (SEQ ID NO: 83), QVAALDANGIIVEGP (SEQ ID NO: 84), LDANGIIVEGPVPIT (SEQ ID NO: 85), LDANGII (SEQ ID NO: 53) or DANGI (SEQ ID NO: 54).

In certain embodiments, the antigen-binding protein comprises: (a) a heavy chain CDR1 comprising the amino acid sequence of: GYTFXXXX (SEQ ID NO: 81) wherein X at position 5 is N, S, R, Q, A or T, X at position 6 is D or S, X at position 7 is H, W, Y or F, and X at position 8 is Y, T or N; (b) a heavy chain CDR2 comprising the amino acid sequence of: IYPYSGGI (SEQ ID NO: 8) or a variant sequence thereof which differs by only one or two amino acids or which has at least or about 70% sequence identity; (c) a heavy chain CDR3 comprising the amino acid sequence of: ARGRGDYFGLFDF (SEQ ID NO: 9) or a variant sequence thereof which differs by only one or two amino acids or which has at least or about 70% sequence identity; (d) a light chain CDR1 comprising the amino acid sequence of: SSLSY (SEQ ID NO: 10) or a variant sequence thereof which differs by only one or two amino acids or which has at least or about 70% sequence identity; (e) a light chain CDR2 comprising the amino acid sequence of: EIS (SEQ ID NO: 11) or a variant sequence thereof which differs by only one or two amino acids; (f) a light chain CDR3 comprising the amino acid sequence of: QQWNYPFT (SEQ ID NO: 12) or a variant sequence thereof which differs by only one or two amino acids or which has at least or about 70% sequence identity; or (g) a combination of any two or moreof (a)-(f). In some embodiments, the antigen-binding protein further binds to an epitope comprising the amino acid sequence of any of THNLQVAALDANGIIVEGPVPIT (SEQ ID NO: 82), THNLQVAALDANGII (SEQ ID NO: 83), QVAALDANGIIVEGP (SEQ ID NO: 84), LDANGIIVEGPVPIT (SEQ ID NO: 85), LDANGII (SEQ ID NO: 53) or DANGI (SEQ ID NO: 54).

In certain embodiments, the antigen-binding protein specifically binds to human cadherin-17 (CDH17) at an epitope comprising the amino acid sequence of any of THNLQVAALDANGIIVEGPVPIT (SEQ ID NO: 82), THNLQVAALDANGII (SEQ ID NO: 83), QVAALDANGIIVEGP (SEQ ID NO: 84), LDANGIIVEGPVPIT (SEQ ID NO: 85), LDANGII (SEQ ID NO: 53) or DANGI (SEQ ID NO: 54).

In certain embodiments, the antigen-binding protein specifically binds to human cadherin-17 (CDH17) at an epitope comprising the amino acid sequence of any of THNLQVAALDANGIIVEGPVPIT (SEQ ID NO: 82), THNLQVAALDANGII (SEQ ID NO: 83), QVAALDANGIIVEGP (SEQ ID NO: 84), LDANGIIVEGPVPIT (SEQ ID NO: 85), LDANGII (SEQ ID NO: 53) or DANGI (SEQ ID NO: 54, wherein binding of the antigen-binding protein to the CDH17 epitope reduces or inhibits CDH17 activity.

In certain embodiments, the antigen-binding protein specifically binds to human cadherin-17 (CDH17) at an epitope comprising the amino acid sequence of any of THNLQVAALDANGIIVEGPVPIT (SEQ ID NO: 82), THNLQVAALDANGII (SEQ ID NO: 83), QVAALDANGIIVEGP (SEQ ID NO: 84), LDANGIIVEGPVPIT (SEQ ID NO: 85), LDANGII (SEQ ID NO: 53) or DANGI (SEQ ID NO: 54, thereby inhibiting tumor growth, reducing tumor size, preventing the recurrence of cancer and/or treating cancer in a subject diagnosed to be a over-expresser of CDH17.

The present disclosure provides a bispecific antigen-binding protein that binds CDH17 and a second antigen, wherein the antigen-binding protein that binds CDH17 is any one of the antigen-binding protein described herein. In some embodiments, the bispecific antigen-binding protein comprises: (a) a heavy chain variable region amino acid sequence set forth in Tables 1-4, or a variant sequence thereof which differs by only one or two amino acids or which has at least or about 70% sequence identity; (b) a light chain variable region amino acid sequence set forth in Tables 1-4, or a variant sequence thereof which differs by only one or two amino acids or which has at least or about 70% sequence identity; or (c) both (a) and (b). In some embodiments, the variant sequence has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity. In some embodiments, the bispecific antigen-binding protein comprises a Fc polypeptide. In some embodiments, the bispecific antigen-binding protein comprises a Fc polypeptide comprising an afucosylated glycan.

In various aspects, a bispecific antigen-binding protein binds CDH17 and a second antigen. In some embodiments, a bispecific antigen-binding protein comprises an antigen-binding fragment of an antibody specific for the second antigen. In various embodiments, the second antigen is a cell surface protein expressed by a T cell, optionally a component of the T-cell receptor (TCR), for example CD3. In some embodiments, the second antigen is CD3. In some embodiments, the second antigen is CD3E. In some embodiments, the CDH17-CD3 bispecific antibody is 07-0653-h43Bs, 07-0646-h7Bs or 07-0663-h7Bs.

In various embodiments, the second antigen is a costimulatory molecule which assists in T-cell activation, e.g., CD40 or 4-1BB (CD137). In various embodiments, the second antigen is an Fc receptor, optionally, a Fc gamma receptor, Fc-alpha receptor, or Fc-epsilon receptor. In some embodiments, the Fc receptor is CD64 (Fc-gamma RI), CD32 (Fc-gamma RIIA), CD16A (Fc-gamma RIIIA), CD16b (Fc-gamma RIIIb), FcERI, CD23 (Fc-epsilon RII), CD89 (Fc-epsilon RI), Fcα/μR, or FcRn. In some embodiments, the Fc receptor is CD16A.

In various embodiments, the second antigen is an immune checkpoint molecule, e.g., a protein involved in the immune checkpoint pathway, optionally, A2AR, B7-H3, B7-H4, BTLA, CTLA4, IDO, KIR, LAG3, NOX2, PD-1, TIM3, VISTA, or SIGLEC7. In some embodiments, the immune checkpoint molecule is PD-1, LAG3, TIM3, or CTLA4. In various embodiments, the bispecific antigen-binding protein comprises an scFv, a Fab, or a F (ab) 2′ of any of the presently disclosed CDH17 antibodies.

In various embodiments, the bispecific antigen-binding protein comprises an antigen-binding protein comprising a sequence set forth in Tables 1-4, or a variant sequence thereof which differs by only 1-5 amino acids or which has at least or about 70% sequence identity. In some embodiments, the variant sequence has at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity. In various embodiments, the bispecific antigen-binding protein comprises a structure of a nanobody, a diabody, a BiTE®, DART, TandAb, CrossMab, or HSAbody.

The present disclosure provides a conjugate comprising an antigen-binding protein or a bispecific antigen-binding protein described herein and a heterologous moiety. In some embodiments, the antigen-binding protein comprises the amino acid sequence set forth in Tables 1-4. In some embodiments, the conjugate comprises a cytotoxic agent or a chemotherapeutic agent. In some embodiments, the chemotherapeutic agent is an anti-mitotic agent which inhibits cell division by blocking tubulin polymerization. In some embodiments, the anti-mitotic agent is an auristatin. In some embodiments, the auristatin is MMAE.

In various embodiments, the conjugate of the present disclosure is conjugated to the antigen-binding protein via a cleavable linker. In some embodiments, the cleavable linker is VC-PAB (e.g., MC-VC-PAB).

In some embodiments, the conjugate comprises an antigen-binding protein that is an antibodythe antibody is a monoclonal antibody, optionally wherein the monoclonal antibody is an IgG antibody. In some embodiments, the antibody is a human antibody, humanized antibody, or a chimeric antibody.

In various embodiments, the conjugate of the present disclosure has an average number of units of the agent conjugated per antigen-binding protein in a range of 1 to 8, preferably wherein the average number of units of the agent conjugated per antigen-binding protein is in a range of 3-8. In some embodiments, the conjugate is a heterogeneous conjugate. In other embodiments, the conjugate is a homogeneous conjugate. In some embodiments, the conjugate comprises a heterologous moiety or an agent, wherein the agent is conjugated at a specific site of the antigen-binding protein. In some embodiments, the specific site is an unpaired cysteine residue.

The present disclosure also provides a fusion protein comprising an antigen-binding protein or a bispecific antigen-binding protein described herein. The present disclosure further provides a nucleic acid comprising a nucleotide sequence encoding an antigen-binding protein, a bispecific antigen-binding protein, a conjugate, or a fusion protein, of the present disclosure. The present disclosure provides a vector comprising the nucleic acid comprising a nucleotide sequence encoding an antigen binding protein, a conjugate, or a fusion protein, of the present disclosure. The present disclosure additionally provides a host cell comprising the nucleic acid or the vector of the present disclosure.

The present disclosure provides a method of producing an antigen-binding protein or a bispecific antigen-binding protein that binds to a CDH17 protein, comprising (i) culturing the host cell of the present disclosure in a cell culture medium, wherein the host cell comprises a nucleic acid comprising a nucleotide sequence encoding an antigen binding protein or a bispecific antigen-binding protein described herein, and (ii) harvesting the antigen-binding protein or a bispecific antigen-binding protein from the cell culture medium. Also, provided is a method of producing a fusion protein comprising an antigen-binding protein or a bispecific antigen-binding protein that binds to a CDH17 protein, comprising (i) culturing the host cell of the present disclosure in a cell culture medium, wherein the host cell comprises a nucleic acid comprising a nucleotide sequence encoding a fusion protein of the present disclosure, and (ii) harvesting the fusion protein from the cell culture medium.

The present disclosure furthermore provides a method of producing a pharmaceutical composition comprising combining an antigen-binding protein, a bispecific antigen-binding protein, a conjugate, a fusion protein, a nucleic acid, a vector, a host cell, of the present disclosure, or a combination thereof, and a pharmaceutically acceptable carrier, diluent or excipient. Also provided are pharmaceutical compositions comprising antigen-binding protein, a bispecific antigen-binding protein, a conjugate, a fusion protein, a nucleic acid, a vector, a host cell, of the present disclosure, or a combination thereof, and a pharmaceutically acceptable carrier, diluent or excipient.

Provided herein is a method of treating a subject with a CDH17-expressing cancer comprising administering to the subject a pharmaceutical composition described herein in an amount effective to treat the cancer. Also provided is a method of inhibiting tumor growth in a subject, comprising administering to the subject a pharmaceutical composition described herein in an amount effective to inhibit tumor growth. The present disclosure provides a method of reducing tumor size in a subject, comprising administering to the subject a pharmaceutical composition described herein in an amount effective to reduce tumor size. Further provided is a method of preventing the recurrence of cancer in a subject, comprising administering to the subject a pharmaceutical composition described herein in an amount effective to prevent the recurrence of cancer.

The present disclosure provides a method of detecting CDH17 in a sample, comprising contacting the sample with an antigen-binding protein, a bispecific antigen-binding protein, a conjugate, or a fusion protein, of the present disclosure, and assaying for an immunocomplex comprising the antigen-binding protein, conjugate or fusion protein bound to CDH17. Also provided herein is a method of diagnosing a CDH17-positive cancer in a subject, comprising contacting a biological sample comprising cells or tissue obtained from the subject with an antigen-binding protein, a bispecific antigen-binding protein, a conjugate, or a fusion protein, of the present disclosure, and assaying for an immunocomplex comprising the antigen-binding protein, conjugate or fusion protein bound to CDH17.

The present disclosure also provides a method of treating cancer in a subject diagnosed to be a low over-expresser of CDH17. In various embodiments, the method comprises administering to the subject a presently disclosed pharmaceutical composition in an amount effective to prevent the recurrence of cancer. In some aspects, the administering induces apoptosis in tumor cells, optionally, the administering induces apoptosis in cells expressing CDH17. In various aspects, the subject has a tumor and the tumor is semi-quantitatively categorized into one of four groups: high expressers, moderate expressers, low expressers, and non-expressers.

The present disclosure also provides a method of producing a conjugate. In some embodiments, the method comprises contacting the antigen-binding protein or a fusion protein thereof with an agent comprising a chemically reactive group that can react with any part of the antigen-binding protein or a fusion protein thereof. In some embodiments, the method comprises contacting the antigen-binding protein or a fusion protein thereof with an agent and a bifunctional linker that can bridge the linker and the antigen-binding protein or a fusion protein thereof. In some embodiments, the agent is a cytotoxic agent and/or a chemotherapeutic agent.

In some embodiments, the method of producing a conjugate comprises (a) culturing a host cell in a cell culture medium, wherein the host cell comprises a nucleotide sequence encoding an antigen binding protein or a fusion protein thereof, (b) harvesting the antigen-binding protein or the fusion protein from the cell culture medium, and (c) attaching the antigen binding protein or a fusion protein to a second moiety so as to produce the conjugate, wherein the second moiety is a cytotoxic agent or a chemotherapeutic agent.

### EXEMPLARY EMBODIMENTS

1. An antigen-binding protein comprising:
a. CDRs 1-3 derived from a heavy chain variable region comprising the amino acid sequence:
   QVQLVQSGAEVKKPGSSVKISCKVSGYTFTDHTIHWMRQAPG QGLEWIGYIFPRDDIVVYAQKFQGRATLTADKSTSTAYMELSS LRSEDTAVYYCARPPYYYSRNFYFDYWGQGTTLTVSS (SEQ ID NO: 49) or a variant sequence thereof which differs by only one or two amino acids or which has at least or about 85% sequence identity; and/or
b. CDRs 1-3 derived from a light chain variable region comprising the amino acid sequence:
   DIQMTQSPSSLSASVGDRVTITCRVSSIISSSKLHWYQQKPGKA PKPLIYGTSTLASGVPSRFSGSGSGTDYTLTISSLQPEDFATYYC QQWSNYPFTFGQGTKLEIK (SEQ ID NO: 50) or a variant sequence thereof which differs by only one or two amino acids or which has at least or about 85% sequence identity.

2. An antigen-binding protein comprising
a. CDRs 1-3 derived from a heavy chain variable region comprising the amino acid sequence:
   QVQLVQSGAEVKKPGASVKMSCKASGYTFTSYNMHWVRQA PGQGLEWIGAIYPGNGDTSYAQKFQGRATLTVDTSTSTAYME LSSLRSEDTAVYYCARGRGRYFEYWGQGTTLTVSS (SEQ ID NO: 45) or a variant sequence thereof which differs by only one or two amino acids or which has at least or about 85% sequence identity; and/or
b. CDRs 1-3 derived from a light chain variable region comprising the amino acid sequence:
   DIQLTQSPSSLSASVGDRVTMTCRASSSVSSSYLHWYQQKPGK APKLLIYSTSNLASGVPSRFSGSGSGTDYTLTISSVQPEDFATY YCQQYDSSPSTFGQGTKLEIK (SEQ ID NO: 46) or a variant sequence thereof which differs by only one or two amino acids or which has at least or about 85% sequence identity.

3. An antigen-binding protein comprising:
a. CDRs 1-3 derived from a heavy chain variable region comprising the amino acid sequence:
   QVQLVQSGAEVKKPGASVKVSCKASGYTFTDYYMNWVRQA PGQGLEWMGVIYPYSGGIGYAQKFQGRVTMTVDKSTSTAYM ELSSLRSEDTAVYYCARGRGDYFGLFDFWGQGTTVTVSS (SEQ ID NO: 47) or a variant sequence thereof which differs by only one or two amino acids or which has at least or about 85% sequence identity; and/or
b. CDRs 1-3 derived from a light chain variable region comprising the amino acid sequence:
   DIQLTQSPSSLSASVGDRVTITCRATSSLSYIHWYQQKPGKAPK PLIYEISKLASGVPSRFSGSGSGTDYTLTISSLQPEDFATYYCQQ WNYPFTFGQGTKLEIK (SEQ ID NO: 48) or a variant sequence thereof which differs by only one or two amino acids or which has at least or about 85% sequence identity.

4. An antigen-binding protein comprising:
a. a heavy chain CDR1 comprising the amino acid sequence of: GYTFTDHT (SEQ ID NO: 13) or a variant sequence thereof which differs by only one or two amino acids or which has at least or about 70% sequence identity;
b. a heavy chain CDR2 comprising the amino acid sequence of: IFPRDDIV (SEQ ID NO: 14) or a variant sequence thereof which differs by only one or two amino acids or which has at least or about 70% sequence identity;
c. a heavy chain CDR3 comprising the amino acid sequence of: ARPPYYYSRNFYFDY (SEQ ID NO: 15) or a variant sequence thereof which differs by only one or two amino acids or which has at least or about 70% sequence identity;
d. a light chain CDR1 comprising the amino acid sequence of: SIISSSK (SEQ ID NO: 16) or a variant sequence thereof which differs by only one or two amino acids or which has at least or about 70% sequence identity;
e. a light chain CDR2 comprising the amino acid sequence of: GTS (SEQ ID NO: 17) or a variant sequence thereof which differs by only one or two amino acids or which has at least or about 70% sequence identity;
f. a light chain CDR3 comprising the amino acid sequence of: QQWSNYPFT (SEQ ID NO: 18) or a variant sequence thereof which differs by only one or two amino acids or which has at least or about 70% sequence identity; or
g. a combination of any two or moreof (a)-(f).

5. An antigen-binding protein comprising:
a. a heavy chain CDR1 comprising the amino acid sequence of: GYTFTSYN (SEQ ID NO: 1) or a variant sequence thereof which differs by only one or two amino acids or which has at least or about 70% sequence identity;
b. a heavy chain CDR2 comprising the amino acid sequence of: IYPGNGDT (SEQ ID NO: 2) or a variant sequence thereof which differs by only one or two amino acids or which has at least or about 70% sequence identity;
c. a heavy chain CDR3 comprising the amino acid sequence of: ARGRGRYFEY (SEQ ID NO: 3) or a variant sequence thereof which differs by only one or two amino acids or which has at least or about 70% sequence identity;
d. a light chain CDR1 comprising the amino acid sequence of: SSVSSSY (SEQ ID NO: 4) or a variant sequence thereof which differs by only one or two amino acids or which has at least or about 70% sequence identity;
e. a light chain CDR2 comprising the amino acid sequence of: STS (SEQ ID NO: 5) or a variant sequence thereof which differs by only one or two amino acids or which has at least or about 70% sequence identity;
f. a light chain CDR3 comprising the amino acid sequence of:
   QQYDSSPST (SEQ ID NO: 6) or a variant sequence thereof which differs by only one or two amino acids or which has at least or about 70% sequence identity; or
g. a combination of any two or moreof (a)-(f).

6. An antigen-binding protein comprising:
a. a heavy chain CDR1 comprising the amino acid sequence of: GYTFTDYY (SEQ ID NO: 7) or a variant sequence thereof which differs by only one or two amino acids or which has at least or about 70% sequence identity;
b. a heavy chain CDR2 comprising the amino acid sequence of: IYPYSGGI (SEQ ID NO: 8) or a variant sequence thereof which differs by only one or two amino acids or which has at least or about 70% sequence identity;
c. a heavy chain CDR3 comprising the amino acid sequence of: ARGRGDYFGLFDF (SEQ ID NO: 9) or a variant sequence thereof which differs by only one or two amino acids or which has at least or about 70% sequence identity;
d. a light chain CDR1 comprising the amino acid sequence of: SSLSY (SEQ ID NO: 10) or a variant sequence thereof which differs by only one or two amino acids or which has at least or about 70% sequence identity;
e. a light chain CDR2 comprising the amino acid sequence of: EIS (SEQ ID NO: 11) or a variant sequence thereof which differs by only one or two amino acids or which has at least or about 70% sequence identity;
f. a light chain CDR3 comprising the amino acid sequence of: QQWNYPFT (SEQ ID NO: 12) or a variant sequence thereof which differs by only one or two amino acids or which has at least or about 70% sequence identity; or
g. a combination of any two or moreof (a)-(f).

7. The antigen-binding protein of 4, additionally comprising:
a. a heavy chain FR1 comprising the amino acid sequence:
   QVQLVQSGAEVKKPGSSVKISCKVS (SEQ ID NO: 37) or a variant sequence thereof which differs by only one or two amino acids or which has at least or about 85%, 90% or 95% sequence identity;
b. a heavy chain FR2 comprising the amino acid sequence: IHWMRQAPGQGLEWIGY (SEQ ID NO: 38) or a variant sequence thereof which differs by only one or two amino acids or which has at least or about 85%, 90% or 95% sequence identity;
c. a heavy chain FR3 comprising the amino acid sequence: VYAQKFQGRATLTADKSTSTAYMELSSLRSEDTAVYYC (SEQ ID NO: 39) or a variant sequence thereof which differs by only one or two amino acids or which has at least or about 85%, 90% or 95% sequence identity;
d. a heavy chain FR4 comprising the amino acid sequence: WGQGTTLTVSS (SEQ ID NO: 40) or a variant sequence thereof which differs by only one or two amino acids or which has at least or about 85%, 90% or 95% sequence identity;
e. a light chain FR1 comprising the amino acid sequence: DIQMTQSPSSLSASVGDR VTITCRVS (SEQ ID NO: 41) or a variant sequence thereof which differs by only one or two amino acids or which has at least or about 85%, 90% or 95% sequence identity;
f. a light chain FR2 comprising the amino acid sequence: LHWYQQKPGKAPKPLIY (SEQ ID NO: 42) or a variant sequence thereof which differs by only one or two amino acids or which has at least or about 85%, 90% or 95% sequence identity;
g. a light chain FR3 comprising the amino acid sequence: TLASGVPSRFSGSGSGTDYTLTISSLQPEDFATYYC (SEQ ID NO: 43) or a variant sequence thereof which differs by only one or two amino acids or which has at least or about 85%, 90% or 95% sequence identity;
h. a light chain FR4 comprising the amino acid sequence: FGQGTKLEIK (SEQ ID NO: 44) or a variant sequence thereof which differs by only one or two amino acids or which has at least or about 85%, 90% or 95% sequence identity; or
i. a combination of any two or moreof (a)-(h).

8. The antigen-binding protein of 5, additionally comprising:
a. a heavy chain FR1 comprising the amino acid sequence: QVQLVQSGAEVKKPGASVKMSCKAS (SEQ ID NO: 21) or a variant sequence thereof which differs by only one or two amino acids or which has at least or about 85%, 90% or 95% sequence identity;
b. a heavy chain FR2 comprising the amino acid sequence: MHWVRQAPGQGLEWIGA (SEQ ID NO: 22) or a variant sequence thereof which differs by only one or two amino acids or which has at least or about 85%, 90% or 95% sequence identity;
c. a heavy chain FR3 comprising the amino acid sequence: SYAQKFQGRATLTVDTSTSTAYMELSSLRSEDTAVYYC (SEQ ID NO: 23) or a variant sequence thereof which differs by only one or two amino acids or which has at least or about 85%, 90% or 95% sequence identity;
d. a heavy chain FR4 comprising the amino acid sequence: WGQGTTLTVSS (SEQ ID NO: 24) or a variant sequence thereof which differs by only one or two amino acids or which has at least or about 85%, 90% or 95% sequence identity;
e. a light chain FR1 comprising the amino acid sequence: DIQLTQSPSSLSASVGDRVTMTCRAS (SEQ ID NO: 25) or a variant sequence thereof which differs by only one or two amino acids or which has at least or about 85%, 90% or 95% sequence identity;
f. a light chain FR2 comprising the amino acid sequence: LHWYQQKPGKAPKLLIY (SEQ ID NO: 26) or a variant sequence thereof which differs by only one or two amino acids or which has at least or about 85%, 90% or 95% sequence identity;
g. a light chain FR3 comprising the amino acid sequence: NLASGVPSRFSGSGSGTDYTLTISSVQPEDFATYYC (SEQ ID NO: 27) or a variant sequence thereof which differs by only one or two amino acids or which has at least or about 85%, 90% or 95% sequence identity;
h. a light chain FR4 comprising the amino acid sequence of: FGQGTKLEIK (SEQ ID NO: 28) or a variant sequence thereof which differs by only one or two amino acids or which has at least or about 85%, 90% or 95% sequence identity; or
i. a combination of any two or moreof (a)-(h).

9. The antigen-binding protein of 6, additionally comprising:
a. a heavy chain FR1 comprising the amino acid sequence: QVQLVQSGAEVKKPGASVKVSCKAS (SEQ ID NO: 29) or a variant sequence thereof which differs by only one or two amino acids or which has at least or about 85%, 90% or 95% sequence identity;
b. a heavy chain FR2 comprising the amino acid sequence of: MNWVRQAPGQGLEWMGV (SEQ ID NO: 30) or a variant sequence thereof which differs by only one or two amino acids or which has at least or about 85%, 90% or 95% sequence identity;
c. a heavy chain FR3 comprising the amino acid sequence: GYAQKFQGR VTMTVDKSTSTAYMELSSLRSEDTAVYYC (SEQ ID NO: 31) or a variant sequence thereof which differs by only one or two amino acids or which has at least or about 85%, 90% or 95% sequence identity;
d. a heavy chain FR4 comprising the amino acid sequence: WGQGTTVTVSS (SEQ ID NO: 32) or a variant sequence thereof which differs by only one or two amino acids or which has at least or about 85%, 90% or 95% sequence identity;
e. a light chain FR1 comprising the amino acid sequence: DIQLTQSPSSLSASVGDRVTITCRAT (SEQ ID NO: 33) or a variant sequence thereof which differs by only one or two amino acids or which has at least or about 85%, 90% or 95% sequence identity;
f. a light chain FR2 comprising the amino acid sequence: IHWYQQKPGKAPKPLIY (SEQ ID NO: 34) or a variant sequence thereof which differs by only one or two amino acids or which has at least or about 85%, 90% or 95% sequence identity;
g. a light chain FR3 comprising the amino acid sequence: KLASGVPSRFSGSGSGTDYTLTISSLQPEDFATYYC (SEQ ID NO: 35) or a variant sequence thereof which differs by only one or two amino acids or which has at least or about 85%, 90% or 95% sequence identity;
h. a light chain FR4 amino acid sequence of: FGQGTKLEIK (SEQ ID NO: 36) or a variant sequence thereof which differs by only one or two amino acids or which has at least or about 85%, 90% or 95% sequence identity; or
i. a combination of any two or moreof (a)-(h).

10. An antigen-binding protein comprising:
a. a heavy chain variable region comprising the amino acid sequence of: QVQLVQSGAEVKKPGSSVKISCKVSGYTFTDHTIHWMRQAPG QGLEWIGYIFPRDDIVVYAQKFQGRATLTADKSTSTAYMELSS LRSEDTAVYYCARPPYYYSRNFYFDYWGQGTTLTVSS (SEQ ID NO: 49) or a variant sequence thereof which differs by only 1-5 amino acids or which has at least or about 85%, 90%, 95%, 98% or 99% sequence identity; and/or
b. a light chain variable region comprising the amino acid sequence: DIQMTQSPSSLSASVGDRVTITCRVSSIISSSKLHWYQQKPGKA PKPLIYGTSTLASGVPSRFSGSGSGTDYTLTISSLQPEDFATYYC QQWSNYPFTFGQGTKLEIK (SEQ ID NO: 50) or a variant sequence thereof which differs by only 1-5 amino acids or which has at least or about 85%, 90%, 95%, 98% or 99% sequence identity.

11. An antigen-binding protein comprising:
a. a heavy chain variable region comprising the amino acid sequence: QVQLVQSGAEVKKPGASVKMSCKASGYTFTSYNMHWVRQA PGQGLEWIGAIYPGNGDTSYAQKFQGRATLTVDTSTSTAYME LSSLRSEDTAVYYCARGRGRYFEYWGQGTTLTVSS (SEQ ID NO: 45) or a variant sequence thereof which differs by only 1-5 amino acids or which has at least or about 85%, 90%, 95%, 98% or 99% sequence identity; and/or
b. a light chain variable region comprising the amino acid sequence: DIQLTQSPSSLSASVGDRVTMTCRASSSVSSSYLHWYQQKPGK APKLLIYSTSNLASGVPSRFSGSGSGTDYTLTISSVQPEDFATY YCQQYDSSPSTFGQGTKLEIK (SEQ ID NO: 46) or a variant sequence thereof which differs by only 1-5 amino acids or which has at least or about 85%, 90%, 95%, 98% or 99% sequence identity.

12. An antigen-binding protein comprising:
a. a heavy chain variable region comprising the amino acid sequence: QVQLVQSGAEVKKPGASVKVSCKASGYTFTDYYMNWVRQA PGQGLEWMGVIYPYSGGIGYAQKFQGRVTMTVDKSTSTAYM ELSSLRSEDTAVYYCARGRGDYFGLFDFWGQGTTVTVSS (SEQ ID NO: 47) or a variant sequence thereof which differs by only 1-5 amino acids or which has at least or about 85%, 90%, 95%, 98% or 99% sequence identity; and/or
b. a light chain variable region comprising the amino acid sequence: DIQLTQSPSSLSASVGDRVTITCRATSSLSYIHWYQQKPGKAPK PLIYEISKLASGVPSRFSGSGSGTDYTLTISSLQPEDFATYYCQQ WNYPFTFGQGTKLEIK (SEQ ID NO: 48) or a variant sequence thereof which differs by only 1-5 amino acids or which has at least or about 85%, 90%, 95%, 98% or 99% sequence identity.

13. An antigen-binding protein that specifically binds to human cadherin-17 (CDH17) comprising:
a. a heavy chain CDR1 comprising the amino acid sequence of: GYTFXDXT (SEQ ID NO: 55) wherein X at position 5 is N, S, R, Q, A or T, and X at position 7 is H, W, Y or F;
b. a heavy chain CDR2 comprising the amino acid sequence of: IFPRDDIV (SEQ ID NO: 14) or a variant sequence thereof which differs by only one or two amino acids or which has at least or about 70% sequence identity;
c. a heavy chain CDR3 comprising the amino acid sequence of: ARPPYYYSRNFYFDY (SEQ ID NO: 15) or a variant sequence thereof which differs by only one or two amino acids or which has at least or about 70% sequence identity;
d. a light chain CDR1 comprising the amino acid sequence of: SIISSSK (SEQ ID NO: 16) or a variant sequence thereof which differs by only one or two amino acids or which has at least or about 70% sequence identity;
e. a light chain CDR2 comprising the amino acid sequence of: GTS (SEQ ID NO: 17) or a variant sequence thereof which differs by only one or two amino acids;
f. a light chain CDR3 comprising the amino acid sequence of: QQWSNYPFT (SEQ ID NO: 18) or a variant sequence thereof which differs by only one or two amino acids or which has at least or about 70% sequence identity; or
g. a combination of any two or moreof (a)-(f).

14. An antigen-binding protein that specifically binds to human cadherin-17 (CDH17) comprising:
a. a heavy chain CDR1 comprising the amino acid sequence of: GYTFXSXN (SEQ ID NO: 56) wherein X at position 5 is N, S, R, Q, A or T, and X at position 7 is H, W, Y or F;
b. a heavy chain CDR2 comprising the amino acid sequence of: IYPGNGDT (SEQ ID NO: 2) or a variant sequence thereof which differs by only one or two amino acids or which has at least or about 70% sequence identity;
c. a heavy chain CDR3 comprising the amino acid sequence of: ARGRGRYFEY (SEQ ID NO: 3) or a variant sequence thereof which differs by only one or two amino acids or which has at least or about 70% sequence identity;
d. a light chain CDR1 comprising the amino acid sequence of: SSVSSSY (SEQ ID NO: 4) or a variant sequence thereof which differs by only one or two amino acids or which has at least or about 70% sequence identity;
e. a light chain CDR2 comprising the amino acid sequence of: STS (SEQ ID NO: 5) or a variant sequence thereof which differs by only one or two amino acids;
f. a light chain CDR3 comprising the amino acid sequence of: QQYDSSPST (SEQ ID NO: 6) or a variant sequence thereof which differs by only one or two amino acids or which has at least or about 70% sequence identity; or
g. a combination of any two or moreof (a)-(f).

15. An antigen-binding protein that specifically binds to human cadherin-17 (CDH17) comprising:
a. a heavy chain CDR1 comprising the amino acid sequence of: GYTFXDXY (SEQ ID NO: 57) wherein X at position 5 is N, S, R, Q, A or T, and X at position 7 is H, W, Y or F;
b. a heavy chain CDR2 comprising the amino acid sequence of: IYPYSGGI (SEQ ID NO: 8) or a variant sequence thereof which differs by only one or two amino acids or which has at least or about 70% sequence identity;
c. a heavy chain CDR3 comprising the amino acid sequence of: ARGRGDYFGLFDF (SEQ ID NO: 9) or a variant sequence thereof which differs by only one or two amino acids or which has at least or about 70% sequence identity;
d. a light chain CDR1 comprising the amino acid sequence of: SSLSY (SEQ ID NO: 10) or a variant sequence thereof which differs by only one or two amino acids or which has at least or about 70% sequence identity;
e. a light chain CDR2 comprising the amino acid sequence of: EIS (SEQ ID NO: 11) or a variant sequence thereof which differs by only one or two amino acids;
f. a light chain CDR3 comprising the amino acid sequence of: QQWNYPFT (SEQ ID NO: 12) or a variant sequence thereof which differs by only one or two amino acids or which has at least or about 70% sequence identity; or
g. a combination of any two or moreof (a)-(f).

16. The antigen-binding protein of any one of the above or those described herein, wherein the variant sequence has at least about 80%, 85%, 90%, 95%, 98% or 99% sequence identity.

17. An antigen-binding protein that specifically binds to human cadherin-17 (CDH17) comprising:
a. an antibody heavy chain comprising the amino acid sequence of:

(SEQ ID NO: 94)
QVQLVQSGAEVKKPGSSVKISCKVSGYTFTDHTIHWMRQAPGQ
GLEWIGYIFPRDDIVVYAQKFQGRATLTADKSTSTAYMELSSLRS
EDTAVYYCARPPYYYSRNFYFDYWGQGTTLTVSSASTKGPSVFP
LAPSSKSTSGGTAALGCLVKDYFPEPVTVSWNSGALTSGVHTFP
AVLQSSGLYSLSSVVTVPSSSLGTQTYICNVNHKPSNTKVDKKVE
PKSCDKTHTCPPCPAPELLGGPSVFLFPPKPKDTLMISRTPEVTCV
VVDVSHEDPEVKFNWYVDGVEVHNAKTKPREEQYNSTYRVVS
VLTVLHQDWLNGKEYKCKVSNKALPAPIEKTISKAKGQPREPQV
YTLPPSRDELTKNQVSLTCLVKGFYPSDIAVEWESNGQPENNYK
TTPPVLDSDGSFFLYSKLTVDKSRWQQGNVFSCSVMHEALHNHY
TQKSLSLSPGK;

and
b. an antibody light chain comprising the amino acid sequence of:

(SEQ ID NO: 95)
DIQMTQSPSSLSASVGDRVTITCRVSSIISSSKLHWYQQKPGKAPK
PLIYGTSTLASGVPSRFSGSGSGTDYTLTISSLQPEDFATYYCQQW
SNYPFTFGQGTKLEIKRTVAAPSVFIFPPSDEQLKSGTASVVCLLN
NFYPREAKVQWKVDNALQSGNSQESVTEQDSKDSTYSLSSTLTL
SKADYEKHKVYACEVTHQGLSSPVTKSFNRGEC.

18. The antigen-binding protein of any one of the above or those described herein, wherein:
a. the antigen-binding protein binds to a human cadherin-17 (CDH17) protein (SEQ ID NO: 19);
b. the antigen-binding protein binds an extracellular domain of human CDH17 with a dissociation constant (K_{D}) of about less than 10 nM, 5 nM, 2.5 nM, 1 nM, 0.5 nM, or 0.25 nM;
c. the antigen-binding protein preferentially binds human CDH17 over mouse CDH17 (SEQ ID NO: 20);
d. the antigen-binding protein does not bind mouse CDH17; or
e. a combination thereof.

19. The antigen-binding protein of any one of the above or those described herein, which specifically binds to human cadherin-17 (CDH17) or a polypeptide comprising the amino acid sequence of any one of

(SEQ ID NO: 82)
THNLQVAALDANGIIVEGPVPIT,
(SEQ ID NO: 83)
THNLQVAALDANGII,
(SEQ ID NO: 84)
QVAALDANGIIVEGP,
(SEQ ID NO: 85)
LDANGIIVEGPVPIT,
(SEQ ID NO: 53)
LDANGII,
and
(SEQ ID NO: 54)
DANGI.

20. The antigen-binding protein of any one of the above or those described herein, wherein binding of the antigen-binding protein to CDH17 reduces, interferes with, or inhibits the CDH17 activity.

21. The antigen-binding protein of any one of the above or those described herein, which is an antibody or antigen-binding antibody fragment.

22. The antigen-binding protein of 21, wherein the antibody is a monoclonal antibody.

23. The antigen-binding protein of 21 or 22, wherein the antibody is a chimeric antibody, a human antibody, or a humanized antibody.

24. The antigen-binding protein of any one of 21-23, wherein the antibody is an IgG.

25. The antigen-binding protein of 24, wherein the IgG is selected from IgG1, IgG2, IgG3 and IgG4.

26. The antigen-binding protein of any one of the above or those described herein, wherein the antigen-binding protein is a bispecific antigen-binding protein or a bispecific T cell engager (BiTE).

27. The antigen-binding protein of 26, wherein the bispecific antigen-binding protein or the BiTE comprises the amino acid sequence set forth in
a. SEQ ID NO: 45 and SEQ ID NO: 46;
b. SEQ ID NO: 47 and SEQ ID NO: 48; or
c. SEQ ID NO: 49 and SEQ ID NO: 50, optionally wherein the bispecific antigen-binding protein is selected from 07-0653-h43Bs, 07-0646-h7Bs, and 07-0663-h7Bs.

28. The antigen-binding protein of 21, wherein the antigen-binding antibody fragment is selected from scFv, F(ab′) 2, Fab, Fab′, and Fv.

29. The antigen-binding protein of 28, wherein the scFv is 07-0653-h43scfv, 07-0646-h7scfv, or 07-0663-h7scfv.

30. The antigen-binding protein of any one of the above or those described herein, which inhibits tumor growth in xenograft mice injected with human cancer cells.

31. The antigen-binding protein of any one of the above or those described herein, comprising a Fc polypeptide comprising an afucosylated glycan.

32. A conjugate comprising an antigen-binding protein of any one of the above or those described herein or those described herein.

33. The conjugate of 32 comprising a detectable marker, a cytotoxic agent, or a chemotherapeutic agent.

34. The conjugate of 33, wherein the chemotherapeutic agent is an anti-mitotic agent which inhibits cell division by blocking tubulin polymerization.

35. The conjugate of 34, wherein the anti-mitotic agent is an auristatin.

36. The conjugate of 35, wherein the auristatin is MMAE.

37. The conjugate of any one of 33-36, wherein the agent or the marker is conjugated to the antigen-binding protein via a cleavable linker or a non-cleavable linker.

38. The conjugate of 37, wherein the cleavable linker is VC-PAB.

39. The conjugate of any one of 32-38, wherein the antigen-binding protein is an antibody,

40. The conjugate of 39, wherein the antibody is a monoclonal antibody.

41. The conjugate of 39 or 40, wherein the antibody is a human antibody, a humanized antibody, or a chimeric antibody.

42. The conjugate of any one of 39-41, wherein the antibody is an IgG antibody, optionally wherein the IgG is IgG1, IgG2, IgG3, or IgG4.

43. The conjugate of any one of 32-42, wherein an average number of units of the agent conjugated per antigen-binding protein is in a range of 1 to 8, preferably wherein the average number of units of the agent conjugated per antigen-binding protein is (a) in a range of 3-8, or (b) 4.

44. The conjugate of any one of 32-43, wherein the conjugate is a heterogeneous conjugate.

45. The conjugate of any one of 32-43, wherein the conjugate is a homogeneous conjugate.

46. The conjugate of any one of 32-45, wherein the agent is conjugated at a specific site of the antigen-binding protein.

47. The conjugate of 46, wherein the specific site is an unpaired cysteine residue.

48. The conjugate of any one of 32-47, wherein the conjugate comprises a polypeptide comprising the amino acid sequence set forth in SEQ ID NO: 45 and SEQ ID NO: 46 conjugated to VC-PAB-MMAE.

49. The conjugate of any one of 32-47, wherein the conjugate comprises a polypeptide comprising the amino acid sequence set forth in SEQ ID NO: 47 and SEQ ID NO: 48 conjugated to VC-PAB-MMAE.

50. The conjugate of any one of 32-47, wherein the conjugate comprises a polypeptide comprising the amino acid sequence set forth in SEQ ID NO: 49 and SEQ ID NO: 50 conjugated to VC-PAB-MMAE.

51. The conjugate of any one of 32-47, wherein the conjugate comprises a polypeptide comprising the amino acid sequence set forth in SEQ ID NO: 94 and SEQ ID NO: 95 conjugated to VC-PAB-MMAE.

52. A fusion protein comprising an antigen-binding protein of any one of the above or those described herein.

53. A nucleic acid comprising a nucleotide sequence encoding an antigen binding protein of any one of 1-31, a conjugate of 32-51, or a fusion protein of 52.

54. The nucleic acid of 53, wherein the nucleic acid is a cDNA.

55. A vector (e.g., expression vector) comprising the nucleic acid of 53 or 54.

56. The vector of 55, additionally comprising an internal ribosome entry site (IRES).

57. A host cell comprising the nucleic acid of 53 or 54, or the vector of 55 or 56.

58. The host cell of 57, wherein the host cell is a bacterial cell.

59. The host cell of 57, wherein the host cell is a eukaryotic cell.

60. The host cell of 59, wherein the eukaryotic cell is a mammalian cell.

61. The host cell of 60, wherein the mammalian cell is a Chinese hamster ovary (CHO) cell.

62. A method of producing an antigen-binding protein that binds to a cadherin-17 (CDH17) protein, comprising (i) culturing the host cell of any one of 57-61 in a cell culture medium, and (ii) harvesting the antigen-binding protein from the cell culture medium.

63. A method of producing a fusion protein comprising an antigen-binding protein that binds to a cadherin-17 (CDH17) protein, comprising (i) culturing the host cell of any one of 57-61 in a cell culture medium, and (ii) harvesting the fusion protein from the cell culture medium.

64. A method of producing a pharmaceutical composition, the method comprising combining (a) an antigen-binding protein of any one of 1-31, a conjugate of any one of 32-51, a fusion protein of 52, a nucleic acid of 53 or 54, a vector of 55 or 56, a host cell of 57, 59, or 60, or any combination thereof; and (b) a pharmaceutically acceptable carrier, diluent and/or excipient

65. A pharmaceutical composition comprising (a) an antigen-binding protein of any one of 1-31, a conjugate of any one of 32-51, a fusion protein of 52, a nucleic acid of 53 or 54, a vector of 55 or 56, a host cell of 57, 59, or 60, or any combination thereof; and (b) a pharmaceutically acceptable carrier, diluent and/or excipient.

66. A method of treating a subject with a CDH17-expressing cancer comprising administering to the subject a pharmaceutical composition of 65 to treat the cancer.

67. A method of inhibiting tumor growth in a subject, comprising administering to the subject a pharmaceutical composition of 65 to inhibit tumor growth.

68. A method of reducing tumor size in a subject, comprising administering to the subject a pharmaceutical composition of 65 to reduce tumor size.

69. A method of preventing the recurrence of cancer in a subject, comprising administering to the subject a pharmaceutical composition of 65 to prevent the recurrence of cancer.

70. A method of treating cancer in a subject diagnosed to be a over-expresser of CDH17, comprising administering to the subject a pharmaceutical composition of 65 to prevent the recurrence of cancer

71. The method of any one of 66-70, wherein the administering induces apoptosis in tumor cells.

72. The method of any one of 66-70, wherein the administering induces apoptosis in cells expressing CDH17.

73. A method of detecting cadherin-17 (CDH17) in a sample, comprising contacting the sample with an antigen-binding protein of any one of 1-31, a conjugate of any one of 32-51, or a fusion protein of 52; and assaying for an immunocomplex comprising the antigen-binding protein, conjugate or fusion protein bound to CDH17.

74. A method of diagnosing a cadherin-17 (CDH17)-positive cancer in a subject, comprising contacting a biological sample comprising cells or tissue obtained from the subject with an antigen-binding protein of any one of 1-31, a conjugate of any one of 32-51, or a fusion protein of 52; and assaying for an immunocomplex comprising the antigen-binding protein, conjugate or fusion protein bound to CDH17.

75. The method of 74, further comprising treating the subject diagnosed to have CDH17-positive cancer by administering to the subject an antigen-binding protein of any one of 1-31, a conjugate of any one of 32-51, or a fusion protein of 52.

76. A method of producing a conjugate of any one of the above or those described herein, the method comprising
a. culturing a host cell of any one of 57-61 in a cell culture medium, wherein the host cell comprises a nucleic acid encoding an antigen binding protein or a fusion protein thereof,
b. harvesting the antigen-binding protein or the fusion protein from the cell culture medium, and
c. attaching the antigen binding protein or a fusion protein to a heterologous moiety so as to produce the conjugate, optionally wherein the heterologous moiety is a cytotoxic agent or a chemotherapeutic agent.

77. A method of activating a T cell to target a CDH17-expressing tumor or cancer cell in a subject, the method comprising administering to the subject a bispecific T cell engager (BiTE) that comprises a first scFv that binds CDH17 and a second scFv which binds CD3, wherein the first scFv that binds CDH17 comprises the VH region and VL region of the antigen-binding protein of any one of the above or those described herein.

78. The method of 77, wherein the BiTE comprises the amino acid sequence set forth in
a. SEQ ID NO: 45 and SEQ ID NO: 46;
b. SEQ ID NO: 47 and SEQ ID NO: 48; or
c. SEQ ID NO: 49 and SEQ ID NO: 50.

79. The method of 77 or 78, wherein the BiTE is selected from 07-0646-h7Bs, 07-0653-h43Bs, and 07-0663-h7Bs, optionally wherein the BiTe is 07-0653-h43Bs.

80. A method of inducing an antibody-dependent cell-mediated cytotoxicity (ADCC) response against a CDH17-expressing tumor or cancer cell in a subject, the method comprising administering to the subject an antigen-binding protein that binds CDH17, wherein the antigen-binding protein comprises an Fc effector function; and the VH region and VL region of the antigen-binding protein of any one of the above or those described herein.

81. The method of 80, wherein the antigen-binding protein that binds CDH17 is 07-646-h7 or 07-663-h7.

82. The method of any one of the above or those described herein, wherein the subject is a mammal, optionally a dog, a cat, a mouse, or a human.

The following examples are given merely to illustrate the present disclosure and not in any way to limit its scope.

### EXAMPLES

### Example 1

This example describes the production of CDH17 specific antibodies.

Mice were immunized with 3T3 cells overexpressing a full length, epitope-tagged human CDH17 protein using a mammalian expression vector encoding a human CDH17 fusion protein.

Splenocytes were harvested from the immunized mice and fused with myeloma lines by BTX Electrofusion (BTX, Holliston, MA) to generate hydridomas. 7000 primary hybridoma cultures were generated and cultured in 384-well plates. The ability of the antibodies to bind peptides and/or human cancer cells that expressed CDH17 was assessed by ELISA assays and/or flow cytometry. Approximately 2000 potential positive antibodies were re-arrayed into 96 well plates further screened by flow cytometry against endogenous and artificial cell line models.

Hybridomas positive for producing antibodies that bind human CDH17 protein were identified, and nucleic acid sequences encoding immunoglobulin light and heavy chain directed against human CDH17 protein isolated. These nucleic acid sequences were used to produce CDH17 antibodies formatted as human full-length IgG antibodies (e.g., IgG1) using ExpiCHO™ expression. The heavy and light chain variable regions of the antibodies were cloned into an antibody expression vector which was engineered in the lab based on a pcDNA™3.4-TOPOR vector (Catalog Number: A14697, ThermoFisher Scientific, USA). Transfection of the antibody expression vector into CHO cells, according to protocol provided in the kit (ExpiCHO™ Expression System, Catalog Number: A29133, ThermoFisher Scientific, USA)), resulted in production of a bicistronic mRNA in which an IRES drives the expression of the second immunoglobulin chain. Notably, the C-terminal lysine of the human IgG1 constant region of the chimeric CDH17 antibodies, but not of the humanized CDH17 antibodies, was removed. Chimeric and humanized antibodies both contained the human IgG1 constant region and a kappa constant region. The produced antibodies were purified using protein A/G resins. Cell surface binding of the antibodies to CDH17 was determined by FACS in which CDH17 antibodies were directly conjugated with Alexa Fluor® 647 NHS Ester (Succinimidyl Ester), Cat #A20106 (ThermoFisher Scientific) following the manufacturer's protocol. The antibody binding to CDH17 was also determined and its EC50 was measured using recombinant CDH17 extracellular domain (ECD) with 6x His tag (SEQ ID NO: 52).

CDH17-expressing cells were used in FACS assays to determine the CDH17 antibody's ability to bind to CDH17 on the surface of cells and to cross-react with other CDH17 family members. HEK293 T cells engineered to express human, monkey, mouse or rat CDH17 fused to a fluorescent protein (mGFP or moxGFP) were used as artificial models of CDH17 expression. SNU-C1 (a human colorectal cancer cell line), PaTu8988s (a human pancreatic cancer cell line), HPAF-II (a human pancreatic cancer cell line) and LS513 (a human colorectal cancer cell line) were used as endogenous models of CDH17 expression, while H524 (a human small cell lung carcinoma cell line), COR-L279 (a human small cell lung carcinoma cell line) and M202 (a human melanoma cell line) were used as endogenous models lacking CDH17 expression.

For each type of cell tested and for each mAb, cells were detached from the surface of the culture flasks by versene (instead of trypsin) in order to protect the cell surface proteins. The detached cells were then incubated with Alexa Fluor®-labeled CDH17 mAbs for 30 min in the dark on ice at a pre-determined concentration. The CDH17 mAbs were directly labeled with Alexa Fluor® 647 NHS Ester (Succinimidyl Ester). Alternatively, unlabeled CDH17 antibodies may be detected using a fluorescently labeled secondary antibody. After washing, the cells were read by a BD Accuri™ Flow Cytometer C6 to detect antibody-antigen protein binding in channel FL4H. Each antibody was tested at varied concentrations to establish a dose-fluorescence curve.

### Example 2

This example demonstrates the humanization of antibodies of the present disclosures.

Antibodies were selected for humanization analysis. The heavy chain variable (VH) and light chain variable (VL) sequences of mouse monoclonal anti-CDH17 antibodies were compared to a library of known human germline sequences from human VH genes and human VLkappa genes (IMGT® the international ImMunoGeneTics information system® www.imgt.org; founder and director: Marie-Paule Lefranc, Montpellier, France); the databases used were IMGT human VH genes (F+ORF, 273 germline sequences) and IMGT human VLkappa genes (F+ORF, 74 germline sequences). The acceptor human germline was chosen from those closest in sequence to the parental antibody.

Alteration of human germline framework (i.e., non-CDR residues in VH and VL; abbreviated as FR) positions to corresponding parental murine sequence might be required to optimize binding of the humanized antibody. The sequences for versions of humanized antibodies are provided in the figures.

Humanized antibodies, CDH17-646-h7, CDH17-653-h42, CDH17-653-h43, CDH17-657-h16, CDH17-663-h7, CDH17-670-h12, CDH17-675-h11 and CDH17-683-h6, were constructed and expressed as essentially described in Example 1. FACS assays were carried out to determine relative antigen binding strengths of the humanized antibodies (at 1 μg) for binding to CDH17 overexpressed in HEK293T cell line. Corresponding Parental antibodies (antibodies prior to humanization) were used as controls and designated with “chim”.

Based on the in vitro antigen binding data, three humanized antibodies (07-0646-h7 (also designated as CDH17-646-h7), 07-0653-h43 (also designated as CDH17-653-h43), and 07-0663-h7 (also designated as CDH17-663-h7)) were selected for further testing and development. The antibodies were derived from CDH17-646 (also designated as CDH17-646-m), CDH17-653 (also designated as CDH17-653-m), and CDH17-663.

In vivo binding studies of the humanized versions of chimeric monoclonal anti-CDH17 antibodies, CDH 17-646, CDH 17-653, CDH 17-657, CDH 17-663, CDH 17-670, CDH 17-675, and CDH 17-683, as well as chimeric monoclonal anti-CDH17 antibodies and as antibody-drug conjugates (ADC; antibody-MMAE conjugate) were carried out in xenograft mice injected with CDH17-positive human colorectal cancer cell lines SNU-C1 and LS513, CDH17-positive human pancreatic cancer cell lines HPAF-II and PaTu8998s, CDH17-negative small cell lung carcinoma cell lines H524 and CORL279, and CDH17-negative human melanoma cell line M202. The chimeric monoclonal CDH17 antibodies comprise mouse anti-CDH17 antibody VH and VL regions fused to human IgG1 constant region and human kappa light chain constant region, respectively, wherein the different numerical designations reflect different mouse anti-CDH17 antibody isolates. Briefly, xenograft models of different human cancer cell lines were established in six-week-old CD-1 athymic nude mice (Charles River Laboratories). After tumors reached an average size of 150 to 300 mm³, mice were randomized into treatment groups. Humanized antibodies were diluted in sterile saline to a working concentration of 1 mg/ml for intravenous tail vein (IV) injection. Tumor xenografts were measured with calipers three times a week, and tumor volume in mm³ was determined by multiplying height× width× length. Mice were treated for 2-7 weeks. At the end of study, animals were euthanized and tumor tissue was excised and divided to be stored as snap-frozen or formalin fixed paraffin embedded (FFPE) tissue for biomarker analysis.

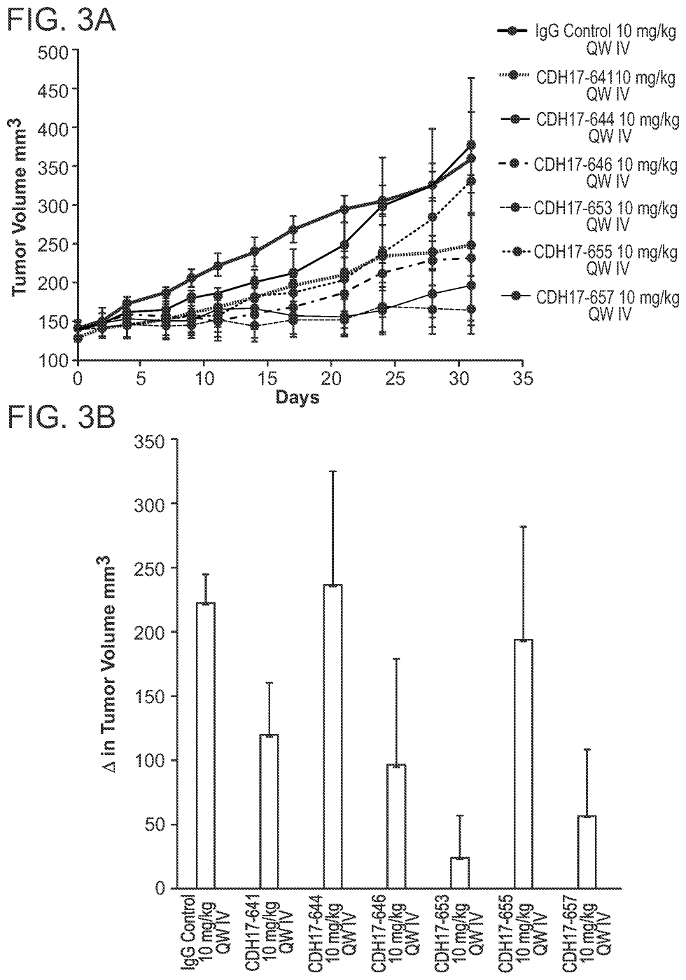

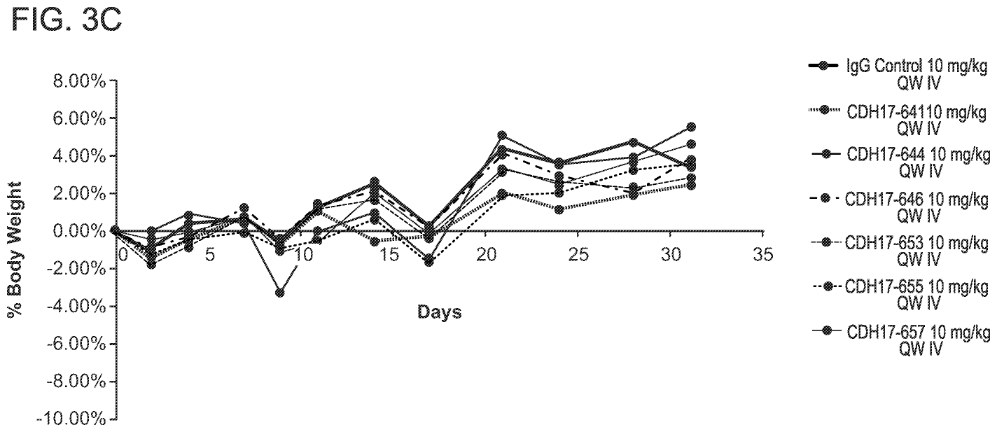

The results of the xenograft assays are shown in FIGS. 3-10.

FIG. 3 shows the xenograft assay results for parental mouse monoclonal antibody directed against CDH17, wherein different anti-CDH17 antibody has different efficacy in inhibiting CDH17-positive colorectal tumor cell, SNU-C1, growth with little effect on body weight of treated mice. Briefly, FIG. 3A shows SNU-C1 (CDH17+) cell line xenografts treated with non-targeting IgG control antibody or 6 different mAbs directed against CDH17. All antibodies were treated by IV tail vein injection at 10 mg antibody/kg once per week for 6 repeat doses with 6 mice per arm. Line represents mean tumor volume+SEM. FIG. 3B is a bar chart representing the mean change in tumor volume (+SEM) in each treatment group over the 32 days of treatment, and FIG. 3C shows mean % changes in mouse body weights during the treatment window.

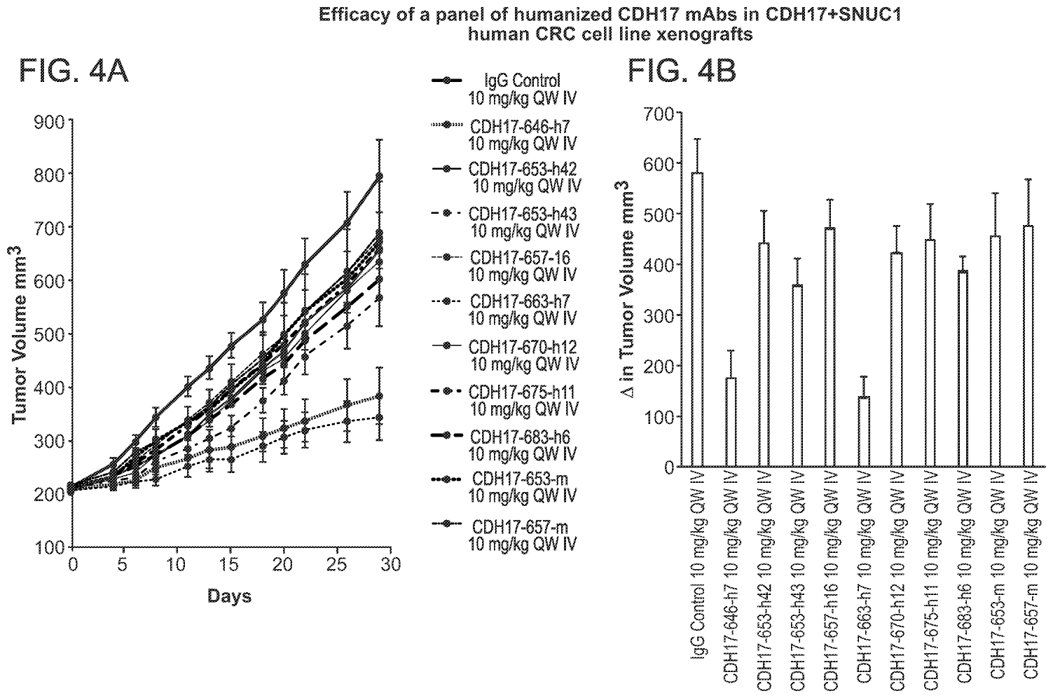

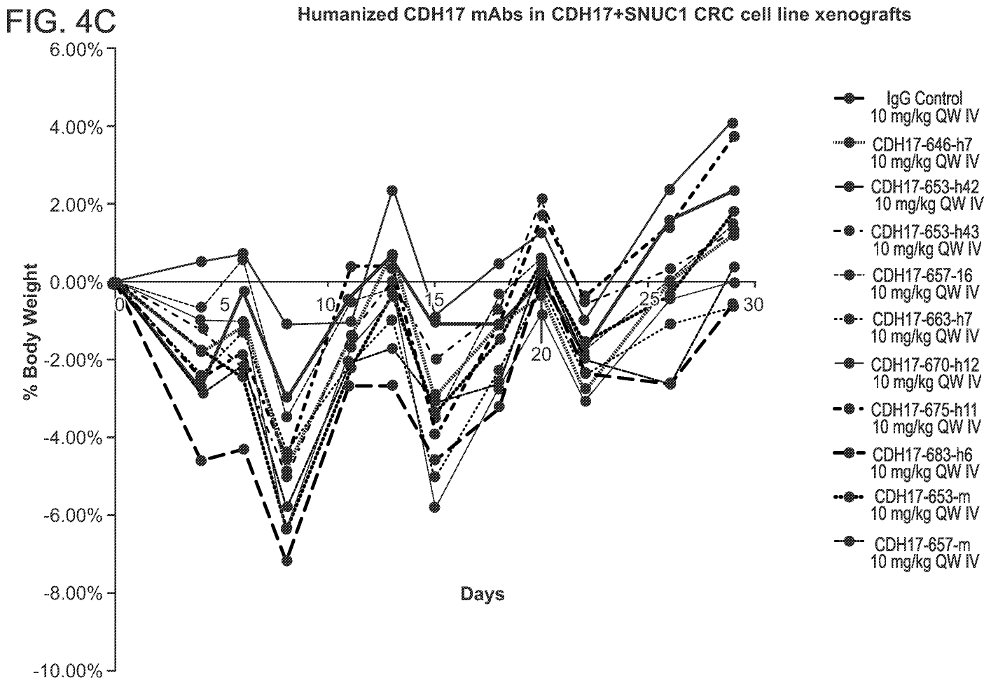

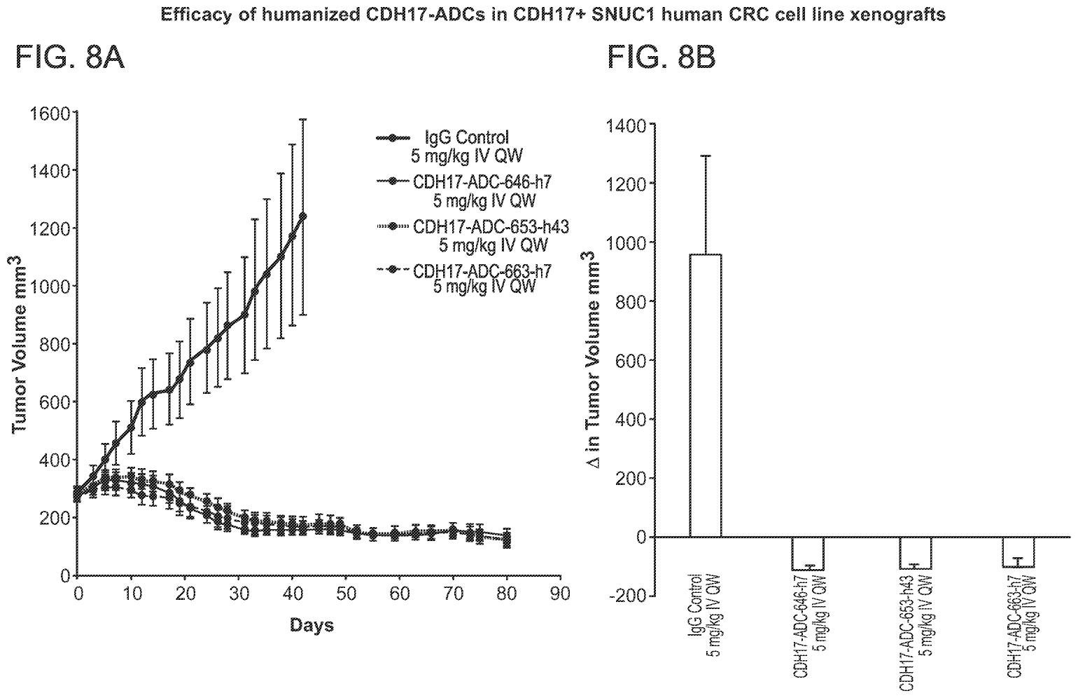

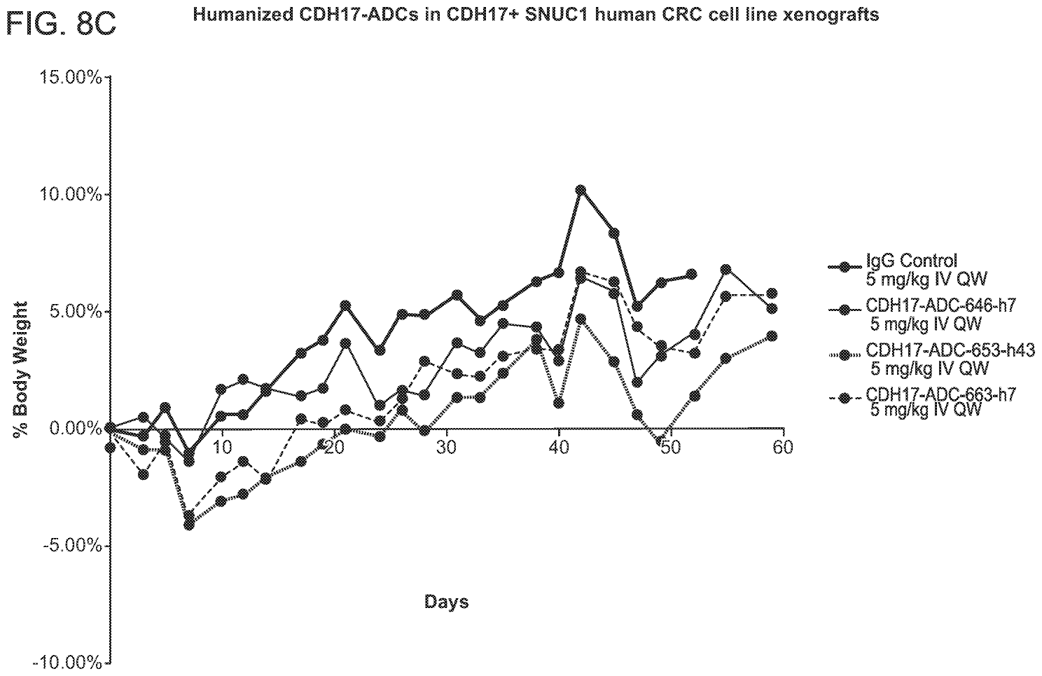

FIG. 4 shows the efficacy of a panel of humanized CDH17 mAbs in CDH17-positive SNUC1 human CRC cell line xenografts. FIG. 4A shows tumor volume (mm³) of SNUC1 (CDH17+) cell line xenografts treated with non-targeting IgG control antibody or 10 different antibodies directed against CDH17, eight humanized anti-CDH17 antibodies (CDH17-646-h7, CDH17-653-h42, CDH17-653-h43, CDH17-657-h16, CDH17-663-h7, CDH17-670-h12, CDH17-675-h11 or CDH17-683-h6) and two parental chimeric monoclonal anti-CDH17 antibodies (CDH17-653-m and CDH17-657-m). Mice were treated by IV tail vein injection of each antibody at 10 mg antibody/kg once per week for 5 repeat doses. 8 mice per arm. Line represents mean tumor volume±SEM. FIG. 4B is a bar chart representing the mean change in tumor volume (+SEM) in each treatment group over the 28 days of treatment. FIG. 8C shows the percentage body weight change over time after administration of the control antibody or 10 different antibodies directed against CDH17, eight humanized anti-CDH17 antibodies (CDH17-646-h7, CDH17-653-h42, CDH17-653-h43, CDH17-657-h16, CDH17-663-h7, CDH17-670-h12, CDH17-675-h11 or CDH17-683-h6) and two parental chimeric monoclonal anti-CDH17 antibodies (CDH17-653-m and CDH17-657-m).

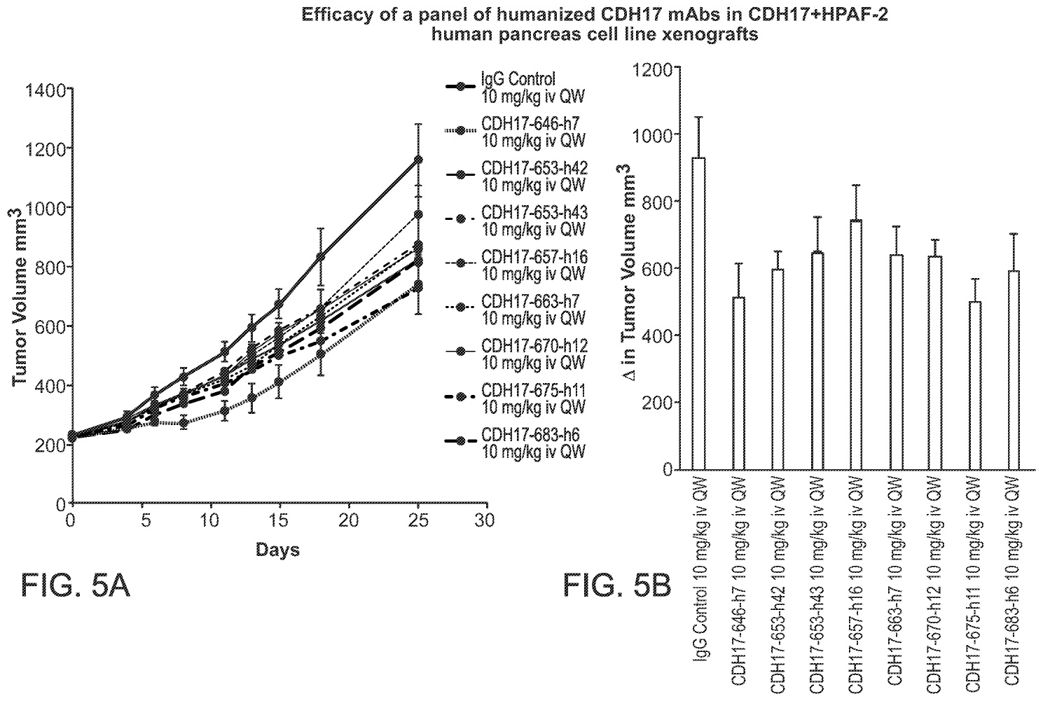

FIG. 5 shows the efficacy of a panel of humanized CDH17 mAbs in CDH17-positive HPAF-2 human pancreas cell line xenografts. FIG. 5A shows HPAF-2 (CDH17+) cell line xenografts treated with non-targeting IgG control antibody or 8 different humanized antibodies directed against CDH17. All mice were treated by IV tail vein injection of each antibody at 10 mg antibody/kg once per week for 5 repeat doses. 8 mice per arm. Line represents mean tumor volume±SEM. FIG. 5B is a bar chart representing the mean change in tumor volume (+SEM) in each treatment group over the 25 days of treatment.

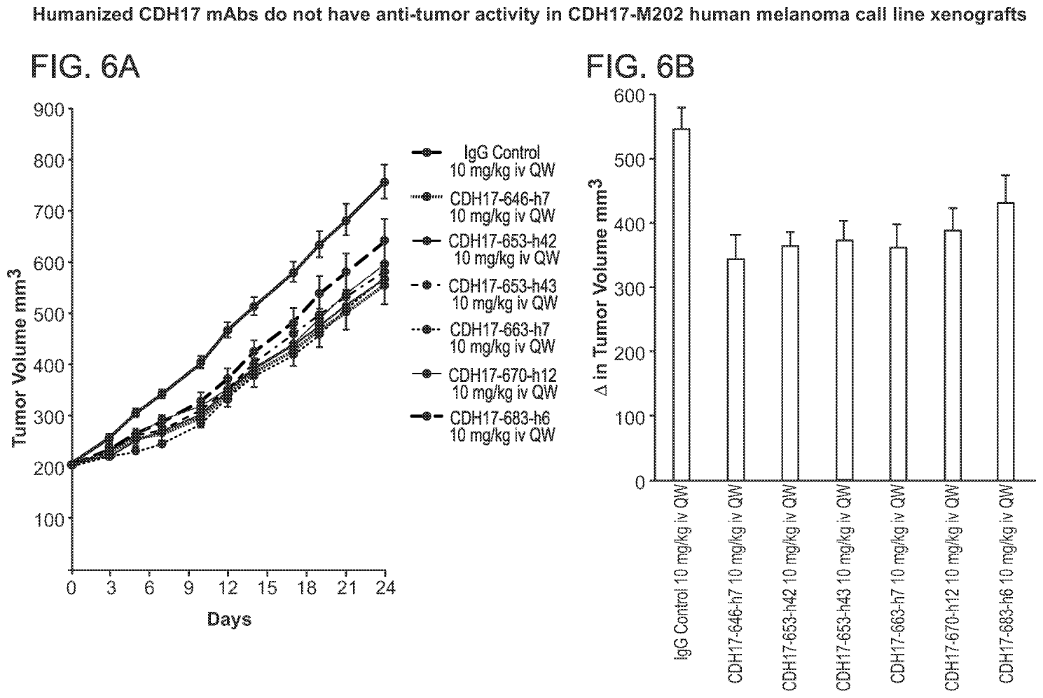

FIG. 6 shows that the humanized CDH17 mAbs do not have anti-tumor activity in CDH17-negative M202 human melanoma cell line xenografts. FIG. 6A shows M202 (CDH17-) cell line xenografts treated with non-targeting IgG control antibody or 5 different humanized antibodies directed against CDH17. All mice were treated by IV tail vein injection of each antibody at 10 mg/kg once per week for 4 repeat doses. 8 mice per arm. Line represents mean tumor volume±SEM. FIG. 6B is a bar chart representing the mean change in tumor volume (+SEM) in each treatment group over the 24 days of treatment.

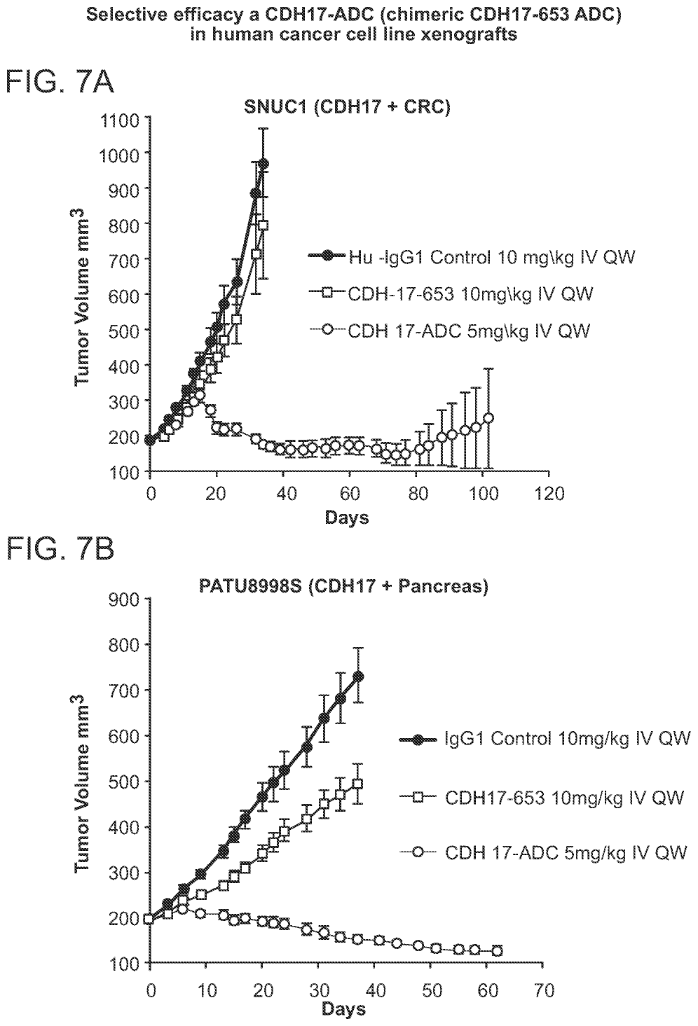

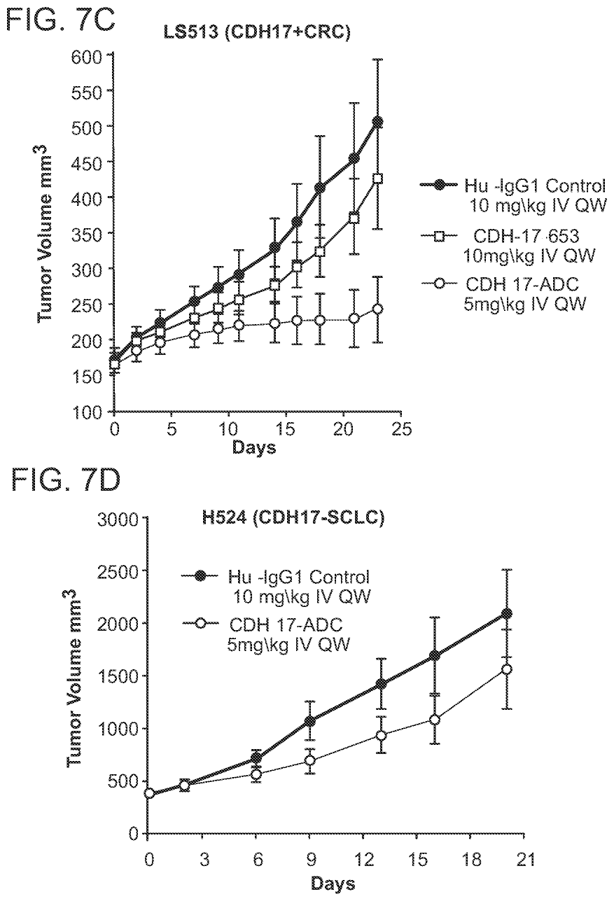

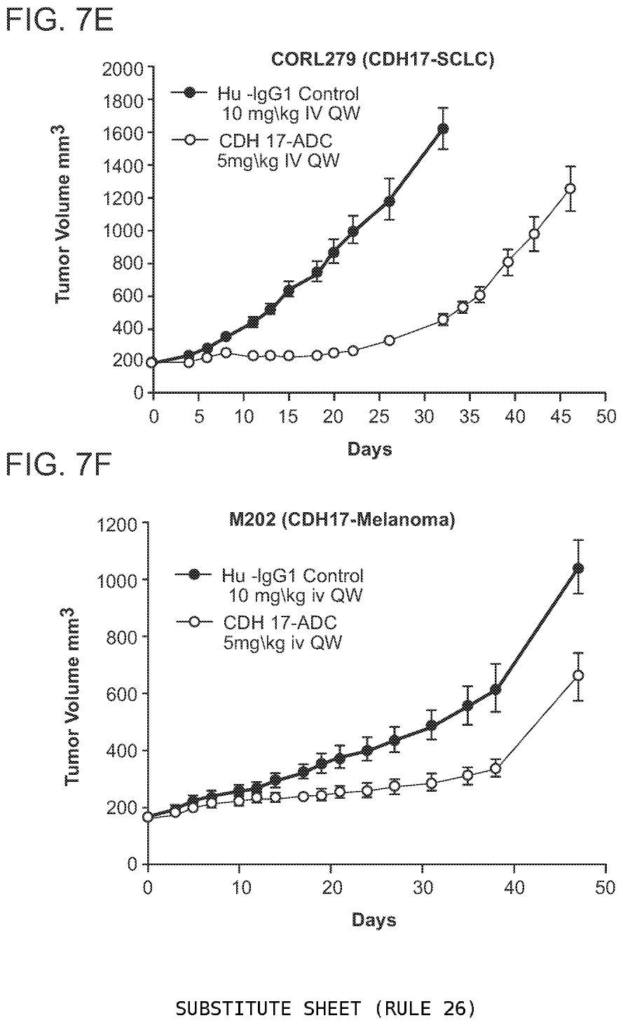

FIG. 7 shows potency of a cytotoxic drug conjugate of a chimeric monoclonal antibody directed to CDH17 protein, CDH17-ADC, in selectively inhibiting and killing a number of different CDH17-positive human cancer cell line xenografts with greatly reduced potency for CDH17-negative human cancer cell line xenografts. FIGS. 7A-C show the results for SNU-C1, PaTu8998s and LS514 (all CDH17+) cell line xenografts treated with non-targeting IgG control antibody, CDH17-653 (chimeric-mAb) or CDH17-ADC generated from the CDH17-653-chimeric-mAb. Mice were treated with control IgG antibody and mAb at 10 mg/kg and ADC at 5 mg/kg by IV tail vein injection for 3 weekly repeat doses. FIG. 7D-F show H524, CORL279 and M202 (all CDH17-) cell line xenografts treated with control or CDH17-ADC as described above. 6-8 mice per arm. Lines represents mean tumor volume±SEM.

FIG. 8 shows the efficacy of humanized CDH17-ADCs in CDH17-positive SNUC1 human CRC cell line xenografts. FIG. 8A shows SNUC1 (CDH17+) cell line xenografts treated with non-targeting IgG control antibody or 3 different CDH17-ADCs generated from humanized CDH17-mAbs (CDH17-ADC-646-h7, CDH17-ADC-653-h43 and CDH17-ADC-663-h7). Mice were administered control antibody and ADCs by IV tail vein injection at 5 mg/kg once per week for 3 repeat doses. 8 mice per arm. Line represents mean tumor volume±SEM. FIG. 8B is a bar chart representing the mean change in tumor volume (+SEM) in each treatment group over the 42 days of study (last day of control arm), showing the effectiveness of the ADCs in promoting tumor regression of CDH17-positive human colorectal tumors in vivo. FIG. 8C shows the percentage body weight change over time after administration of the control antibody or the 3 different CDH17-ADCs generated from humanized CDH17-mAbs (CDH17-ADC-646-h7, CDH17-ADC-653-h43 and CDH17-ADC-663-h7).

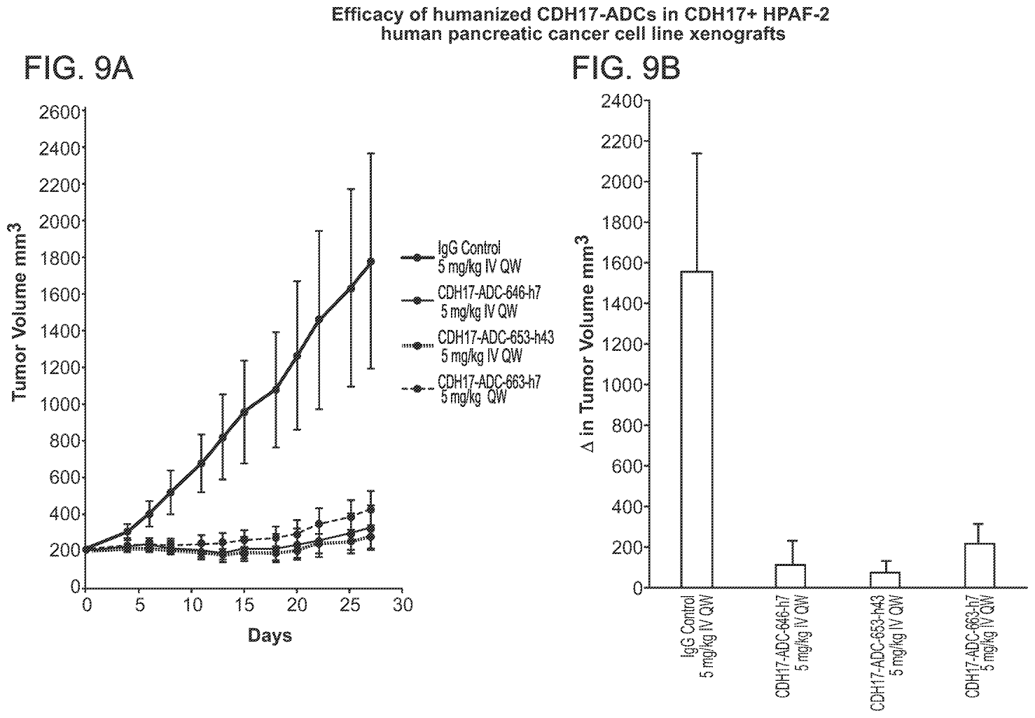

FIG. 9 shows the efficacy of humanized CDH17-ADCs in CDH17-positive HPAF-II human pancreatic cancer cell line xenografts. FIG. 9A shows HPAF-II (CDH17+) cell line xenografts treated with non-targeting IgG control antibody or 3 different CDH17-ADCs generated from humanized CDH17-mAbs (CDH17-ADC-646-h7, CDH17-ADC-653-h43 and CDH17-ADC-663-h7). Mice were treated with control antibody and ADCs by IV tail vein injection at 5 mg/kg once per week for 3 repeat doses. 8 mice per arm. Line represents mean tumor volume±SEM. FIG. 9B is a bar chart representing the mean change in tumor volume (+SEM) in each treatment group over the 27 days of study (last day of control arm).

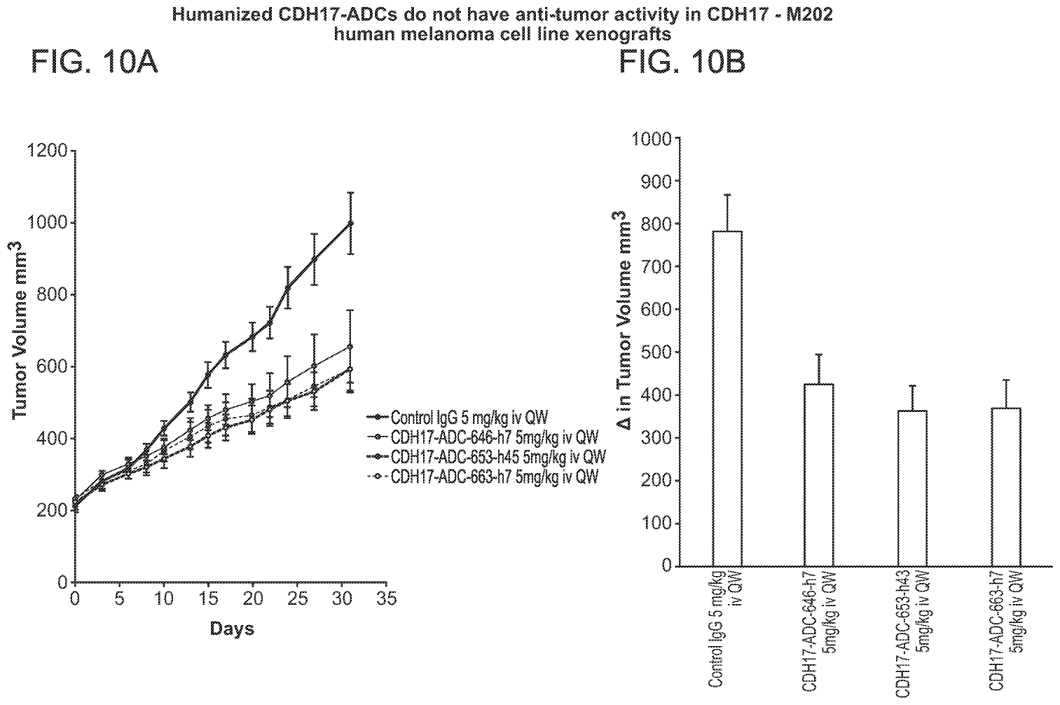

FIG. 10 shows that humanized CDH17-ADCs do not have anti-tumor activity in CDH17-negative M202 human melanoma cell line xenografts. FIG. 10A shows M202 (CDH17-) cell line xenografts treated with non-targeting IgG control antibody or 3 different CDH17-ADCs generated from humanized CDH17-mAbs (CDH17-ADC-646-h7, CDH17-ADC-653-h43 and CDH17-ADC-663-h7). Mice were treated with control antibody and ADCs by IV tail vein injection at 5 mg/kg once per week for 3 repeat doses. 8 mice per arm. Line represents mean tumor volume±SEM. FIG. 10B is a bar chart representing the mean change in tumor volume (+SEM) in each treatment group over the 31 days of study (last day of control arm).

CDH17 antibody drug conjugates (ADCs) presented herein have been conjugated to a cytotoxic agent MMAE via a cleavable linker VC-PAB (thus comprising VC-PAB-MMAE). The average number of MMAE per antibody is 4, as measured by HIC and/or MS. ADCs are heterogenous conjugates with ˜90% of MMAEs conjugated to the interchain disulfides between the heavy chain and light chain of the IgG (reacted with the thiol of the cysteines from the reduced interchain disulfides). ADCs have been prepared at Wuxi Bio with their DAR4 technology.

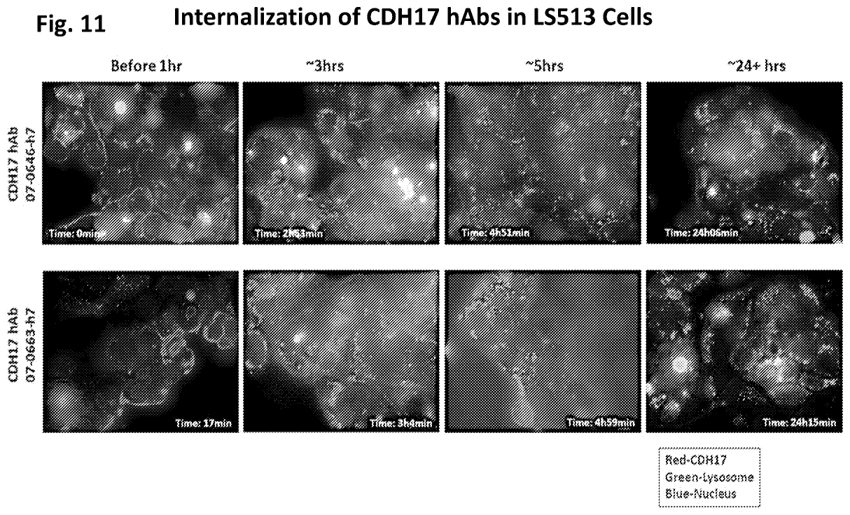

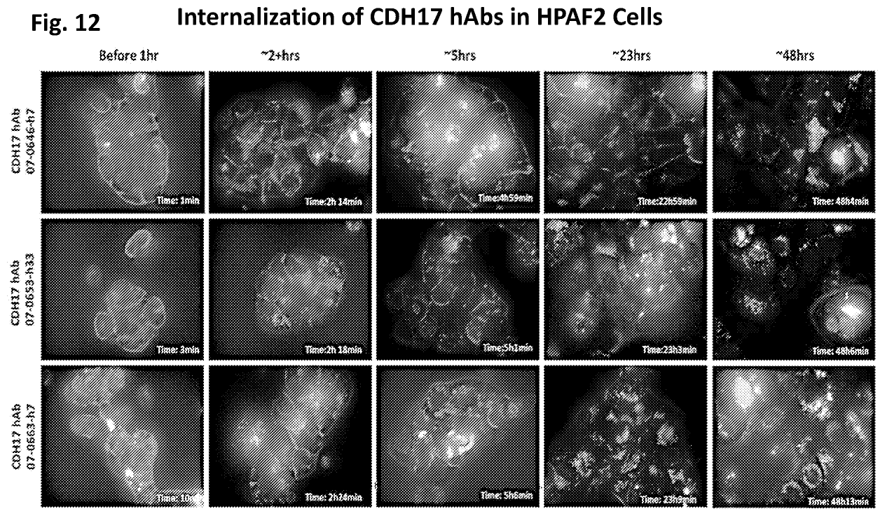

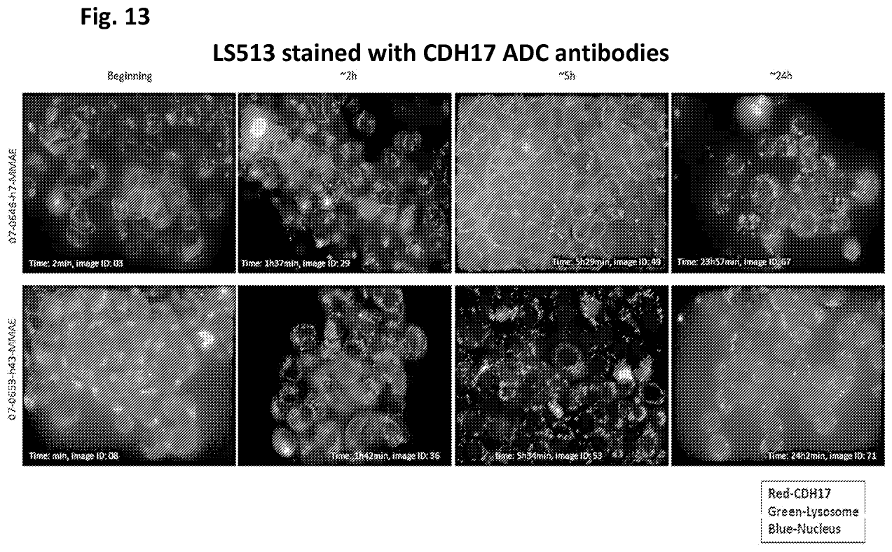

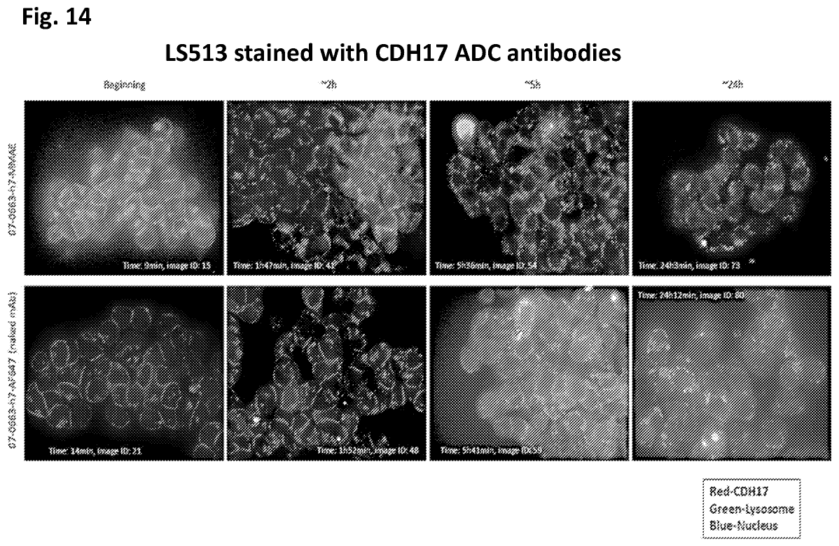

Time course of humanized anti-CDH17 antibody (07-0646-h7, 07-0653-h33 and/or 07-0663-h7) internalization in CDH17-positive human cancer cell lines, HPAF-II human pancreatic cancer cell line and LS513 human colorectal cancer cell line are shown for “naked” unconjugated antibodies in FIGS. 11 and 12 and in LS513 human colorectal cancer cell line for MMAE-conjugated antibodies (07-0646-h7-MMAE, 07-0653-h43-MMAE and 07-0663-h7-MMAE) in FIGS. 13-14. Additionally, FIG. 14 shows the time course of Alexa Fluor 647, AF647, conjugated 07-0663-h7 antibody (07-0663-h7-AF647), internalization in LS513 cell line in vitro.

All three naked antibodies and their ADCs could internalize. The internalization can be captured by imaging after 2 hr antibody binding and is finished within 24 hrs.

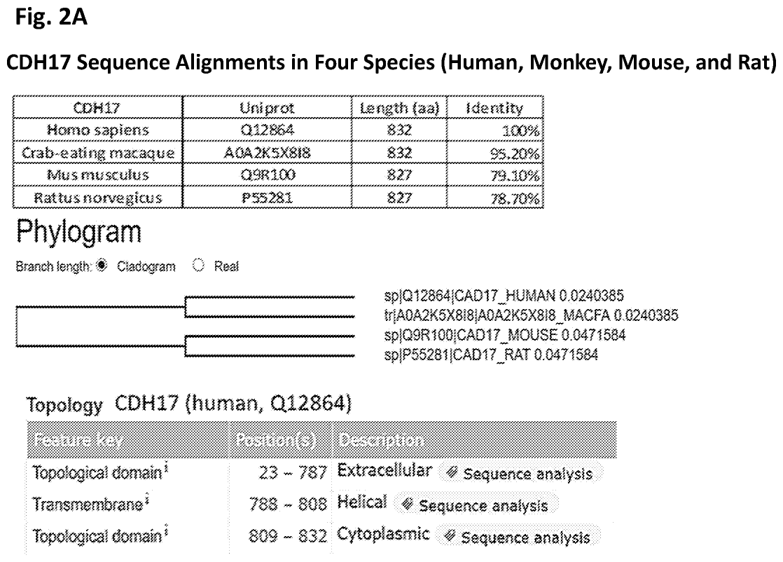

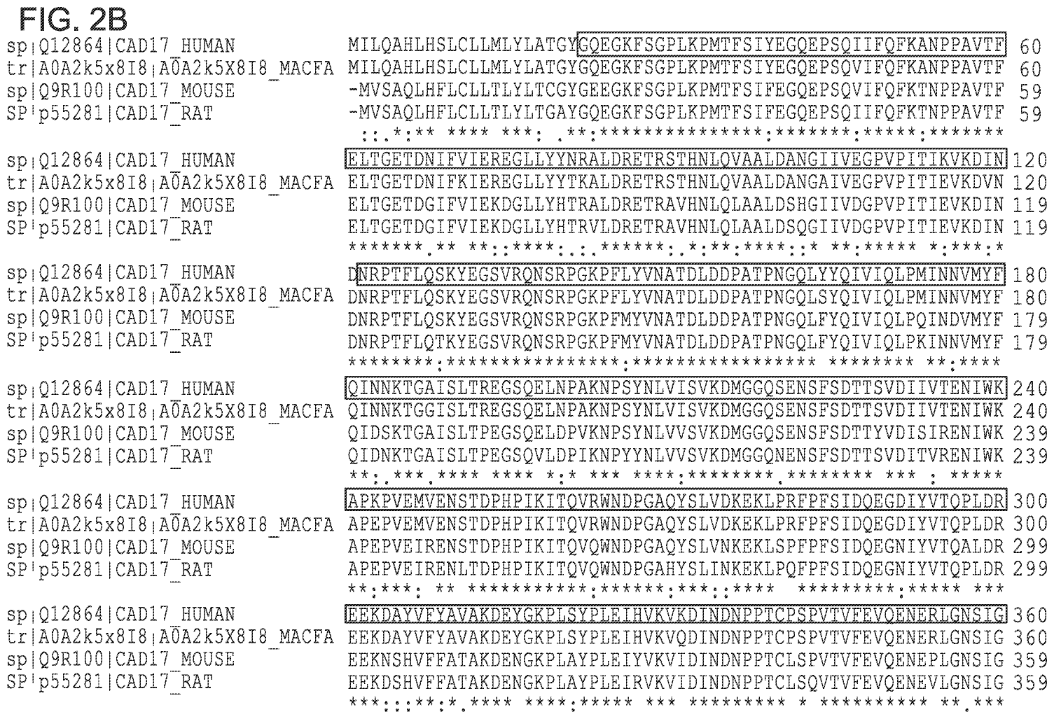

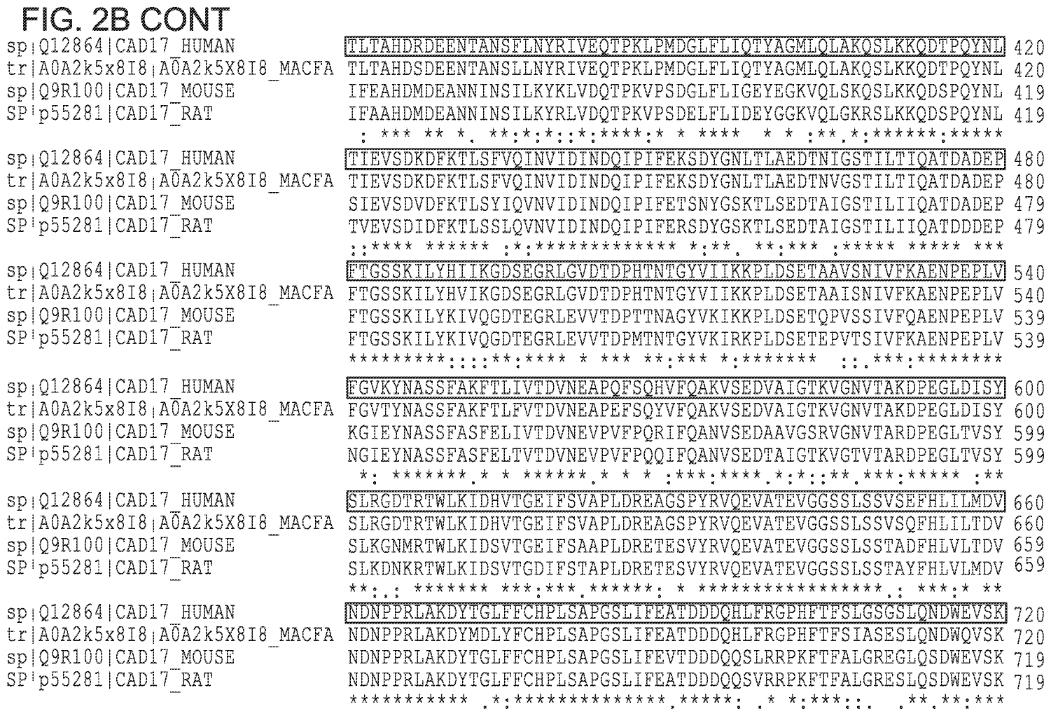

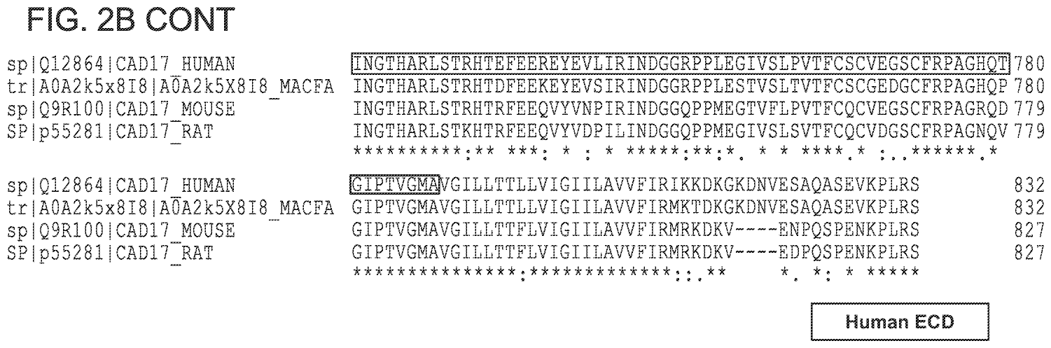

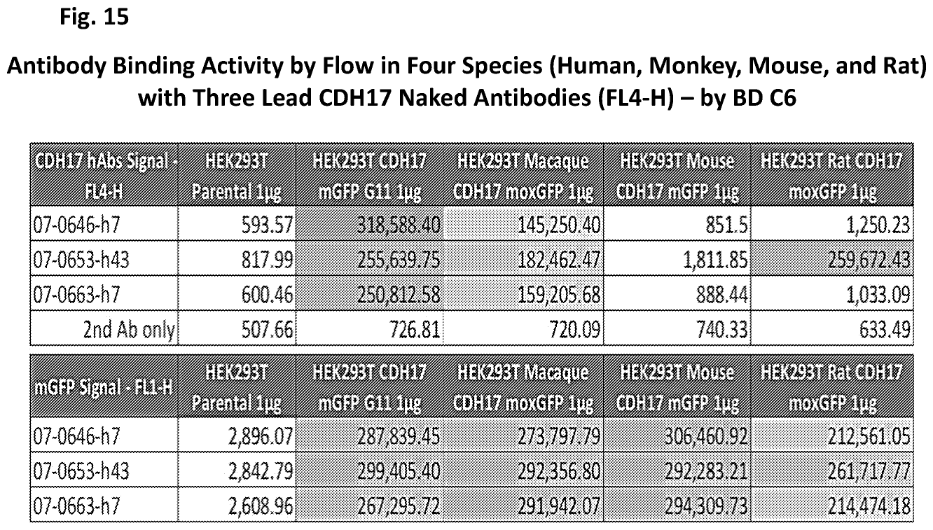

To determine relative antigen binding strengths of the humanized antibodies, FACS assays were carried out as essentially described in Example 1. Briefly, CDH17-overexpressing cells (HEK293T cells transfected with an expression plasmid for human, monkey, mouse or rat CDH17 fused to a naturally fluorescent protein (mGFP or mox GFP)) or untransfected HEK293T control cells were incubated with 1 μg of humanized anti-CDH17 antibodies (07-0646-h7, 07-0653-h43 and 07-0663-h7) or Alexa Fluor® 647 conjugated anti-human IgG Fc secondary antibody from BioLegend (409320) with ˜150,000 cells in 50 μL 2% FBS/PBS on ice for 30 min, and, after washing with 2% FBS/PBS, incubated with the Alexa Fluor® 647 anti-IgG Fc secondary antibody for 30 min on ice. Using a BD Biosciences Accuri™ C6 Flow Cytometer (San Jose, CA), antibody binding based on Alexa Fluor® 647 fluorescence was measured in the FL4-H channel and fusion protein overexpression based on GFP fluorescence was measured in the FL1-H channel. FIG. 15 shows preferential binding of all three humanized antibodies to human CDH17 protein with less binding to monkey (Macaque) CDH17 protein. For two humanized anti-CDH17 antibodies, 07-0646-h7 and 07-0663-h7, there appears to be little or no binding to mouse or rat CDH17 protein; whereas, the humanized anti-CDH17 antibody, 07-0653-h3, has detectable affinity for mouse CDH17 protein with significant binding (cross reactivity) to rat CDH17 reaching a level similar to that for the human CDH17 protein. Similarity in amino acid sequence of the CDH17 proteins from four different species is shown in FIG. 2.

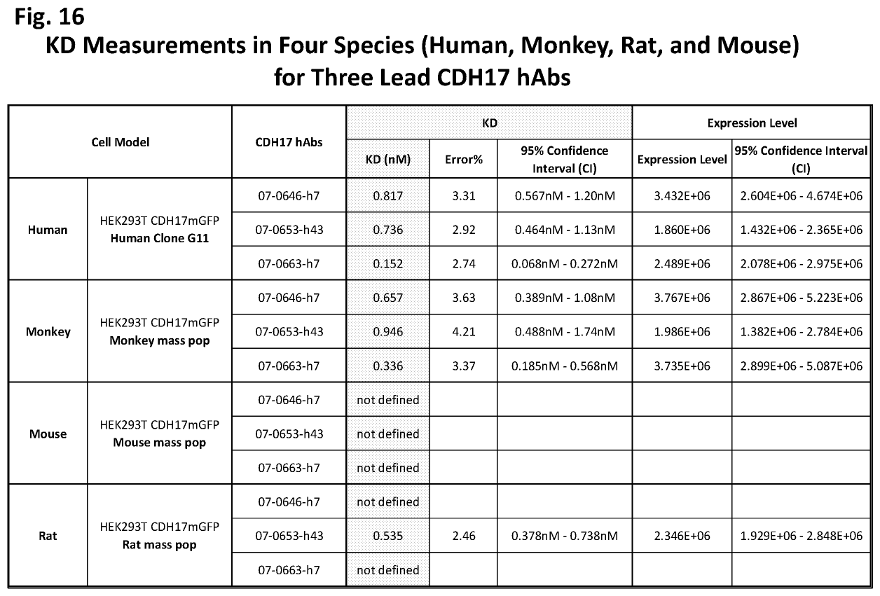

FIG. 16 shows dissociation constant (K_{D}) measurements of humanized anti-CDH17 antibody-CDH17 protein, the latter from four different species. The cell-based antibody affinity (KD) of the anti-CDH17 antibodies was measured by KinExA 4000 (Sapidyne Instrument, Boise, Idaho). Briefly, HEK293T overexpressing CDH17-fluorescent protein fusion (i.e., human CDH17-mGFP, monkey CDH17-mGFP, mouse CDH17-mGFP or rat CDH17-mGFP) were detached with Versene and equilibrated with either 50 pM, 200 pM, 20 nM, or 50 nM antibody in 2% FBS/DMEM at 4° C. overnight. The cell concentration started at 5×10⁶ cells/mL or 1×10⁷ cells/mL and 2-fold serial dilutions were performed up to 10 points. The next day, the cells were centrifuged at 1500 rpm for 10 min and the supernatants were saved. The PMMA beads (Sapidye Instrument, Cat. #440176) were pre-coated with goat-anti-human IgG (Jackson ImmunoResearch Labs (Cat. #109-005-003) at 30 μg/mL. Fluorescent secondary antibody Alexa Fluor® 647 AffiniPure Goat Anti-Human IgG (Jackson ImmunoResearch Labs, Cat. #109-605-088) was diluted in 1% BSA/PBS at 0.5 μg/mL. The antibody solution only (Signal 100%) and nonspecific binding (NSB, buffer only) controls were also included in the measurements. The Kp was calculated using two antibody curves analyzed by the n-curve analysis. As seen in FIG. 16, the humanized anti-CDH17 antibodies, 07-0646-h7 and 07-0663-h7, has a Kp of less than about 1 nM for human and monkey CDH17 protein with no measurable binding to mouse or rat CDH17 protein. In contrast, while the humanized anti-CDH17 antibody, 07-0653-h43, fails to bind mouse CDH17 protein, it binds rat CDH17 protein equally well as to human and monkey CDH17 protein such that its Kp is less than about 1 nM for human, monkey and rat CDH17 proteins with overlapping 95% confidence interval values.

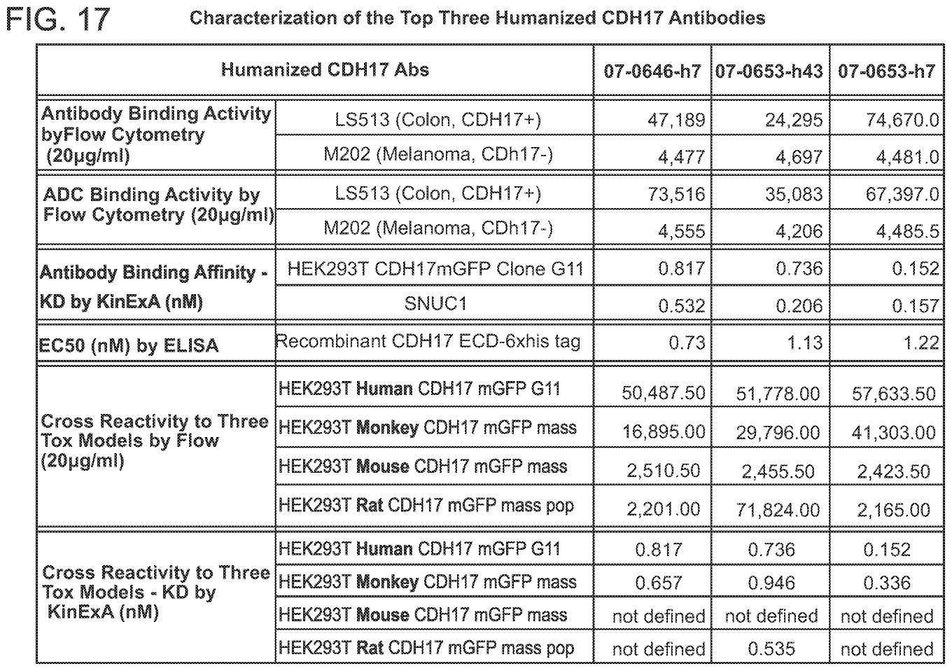

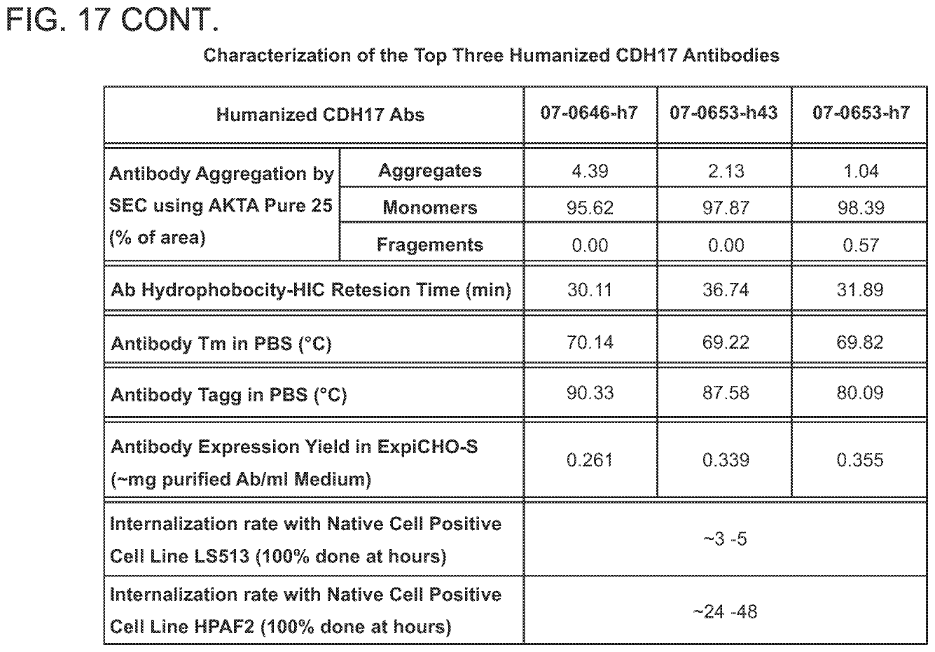

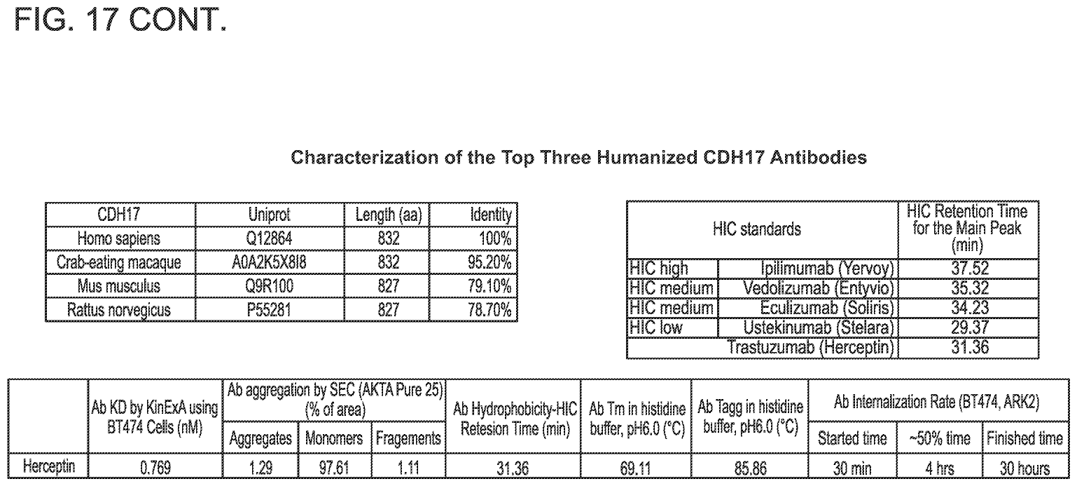

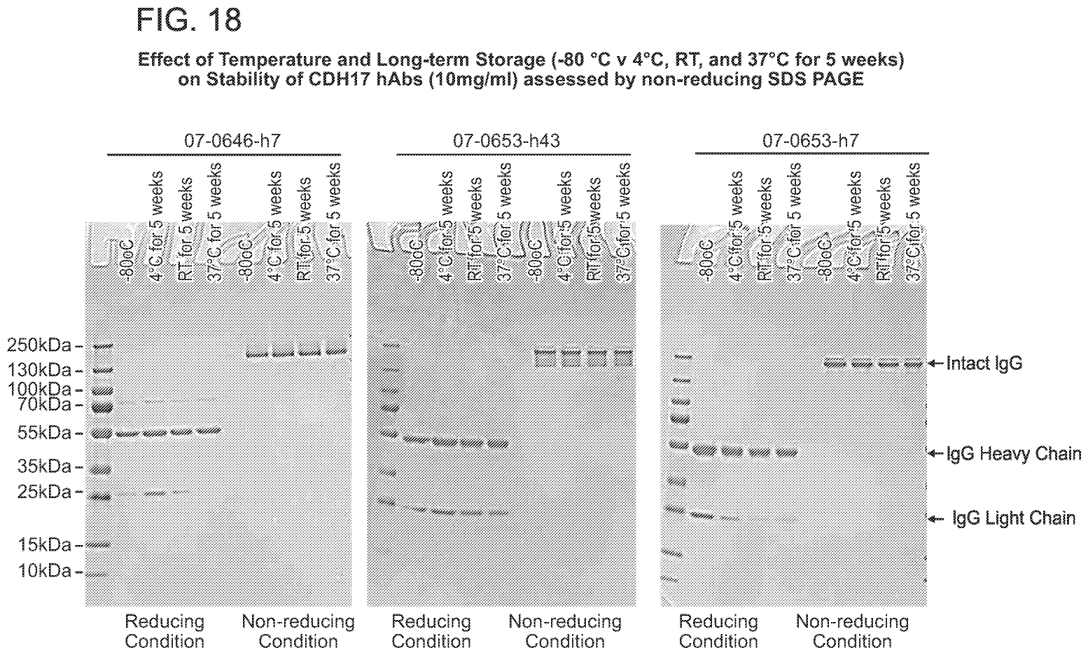

FIG. 17 summarizes biochemical, biophysical and cell biological characteristics of three lead humanized anti-CDH17 antibodies along with properties of some commercial antibodies measured under the same conditions, while FIG. 18 shows general stability of the three lead anti-CDH17 antibodies (07-0646-h7, 07-0653-h43 and 07-0663-h7) across a wide range of temperatures from −80° C. to 37° C. over an extended period of 5 weeks as analyzed by non-reducing SDS-PAGE of stored samples subjected to reducing or non-reducing conditions just prior to electrophoresis.

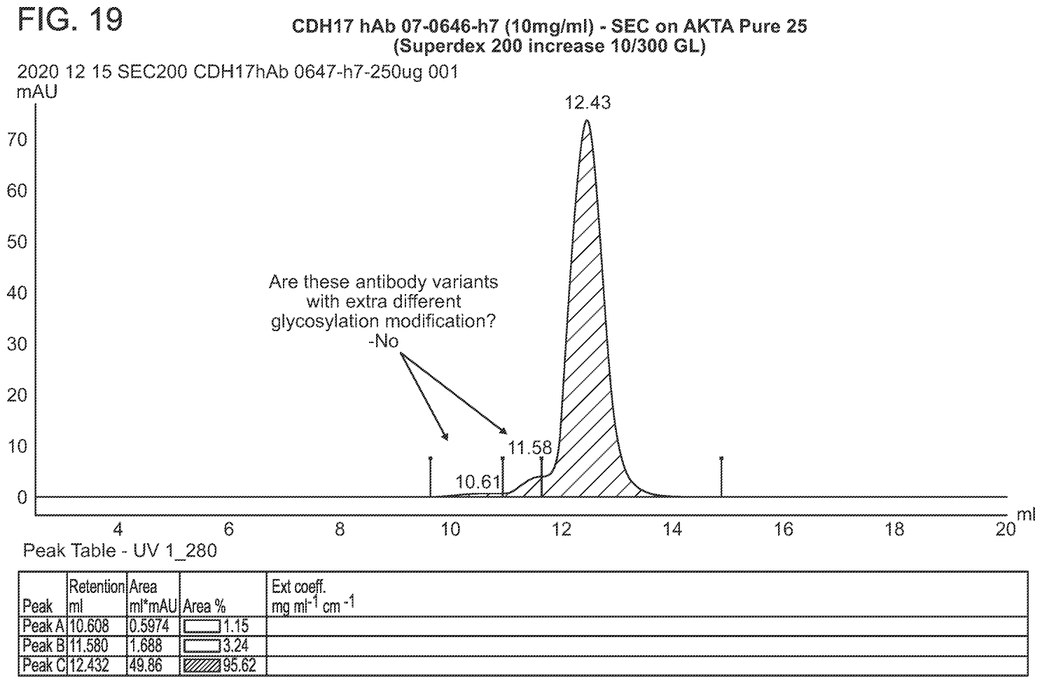

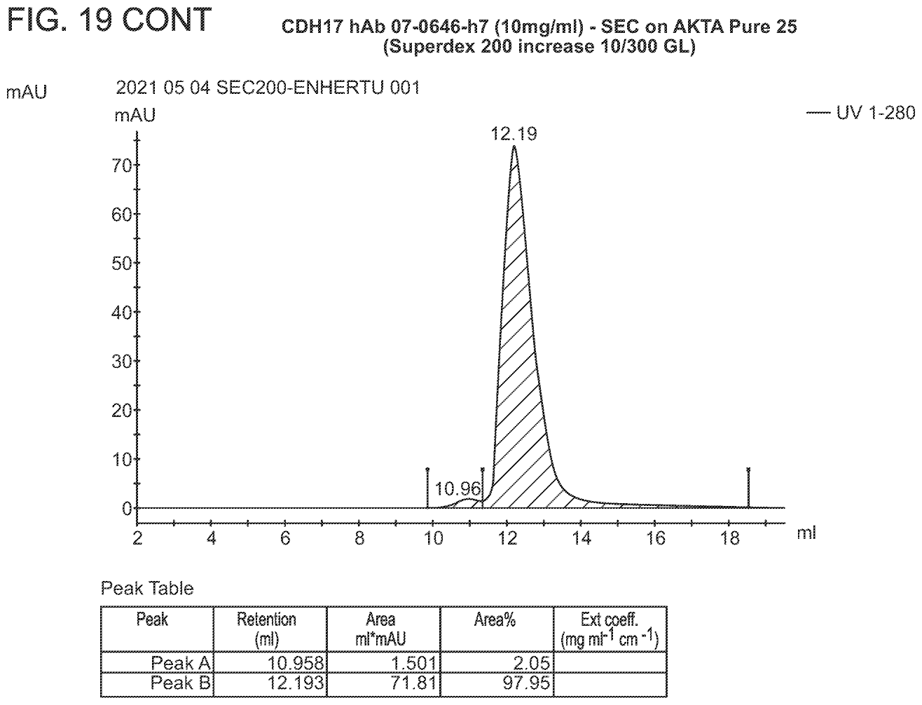

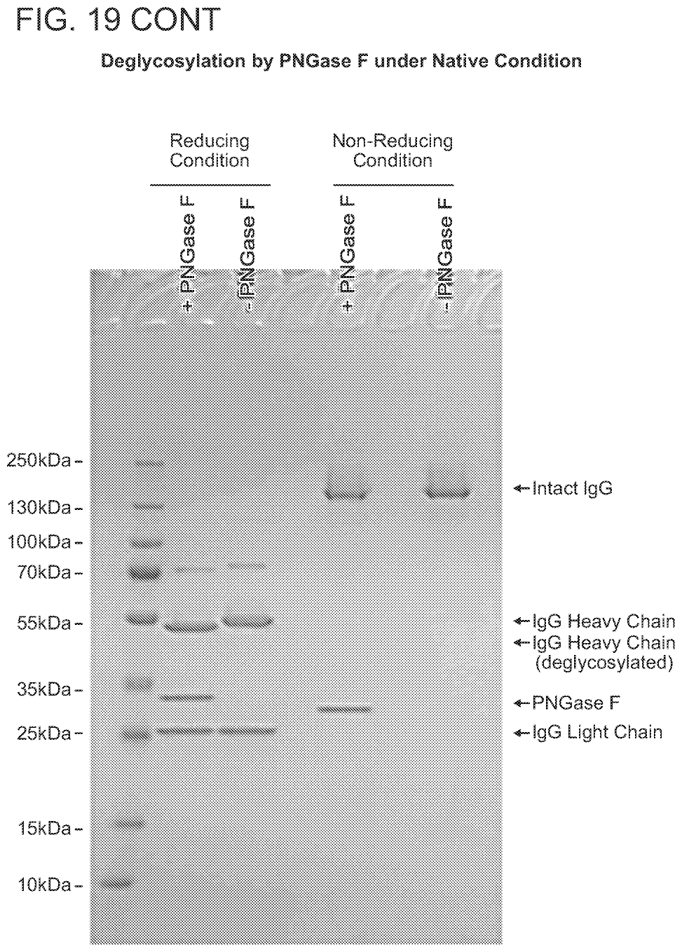

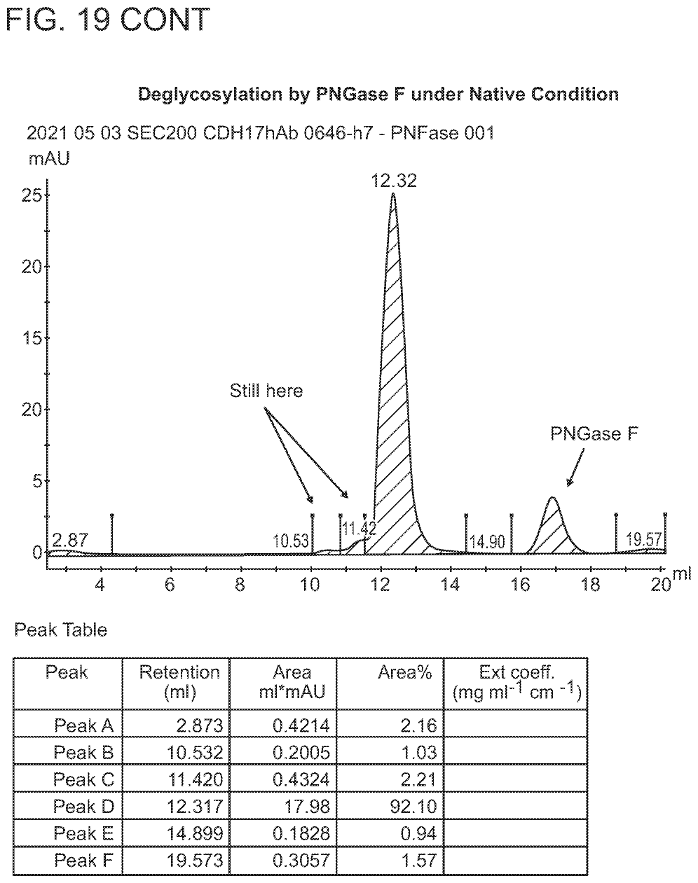

Results of size exclusion chromatography and SDS-PAGE analysis of untreated or Peptide-N-Glycosidase F (PNGase F)-treated humanized anti-CDH7 antibody, 07-0646-h7, are shown in FIG. 19. Note that the antibody preparation is fairly homogenous with about 95% being monomeric and a small percent as aggregates, which is also summarized in FIG. 17.

The humanized anti-CDH17 antibodies of the present invention target human CDH17 protein with nanomolar affinity and can inhibit growth of CDH17-positive human tumors. The antibodies stain CDH17-positive cells and undergo internalization in hours varying from about 2-24 hrs depending on tumor cell line. Different human tumors vary in their level of CDH17 expression (FIG. 1) which may influence effectiveness of anti-CDH17 antibodies on growth inhibition and antibody internalization. As anti-CDH17 antibody-drug conjugates, the ADCs are shown to not only inhibit CDH17-positive tumor growth but can also cause tumor regression-an effect not seen in CDH17-negative tumors.

### Example 3

This example describes mapping antibody binding epitopes for CDH17 hAb 0663-h7.

Peptides of 15 amino acids were designed spanning the ECD of CDH17 (SEQ ID NO: 51). The peptides were offset by 4 amino acids i.e., each peptide was overlapping another peptide preceeding and/or following it by 11 amino acids. A total of 189 peptides were designed and were manufactured with biotin labels by JPT Peptide Technologies. Biotinylated peptides were placed into wells of 96 well plates and dissolved in DMSO to a concentration of ˜0.45 μg/μl (each peptide has ˜25 nmol/well but as each peptide has different molecular weight, the concentrations will vary).

For the ELISA, 1 μl of each biotinylated peptide plus 99 μl of peptide coating buffer (40% DMSO and 0.05% Tween20 in PBS) was added to a streptavidin-coated microtiter plate well. The plate was incubated overnight at 4° C. without shaking. The plate was then washed 4 times with 300 μl washing buffer (PBS with 0.05% Tween20). 200 μl blocking buffer (PBS with 400 μM biotin and 20% sucrose) was added to each well and the plate incubated for 30 min at room temperature without shaking. After 3 times washing with washing buffer, 300 μl of SuperBlock T20 (TBS) Blocking Buffer (Thermo Scientific, #37536) was applied per well and incubated at room temperature for 1 hour. Followed by 3 times washing, one dose of CDH17 hAb at 200 ng/ml per peptide was added to the wells. Peptides that show positive signals were repeated for titration ELISA using CDH17 hAb starting 500 ng/ml, 1 to 2 dilution for 11 points (500, 250, 125, 62.5, 31.25, 15.625, 7.8125, 3.906, 1.953, 0.977, 0.488 ng/ml, respectively).

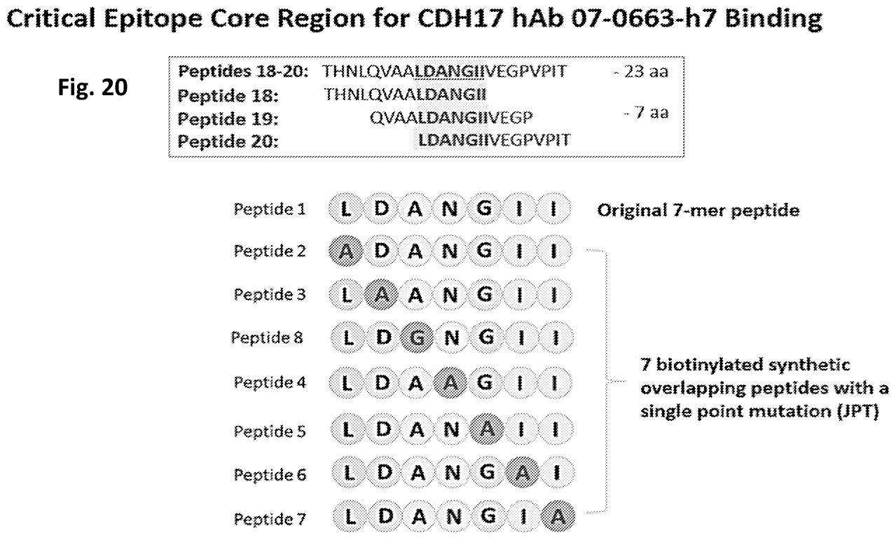

The titration ELISA identified overlapping peptides 18-20 with a 7 amino acid (LDANGII, SEQ ID NO: 53) core region for CDH17 hAb 07-0663-h7 binding (FIG. 20).

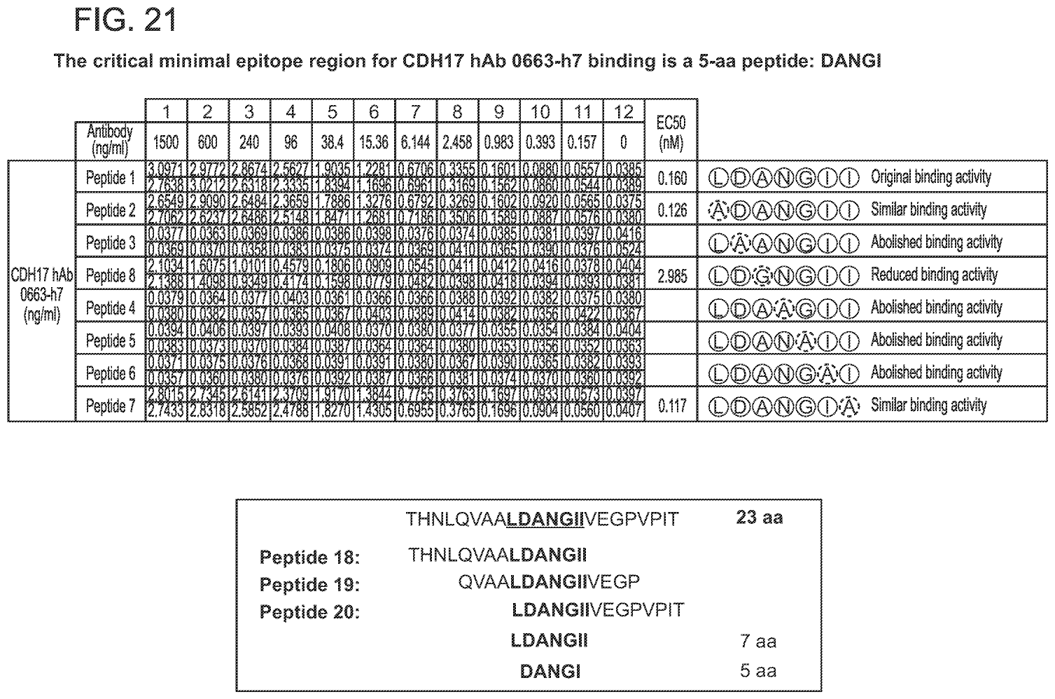

The 7 amino acid epitope core for CDH17 hAb 07-0663-h7 binding was further analyzed for amino acids critical to binding. Single point mutations were designed in the 7-mer amino acid epitope as shown in FIG. 20. Biotinylated peptides were manufactured and assayed by ELISA as described supra. Mutation of 5 amino acids (DANGI, SEQ ID NO: 54), reduced or abolished binding activity to CDH17 hAb 07-0663-h7. Hence, the critical minimual epitope region for antibody binding is the 5 amino acid region DANGI (FIG. 21).

### Example 4

Selected CDH17 antibodies, 07-646-h7 and 07-663-h7 were assessed for their ability to induce antibody-dependent cellular cytotoxicity (ADCC) in CDH17 positive cell lines.

CDH17 positive cell lines (LS513 and SNUC1) and a CDH17 negative cell line (M202) were incubated with various concentrations of CDH17 antibodies or control hIgG1 from about 0.1 pM to 10 μM for 16 hours. Jurkat-Lucia™ NFAT-CD16 effector cells (InvivoGen, San Diego, USA) were then co-incubated with targets cells for 6 hours.

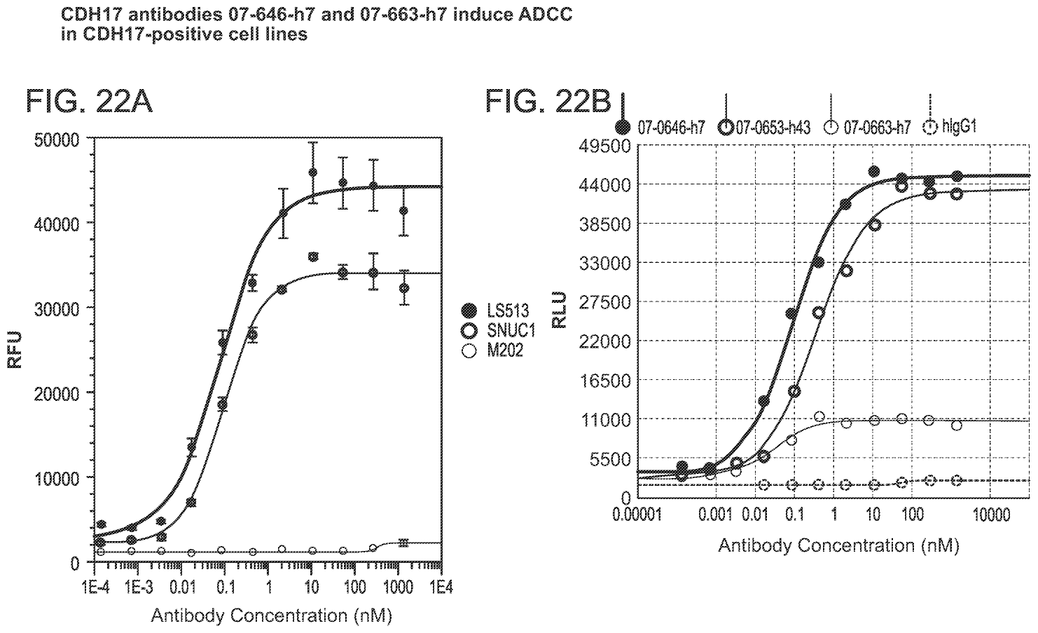

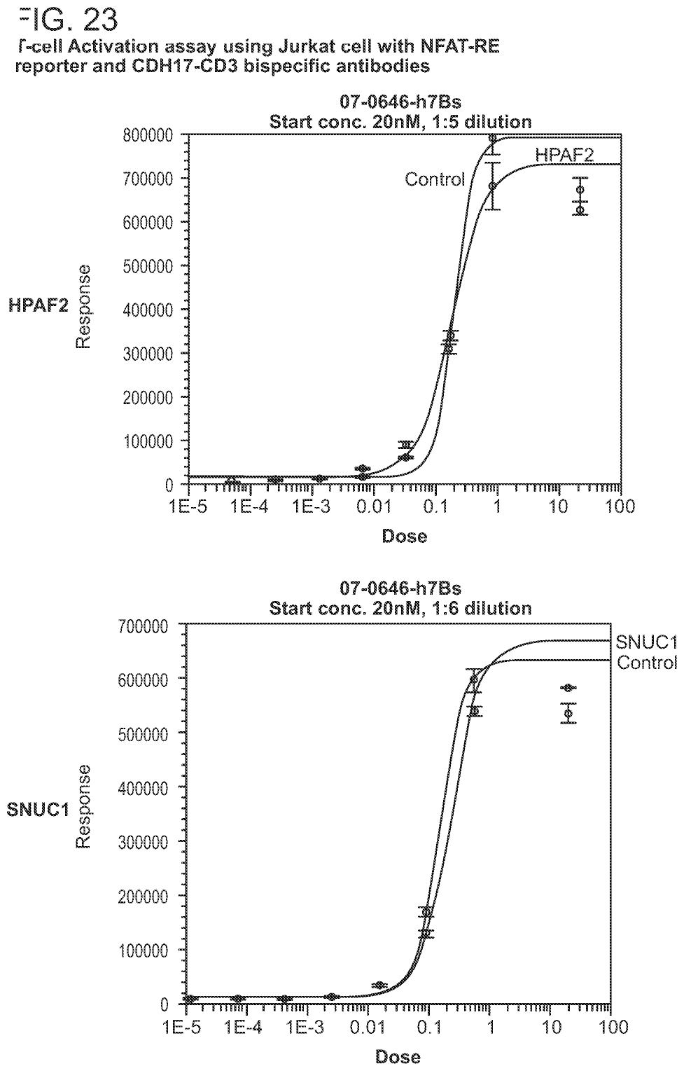

As shown in FIG. 22, NFAT activation, which indicates the induced ADCC response, was assessed by determining Lucia luciferase activity in the supernatant. CDH17 antibodies, in particular 07-646-h7 and 07-663-h7, induced ADCC in CDH17 positive cell lines.

### Example 5

The activity of the CDH17-CD3 bispecific antibodies was assessed by the T-Cell activation assay using the Jurkat cells with NFAT-RE reporter.

T-cell activation was measured using Promega's T Cell Activation Bioassay kits (NFAT-RE J1621). A couple of CDH17 positive cell lines (HPAF2 and SNUC1) were seeded at a density of 20,000-40,000 cells/well in white 96 well plate wells and incubated at 37° C. overnight, after which thaw-and-use Jurkat T cells included in the assay kit were added to the seeded cells (1:1 cell ratio) and treated with CDH17-CD3 bispecific antibodies (07-0653-h43Bs, 07-0646-h7Bs, and 07-0663-h7Bs, each of which comprises a C-terminal (HIS) 6 tag). A control containing no target positive cells was included for comparison. Treated plates were incubated in 37° C. for 6 hours, after which Bio-Glo reagent (included in kit) was added and immediately read on the Varioskan LUX plate reader. The units were RLU.

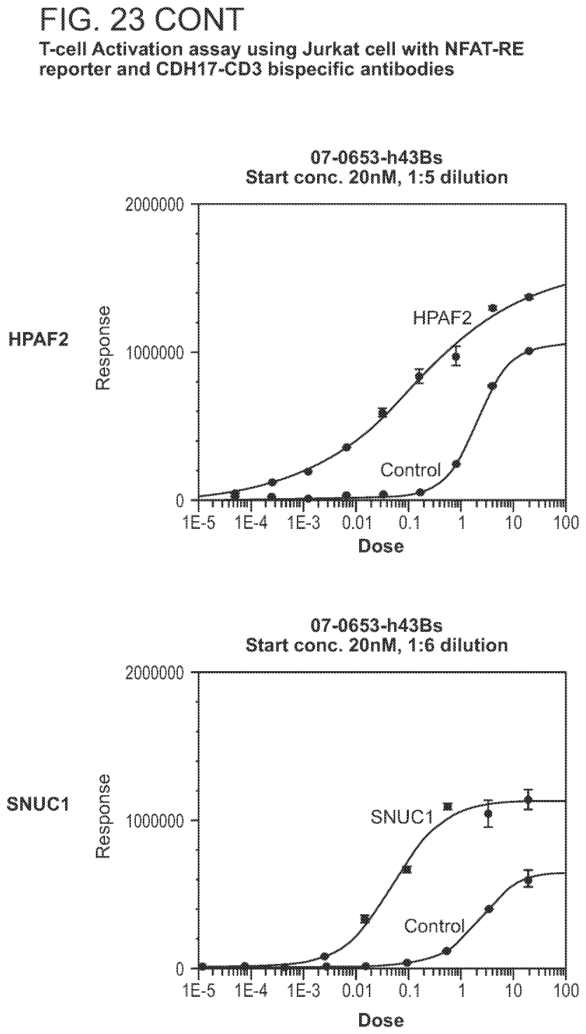

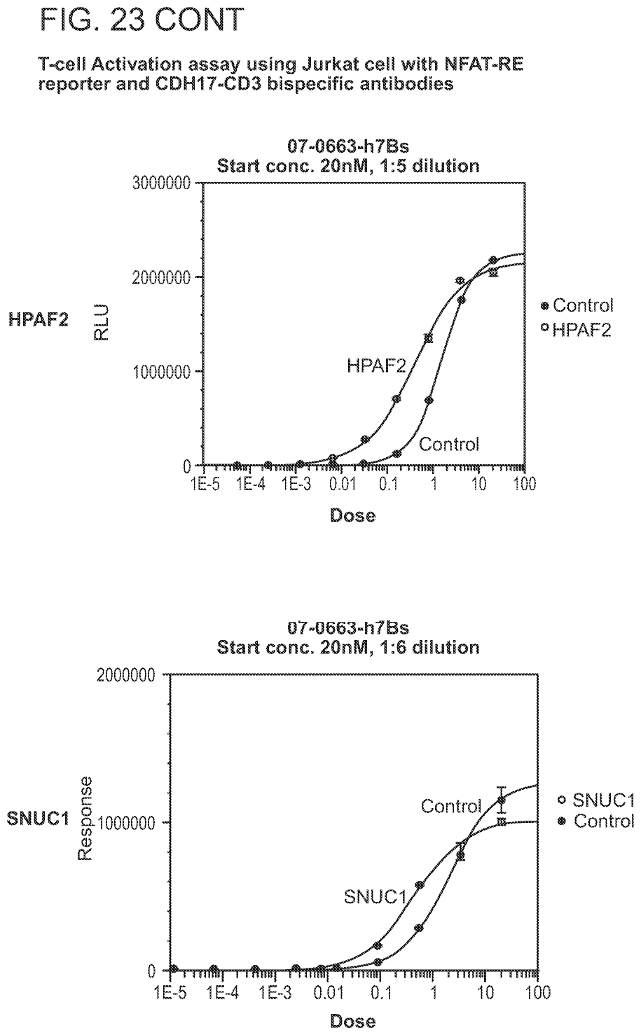

As shown in FIG. 23, treatment of CDH17 positive cell lines (HPAF2 and SNUC1) with the 07-0653-h43Bs bispecific antibody resulted in T-cell activation compared to no target cell control. By contrast, there was no difference in the T-cell activation level between CDH17 positive cells and a no-target cell control for the 07-0646-h7Bs and 07-0663-h7Bs bispecific antibodies. The bispecific antibodies used herein further comprises a C-terminal (HIS) 6 tag.

### Example 6

Bispecific antibodies and scFv fragments were assessed for their binding effect to CDH17 positive cells by an indirect flow cytometry assay iQue® (Sartorius, Gottingen, Germany). The asssays were performed with CDH17 positive LS513 cells or CDH17 negative M202 cells using the primary antibody and fluorochrome-labeled anti-His secondary antibody using the iQue® where FL4-H values acquired by iQue® software were reported.

Table 8B shows the CDH17 scFvs and BiTEs that demonstrated selectively binding to the CDH17 positive LS513 cells and not to the CDH17 negative M202 cells

TABLE 1

| Seq ID | |
|---|---|
| NO.: | Sequence |
| 01 | GYTFTSYN |
| 02 | IYPGNGDT |
| 03 | ARGRGRYFEY |
| 04 | SSVSSSY |
| 05 | STS |
| 06 | QQYDSSPST |
| 07 | GYTFTDYY |
| 08 | IYPYSGGI |
| 09 | ARGRGDYFGLFDF |
| 10 | SSLSY |
| 11 | EIS |
| 12 | QQWNYPFT |
| 13 | GYTFTDHT |
| 14 | IFPRDDIV |
| 15 | ARPPYYYSRNFYFDY |
| 16 | SIISSSK |
| 17 | GTS |
| 18 | QQWSNYPFT |
| 19 | human cadherin-17; UniProt Accession NO Q12864 |
| | MILQAHLHSLCLLMLYLATGYGQEGKFSGPLKPMTFSIYEGQEPSQIIFQFKA |
| | NPPAVTFELTGETDNIFVIEREGLLYYNRALDRETRSTHNLQVAALDANGIIV |
| | EGPVPITIKVKDINDNRPTFLQSKYEGSVRQNSRPGKPFLYVNATDLDDPATP |
| | NGQLYYQIVIQLPMINNVMYFQINNKTGAISLTREGSQELNPAKNPSYNLVIS |
| | VKDMGGQSENSFSDTTSVDIIVTENIWKAPKPVEMVENSTDPHPIKITQVRW |
| | NDPGAQYSLVDKEKLPRFPFSIDQEGDIYVTQPLDREEKDAYVFYAVAKDEY |
| | GKPLSYPLEIHVKVKDINDNPPTCPSPVTVFEVQENERLGNSIGTLTAHDRDE |
| | ENTANSFLNYRIVEQTPKLPMDGLFLIQTYAGMLQLAKQSLKKQDTPQYNL |
| | TIEVSDKDFKTLCFVQINVIDINDQIPIFEKSDYGNLTLAEDTNIGSTILTIQATD |
| | ADEPFTGSSKILYHIIKGDSEGRLGVDTDPHTNTGYVIIKKPLDFETAAVSNIV |
| | FKAENPEPLVFGVKYNASSFAKFTLIVTDVNEAPQFSQHVFQAKVSEDVAIG |
| | TKVGNVTAKDPEGLDISYSLRGDTRGWLKIDHVTGEIFSVAPLDREAGSPYR |
| | VQVVATEVGGSSLSSVSEFHLILMDVNDNPPRLAKDYTGLFFCHPLSAPGSLI |
| | FEATDDDQHLFRGPHFTFSLGSGSLQNDWEVSKINGTHARLSTRHTEFEERE |
| | YVVLIRINDGGRPPLEGIVSLPVTFCSCVEGSCFRPAGHQTGIPTVGMAVGILL |
| | TTLLVIGIILAVVFIRIKKDKGKDNVESAQASEVKPLRS |
| 20 | mouse (Mus musculus) cadherin-17; UniProt Accession NO Q9R100) |
| | MVSAQLHFLCLLTLYLTCGYGEEGKFSGPLKPMTFSIFEGQEPSQVIFQFKTN |
| | PPAVTFELTGETDGIFKIEKDGLLYHTRALDRETRAVHHLQLAALDSHGAIV |
| | DGPVPITIEVKDINDNRPTFLQSKYEGSVRQNSRPGKPFMYVNATDLDDPAT |
| | PNGQLFYQIVIQLPQINDVMYFQIDSKTGAISLTPEGSQELDPVKNPSYNLVV |
| | SVKDMGGQSENSFSDTTYVDISIRENIWKAPEPVEIRENSTDPHPIKITQVQW |
| | NDPGAQYSLVNKEKLSPFPFSIDQEGNIYVTQALDREEKNSHVFFATAKDEN |
| | GKPLAYPLEIYVKVIDINDNPPTCLSPVTVFEVQENEPLGNSIGIFEAHDMDEA |
| | NNINSILKYKLVDQTPKVPSDGLFLIGEYEGKVQLSKQSLKKQDSPQYNLS |
| | IEVSDVDFKTLCYIQVNVIDINDQIPIFETSNYGSKTLSEDTAIGSTILIIQATDA |
| | DEPFTGSSKILYKIVQGDTEGRLEVVTDPTTNAGYVKIKKPLDFETQPVSSIVF |
| | QAENPEPLVKGIEYNASSFASFELIVTDVNEVPVFPQRIFQANVSEDAAVGSR |
| | VGNVTARDPEGLTVSYSLKGNMRGWLKIDSVTGEIFSAAPLDRETESVYRV |
| | QVVATEVGGSSLSSTADFHLVLTDVNDNPPRLAKDYTGLFFCHPLSAPGSLIF |
| | EVTDDDQQSLRRPKFTFALGREGLQSDWEVSKINGTHARLSTRHTRFEEQVY |
| | NIPIRINDGGQPPMEGTVFLPVTFCQCVEGSCFRPAGRQDGIPTVGMAVGILL |
| | TTFLVIGIILAVVFIRMRKDKVENPQSPENKPLRS |
| 21 | QVQLVQSGAEVKKPGASVKMSCKAS |
| 22 | MHWVRQAPGQGLEWIGA |
| 23 | SYAQKFQGRATLTVDTSTSTAYMELSSLRSEDTAVYYC |
| 24 | WGQGTTLTVSS |
| 25 | DIQLTQSPSSLSASVGDRVTMTCRAS |
| 26 | LHWYQQKPGKAPKLLIY |
| 27 | NLASGVPSRFSGSGSGTDYTLTISSVQPEDFATYYC |
| 28 | FGQGTKLEIK |
| 29 | QVQLVQSGAEVKKPGASVKVSCKAS |
| 30 | MNWVRQAPGQGLEWMGV |
| 31 | GYAQKFQGRVTMTVDKSTSTAYMELSSLRSEDTAVYYC |
| 32 | WGQGTTVTVSS |
| 33 | DIQLTQSPSSLSASVGDRVTITCRAT |
| 34 | IHWYQQKPGKAPKPLIY |
| 35 | KLASGVPSRFSGSGSGTDYTLTISSLQPEDFATYYC |
| 36 | FGQGTKLEIK |
| 37 | QVQLVQSGAEVKKPGSSVKISCKVS |
| 38 | IHWMRQAPGQGLEWIGY |
| 39 | VYAQKFQGRATLTADKSTSTAYMELSSLRSEDTAVYYC |
| 40 | WGQGTTLTVSS |
| 41 | DIQMTQSPSSLSASVGDRVTITCRVS |
| 42 | LHWYQQKPGKAPKPLIY |
| 43 | TLASGVPSRFSGSGSGTDYTLTISSLQPEDFATYYC |
| 44 | FGQGTKLEIK |
| 45 | QVQLVQSGAEVKKPGASVKMSCKASGYTFTSYNMHWVRQAPGQGLE |
| | WIGAIYPGNGDTSYAQKFQGRATLTVDTSTSTAYMELSSLRSEDTAVYY |
| | CARGRGRYFEYWGQGTTLTVSS |
| 46 | DIQLTQSPSSLSASVGDRVTMTCRASSSVSSSYLHWYQQKPGKAPKLLIY |
| | STSNLASGVPSRFSGSGSGTDYTLTISSVQPEDFATYYCQQYDSSPSTFGQ |
| | GTKLEIK |
| 47 | QVQLVQSGAEVKKPGASVKVSCKASGYTFTDYYMNWVRQAPGQGLE |
| | WMGVIYPYSGGIGYAQKFQGRVTMTVDKSTSTAYMELSSLRSEDTAVY |
| | YCARGRGDYFGLFDFWGQGTTVTVSS |
| 48 | DIQLTQSPSSLSASVGDRVTITCRATSSLSYIHWYQQKPGKAPKPLIYEISK |
| | LASGVPSRFSGSGSGTDYTLTISSLQPEDFATYYCQQWNYPFTFGQGTKL |
| | EIK |
| 49 | QVQLVQSGAEVKKPGSSVKISCKVSGYTFTDHTIHWMRQAPGQGLEWI |
| | GYIFPRDDIVVYAQKFQGRATLTADKSTSTAYMELSSLRSEDTAVYYCA |
| | RPPYYYSRNFYFDYWGQGTTLTVSS |
| 50 | DIQMTQSPSSLSASVGDRVTITCRVSSIISSSKLHWYQQKPGKAPKPLIYG |
| | TSTLASGVPSRFSGSGSGTDYTLTISSLQPEDFATYYCQQWSNYPFTFGQ |
| | GTKLEIK |
| 51 | QEGKFSGPLKPMTFSIYEGQEPSQIIFQFKANPPAVTFELTGETDNIFVIER |
| | EGLLYYNRALDRETRSTHNLQVAALDANGIIVEGPVPITIKVKDINDNRP |
| | TFLQSKYEGSVRQNSRPGKPFLYVNATDLDDPATPNGQLYYQIVIQLPMI |
| | NNVMYFQINNKTGAISLTREGSQELNPAKNPSYNLVISVKDMGGQSENS |
| | FSDTTSVDIIVTENIWKAPKPVEMVENSTDPHPIKITQVRWNDPGAQYSL |
| | VDKEKLPRFPFSIDQEGDIYVTQPLDREEKDAYVFYAVAKDEYGKPLSYP |
| | LEIHVKVKDINDNPPTCPSPVTVFEVQENERLGNSIGTLTAHDRDEENTA |
| | NSFLNYRIVEQTPKLPMDGLFLIQTYAGMLQLAKQSLKKQDTPQYNLTIE |
| | VSDKDFKTLCFVQINVIDINDQIPIFEKSDYGNLTLAEDTNIGSTILTIQAT |
| | DADEPFTGSSKILYHIIKGDSEGRLGVDTDPHTNTGYVIIKKPLDFETAAV |
| | SNIVFKAENPEPLVFGVKYNASSFAKFTLIVTDVNEAPQFSQHVFQAKVS |
| | EDVAIGTKVGNVTAKDPEGLDISYSLRGDTRGWLKIDHVTGEIFSVAPLD |
| | REAGSPYRVQVVATEVGGSSLSSVSEFHLILMDVNDNPPRLAKDYTGLF |
| | FCHPLSAPGSLIFEATDDDQHLFRGPHFTFSLGSGSLQNDWEVSKINGTH |
| | ARLSTRHTEFEEREYVVLIRINDGGRPPLEGIVSLPVTFCSCVEGSCFRPA |
| | GHQTGIPTVGM |
| 52 | QEGKFSGPLKPMTFSIYEGQEPSQIIFQFKANPPAVTFELTGETDNIFVIER |
| | EGLLYYNRALDRETRSTHNLQVAALDANGIIVEGPVPITIKVKDINDNRP |
| | TFLQSKYEGSVRQNSRPGKPFLYVNATDLDDPATPNGQLYYQIVIQLPMI |
| | NNVMYFQINNKTGAISLTREGSQELNPAKNPSYNLVISVKDMGGQSENS |
| | FSDTTSVDIIVTENIWKAPKPVEMVENSTDPHPIKITQVRWNDPGAQYSL |
| | VDKEKLPRFPFSIDQEGDIYVTQPLDREEKDAYVFYAVAKDEYGKPLSYP |
| | LEIHVKVKDINDNPPTCPSPVTVFEVQENERLGNSIGTLTAHDRDEENTA |
| | NSFLNYRIVEQTPKLPMDGLFLIQTYAGMLQLAKQSLKKQDTPQYNLTIE |
| | VSDKDFKTLCFVQINVIDINDQIPIFEKSDYGNLTLAEDTNIGSTILTIQAT |
| | DADEPFTGSSKILYHIIKGDSEGRLGVDTDPHTNTGYVIIKKPLDFETAAV |
| | SNIVFKAENPEPLVFGVKYNASSFAKFTLIVTDVNEAPQFSQHVFQAKVS |
| | EDVAIGTKVGNVTAKDPEGLDISYSLRGDTRGWLKIDHVTGEIFSVAPLD |
| | REAGSPYRVQVVATEVGGSSLSSVSEFHLILMDVNDNPPRLAKDYTGLF |
| | FCHPLSAPGSLIFEATDDDQHLFRGPHFTFSLGSGSLQNDWEVSKINGTH |
| | ARLSTRHTEFEEREYVVLIRINDGGRPPLEGIVSLPVTFCSCVEGSCFRPA |
| | GHQTGIPTVGMGPEQKLISEEDLNSAVDHHHHHH |
| 53 | LDANGII |
| 54 | DANGI |
| 55 | GYTFXDXT |
| 56 | GYTFXSXN |
| 57 | GYTFXDXY |
| 58 | GYTFNDHT |
| 59 | GYTFNDWT |
| 60 | GYTFNDYT |
| 61 | GYTFNDFT |
| 62 | GYTFSDHT |
| 63 | GYTFSDWT |
| 64 | GYTFSDYT |
| 65 | GYTFSDFT |
| 66 | GYTFRDHT |
| 67 | GYTFRDWT |
| 68 | GYTFRDYT |
| 69 | GYTFRDFT |
| 70 | GYTFQDHT |
| 71 | GYTFQDWT |
| 72 | GYTFQDYT |
| 73 | GYTFQDFT |
| 74 | GYTFADHT |
| 75 | GYTFADWT |
| 76 | GYTFADYT |
| 77 | GYTFADFT |
| 78 | GYTFTDWT |
| 79 | GYTFTDYT |
| 80 | GYTFTDFT |
| 81 | GYTFXXXX |
| 82 | THNLQVAALDANGIIVEGPVPIT |
| 83 | THNLQVAALDANGII |
| 84 | QVAALDANGIIVEGP |
| 85 | LDANGIIVEGPVPIT |
| 86 | Antibody 07-0663-h7_Heavy Chain (HC) amino acid sequence with a signal peptide |
| | sequence (encoded by SEQ ID NO: 88) |
| | MGWSCIILFLVATATGVHSQVQLVQSGAEVKKPGSSVKISCKVSGYTFTDHT |
| | IHWMRQAPGQGLEWIGYIFPRDDIVVYAQKFQGRATLTADKSTSTAYMELS |
| | SLRSEDTAVYYCARPPYYYSRNFYFDYWGQGTTLTVSSASTKGPSVFPLAPS |
| | SKSTSGGTAALGCLVKDYFPEPVTVSWNSGALTSGVHTFPAVLQSSGLYSLS |
| | SVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKS |
| | LSLSPGK |
| 87 | Antibody 07-0663-h7_Light Chain (LC) amino acid sequence with a signal peptide |
| | sequence (encoded by SEQ ID NO: 89) |
| | METDTLLLWVLLLWVPGSTGDIQMTQSPSSLSASVGDRVTITCRVSSIISSSK |
| | LHWYQQKPGKAPKPLIYGTSTLASGVPSRFSGSGSGTDYTLTISSLQPEDFAT |
| | YYCQQWSNYPFTFGQGTKLEIKRTVAAPSVFIFPPSDEQLKSGTASVVCLLN |
| | NFYPREAKVQWKVDNALQSGNSQESVTEQDSKDSTYSLSSTLTLSKADYEK |
| | HKVYACEVTHQGLSSPVTKSFNRGEC |
| 88 | Nucleic acid sequence encoding antibody 07-0663-h7_Heavy Chain (HC) with a |
| | signal peptide sequence |
| | ATGGGATGGTCATGTATCATCCTTTTTCTGGTAGCAACTGCAACTGGAGT |
| | ACATAGCCAGGTTCAGCTAGTTCAATCCGGGGCCGAGGTTAAAAAGCCA |
| | GGTTCAAGCGTCAAAATCTCCTGCAAAGTCTCCGGATACACATTCACCGA |
| | TCATACCATCCACTGGATGCGACAGGCTCCTGGACAAGGCCTGGAGTGG |
| | ATTGGCTACATTTTTCCACGGGACGACATTGTCGTTTACGCACAAAAATT |
| | CCAGGGCCGGGCCACACTTACCGCCGACAAAAGCACTTCAACCGCATAC |
| | ATGGAACTCTCTTCTCTGCGTTCCGAGGACACTGCCGTCTACTACTGTGC |
| | ACGGCCTCCTTATTATTACTCTCGGAATTTCTACTTCGACTACTGGGGCCA |
| | AGGCACAACCCTGACGGTGTCCTCCGCTAGCACCAAGGGCCCATCGGTCT |
| | TCCCCCTGGCACCCTCCTCCAAGAGCACCTCTGGGGGCACAGCGGCCCTG |
| | GGCTGCCTGGTCAAGGACTACTTCCCCGAACCGGTGACGGTGTCGTGGA |
| | ACTCAGGCGCCCTGACCAGCGGCGTGCACACCTTCCCGGCCGTCCTACAG |
| | TCCTCAGGACTCTACTCCCTCAGCAGCGTGGTGACCGTGCCCTCCAGCAG |
| | CTTGGGCACCCAGACCTACATCTGCAACGTGAATCACAAGCCCAGCAAC |
| | ACCAAGGTGGACAAGAAGGTTGAGCCCAAATCTTGTGACAAAACTCACA |
| | CATGCCCACCGTGCCCAGCACCTGAACTCCTGGGGGGACCGTCAGTCTTC |
| | CTCTTCCCCCCAAAACCCAAGGACACCCTCATGATCTCCCGGACCCCTGA |
| | GGTCACATGCGTGGTGGTGGACGTGAGCCACGAAGACCCTGAGGTCAAG |
| | TTCAACTGGTACGTGGACGGCGTGGAGGTGCATAATGCCAAGACAAAGC |
| | CGCGGGAGGAGCAGTACAACAGCACGTACCGTGTGGTCAGCGTCCTCAC |
| | CGTCCTGCACCAGGACTGGCTGAATGGCAAGGAGTACAAGTGCAAGGTC |
| | TCCAACAAAGCCCTCCCAGCCCCCATCGAGAAAACCATCTCCAAAGCCA |
| | AAGGGCAGCCCCGAGAACCACAGGTGTACACCCTGCCCCCATCCCGGGA |
| | CGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGA |
| | ACAACTACAAGACCACGCCTCCCGTGCTGGACTCCGACGGCTCCTTCTTC |
| | CTCTACAGCAAGCTCACCGTGGACAAGAGCAGGTGGCAGCAGGGGAACG |
| | TCTTCTCATGCTCCGTGATGCATGAGGCTCTGCACAACCACTACACGCAG |
| | AAGAGCCTCTCCCTGTCTCCGGGCAAA |
| 89 | Nucleic acid sequence encoding antibody 07-0663-h7_Light Chain (LC) with a |
| | signal peptide sequence |
| | ATGGAGACAGACACACTCCTGCTATGGGTACTGCTGCTCTGGGTTCCAGG |
| | CTCCACCGGCGATATTCAGATGACCCAGAGCCCCTCTAGCCTCTCCGCCT |
| | CTGTTGGGGACAGAGTGACCATTACATGCCGAGTATCTAGCATCATCTCT |
| | TCCTCCAAACTGCACTGGTACCAGCAAAAGCCTGGCAAAGCCCCTAAGC |
| | CTCTGATTTATGGGACTTCTACTTTAGCCTCCGGCGTCCCAAGTCGGTTCT |
| | CTGGAAGTGGCTCCGGCACCGACTACACTCTGACTATCTCCAGTCTGCAG |
| | CCCGAGGACTTCGCTACATACTACTGCCAACAATGGTCCAACTATCCTTT |
| | TACATTCGGACAAGGCACTAAGCTGGAAATCAAACGTACGGTGGCTGCA |
| | CCATCTGTCTTCATCTTCCCGCCATCTGATGAGCAGTTGAAATCTGGAAC |
| | TGCCTCTGTTGTGTGCCTGCTGAATAACTTCTATCCCAGAGAGGCCAAAG |
| | TACAGTGGAAGGTGGATAACGCCCTCCAATCGGGTAACTCCCAGGAGAG |
| | TGTCACAGAGCAGGACAGCAAGGACAGCACCTACAGCCTCAGCAGCACC |
| | CTGACGCTGAGCAAAGCAGACTACGAGAAACACAAAGTCTACGCCTGCG |
| | AAGTCACCCATCAGGGCCTGAGCTCGCCCGTCACAAAGAGCTTCAACAG |
| | GGGAGAGTGT |
| 90 | Antibody 07-0663-h7_Heavy Chain (HC) amino acid sequence with a signal peptide |
| | sequence (encoded by SEQ ID NO: 92) |
| | MEFGLSWVFLVALFRGVQCQVQLVQSGAEVKKPGSSVKISCKVSGYTFTDH |
| | TIHWMRQAPGQGLEWIGYIFPRDDIVVYAQKFQGRATLTADKSTSTAYMEL |
| | SSLRSEDTAVYYCARPPYYYSRNFYFDYWGQGTTLTVSSASTKGPSVFPLAP |
| | SSKSTSGGTAALGCLVKDYFPEPVTVSWNSGALTSGVHTFPAVLQSSGLYSL |
| | SSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKS |
| | LSLSPGK |
| 91 | Antibody 07-0663-h7_Light Chain (LC) amino acid sequence with a signal peptide |
| | sequence (encoded by SEQ ID NO: 93) |
| | MDMRVPAQLLGLLLLWLSGARCDIQMTQSPSSLSASVGDRVTITCRVSSIISS |
| | SKLHWYQQKPGKAPKPLIYGTSTLASGVPSRFSGSGSGTDYTLTISSLQPEDF |
| | ATYYCQQWSNYPFTFGQGTKLEIKRTVAAPSVFIFPPSDEQLKSGTASVVCLL |
| | NNFYPREAKVQWKVDNALQSGNSQESVTEQDSKDSTYSLSSTLTLSKADYE |
| | KHKVYACEVTHQGLSSPVTKSFNRGEC |
| 92 | Nucleic acid sequence encoding antibody 07-0663-h7_Heavy Chain (HC) with a |
| | signal peptide sequence (p07-0663-h7, ATCC Accession No: to be determined) |
| | ATGGAGTTTGGGCTGAGCTGGGTTTTCCTCGTTGCTCTTTTTAGAGGTGTC |
| | CAGTGTCAAGTGCAGCTGGTGCAGAGCGGAGCCGAGGTGAAAAAGCCAG |
| | GCAGCTCTGTGAAGATCAGCTGCAAGGTGTCTGGCTACACCTTCACCGAC |
| | CACACCATCCACTGGATGCGGCAGGCCCCTGGCCAGGGCCTGGAATGGA |
| | TCGGCTACATCTTTCCTAGAGATGACATCGTGGTCTACGCCCAGAAGTTC |
| | CAGGGCAGAGCCACACTGACCGCTGATAAGTCTACAAGCACAGCTTACA |
| | TGGAACTGAGCTCCCTGCGGAGCGAGGACACCGCCGTGTACTATTGTGCC |
| | AGACCTCCCTACTACTACAGCAGAAACTTCTACTTCGACTACTGGGGCCA |
| | GGGAACCACCCTGACAGTGTCCAGCGCTAGCACCAAGGGCCCATCGGTC |
| | TTCCCCCTGGCACCCTCCTCCAAGAGCACCTCTGGGGGCACAGCGGCCCT |
| | GGGCTGCCTGGTCAAGGACTACTTCCCCGAACCGGTGACGGTGTCGTGG |
| | AACTCAGGCGCCCTGACCAGCGGCGTGCACACCTTCCCGGCTGTCCTACA |
| | GTCCTCAGGACTCTACTCCCTCAGCAGCGTGGTGACCGTGCCCTCCAGCA |
| | GCTTGGGCACCCAGACCTACATCTGCAACGTGAATCACAAGCCCAGCAA |
| | CACCAAGGTGGACAAGAAAGTTGAGCCCAAATCTTGTGACAAAACTCAC |
| | ACATGCCCACCGTGCCCAGCACCTGAACTCCTGGGGGGACCGTCAGTCTT |
| | CCTCTTCCCCCCAAAACCCAAGGACACCCTCATGATCTCCCGGACCCCTG |
| | AGGTCACATGCGTGGTGGTGGACGTGAGCCACGAAGACCCTGAGGTCAA |
| | GTTCAACTGGTACGTGGACGGCGTGGAGGTGCATAATGCCAAGACAAAG |
| | CCGCGGGAGGAGCAGTACAACAGCACGTACCGGGTGGTCAGCGTCCTCA |
| | CCGTCCTGCACCAGGACTGGCTGAATGGCAAGGAGTACAAGTGCAAGGT |
| | GTCCAACAAAGCCCTCCCAGCCCCCATCGAGAAAACCATCTCCAAAGCC |
| | AAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTT |
| | CTATCCCAGCGACATCGCCGTGGAGTGGGAGAGCAATGGGCAGCCGGAG |
| | AACAACTACAAGACCACGCCTCCCGTGCTGGACTCCGACGGCTCCTTCTT |
| | CCTCTACAGCAAGCTCACCGTGGACAAGAGCAGGTGGCAGCAGGGGAAC |
| | GTCTTCTCATGCTCCGTGATGCATGAGGCTCTGCACAACCACTACACGCA |
| | GAAGAGCCTCTCCCTGTCTCCGGGTAAATGA |
| 93 | Nucleic acid sequence encoding antibody 07-0663-h7_Light Chain (LC) with a |
| | signal peptide sequence (p07-0663-h7, ATCC Accession No: to be determined) |
| | ATGGACATGAGGGTCCCTGCTCAGCTCCTGGGGCTCCTGCTGCTCTGGCT |
| | CTCAGGTGCCAGATGTGACATCCAGATGACCCAGAGCCCTAGCAGCCTG |
| | AGCGCCAGCGTGGGAGATAGAGTCACAATCACCTGTAGAGTGTCCTCCA |
| | TCATCAGCTCTTCTAAGCTGCACTGGTATCAGCAGAAACCAGGCAAGGCC |
| | CCTAAGCCTCTGATCTACGGCACAAGCACCCTGGCTTCTGGCGTGCCCAG |
| | CCGGTTCAGCGGCAGCGGATCTGGCACCGACTACACCCTGACCATTAGC |
| | AGCCTGCAGCCTGAGGACTTCGCCACATACTACTGCCAGCAATGGTCCAA |
| | CTACCCCTTTACATTCGGCCAGGGCACCAAGCTGGAAATCAAGCGTACG |
| | GTGGCGGCGCCATCTGTCTTCATCTTCCCGCCATCTGATGAGCAGTTGAA |
| | ATCTGGAACTGCCTCTGTTGTGTGCCTGCTGAATAACTTCTATCCCAGAG |
| | AGGCCAAAGTACAGTGGAAGGTGGATAACGCCCTCCAATCGGGTAACTC |
| | CCAGGAGAGTGTCACAGAGCAGGACAGCAAGGACAGCACCTACAGCCTC |
| | AGCAGCACCCTGACGCTGAGCAAAGCAGACTACGAGAAACACAAAGTCT |
| | ACGCCTGCGAAGTCACCCATCAGGGCCTGAGCTCGCCCGTCACAAAGAG |
| | CTTCAACAGGGGAGAGTGTTAG |
| 94 | Antibody 07-0663-h7_Heavy Chain (HC) amino acid sequence (without the leader |
| | sequence) |
| | QVQLVQSGAEVKKPGSSVKISCKVSGYTFTDHTIHWMRQAPGQGLEWIGYI |
| | FPRDDIVVYAQKFQGRATLTADKSTSTAYMELSSLRSEDTAVYYCARPPYY |
| | YSRNFYFDYWGQGTTLTVSSASTKGPSVFPLAPSSKSTSGGTAALGCLVKDY |
| | FPEPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRDE |
| | LTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 95 | Antibody 07-0663-h7_Light Chain (LC) amino acid sequence (without the leader |
| | sequence) |
| | DIQMTQSPSSLSASVGDRVTITCRVSSIISSSKLHWYQQKPGKAPKPLIYGTST |
| | LASGVPSRFSGSGSGTDYTLTISSLQPEDFATYYCQQWSNYPFTFGQGTKLEI |
| | KRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSF |
| | NRGEC |
| 96 | Amino acid sequence of 07-0646-h7scfv anti-CDH17 scFv antibody |
| | DIQLTQSPSSLSASVGDRVTMTCRASSSVSSSYLHWYQQKPGKAPKLLIYSTS |
| | NLASGVPSRFSGSGSGTDYTLTISSVQPEDFATYYCQQYDSSPSTFGQGTKLE |
| | IKGGGGSGGGGSGGGGSQVQLVQSGAEVKKPGASVKMSCKASGYTFTSYN |
| | MHWVRQAPGQGLEWIGAIYPGNGDTSYAQKFQGRATLTVDTSTSTAYMEL |
| | SSLRSEDTAVYYCARGRGRYFEYWGQGTTLTVSS |
| | The scFv may optionally further comprise at its C-terminus a (His)₆- |
| | epitope tag. |
| 97 | Amino acid sequence of 07-0653-h43scfv anti-CDH17 scFv antibody |
| | DIQLTQSPSSLSASVGDRVTITCRATSSLSYIHWYQQKPGKAPKPLIYEISKLA |
| | SGVPSRFSGSGSGTDYTLTISSLQPEDFATYYCQQWNYPFTFGQGTKLEIKGG |
| | GGSGGGGGGGGSQVQLVQSGAEVKKPGASVKVSCKASGYTFTDYYMNW |
| | VRQAPGQGLEWMGVIYPYSGGIGYAQKFQGRVTMTVDKSTSTAYMELSSL |
| | RSEDTAVYYCARGRGDYFGLFDFWGQGTTVTVSS |
| | The scFv may optionally further comprise at its C-terminus a (His)₆- |
| | epitope tag. |
| 98 | Amino acid sequence of 07-0663-h7scfv anti-CDH17 scFv antibody |
| | DIQMTQSPSSLSASVGDRVTITCRVSSIISSSKLHWYQQKPGKAPKPLIYGTST |
| | LASGVPSRFSGSGSGTDYTLTISSLQPEDFATYYCQQWSNYPFTFGQGTKLEI |
| | KGGGGSGGGGSGGGGSQVQLVQSGAEVKKPGSSVKISCKVSGYTFTDHTIH |
| | WMRQAPGQGLEWIGYIFPRDDIVVYAQKFQGRATLTADKSTSTAYMELSSL |
| | RSEDTAVYYCARPPYYYSRNFYFDYWGQGTTLTVSS |
| | The scFv may optionally further comprise at its C-terminus a (His)₆- |
| | epitope tag. |
| 99 | Amino acid sequence of 07-0646-h7Bs CDH17-CD3 bispecific antibody, which is |
| | also a bispecific T-cell engager (BiTE) |
| | DIQLTQSPSSLSASVGDRVTMTCRASSSVSSSYLHWYQQKPGKAPKLLIYSTS |
| | NLASGVPSRFSGSGSGTDYTLTISSVQPEDFATYYCQQYDSSPSTFGQGTKLE |
| | IKGGGGSGGGGSGGGGSQVQLVQSGAEVKKPGASVKMSCKASGYTFTSYN |
| | MHWVRQAPGQGLEWIGAIYPGNGDTSYAQKFQGRATLTVDTSTSTAYMEL |
| | SSLRSEDTAVYYCARGRGRYFEYWGQGTTLTVSSGGGGSDIKLQQSGAELA |
| | RPGASVKMSCKTSGYTFTRYTMHWVKQRPGQGLEWIGYINPSRGYTNYNQ |
| | KFKDKATLTTDKSSSTAYMQLSSLTSEDSAVYYCARYYDDHYCLDYWGQG |
| | TTLTVSSVEGGSGGSGGSGGSGGVDDIQLTQSPAIMSASPGEKVTMTCRASS |
| | SVSYMNWYQQKSGTSPKRWIYDTSKVASGVPYRFSGSGSGTSYSLTISSMEA |
| | EDAATYYCQQWSSNPLTFGAGTKLELK |
| | The bispecific antibody may optionally further comprise at its C-terminus |
| | a (His)₆-epitope tag. |
| 100 | Amino acid sequence of 07-0653-h43Bs CDH17-CD3 bispecific antibody, which is |
| | also a bispecific T-cell engager (BiTE) |
| | DIQLTQSPSSLSASVGDRVTITCRATSSLSYIHWYQQKPGKAPKPLIYEISKLA |
| | SGVPSRFSGSGSGTDYTLTISSLQPEDFATYYCQQWNYPFTFGQGTKLEIKGG |
| | GGSGGGGSGGGGSQVQLVQSGAEVKKPGASVKVSCKASGYTFTDYYMNW |
| | VRQAPGQGLEWMGVIYPYSGGIGYAQKFQGRVTMTVDKSTSTAYMELSSL |
| | RSEDTAVYYCARGRGDYFGLFDFWGQGTTVTVSSGGGGSDIKLQQSGAELA |
| | RPGASVKMSCKTSGYTFTRYTMHWVKQRPGQGLEWIGYINPSRGYTNYNQ |
| | KFKDKATLTTDKSSSTAYMQLSSLTSEDSAVYYCARYYDDHYCLDYWGQG |
| | TTLTVSSVEGGSGGSGGSGGSGGVDDIQLTQSPAIMSASPGEKVTMTCRASS |
| | SVSYMNWYQQKSGTSPKRWIYDTSKVASGVPYRFSGSGSGTSYSLTISSMEA |
| | EDAATYYCQQWSSNPLTFGAGTKLELK |
| | The bispecific antibody may optionally further comprise at its C-terminus |
| | a (His)₆-epitope tag. |
| 101 | Amino acid sequence of 07-0663-h7Bs CDH17-CD3 bispecific antibody, which is |
| | also a bispecific T-cell engager (BiTE) |
| | DIQMTQSPSSLSASVGDRVTITCRVSSIISSSKLHWYQQKPGKAPKPLIYGTST |
| | LASGVPSRFSGSGSGTDYTLTISSLQPEDFATYYCQQWSNYPFTFGQGTKLEI |
| | KGGGGSGGGGSGGGGSQVQLVQSGAEVKKPGSSVKISCKVSGYTFTDHTIH |
| | WMRQAPGQGLEWIGYIFPRDDIVVYAQKFQGRATLTADKSTSTAYMELSSL |
| | RSEDTAVYYCARPPYYYSRNFYFDYWGQGTTLTVSSGGGGSDIKLQQSGAE |
| | LARPGASVKMSCKTSGYTFTRYTMHWVKQRPGQGLEWIGYINPSRGYTNY |
| | NQKFKDKATLTTDKSSSTAYMQLSSLTSEDSAVYYCARYYDDHYCLDYWG |
| | QGTTLTVSSVEGGSGGSGGSGGSGGVDDIQLTQSPAIMSASPGEKVTMTCRA |
| | SSSVSYMNWYQQKSGTSPKRWIYDTSKVASGVPYRFSGSGSGTSYSLTISSM |
| | EAEDAATYYCQQWSSNPLTFGAGTKLELK |
| | The bispecific antibody may optionally further comprise at its C-terminus |
| | a (His)₆-epitope tag. |
| 102 | Nucleic acid sequence encoding 07-0646-h7scfv anti-CDH17 scFv |
| | GATATCCAGCTGACACAGAGCCCTAGCAGCCTGAGCGCCAGCGTGGGCG |
| | ACCGGGTGACCATGACCTGTAGAGCCTCTAGCAGCGTGTCCTCCAGCTAC |
| | CTGCACTGGTATCAGCAAAAGCCCGGCAAAGCCCCTAAGCTCCTGATCTA |
| | CAGCACCAGCAACCTGGCTTCTGGAGTGCCCAGCAGATTCAGCGGATCT |
| | GGCAGCGGCACAGATTACACCCTGACCATCAGCTCTGTCCAGCCTGAGG |
| | ACTTCGCCACCTACTACTGCCAGCAGTACGACAGCTCCCCATCTACATTT |
| | GGCCAGGGCACCAAGCTGGAAATCAAGGGTGGTGGTGGTTCTGGAGGAG |
| | GAGGATCTGGAGGGGGGGGGTCCCAAGTGCAGCTGGTCCAGAGCGGCGC |
| | CGAGGTGAAAAAGCCTGGAGCTTCTGTGAAGATGAGCTGCAAGGCCTCT |
| | GGCTACACCTTCACCAGCTACAACATGCACTGGGTGCGGCAGGCCCCTG |
| | GCCAGGGCCTGGAATGGATCGGCGCTATCTACCCCGGCAACGGCGATAC |
| | ATCTTATGCCCAGAAGTTTCAGGGAAGAGCCACACTGACCGTGGACACC |
| | AGCACCTCCACCGCCTACATGGAACTGAGCAGCCTGAGAAGCGAGGACA |
| | CAGCCGTGTACTACTGTGCCAGAGGAAGAGGCCGGTACTTCGAGTACTG |
| | GGGCCAGGGCACCACCCTGACAGTGTCCAGC |
| 103 | Nucleic acid sequence encoding 07-0653-h43scfv anti-CDH17 scFv |
| | GATATCCAGCTGACACAGAGCCCTAGCAGCCTCTCCGCCAGCGTGGGCG |
| | ACCGGGTGACCATCACCTGTAGAGCCACCAGCAGCCTGAGCTACATCCA |
| | CTGGTATCAGCAGAAACCCGGCAAGGCCCCTAAGCCTCTGATCTACGAG |
| | ATTAGCAAGCTGGCTTCTGGAGTGCCATCTAGATTCAGCGGCAGCGGATC |
| | TGGCACCGACTACACCCTGACCATCTCCTCCCTGCAGCCTGAGGACTTCG |
| | CCACATACTACTGCCAGCAATGGAACTACCCCTTCACCTTTGGCCAGGGC |
| | ACAAAGCTGGAAATCAAGGGTGGTGGTGGTTCTGGAGGAGGAGGATCTG |
| | GAGGGGGGGGGTCCCAAGTCCAACTCGTCCAATCCGGTGCTGAAGTCAA |
| | GAAACCTGGTGCATCCGTCAAAGTCTCCTGTAAAGCAAGTGGTTATACAT |
| | TTACTGATTATTATATGAATTGGGTCCGCCAAGCACCTGGGCAAGGGCTC |
| | GAATGGATGGGTGTAATTTATCCATATTCTGGCGGAATAGGATATGCTCA |
| | AAAGTTTCAAGGGCGAGTAACAATGACAGTTGATAAATCCACATCAACT |
| | GCTTACATGGAATTGTCCTCACTCCGAAGTGAAGATACGGCTGTTTATTA |
| | TTGTGCAAGAGGGCGTGGGGATTATTTTGGACTCTTTGATTTCTGGGGAC |
| | AAGGAACGACTGTAACAGTCTCTTCC |
| 104 | Nucleic acid sequence encoding 07-0663-h7scfv anti-CDH17 scFv |
| | GACATCCAGATGACCCAGAGCCCTAGCAGCCTGAGCGCCAGCGTGGGAG |
| | ATAGAGTCACAATCACCTGTAGAGTGTCCTCCATCATCAGCTCTTCTAAG |
| | CTGCACTGGTATCAGCAGAAACCAGGCAAGGCCCCTAAGCCTCTGATCT |
| | ACGGCACAAGCACCCTGGCTTCTGGCGTGCCCAGCCGGTTCAGCGGCAG |
| | CGGATCTGGCACCGACTACACCCTGACCATTAGCAGCCTGCAGCCTGAG |
| | GACTTCGCCACATACTACTGCCAGCAATGGTCCAACTACCCCTTTACATT |
| | CGGCCAGGGCACCAAGCTGGAAATCAAGGGTGGTGGTGGTTCTGGAGGA |
| | GGAGGATCTGGAGGGGGGGGGTCCCAAGTGCAGCTGGTGCAGAGCGGA |
| | GCCGAGGTGAAAAAGCCAGGCAGCTCTGTGAAGATCAGCTGCAAGGTGT |
| | CTGGCTACACCTTCACCGACCACACCATCCACTGGATGCGGCAGGCCCCT |
| | GGCCAGGGCCTGGAATGGATCGGCTACATCTTTCCTAGAGATGACATCGT |
| | GGTCTACGCCCAGAAGTTCCAGGGCAGAGCCACACTGACCGCTGATAAG |
| | TCTACAAGCACAGCTTACATGGAACTGAGCTCCCTGCGGAGCGAGGACA |
| | CCGCCGTGTACTATTGTGCCAGACCTCCCTACTACTACAGCAGAAACTTC |
| | TACTTCGACTACTGGGGCCAGGGAACCACCCTGACAGTGTCCAGC |
| 105 | Nucleic acid sequence encoding 07-0646-h7Bs CDH17-CD3 bispecific antibody, |
| | which is also a bispecific T-cell engager (BiTE) |
| | GATATCCAGCTGACACAGAGCCCTAGCAGCCTGAGCGCCAGCGTGGGCG |
| | ACCGGGTGACCATGACCTGTAGAGCCTCTAGCAGCGTGTCCTCCAGCTAC |
| | CTGCACTGGTATCAGCAAAAGCCCGGCAAAGCCCCTAAGCTCCTGATCTA |
| | CAGCACCAGCAACCTGGCTTCTGGAGTGCCCAGCAGATTCAGCGGATCT |
| | GGCAGCGGCACAGATTACACCCTGACCATCAGCTCTGTCCAGCCTGAGG |
| | ACTTCGCCACCTACTACTGCCAGCAGTACGACAGCTCCCCATCTACATTT |
| | GGCCAGGGCACCAAGCTGGAAATCAAGGGTGGTGGTGGTTCTGGAGGAG |
| | GAGGATCTGGAGGGGGGGGGTCCCAAGTGCAGCTGGTCCAGAGCGGCGC |
| | CGAGGTGAAAAAGCCTGGAGCTTCTGTGAAGATGAGCTGCAAGGCCTCT |
| | GGCTACACCTTCACCAGCTACAACATGCACTGGGTGCGGCAGGCCCCTG |
| | GCCAGGGCCTGGAATGGATCGGCGCTATCTACCCCGGCAACGGCGATAC |
| | ATCTTATGCCCAGAAGTTTCAGGGAAGAGCCACACTGACCGTGGACACC |
| | AGCACCTCCACCGCCTACATGGAACTGAGCAGCCTGAGAAGCGAGGACA |
| | CAGCCGTGTACTACTGTGCCAGAGGAAGAGGCCGGTACTTCGAGTACTG |
| | GGGCCAGGGCACCACCCTGACAGTGTCCAGCGGCGGCGGCGGCAGCGAC |
| | ATCAAGCTGCAGCAGAGCGGCGCCGAGCTGGCCAGGCCCGGCGCCAGCG |
| | TGAAGATGAGCTGCAAGACCAGCGGCTACACCTTCACCAGGTACACCAT |
| | GCACTGGGTGAAGCAGAGGCCCGGCCAGGGCCTGGAGTGGATCGGCTAC |
| | ATCAACCCCAGCAGGGGCTACACCAACTACAACCAGAAGTTCAAGGACA |
| | AGGCCACCCTGACCACCGACAAGAGCAGCAGCACCGCCTACATGCAGCT |
| | GAGCAGCCTGACCAGCGAGGACAGCGCCGTGTACTACTGCGCCAGGTAC |
| | TACGACGACCACTACTGCCTGGACTACTGGGGCCAGGGCACCACCCTGA |
| | CCGTGAGCAGCGTGGAGGGCGGCAGCGGCGGCAGCGGCGGCAGCGGCG |
| | GCAGCGGCGGCGTGGACGACATCCAGCTGACCCAGAGCCCCGCCATCAT |
| | GAGCGCCAGCCCCGGCGAGAAGGTGACCATGACCTGCAGGGCCAGCAGC |
| | AGCGTGAGCTACATGAACTGGTACCAGCAGAAGAGCGGCACCAGCCCCA |
| | AGAGGTGGATCTACGACACCAGCAAGGTGGCCAGCGGCGTGCCCTACAG |
| | GTTCAGCGGCAGCGGCAGCGGCACCAGCTACAGCCTGACCATCAGCAGC |
| | ATGGAGGCCGAGGACGCCGCCACCTACTACTGCCAGCAGTGGAGCAGCA |
| | ACCCCCTGACCTTCGGCGCCGGCACCAAGCTGGAGCTGAAG |
| 106 | Nucleic acid sequence encoding 07-0653-h43Bs CDH17-CD3 bispecific antibody, |
| | which is also a bispecific T-cell engager (BiTE) |
| | GATATCCAGCTGACACAGAGCCCTAGCAGCCTCTCCGCCAGCGTGGGCG |
| | ACCGGGTGACCATCACCTGTAGAGCCACCAGCAGCCTGAGCTACATCCA |
| | CTGGTATCAGCAGAAACCCGGCAAGGCCCCTAAGCCTCTGATCTACGAG |
| | ATTAGCAAGCTGGCTTCTGGAGTGCCATCTAGATTCAGCGGCAGCGGATC |
| | TGGCACCGACTACACCCTGACCATCTCCTCCCTGCAGCCTGAGGACTTCG |
| | CCACATACTACTGCCAGCAATGGAACTACCCCTTCACCTTTGGCCAGGGC |
| | ACAAAGCTGGAAATCAAGGGTGGTGGTGGTTCTGGAGGAGGAGGATCTG |
| | GAGGGGGGGGGTCCCAAGTCCAACTCGTCCAATCCGGTGCTGAAGTCAA |
| | GAAACCTGGTGCATCCGTCAAAGTCTCCTGTAAAGCAAGTGGTTATACAT |
| | TTACTGATTATTATATGAATTGGGTCCGCCAAGCACCTGGGCAAGGGCTC |
| | GAATGGATGGGTGTAATTTATCCATATTCTGGCGGAATAGGATATGCTCA |
| | AAAGTTTCAAGGGCGAGTAACAATGACAGTTGATAAATCCACATCAACT |
| | GCTTACATGGAATTGTCCTCACTCCGAAGTGAAGATACGGCTGTTTATTA |
| | TTGTGCAAGAGGGCGTGGGGATTATTTTGGACTCTTTGATTTCTGGGGAC |
| | AAGGAACGACTGTAACAGTCTCTTCCGGCGGCGGCGGCAGCGACATCAA |
| | GCTGCAGCAGAGCGGCGCCGAGCTGGCCAGGCCCGGCGCCAGCGTGAAG |
| | ATGAGCTGCAAGACCAGCGGCTACACCTTCACCAGGTACACCATGCACT |
| | GGGTGAAGCAGAGGCCCGGCCAGGGCCTGGAGTGGATCGGCTACATCAA |
| | CCCCAGCAGGGGCTACACCAACTACAACCAGAAGTTCAAGGACAAGGCC |
| | ACCCTGACCACCGACAAGAGCAGCAGCACCGCCTACATGCAGCTGAGCA |
| | GCCTGACCAGCGAGGACAGCGCCGTGTACTACTGCGCCAGGTACTACGA |
| | CGACCACTACTGCCTGGACTACTGGGGCCAGGGCACCACCCTGACCGTG |
| | AGCAGCGTGGAGGGCGGCAGCGGCGGCAGCGGCGGCAGCGGCGGCAGC |
| | GGCGGCGTGGACGACATCCAGCTGACCCAGAGCCCCGCCATCATGAGCG |
| | CCAGCCCCGGCGAGAAGGTGACCATGACCTGCAGGGCCAGCAGCAGCGT |
| | GAGCTACATGAACTGGTACCAGCAGAAGAGCGGCACCAGCCCCAAGAGG |
| | TGGATCTACGACACCAGCAAGGTGGCCAGCGGCGTGCCCTACAGGTTCA |
| | GCGGCAGCGGCAGCGGCACCAGCTACAGCCTGACCATCAGCAGCATGGA |
| | GGCCGAGGACGCCGCCACCTACTACTGCCAGCAGTGGAGCAGCAACCCC |
| | CTGACCTTCGGCGCCGGCACCAAGCTGGAGCTGAAG |
| 107 | Nucleic acid sequence encoding 07-0663-h7Bs CDH17-CD3 bispecific antibody, |
| | which is also a bispecific T-cell engager (BiTE) |
| | GACATCCAGATGACCCAGAGCCCTAGCAGCCTGAGCGCCAGCGTGGGAG |
| | ATAGAGTCACAATCACCTGTAGAGTGTCCTCCATCATCAGCTCTTCTAAG |
| | CTGCACTGGTATCAGCAGAAACCAGGCAAGGCCCCTAAGCCTCTGATCT |
| | ACGGCACAAGCACCCTGGCTTCTGGCGTGCCCAGCCGGTTCAGCGGCAG |
| | CGGATCTGGCACCGACTACACCCTGACCATTAGCAGCCTGCAGCCTGAG |
| | GACTTCGCCACATACTACTGCCAGCAATGGTCCAACTACCCCTTTACATT |
| | CGGCCAGGGCACCAAGCTGGAAATCAAGGGTGGTGGTGGTTCTGGAGGA |
| | GGAGGATCTGGAGGGGGGGGGTCCCAAGTGCAGCTGGTGCAGAGCGGA |
| | GCCGAGGTGAAAAAGCCAGGCAGCTCTGTGAAGATCAGCTGCAAGGTGT |
| | CTGGCTACACCTTCACCGACCACACCATCCACTGGATGCGGCAGGCCCCT |
| | GGCCAGGGCCTGGAATGGATCGGCTACATCTTTCCTAGAGATGACATCGT |
| | GGTCTACGCCCAGAAGTTCCAGGGCAGAGCCACACTGACCGCTGATAAG |
| | TCTACAAGCACAGCTTACATGGAACTGAGCTCCCTGCGGAGCGAGGACA |
| | CCGCCGTGTACTATTGTGCCAGACCTCCCTACTACTACAGCAGAAACTTC |
| | TACTTCGACTACTGGGGCCAGGGAACCACCCTGACAGTGTCCAGCGGCG |
| | GCGGCGGCAGCGACATCAAGCTGCAGCAGAGCGGCGCCGAGCTGGCCAG |
| | GCCCGGCGCCAGCGTGAAGATGAGCTGCAAGACCAGCGGCTACACCTTC |
| | ACCAGGTACACCATGCACTGGGTGAAGCAGAGGCCCGGCCAGGGCCTGG |
| | AGTGGATCGGCTACATCAACCCCAGCAGGGGCTACACCAACTACAACCA |
| | GAAGTTCAAGGACAAGGCCACCCTGACCACCGACAAGAGCAGCAGCACC |
| | GCCTACATGCAGCTGAGCAGCCTGACCAGCGAGGACAGCGCCGTGTACT |
| | ACTGCGCCAGGTACTACGACGACCACTACTGCCTGGACTACTGGGGCCA |
| | GGGCACCACCCTGACCGTGAGCAGCGTGGAGGGCGGCAGCGGCGGCAGC |
| | GGCGGCAGCGGCGGCAGCGGCGGCGTGGACGACATCCAGCTGACCCAGA |
| | GCCCCGCCATCATGAGCGCCAGCCCCGGCGAGAAGGTGACCATGACCTG |
| | CAGGGCCAGCAGCAGCGTGAGCTACATGAACTGGTACCAGCAGAAGAGC |
| | GGCACCAGCCCCAAGAGGTGGATCTACGACACCAGCAAGGTGGCCAGCG |
| | GCGTGCCCTACAGGTTCAGCGGCAGCGGCAGCGGCACCAGCTACAGCCT |
| | GACCATCAGCAGCATGGAGGCCGAGGACGCCGCCACCTACTACTGCCAG |
| | CAGTGGAGCAGCAACCCCCTGACCTTCGGCGCCGGCACCAAGCTGGAGC |
| | TGAAG |

*SEQ ID NOs: 86-89 (set 1) and SEQ ID NOs: 90-93 (set 2) all have been used to express the HC and LC of 07-0663-h7. The sequences in set 1 and set 2 differ in the usage of different signal peptides (which gets cleaved off from the mature protein) and the presence of different silent nucleic acid mutations that do not alter the amino acid sequences of the translated protein; SEQ ID NOs 86-89 have been codon-optimized.
*The scFvs and bispecific antibodies (e.g., BiTEs) used herein further comprises a C-terminal (His)₆ epitope tag, which facilitated purification and detection.
*It is well understood in the art that a newly synthesized polypeptide destined for the secretory pathway (e.g., a protein that is secreted or inserted into a membrane) comprises a signal peptide. Thus, any one of the polypeptides presented herein may further comprise a signal peptide, variations of amino acid sequences of which are well known in the art.

TABLE 2A
Amino Acid Sequence of Humanized CDH17 Antibody 07-0646-h7
(IMGT Numbering)

| | | |
|---|---|---|
| Name of antibodies | 07-0646-h7 | |
| name of expression | p07-0646-h7 | |
| vector | | |
| Name of VH or VL | 07-0646-h7_VH | 07-0646-h7_VL |
| FR1-IMGT | QVQLVQSGAEVKKPGASVKMSCK | DIQLTQSPSSLSASVGDRVTMTC |
| | AS | RAS |
| CDR1-IMGT | GYTFTSYN | SSVSSSY |
| FR2-IMGT | MHWVRQAPGQGLEWIGA | LHWYQQKPGKAPKLLIY |
| CDR2-IMGT | IYPGNGDT | STS |
| FR3-IMGT | SYAQKFQGRATLTVDTSTSTAYME | NLASGVPSRFSGSGSGTDYTLTIS |
| | LSSLRSEDTAVYYC | SVQPEDFATYYC |
| CDR3-IMGT | ARGRGRYFEY | QQYDSSPST |
| FR4-IMGT | WGQGTTLTVSS | FGQGTKLEIK |
| VH or VL sequence | QVQLVQSGAEVKKPGASVKMSCK | DIQLTQSPSSLSASVGDRVTMTC |
| | ASGYTFTSYNMHWVRQAPGQGLE | RASSSVSSSYLHWYQQKPGKAPK |
| | WIGAIYPGNGDTSYAQKFQGRATL | LLIYSTSNLASGVPSRFSGSGSGT |
| | TVDTSTSTAYMELSSLRSEDTAVYY | DYTLTISSVQPEDFATYYCQQYDS |
| | CARGRGRYFEYWGQGTTLTVSS | SPSTFGQGTKLEIK |
| Name of VH or VL | 07-0646-h7_VH | 07-0646-h7_VL |
| Project of antibody | CDH17 | CDH17 |

TABLE 2B
Amino Acid Sequence of Humanized CDH17 Antibody 07-0646-h7
(Kabat Numbering)

| Name of VH | 07-0646-h7_VH | 07-0646- | 07-0646- |
|---|---|---|---|
| | | h7_VH | h7_VH |
| Method | Kabat | Kabat | Kabat |
| Region | Sequence Fragment | Residues | Length |
| HFR1 | QVQLVQSGAEVKKPGASVKMSCKASGYTFT | 1-30 | 30 |
| CDR-H1 | SYNMH | 31-35 | 5 |
| HFR2 | WVRQAPGQGLEWIG | 36-49 | 14 |
| CDR-H2 | AIYPGNGDTSYAQKFQG | 50-66 | 17 |
| HFR3 | RATLTVDTSTSTAYMELSSLRSEDTAVYYCAR | 67-98 | 32 |
| CDR-H3 | GRGRYFEY | 99-106 | 8 |
| HFR4 | WGQGTTLTVSS | 107-117 | 11 |
| VH sequence | QVQLVQSGAEVKKPGASVKMSCKASGYTFTSYNMH | | |
| | WVRQAPGQGLEWIGAIYPGNGDTSYAQKFQGRATL | | |
| | TVDTSTSTAYMELSSLRSEDTAVYYCARGRGRYFEYW | | |
| | GQGTTLTVSS | | |
| Name of VH | 07-0646-h7_VH | 07-0646- | 07-0646- |
| | | h7_VH | h7_VH |
| Name of VL | 07-0646-h7_VL | 07-0646- | 07-0646- |
| | | h7_VL | h7_VL |
| Method | Kabat | Kabat | Kabat |
| Region | Sequence Fragment | Residues | Length |
| LFR1 | DIQLTQSPSSLSASVGDRVTMTC | 1-23 | 23 |
| CDR-L1 | RASSSVSSSYLH | 24-35 | 12 |
| LFR2 | WYQQKPGKAPKLLIY | 36-50 | 15 |
| CDR-L2 | STSNLAS | 51-57 | 7 |
| LFR3 | GVPSRFSGSGSGTDYTLTISSVQPEDFATYYC | 58-89 | 32 |
| CDR-L3 | QQYDSSPST | 90-98 | 9 |
| LFR4 | FGQGTKLEIKRTV | 99-111 | 13 |
| VL sequence | DIQLTQSPSSLSASVGDRVTMTCRASSSVSSSYLHWY | | |
| | QQKPGKAPKLLIYSTSNLASGVPSRFSGSGSGTDYTLT | | |
| | ISSVQPEDFATYYCQQYDSSPSTFGQGTKLEIKRTV | | |
| Name of VL | 07-0646-h7_VL | 07-0646- | 07-0646- |
| | | h7_VL | h7_VL |

TABLE 2C
Amino Acid Sequence of Humanized CDH17 Antibody 07-0646-h7
(AbM Numbering)

| Name of VH | 07-0646-h7_VH | 07-0646- | 07-0646- |
|---|---|---|---|
| | | h7_VH | h7_VH |
| Method | AbM | AbM | AbM |
| Region | Sequence Fragment | Residues | Length |
| HFR1 | QVQLVQSGAEVKKPGASVKMSCKAS | 1-25 | 25 |
| CDR-H1 | GYTFTSYNMH | 26-35 | 10 |
| HFR2 | WVRQAPGQGLEWIG | 36-49 | 14 |
| CDR-H2 | AIYPGNGDTS | 50-59 | 10 |
| HFR3 | YAQKFQGRATLTVDTSTSTAYMELSSLRSEDTAVYYC | 60-98 | 39 |
| | AR | | |
| CDR-H3 | GRGRYFEY | 99-106 | 8 |
| HFR4 | WGQGTTLTVSS | 107-117 | 11 |
| VH sequence | QVQLVQSGAEVKKPGASVKMSCKASGYTFTSYNMH | | |
| | WVRQAPGQGLEWIGAIYPGNGDTSYAQKFQGRATL | | |
| | TVDTSTSTAYMELSSLRSEDTAVYYCARGRGRYFEYW | | |
| | GQGTTLTVSS | | |
| Name of VH | 07-0646-h7_VH | 07-0646- | 07-0646- |
| | | h7_VH | h7_VH |
| Name of VL | 07-0646-h7_VL | 07-0646- | 07-0646- |
| | | h7_VL | h7_VL |
| Method | AbM | AbM | AbM |
| Region | Sequence Fragment | Residues | Length |
| LFR1 | DIQLTQSPSSLSASVGDRVTMTC | 1-23 | 23 |
| CDR-L1 | RASSSVSSSYLH | 24-35 | 12 |
| LFR2 | WYQQKPGKAPKLLIY | 36-50 | 15 |
| CDR-L2 | STSNLAS | 51-57 | 7 |
| LFR3 | GVPSRFSGSGSGTDYTLTISSVQPEDFATYYC | 58-89 | 32 |
| CDR-L3 | QQYDSSPST | 90-98 | 9 |
| LFR4 | FGQGTKLEIKRTV | 99-111 | 13 |
| VL sequence | DIQLTQSPSSLSASVGDRVTMTCRASSSVSSSYLHWY | | |
| | QQKPGKAPKLLIYSTSNLASGVPSRFSGSGSGTDYTLT | | |
| | ISSVQPEDFATYYCQQYDSSPSTFGQGTKLEIKRTV | | |
| Name of VL | 07-0646-h7_VL | 07-0646- | 07-0646- |
| | | h7_VL | h7_VL |

TABLE 3A
Amino Acid Sequence of Humanized CDH17 Antibody 07-0653-h43
(IMGT Numbering)

| | | |
|---|---|---|
| Name of antibodies | 07-0653-h43 | |
| name of expression | p07-0653-h43 | |
| vector | | |
| Name of VH or VL | 07-0653-h43_VH | 07-0653-h43_VL |
| FR1-IMGT | QVQLVQSGAEVKKPGASVKVSCK | DIQLTQSPSSLSASVGDRVTITCRA |
| | AS | T |
| CDR1-IMGT | GYTFTDYY | SSLSY |
| FR2-IMGT | MNWVRQAPGQGLEWMGV | IHWYQQKPGKAPKPLIY |
| CDR2-IMGT | IYPYSGGI | EIS |
| FR3-IMGT | GYAQKFQGRVTMTVDKSTSTAY | KLASGVPSRFSGSGSGTDYTLTISS |
| | MELSSLRSEDTAVYYC | LQPEDFATYYC |
| CDR3-IMGT | ARGRGDYFGLFDF | QQWNYPFT |
| FR4-IMGT | WGQGTTVTVSS | FGQGTKLEIK |
| VH or VL sequence | QVQLVQSGAEVKKPGASVKVSCK | DIQLTQSPSSLSASVGDRVTITCRA |
| | ASGYTFTDYYMNWVRQAPGQGL | TSSLSYIHWYQQKPGKAPKPLIYEI |
| | EWMGVIYPYSGGIGYAQKFQGRV | SKLASGVPSRFSGSGSGTDYTLTIS |
| | TMTVDKSTSTAYMELSSLRSEDTA | SLQPEDFATYYCQQWNYPFTFGQ |
| | VYYCARGRGDYFGLFDFWGQGTT | GTKLEIK |
| | VTVSS | |
| Name of VH or VL | 07-0653-h43_VH | 07-0653-h43_VL |
| Project of antibody | CDH17 | CDH17 |

TABLE 3B
Amino Acid Sequence of Humanized CDH17 Antibody 07-0653-h43
(Kabat Numbering)

| Name of VH | 07-0653-h43_VH | 07-0653- | 07-0653- |
|---|---|---|---|
| | | h43_VH | h43_VH |
| Method | Kabat | Kabat | Kabat |
| Region | Sequence Fragment | Residues | Length |
| HFR1 | QVQLVQSGAEVKKPGASVKVSCKASGYTFT | 1-30 | 30 |
| CDR-H1 | DYYMN | 31-35 | 5 |
| HFR2 | WVRQAPGQGLEWMG | 36-49 | 14 |
| CDR-H2 | VIYPYSGGIGYAQKFQG | 50-66 | 17 |
| HFR3 | RVTMTVDKSTSTAYMELSSLRSEDTAVYYCAR | 67-98 | 32 |
| CDR-H3 | GRGDYFGLFDF | 99-109 | 11 |
| HFR4 | WGQGTTVTVSS | 110-120 | 11 |
| VH sequence | QVQLVQSGAEVKKPGASVKVSCKASGYTFTDYYMN | | |
| | WVRQAPGQGLEWMGVIYPYSGGIGYAQKFQGRVT | | |
| | MTVDKSTSTAYMELSSLRSEDTAVYYCARGRGDYFG | | |
| | LFDFWGQGTTVTVSS | | |
| Name of VH | 07-0653-h43_VH | 07-0653- | 07-0653- |
| | | h43_VH | h43_VH |
| Name of VL | 07-0653-h43_VL | 07-0653- | 07-0653- |
| | | h43_VL | h43_VL |
| Method | Kabat | Kabat | Kabat |
| Region | Sequence Fragment | Residues | Length |
| LFR1 | DIQLTQSPSSLSASVGDRVTITC | 1-23 | 23 |
| CDR-L1 | RATSSLSYIH | 24-33 | 10 |
| LFR2 | WYQQKPGKAPKPLIY | 34-48 | 15 |
| CDR-L2 | EISKLAS | 49-55 | 7 |
| LFR3 | GVPSRFSGSGSGTDYTLTISSLQPEDFATYYC | 56-87 | 32 |
| CDR-L3 | QQWNYPFT | 88-95 | 8 |
| LFR4 | FGQGTKLEIKRTV | 96-108 | 13 |
| VL sequence | DIQLTQSPSSLSASVGDRVTITCRATSSLSYIHWYQQK | | |
| | PGKAPKPLIYEISKLASGVPSRFSGSGSGTDYTLTISSLQ | | |
| | PEDFATYYCQQWNYPFTFGQGTKLEIKRTV | | |
| Name of VL | 07-0653-h43_VL | 07-0653- | 07-0653- |
| | | h43_VL | h43_VL |

TABLE 3C
Amino Acid Sequence of Humanized CDH17 Antibody 07-0653-h43
(AbM Numbering)

| Name of VH | 07-0653-h43_VH | 07-0653- | 07-0653- |
|---|---|---|---|
| | | h43_VH | h43_VH |
| Method | AbM | AbM | AbM |
| Region | Sequence Fragment | Residues | Length |
| HFR1 | QVQLVQSGAEVKKPGASVKVSCKAS | 1-25 | 25 |
| CDR-H1 | GYTFTDYYMN | 26-35 | 10 |
| HFR2 | WVRQAPGQGLEWMG | 36-49 | 14 |
| CDR-H2 | VIYPYSGGIG | 50-59 | 10 |
| HFR3 | YAQKFQGRVTMTVDKSTSTAYMELSSLRSEDTAVYY | 60-98 | 39 |
| | CAR | | |
| CDR-H3 | GRGDYFGLFDF | 99-109 | 11 |
| HFR4 | WGQGTTVTVSS | 110-120 | 11 |
| VH sequence | QVQLVQSGAEVKKPGASVKVSCKASGYTFTDYYMN | | |
| | WVRQAPGQGLEWMGVIYPYSGGIGYAQKFQGRVT | | |
| | MTVDKSTSTAYMELSSLRSEDTAVYYCARGRGDYFG | | |
| | LFDFWGQGTTVTVSS | | |
| Name of VH | 07-0653-h43_VH | 07-0653- | 07-0653- |
| | | h43_VH | h43_VH |
| Name of VL | 07-0653-h43_VL | 07-0653- | 07-0653- |
| | | h43_VL | h43_VL |
| Method | AbM | AbM | AbM |
| Region | Sequence Fragment | Residues | Length |
| LFR1 | DIQLTQSPSSLSASVGDRVTITC | 1-23 | 23 |
| CDR-L1 | RATSSLSYIH | 24-33 | 10 |
| LFR2 | WYQQKPGKAPKPLIY | 34-48 | 15 |
| CDR-L2 | EISKLAS | 49-55 | 7 |
| LFR3 | GVPSRFSGSGSGTDYTLTISSLQPEDFATYYC | 56-87 | 32 |
| CDR-L3 | QQWNYPFT | 88-95 | 8 |
| LFR4 | FGQGTKLEIKRTV | 96-108 | 13 |
| VL sequence | DIQLTQSPSSLSASVGDRVTITCRATSSLSYIHWYQQK | | |
| | PGKAPKPLIYEISKLASGVPSRFSGSGSGTDYTLTISSLQ | | |
| | PEDFATYYCQQWNYPFTFGQGTKLEIKRTV | | |
| Name of VL | 07-0653-h43_VL | 07-0653- | 07-0653- |
| | | h43_VL | h43_VL |

TABLE 4A
Amino Acid Sequence of Humanized CDH17 Antibody 07-0663-h7
(IMGT Numbering)

| Name of antibodies | 07-0663-h7 | |
|---|---|---|
| name of expression | p07-0663-h7 (p07-0663-h7 was deposited with ATCC. ATCC | |
| vector | Accession Number: to be determined) | |
| Name of VH or VL | 07-0663-h7_VH | 07-0663-h7_VL |
| FR1-IMGT | QVQLVQSGAEVKKPGSSVKISCKVS | DIQMTQSPSSLSASVGDRVTITCR |
| | | VS |
| CDR1-IMGT | GYTFTDHT | SIISSSK |
| FR2-IMGT | IHWMRQAPGQGLEWIGY | LHWYQQKPGKAPKPLIY |
| CDR2-IMGT | IFPRDDIV | GTS |
| FR3-IMGT | VYAQKFQGRATLTADKSTSTAYME | TLASGVPSRFSGSGSGTDYTLTISS |
| | LSSLRSEDTAVYYC | LQPEDFATYYC |
| CDR3-IMGT | ARPPYYYSRNFYFDY | QQWSNYPFT |
| FR4-IMGT | WGQGTTLTVSS | FGQGTKLEIK |
| VH or VL sequence | QVQLVQSGAEVKKPGSSVKISCKVS | DIQMTQSPSSLSASVGDRVTITCR |
| | GYTFTDHTIHWMRQAPGQGLEWI | VSSIISSSKLHWYQQKPGKAPKPL |
| | GYIFPRDDIVVYAQKFQGRATLTAD | IYGTSTLASGVPSRFSGSGSGTDY |
| | KSTSTAYMELSSLRSEDTAVYYCAR | TLTISSLQPEDFATYYCQQWSNY |
| | PPYYYSRNFYFDYWGQGTTLTVSS | PFTFGQGTKLEIK |
| Name of VH or VL | 07-0663-h7_VH | 07-0663-h7_VL |
| Project of antibody | CDH17 | CDH17 |

TABLE 4B
Amino Acid Sequence of Humanized CDH17 Antibody 07-0663-h7
(Kabat Numbering)

| Name of VH | 07-0663-h7_VH | 07-0663- | 07-0663- |
|---|---|---|---|
| | | h7_VH | h7_VH |
| Method | Kabat | Kabat | Kabat |
| Region | Sequence Fragment | Residues | Length |
| HFR1 | QVQLVQSGAEVKKPGSSVKISCKVSGYTFT | 1-30 | 30 |
| CDR-H1 | DHTIH | 31-35 | 5 |
| HFR2 | WMRQAPGQGLEWIG | 36-49 | 14 |
| CDR-H2 | YIFPRDDIVVYAQKFQG | 50-66 | 17 |
| HFR3 | RATLTADKSTSTAYMELSSLRSEDTAVYYCAR | 67-98 | 32 |
| CDR-H3 | PPYYYSRNFYFDY | 99-111 | 13 |
| HFR4 | WGQGTTLTVSS | 112-122 | 11 |
| VH sequence | QVQLVQSGAEVKKPGSSVKISCKVSGYTFTDHTIHW | | |
| | MRQAPGQGLEWIGYIFPRDDIVVYAQKFQGRATLTA | | |
| | DKSTSTAYMELSSLRSEDTAVYYCARPPYYYSRNFYFD | | |
| | YWGQGTTLTVSS | | |
| Name of VH | 07-0663-h7_VH | 07-0663- | 07-0663- |
| | | h7_VH | h7_VH |
| Name of VL | 07-0663-h7_VL | 07-0663- | 07-0663- |
| | | h7_VL | h7_VL |
| Method | Kabat | Kabat | Kabat |
| Region | Sequence Fragment | Residues | Length |
| LFR1 | DIQMTQSPSSLSASVGDRVTITC | 1-23 | 23 |
| CDR-L1 | RVSSIISSSKLH | 24-35 | 12 |
| LFR2 | WYQQKPGKAPKPLIY | 36-50 | 15 |
| CDR-L2 | GTSTLAS | 51-57 | 7 |
| LFR3 | GVPSRFSGSGSGTDYTLTISSLQPEDFATYYC | 58-89 | 32 |
| CDR-L3 | QQWSNYPFT | 90-98 | 9 |
| LFR4 | FGQGTKLEIKRTV | 99-111 | 13 |
| VL sequence | DIQMTQSPSSLSASVGDRVTITCRVSSIISSSKLHWYQ | | |
| | QKPGKAPKPLIYGTSTLASGVPSRFSGSGSGTDYTLTIS | | |
| | SLQPEDFATYYCQQWSNYPFTFGQGTKLEIKRTV | | |
| Name of VL | 07-0663-h7_VL | 07-0663- | 07-0663- |
| | | h7_VL | h7_VL |

TABLE 4C
Amino Acid Sequence of Humanized CDH17 Antibody 07-0663-h7
(AbM Numbering)

| Name of VH | 07-0663-h7_VH | 07-0663- | 07-0663- |
|---|---|---|---|
| | | h7_VH | h7_VH |
| Method | AbM | AbM | AbM |
| Region | Sequence Fragment | Residues | Length |
| HFR1 | QVQLVQSGAEVKKPGSSVKISCKVS | 1-25 | 25 |
| CDR-H1 | GYTFTDHTIH | 26-35 | 10 |
| HFR2 | WMRQAPGQGLEWIG | 36-49 | 14 |
| CDR-H2 | YIFPRDDIVV | 50-59 | 10 |
| HFR3 | YAQKFQGRATLTADKSTSTAYMELSSLRSEDTAVYYC | 60-98 | 39 |
| | AR | | |
| CDR-H3 | PPYYYSRNFYFDY | 99-111 | 13 |
| HFR4 | WGQGTTLTVSS | 112-122 | 11 |
| VH sequence | QVQLVQSGAEVKKPGSSVKISCKVSGYTFTDHTIHW | | |
| | MRQAPGQGLEWIGYIFPRDDIVVYAQKFQGRATLTA | | |
| | DKSTSTAYMELSSLRSEDTAVYYCARPPYYYSRNFYFD | | |
| | YWGQGTTLTVSS | | |
| Name of VH | 07-0663-h7_VH | 07-0663- | 07-0663- |
| | | h7_VH | h7_VH |
| Name of VL | 07-0663-h7_VL | 07-0663- | 07-0663- |
| | | h7_VL | h7_VL |
| Method | AbM | AbM | AbM |
| Region | Sequence Fragment | Residues | Length |
| LFR1 | DIQMTQSPSSLSASVGDRVTITC | 1-23 | 23 |
| CDR-L1 | RVSSIISSSKLH | 24-35 | 12 |
| LFR2 | WYQQKPGKAPKPLIY | 36-50 | 15 |
| CDR-L2 | GTSTLAS | 51-57 | 7 |
| LFR3 | GVPSRFSGSGSGTDYTLTISSLQPEDFATYYC | 58-89 | 32 |
| CDR-L3 | QQWSNYPFT | 90-98 | 9 |
| LFR4 | FGQGTKLEIKRTV | 99-111 | 13 |
| VL sequence | DIQMTQSPSSLSASVGDRVTITCRVSSIISSSKLHWYQ | | |
| | QKPGKAPKPLIYGTSTLASGVPSRFSGSGSGTDYTLTIS | | |
| | SLQPEDFATYYCQQWSNYPFTFGQGTKLEIKRTV | | |
| Name of VL | 07-0663-h7_VL | 07-0663- | 07-0663- |
| | | h7_VL | h7_VL |

TABLE 5
Nucleic Acid Sequence of Humanized CDH17 Antibodies

| Name | Description | Sequence |
|---|---|---|
| 07-0663- | CDH17 Lead | GACATCCAGATGACCCAGAGCCCTAGCAGCCTGAGCGCCAGCGTGGG |
| h7_VL | antibody: | AGATAGAGTCACAATCACCTGTAGAGTGTCCTCCATCATCAGCTCTTCT |
| | light chain | AAGCTGCACTGGTATCAGCAGAAACCAGGCAAGGCCCCTAAGCCTCTG |
| | | ATCTACGGCACAAGCACCCTGGCTTCTGGCGTGCCCAGCCGGTTCAGC |
| | | GGCAGCGGATCTGGCACCGACTACACCCTGACCATTAGCAGCCTGCAG |
| | | CCTGAGGACTTCGCCACATACTACTGCCAGCAATGGTCCAACTACCCCT |
| | | TTACATTCGGCCAGGGCACCAAGCTGGAAATCAAG |
| 07-0663- | CDH17 Lead | CAAGTGCAGCTGGTGCAGAGCGGAGCCGAGGTGAAAAAGCCAGGCA |
| h7_VH | antibody: | GCTCTGTGAAGATCAGCTGCAAGGTGTCTGGCTACACCTTCACCGACC |
| | heavy chain | ACACCATCCACTGGATGCGGCAGGCCCCTGGCCAGGGCCTGGAATGG |
| | | ATCGGCTACATCTTTCCTAGAGATGACATCGTGGTCTACGCCCAGAAG |
| | | TTCCAGGGCAGAGCCACACTGACCGCTGATAAGTCTACAAGCACAGCT |
| | | TACATGGAACTGAGCTCCCTGCGGAGCGAGGACACCGCCGTGTACTAT |
| | | TGTGCCAGACCTCCCTACTACTACAGCAGAAACTTCTACTTCGACTACT |
| | | GGGGCCAGGGAACCACCCTGACAGTGTCCAGC |
| 07-0653- | CDH17 lead | GATATCCAGCTGACACAGAGCCCTAGCAGCCTCTCCGCCAGCGTGGGC |
| h43_VL | antibody | GACCGGGTGACCATCACCTGTAGAGCCACCAGCAGCCTGAGCTACATC |
| | Light chain | CACTGGTATCAGCAGAAACCCGGCAAGGCCCCTAAGCCTCTGATCTAC |
| | | GAGATTAGCAAGCTGGCTTCTGGAGTGCCATCTAGATTCAGCGGCAGC |
| | | GGATCTGGCACCGACTACACCCTGACCATCTCCTCCCTGCAGCCTGAG |
| | | GACTTCGCCACATACTACTGCCAGCAATGGAACTACCCCTTCACCTTTG |
| | | GCCAGGGCACAAAGCTGGAAATCAAG |
| 07-0653- | CDH17 lead | CAAGTCCAACTCGTCCAATCCGGTGCTGAAGTCAAGAAACCTGGTGCA |
| h43_VH | antibody | TCCGTCAAAGTCTCCTGTAAAGCAAGTGGTTATACATTTACTGATTATT |
| | Heavy chain | ATATGAATTGGGTCCGCCAAGCACCTGGGCAAGGGCTCGAATGGATG |
| | | GGTGTAATTTATCCATATTCTGGCGGAATAGGATATGCTCAAAAGTTTC |
| | | AAGGGCGAGTAACAATGACAGTTGATAAATCCACATCAACTGCTTACA |
| | | TGGAATTGTCCTCACTCCGAAGTGAAGATACGGCTGTTTATTATTGTGC |
| | | AAGAGGGCGTGGGGATTATTTTGGACTCTTTGATTTCTGGGGACAAG |
| | | GAACGACTGTAACAGTCTCTTCC |
| 07-0646- | CDH17 Lead | GATATCCAGCTGACACAGAGCCCTAGCAGCCTGAGCGCCAGCGTGGG |
| h7_VL | antibody: | CGACCGGGTGACCATGACCTGTAGAGCCTCTAGCAGCGTGTCCTCCAG |
| | light chain | CTACCTGCACTGGTATCAGCAAAAGCCCGGCAAAGCCCCTAAGCTCCT |
| | | GATCTACAGCACCAGCAACCTGGCTTCTGGAGTGCCCAGCAGATTCAG |
| | | CGGATCTGGCAGCGGCACAGATTACACCCTGACCATCAGCTCTGTCCA |
| | | GCCTGAGGACTTCGCCACCTACTACTGCCAGCAGTACGACAGCTCCCC |
| | | ATCTACATTTGGCCAGGGCACCAAGCTGGAAATCAAG |
| 07-0646- | CDH17 Lead | CAAGTGCAGCTGGTCCAGAGCGGCGCCGAGGTGAAAAAGCCTGGAG |
| h7_VH | antibody: | CTTCTGTGAAGATGAGCTGCAAGGCCTCTGGCTACACCTTCACCAGCT |
| | heavy chain | ACAACATGCACTGGGTGCGGCAGGCCCCTGGCCAGGGCCTGGAATGG |
| | | ATCGGCGCTATCTACCCCGGCAACGGCGATACATCTTATGCCCAGAAG |
| | | TTTCAGGGAAGAGCCACACTGACCGTGGACACCAGCACCTCCACCGCC |
| | | TACATGGAACTGAGCAGCCTGAGAAGCGAGGACACAGCCGTGTACTA |
| | | CTGTGCCAGAGGAAGAGGCCGGTACTTCGAGTACTGGGGCCAGGGCA |
| | | CCACCCTGACAGTGTCCAGC |

TABLE 6
Binding Activity of the Antibodies and ADCs

| Humanized CDH17 Abs | | 07-0646-h7 | 07-0653-h43 | 07-0663-h7 |
|---|---|---|---|---|
| Antibody Binding | LS513 (Colon, CDH17+) | 47,189 | 24,295 | 74,670.0 |
| Activity by Flow | M202 (Melanoma, CDh17−) | 4,477 | 4,697 | 4,481.0 |
| Cytometry (20 μg/ml) | | | | |
| ADC Binding Activity | LS513 (Colon, CDh17+) | 73,516 | 35,083 | 67,397.0 |
| by Flow Cytometry | M202 (Melanoma, CDH17−) | 4,555 | 4,206 | 4,485.5 |
| (20 μg/ml) | | | | |
| Antibody Binding | HEK293T CDH17mGFP Clone G11 | 0.817 | 0.736 | 0.152 |
| Affinity - KD by | SNUC1 | 0.532 | 0.206 | 0.157 |
| KinExA (nM) | | | | |
| EC50 (nM) by ELISA | Recombinant CDH17 ECD-6xhis tag | 0.73 | 1.13 | 1.22 |
| Cross Reactivity to | HEK293T Human CDH17mGFP G11 | 50,487.50 | 51,778.00 | 57,633.50 |
| Three Tox Models by | HEK293T Monkey CDH17mGFP mass | 16,895.00 | 29,796.00 | 41,303.00 |
| Flow (20 μg/ml) | HEK293T Mouse CDH17mGFP mass | 2,510.50 | 2,455.50 | 2,423.50 |
| | HEK293T Rat CDH17mGFP mass pop | 2,201.00 | 71,824.00 | 2,165.00 |
| Cross Reactivity to | HEK293T Human CDH17mGFP G11 | 0.817 | 0.736 | 0.152 |
| Three Tox Models - KD | HEK293T Monkey CDH17mGFP mass | 0.657 | 0.946 | 0.336 |
| by KinExA (nM) | HEK293T Mouse CDH17mGFP mass | not defined | not defined | not defined |
| | HEK293T Rat CDH17mGFP mass pop | not defined | 0.535 | not defined |

TABLE 7
Characteristics of the Antibodies

| Humanized CDH17 Abs | | 07-0646-h7 | 07-0653-h43 | 07-0663-h7 |
|---|---|---|---|---|
| Antibody | Aggregates | 4.39 | 2.13 | 1.04 |
| Aggregation | Monomers | 95.62 | 97.87 | 98.39 |
| by SEC using | Fragements | 0.00 | 0.00 | 0.57 |
| AKTA Pure | | | | |
| 25 (% of area) | | | | |

| Ab Hydrophobicity-HIC | 30.11 | 36.74 | 31.89 |
|---|---|---|---|
| Retesion Time (min) | | | |
| Antibody Tm in PBS (° C.) | 70.14 | 69.22 | 69.82 |
| Antibody Tagg in PBS (° C.) | 90.33 | 87.58 | 80.09 |
| Antibody Expression Yield in | 0.261 | 0.339 | 0.355 |
| ExpiCHO-S (~mg purified | | | |
| Ab/ml Medium) | | | |
| Internalization rate with | ~3-5 | | |
| Native Cell Positive Cell Line | | | |
| LS513 (100% done at hours) | | | |
| Internalization rate with | ~24-48 | | |
| Native Positive Cell Line | | | |
| HPAF2 (100% done at hours) | | | |

TABLE 8A
Binding Activity of the Antibodies by Flow

| | | | | | | 2nd Ab |
|---|---|---|---|---|---|---|
| Representative Three CDH17 hAbs | | | 07-0663-h7 | 07-0646-h7 | 07-0653-h43 | only |
| Antibody Binding | HEK293T | 1 μg Ab | 1,686.0 | 1,383.0 | 2,375.0 | 1,634.0 |
| Activity by Flow | Parental | 0.2 μg | 1,720.0 | 1,228.0 | 1,723.0 | |
| iQue ® (artifical | | Ab | | | | |
| cell line vs | HEK293T | 1 μg Ab | 171,593.5 | 180,683.0 | 191,092.0 | 1,958.5 |
| its parental) | CDH17 | 0.2 μg | 57,633.5 | 50,487.5 | 51,778.0 | |
| | mGFP Clone | Ab | | | | |
| | G11 (human) | | | | | |
| Antibody Binding | LS513 | 1 μg Ab | 2,298,719.0 | 2,660,407.0 | 1,828,584.5 | 4,902.0 |
| Activity by Flow | (colon | 0.2 μg | 2,239,829.0 | 2,857,996.0 | 1,790,850.5 | |
| iQue ® (native | positive cell | Ab | | | | |
| positive/negative | line)* | | | | | |
| cell lines) | PATU8988S | 1 μg Ab | 101,178.0 | 95,584.5 | 52,178.0 | 5,926.0 |
| | (pancreatic | 0.2 μg | 101,809.0 | 21,403.0 | 48,106.5 | |
| | positive cell | Ab | | | | |
| | line) | | | | | |
| | SNUC1 | 1 μg Ab | 76,052.0 | 74,902.0 | 61,407.0 | 3,899.5 |
| | (colon | 0.2 μg | 163,433.0 | 120,394.0 | 77,220.0 | |
| | positive cell | Ab | | | | |
| | line) | | | | | |
| | M202 | 1 μg Ab | 2,580.0 | 3,002.5 | 2,992.0 | 2,338.0 |
| | (negative | 0.2 μg | 2,319.0 | 2,390.0 | 2,691.0 | |
| | cell line) | Ab | | | | |

TABLE 8B
CDH17 sc-fvs and BiTEs selectively bind to CDH17 positive cells

| | | LS513 (CDH17+) | M202 (CDH17−) |
|---|---|---|---|
| Antibody | | | |
| CDH17 | 07-0646-h7scFv | 863,541.50 | 8,584.00 |
| scFv | 07-0653-h43scFv | 2,205,108.50 | 104,299.00 |
| | 07-0663-h7scFv | 2,050,063.00 | 20,383.00 |
| CDH17 | 07-0646-h7Bs | 3,296,926.00 | 40,014.00 |
| BiTE | 07-0653-h43Bs | 1,599,671.00 | 57,589.00 |
| | 07-0663-h7Bs | 2,271,484.50 | 17,007.00 |

| | | |
|---|---|---|
| Control | | |
| AF647-anti-his tag 2° Ab | 33,158.50 | 16,139.00 |
| no ab (cells only) | 3,576.50 | 2,612.00 |

TABLE 9
Alternative CDH17 antibody names and expression
vectors for production of the CDH17 antibodies

| | Antibody name | Antibody expression |
|---|---|---|
| Antibody | (m: mouse VHVL) | vector name |
| chimeric | CDH17-641 (also | p07-0641m (also |
| anti-CDH17 | called 07-0641m | called p07-0641-m) |
| antibody* | or 07-0641-m)) | |
| chimeric | CDH17-644 (also | p07-0644m (also |
| anti-CDH17 | called 07-0644m | called p07-0644-m) |
| antibody* | or 07-0644-m) | |
| chimeric | CDH17-646 (also | p07-646m (also |
| anti-CDH17 | called 07-0646m | called p07-0646-m) |
| antibody* | or 07-0646-m) | |
| chimeric | CDH17-653 (also | p07-0653m (also |
| anti-CDH17 | called 04-0653m | called p07-0653-m) |
| antibody* | or 04-0653-m) | |
| chimeric | CDH17-655 (also | p07-0655m (also |
| anti-CDH17 | called 07-0655m | called p07-0655-m) |
| antibody* | or 07-0655-m) | |
| chimeric | CDH17-657 (also | p07-0657m (also |
| anti-CDH17 | called 07-0657m | called p07-0657-m) |
| antibody* | or 07-0657-m) | |
| humanized | CDH17-646-h7 | p07-0646-h7 |
| anti-CDH17 | (also called | |
| antibody (IgG1) | 07-0646-h7) | |
| humanized | CDH17-653-h33 | p07-0653-h33 |
| anti-CDH17 | (also called | |
| antibody (IgG1) | 07-0653-h33) | |
| humanized | CDH17-653-h42 | p07-0653-h42 |
| anti-CDH17 | (also called | |
| antibody (IgG1) | 07-0653-h42) | |
| humanized | CDH17-653-h43 | p07-0653-h43 |
| anti-CDH17 | (also called | |
| antibody (IgG1) | 07-0653-h43) | |
| humanized | CDH17-657-h16 | p07-0657-h16 |
| anti-CDH17 | (also called | |
| antibody (IgG1) | 07-0657-h16) | |
| humanized | CDH17-663-h7 | p07-0663-h7 (deposited |
| anti-CDH17 | (also called | with ATCC. ATCC |
| antibody (IgG1) | 07-0663-h7) | Accession Number: |
| | | to be determined) |
| humanized | CDH17-670-h12 | p07-0670-h12 |
| anti-CDH17 | (also called | |
| antibody (IgG1) | 07-0670-h12) | |
| humanized | CDH17-675-h11 | p07-0675-h11 |
| anti-CDH17 | (also called | |
| antibody (IgG1) | 07-0675-h11) | |
| humanized | CDH17-683-h6 | p07-0683-h6 |
| anti-CDH17 | (also called | |
| antibody (IgG1) | 07-0683-h6) | |
| non-targeting | IgG (also called | pIgG (also called |
| human IgG1 | Hu-IgG1) | pHu-IgG1) |
| negative control | | |
| antibody | | |
| scFv comprising | 07-0646-h7scFv | p07-0646-h7scfv |
| light and heavy | (also called | (also called |
| chain variable | CDH17-646-h7scfv) | pCDH17-646-h7scfv) |
| regions of | | |
| 07-0646-h7 | | |
| antibody | | |
| scFv comprising | 07-0653-h43scFv | p07-0653-h43scfv |
| light and heavy | (also called | (also called |
| chain variable | CDH17-653-h43scfv) | p CDH17-653-h43scfv) |
| regions of | | |
| 04-0561-F1 | | |
| antibody | | |
| scFv comprising | 07-0663-h7scFv | p07-0663-h7scfv |
| light and heavy | (also called | (also called |
| chain variable | CDH17-663-h7scfv) | pCDH17-663-h7scfv) |
| regions of | | |
| 04-0562-h10 | | |
| antibody | | |
| bispecific T-cell | 07-0646-h7Bs | p07-0646-h7Bs |
| engager (BiTE) | (also called | (also called |
| Fv comprising | CDH17-646-h7Bs) | pCDH17-646-h7Bs) |
| 07-0646-h7scFv | | |
| antigen-binding | | |
| portion and scFv | | |
| that binds CD3 on | | |
| surface of T cells | | |
| bispecific T-cell | 07-0653-h43Bs | p07-0653-h43Bs |
| engager (BiTE) | (also called | (also called |
| Fv comprising | CDH17-653-h43Bs) | pCDH17-653-h43Bs) |
| 07-0653-h43scFv | | |
| antigen-binding | | |
| portion and scFv | | |
| that binds CD3 on | | |
| surface of T cells | | |
| bispecific T-cell | 07-0663-h7Bs | p07-0663-h7Bs |
| engager (BiTE) | (also called | (also called |
| Fv comprising | CDH17-663-h7Bs) | pCDH17-663-h7Bs) |
| 07-0663-h7scFv | | |
| antigen-binding | | |
| portion and scFv | | |
| that binds CD3 on | | |
| surface of T cells | | |

*Chimeric anti-CDH17 antibody comprises mouse light and heavy chain variable regions and human IgG1 constant region, where human IgG1 light and heavy chain variable regions were replaced with the variable regions of the mouse anti-CDH17 antibody.
*A plasmid comprising the DNA encoding the humanized anti-CDH17 antibody, p07-0663-h7, was deposited with the American Type Culture Collection (ATCC), 10801 University Boulevard, Manassas, Virginia 20110-2209, on Dec. 7, 2022, and assigned Accession Number . This deposit will be maintained under the terms of the Budapest Treaty on the International Recognition of the Deposit of Microorganisms for the Purposes of Patent Procedure.

TABLE 10
CDH17 antibody conjugates

| Antibody Conjugate^{@} | Description |
|---|---|
| 07-0646-h7-VC-PAB-MMAE (also | 07-0646-h7 antibody |
| called CDH17-ADC-646-h7) | conjugated to MMAE |
| 07-0653-h43-VC-PAB-MMAE (also | 07-0653-h43 antibody |
| called CDH17-ADC-653-h43) | conjugated to MMAE |
| 07-0663-h7-VC-PAB-MMAE (also | 07-0663-h7 antibody |
| called CDH17-ADC-663-h7) | conjugated to MMAE |
| 07-0663-h7-AF647 | 07-0663-h7 conjugated |
| | to Alexa Fluor ® 647 |

^{@}MMAE conjugation is performed at a free thiol group of a cysteine residue using MC-VC-PAB-MMAE.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. As used herein, the term “about” when used before a numerical designation, e.g., temperature, time, amount, concentration, and such other, including a range, indicates approximations which may vary by (+) or (−) 10%, 9%, 8%, 7%, 6%, 5%, 4%, 3%, 2%, or 1%.

All methods described herein can be performed in any suitable order unless otherwise indicated herein or otherwise clearly contradicted by context. The use of any and all examples, or various language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. An antigen-binding protein that specifically binds to human cadherin-17 (CDH17) comprising:
a. CDRs 1-3 derived from a heavy chain variable region comprising the amino acid sequence:
QVQLVQSGAEVKKPGSSVKISCKVSGYTFTDHTIHWMRQAPGQGLE WIGYIFPRDDIVVYAQKFQGRATLTADKSTSTAYMELSSLRSEDTAVY YCARPPYYYSRNFYFDYWGQGTTLTVSS (SEQ ID NO: 49) or a variant sequence thereof which differs by only one or two amino acids or which has at least or about 85% sequence identity; and/or
b. CDRs 1-3 derived from a light chain variable region comprising the amino acid sequence:
DIQMTQSPSSLSASVGDRVTITCRVSSIISSSKLHWYQQKPGKAPKPLI YGTSTLASGVPSRFSGSGSGTDYTLTISSLQPEDFATYYCQQWSNYPF TFGQGTKLEIK (SEQ ID NO: 50) or a variant sequence thereof which differs by only one or two amino acids or which has at least or about 85% sequence identity.

2. An antigen-binding protein comprising
a. CDRs 1-3 derived from a heavy chain variable region comprising the amino acid sequence:
QVQLVQSGAEVKKPGASVKMSCKASGYTFTSYNMHWVRQAPGQGL EWIGAIYPGNGDTSYAQKFQGRATLTVDTSTSTAYMELSSLRSEDTA VYYCARGRGRYFEYWGQGTTLTVSS (SEQ ID NO: 45) or a variant sequence thereof which differs by only one or two amino acids or which has at least or about 85% sequence identity; and/or
b. CDRs 1-3 derived from a light chain variable region comprising the amino acid sequence:
DIQLTQSPSSLSASVGDRVTMTCRASSSVSSSYLHWYQQKPGKAPKLL IYSTSNLASGVPSRFSGSGSGTDYTLTISSVQPEDFATYYCQQYDSSPS TFGQGTKLEIK (SEQ ID NO: 46) or a variant sequence thereof which differs by only one or two amino acids or which has at least or about 85% sequence identity.

3. An antigen-binding protein comprising:
a. CDRs 1-3 derived from a heavy chain variable region comprising the amino acid sequence:
QVQLVQSGAEVKKPGASVKVSCKASGYTFTDYYMNWVRQAPGQGL EWMGVIYPYSGGIGYAQKFQGRVTMTVDKSTSTAYMELSSLRSEDTA VYYCARGRGDYFGLFDFWGQGTTVTVSS (SEQ ID NO: 47) or a variant sequence thereof which differs by only one or two amino acids or which has at least or about 85% sequence identity; and/or
b. CDRs 1-3 derived from a light chain variable region comprising the amino acid sequence:
DIQLTQSPSSLSASVGDRVTITCRATSSLSYIHWYQQKPGKAPKPLIYEI SKLASGVPSRFSGSGSGTDYTLTISSLQPEDFATYYCQQWNYPFTFGQ GTKLEIK (SEQ ID NO: 48) or a variant sequence thereof which differs by only one or two amino acids or which has at least or about 85% sequence identity.

4. The antigen-binding protein of claim 1 comprising:
a. a heavy chain CDR1 comprising the amino acid sequence of: GYTFTDHT (SEQ ID NO: 13);
b. a heavy chain CDR2 comprising the amino acid sequence of: IFPRDDIV (SEQ ID NO: 14);
c. a heavy chain CDR3 comprising the amino acid sequence of: ARPPYYYSRNFYFDY (SEQ ID NO: 15);
d. a light chain CDR1 comprising the amino acid sequence of: SIISSSK (SEQ ID NO: 16);
e. a light chain CDR2 comprising the amino acid sequence of: GTS (SEQ ID NO: 17); and
f. a light chain CDR3 comprising the amino acid sequence of: QQWSNYPFT (SEQ ID NO: 18).

5-6. (canceled)

7. The antigen-binding protein of claim 4, additionally comprising:
a. a heavy chain FR1 comprising the amino acid sequence: QVQLVQSGAEVKKPGSSVKISCKVS (SEQ ID NO: 37) or a variant sequence thereof which differs by only one or two amino acids or which has at least or about 95% sequence identity;
b. a heavy chain FR2 comprising the amino acid sequence: IHWMRQAPGQGLEWIGY (SEQ ID NO: 38) or a variant sequence thereof which differs by only one or two amino acids or which has at least or about 95% sequence identity;
c. a heavy chain FR3 comprising the amino acid sequence: VYAQKFQGRATLTADKSTSTAYMELSSLRSEDTAVYYC (SEQ ID NO: 39) or a variant sequence thereof which differs by only one or two amino acids or which has at least or about 95% sequence identity;
d. a heavy chain FR4 comprising the amino acid sequence: WGQGTTLTVSS (SEQ ID NO: 40) or a variant sequence thereof which differs by only one or two amino acids or which has at least or about 95% sequence identity;
e. a light chain FR1 comprising the amino acid sequence: DIQMTQSPSSLSASVGDRVTITCRVS (SEQ ID NO: 41) or a variant sequence thereof which differs by only one or two amino acids or which has at least or about 95% sequence identity;
f. a light chain FR2 comprising the amino acid sequence: LHWYQQKPGKAPKPLIY (SEQ ID NO: 42) or a variant sequence thereof which differs by only one or two amino acids or which has at least or about 95% sequence identity;
g. a light chain FR3 comprising the amino acid sequence: TLASGVPSRFSGSGSGTDYTLTISSLQPEDFATYYC (SEQ ID NO: 43) or a variant sequence thereof which differs by only one or two amino acids or which has at least or about 95% sequence identity; and
h. a light chain FR4 comprising the amino acid sequence: FGQGTKLEIK (SEQ ID NO: 44) or a variant sequence thereof which differs by only one or two amino acids or which has at least or about 95% sequence identity.

8-12. (canceled)

13. An antigen-binding protein that specifically binds to human cadherin-17 (CDH17) comprising:
a. a heavy chain CDR1 comprising the amino acid sequence of: GYTFXDXT (SEQ ID NO: 55) wherein X at position 5 is N, S, R, Q, A or T, and X at position 7 is H, W, Y or F;
b. a heavy chain CDR2 comprising the amino acid sequence of: IFPRDDIV (SEQ ID NO: 14);
c. a heavy chain CDR3 comprising the amino acid sequence of: ARPPYYYSRNFYFDY (SEQ ID NO: 15);
d. a light chain CDR1 comprising the amino acid sequence of: SIISSSK (SEQ ID NO: 16);
e. a light chain CDR2 comprising the amino acid sequence of: GTS (SEQ ID NO: 17); and
f. a light chain CDR3 comprising the amino acid sequence of: QQWSNYPFT (SEQ ID NO: 18).

14-17. (canceled)

18. The antigen-binding protein of claim 1, wherein:
a. the antigen-binding protein binds to a human cadherin-17 (CDH17) protein (SEQ ID NO: 19);
b. the antigen-binding protein binds an extracellular domain of human CDH17 with a dissociation constant (K_{D}) of about less than 10 nM, 5 nM, 2.5 nM, 1 nM, 0.5 nM, or 0.25 nM;
c. the antigen-binding protein preferentially binds human CDH17 over mouse CDH17 (SEQ ID NO: 20);
d. the antigen-binding protein does not bind mouse CDH17; or
e. a combination thereof.

19. The antigen-binding protein of claim 1 which specifically binds to human cadherin-17 (CDH17) or a polypeptide comprising the amino acid sequence of any one of THNLQVAALDANGIIVEGPVPIT (SEQ ID NO: 82), THNLQVAALDANGII (SEQ ID NO: 83), QVAALDANGIIVEGP (SEQ ID NO: 84), LDANGIIVEGPVPIT (SEQ ID NO: 85), LDANGII (SEQ ID NO: 53), and DANGI (SEQ ID NO: 54.

20. (canceled)

21. The antigen-binding protein of claim 1, which is an antibody or antigen-binding antibody fragment.

22. The antigen-binding protein of claim 21, wherein the antibody is a monoclonal antibody.

23. The antigen-binding protein of claim 21, wherein the antibody is a chimeric antibody, a human antibody, or a humanized antibody.

24-25. (canceled)

26. The antigen-binding protein of claim 1, wherein the antigen-binding protein is a bispecific antigen-binding protein or a bispecific T cell engager (BiTE®).

27. The antigen-binding protein of claim 26, wherein the bispecific antigen-binding protein or the BiTE® comprises the amino acid sequence set forth in
07-0663-h7Bs.

28-31. (canceled)

32. A conjugate comprising an antigen-binding protein of claim 1 and a detectable marker, a cytotoxic agent, or a chemotherapeutic agent.

33-42. (canceled)

43. The conjugate of claim 32, wherein an average number of units of the agent conjugated per antigen-binding protein is in a range of 1 to 8, preferably wherein the average number of units of the agent conjugated per antigen-binding protein is (a) in a range of 3-8, or (b) 4.

44-49. (canceled)

50. The conjugate of claim 32, wherein the conjugate comprises a polypeptide comprising the amino acid sequence set forth in SEQ ID NO: 49 and SEQ ID NO: 50 conjugated to VC-PAB-MMAE.

51. (canceled)

52. A fusion protein comprising an antigen-binding protein of claim 1.

53. A nucleic acid comprising a nucleotide sequence encoding an antigen binding protein of claim 1.

54. (canceled)

55. A vector comprising the nucleic acid of claim 53.

56. (canceled)

57. A host cell comprising the nucleic acid of claim 53.

58-61. (canceled)

62. A method of producing an antigen-binding protein that binds to a cadherin-17 (CDH17) protein, comprising (i) culturing the host cell of claim 57 in a cell culture medium, and (ii) harvesting the antigen-binding protein from the cell culture medium.

63-64. (canceled)

65. A pharmaceutical composition comprising (a) an antigen-binding protein of claim 1; and (b) a pharmaceutically acceptable carrier, diluent and/or excipient.

66. A method of treating a subject with a CDH17-expressing cancer comprising administering to the subject a pharmaceutical composition of claim 65 to treat the cancer.

67. (canceled)

68. A method of reducing tumor size in a subject, comprising administering to the subject a pharmaceutical composition of claim 65 to reduce tumor size.

69-72. (canceled)

73. A method of detecting cadherin-17 (CDH17) in a sample, comprising contacting the sample with an antigen-binding protein of claim 1; and assaying for an immunocomplex comprising the antigen-binding protein bound to CDH17.

74-76. (canceled)

77. A method of activating a T cell to target a CDH17-expressing tumor or cancer cell in a subject, the method comprising administering to the subject a bispecific T cell engager (BiTE®) that comprises a first scFv that binds CDH17 and a second scFv which binds CD3, wherein the first scFv that binds CDH17 comprises the VH region and VL region of the antigen-binding protein of claim 1.

78. (canceled)

79. The method of claim 77, wherein the BiTE® is selected 07-0663-h7Bs.

80-82. (canceled)


SEQUENCE LISTING (281 sequences)

SEQ ID NO: 1 - protein, 8 aa, synthetic construct
GYTFTSYN

SEQ ID NO: 2 - protein, 8 aa, synthetic construct
IYPGNGDT

SEQ ID NO: 3 - protein, 10 aa, synthetic construct
ARGRGRYFEY

SEQ ID NO: 4 - protein, 7 aa, synthetic construct
SSVSSSY

SEQ ID NO: 5 - skipped

SEQ ID NO: 6 - protein, 9 aa, synthetic construct
QQYDSSPST

SEQ ID NO: 7 - protein, 8 aa, synthetic construct
GYTFTDYY

SEQ ID NO: 8 - protein, 8 aa, synthetic construct
IYPYSGGI

SEQ ID NO: 9 - protein, 13 aa, synthetic construct
ARGRGDYFGLFDF

SEQ ID NO: 10 - protein, 5 aa, synthetic construct
SSLSY

SEQ ID NO: 11 - skipped

SEQ ID NO: 12 - protein, 8 aa, synthetic construct
QQWNYPFT

SEQ ID NO: 13 - protein, 8 aa, synthetic construct
GYTFTDHT

SEQ ID NO: 14 - protein, 8 aa, synthetic construct
IFPRDDIV

SEQ ID NO: 15 - protein, 15 aa, synthetic construct
ARPPYYYSRNFYFDY

SEQ ID NO: 16 - protein, 7 aa, synthetic construct
SIISSSK

SEQ ID NO: 17 - skipped

SEQ ID NO: 18 - protein, 9 aa, synthetic construct
QQWSNYPFT

SEQ ID NO: 19 - protein, 832 aa, Homo sapiens
MILQAHLHSLCLLMLYLATGYGQEGKFSGPLKPMTFSIYEGQEPSQIIFQFKANPPAVTFELTGETDNIFVIEREGLLYYNRALDRETRSTHNLQVAALDANGIIVEGPVPITIKVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTVGMAVGILLTTLLVIGIILAVVFIRIKKDKGKDNVESAQASEVKPLRS

SEQ ID NO: 20 - protein, 827 aa, Mus musculus
MVSAQLHFLCLLTLYLTCGYGEEGKFSGPLKPMTFSIFEGQEPSQVIFQFKTNPPAVTFELTGETDGIFKIEKDGLLYHTRALDRETRAVHHLQLAALDSHGAIVDGPVPITIEVKDINDNRPTFLQSKYEGSVRQNSRPGKPFMYVNATDLDDPATPNGQLFYQIVIQLPQINDVMYFQIDSKTGAISLTPEGSQELDPVKNPSYNLVVSVKDMGGQSENSFSDTTYVDISIRENIWKAPEPVEIRENSTDPHPIKITQVQWNDPGAQYSLVNKEKLSPFPFSIDQEGNIYVTQALDREEKNSHVFFATAKDENGKPLAYPLEIYVKVIDINDNPPTCLSPVTVFEVQENEPLGNSIGIFEAHDMDEANNINSILKYKLVDQTPKVPSDGLFLIGEYEGKVQLSKQSLKKQDSPQYNLSIEVSDVDFKTLCYIQVNVIDINDQIPIFETSNYGSKTLSEDTAIGSTILIIQATDADEPFTGSSKILYKIVQGDTEGRLEVVTDPTTNAGYVKIKKPLDFETQPVSSIVFQAENPEPLVKGIEYNASSFASFELIVTDVNEVPVFPQRIFQANVSEDAAVGSRVGNVTARDPEGLTVSYSLKGNMRGWLKIDSVTGEIFSAAPLDRETESVYRVQVVATEVGGSSLSSTADFHLVLTDVNDNPPRLAKDYTGLFFCHPLSAPGSLIFEVTDDDQQSLRRPKFTFALGREGLQSDWEVSKINGTHARLSTRHTRFEEQVYNIPIRINDGGQPPMEGTVFLPVTFCQCVEGSCFRPAGRQDGIPTVGMAVGILLTTFLVIGIILAVVFIRMRKDKVENPQSPENKPLRS

SEQ ID NO: 21 - protein, 25 aa, synthetic construct
QVQLVQSGAEVKKPGASVKMSCKAS

SEQ ID NO: 22 - protein, 17 aa, synthetic construct
MHWVRQAPGQGLEWIGA

SEQ ID NO: 23 - protein, 38 aa, synthetic construct
SYAQKFQGRATLTVDTSTSTAYMELSSLRSEDTAVYYC

SEQ ID NO: 24 - protein, 11 aa, synthetic construct
WGQGTTLTVSS

SEQ ID NO: 25 - protein, 26 aa, synthetic construct
DIQLTQSPSSLSASVGDRVTMTCRAS

SEQ ID NO: 26 - protein, 17 aa, synthetic construct
LHWYQQKPGKAPKLLIY

SEQ ID NO: 27 - protein, 36 aa, synthetic construct
NLASGVPSRFSGSGSGTDYTLTISSVQPEDFATYYC

SEQ ID NO: 28 - protein, 10 aa, synthetic construct
FGQGTKLEIK

SEQ ID NO: 29 - protein, 25 aa, synthetic construct [sequence omitted]

SEQ ID NO: 30 - protein, 17 aa, synthetic construct
MNWVRQAPGQGLEWMGV

SEQ ID NO: 31 - protein, 38 aa, synthetic construct
GYAQKFQGRVTMTVDKSTSTAYMELSSLRSEDTAVYYC

SEQ ID NO: 32 - protein, 11 aa, synthetic construct
WGQGTTVTVSS

SEQ ID NO: 33 - protein, 26 aa, synthetic construct
DIQLTQSPSSLSASVGDRVTITCRAT

SEQ ID NO: 34 - protein, 17 aa, synthetic construct
IHWYQQKPGKAPKPLIY

SEQ ID NO: 35 - protein, 36 aa, synthetic construct
KLASGVPSRFSGSGSGTDYTLTISSLQPEDFATYYC

SEQ ID NO: 36 - protein, 10 aa, synthetic construct
FGQGTKLEIK

SEQ ID NO: 37 - protein, 25 aa, synthetic construct
QVQLVQSGAEVKKPGSSVKISCKVS

SEQ ID NO: 38 - protein, 17 aa, synthetic construct
IHWMRQAPGQGLEWIGY

SEQ ID NO: 39 - protein, 38 aa, synthetic construct
VYAQKFQGRATLTADKSTSTAYMELSSLRSEDTAVYYC

SEQ ID NO: 40 - protein, 11 aa, synthetic construct
WGQGTTLTVSS

SEQ ID NO: 41 - protein, 26 aa, synthetic construct
DIQMTQSPSSLSASVGDRVTITCRVS

SEQ ID NO: 42 - protein, 17 aa, synthetic construct
LHWYQQKPGKAPKPLIY

SEQ ID NO: 43 - protein, 36 aa, synthetic construct
TLASGVPSRFSGSGSGTDYTLTISSLQPEDFATYYC

SEQ ID NO: 44 - protein, 10 aa, synthetic construct
FGQGTKLEIK

SEQ ID NO: 45 - protein, 117 aa, synthetic construct
QVQLVQSGAEVKKPGASVKMSCKASGYTFTSYNMHWVRQAPGQGLEWIGAIYPGNGDTSYAQKFQGRATLTVDTSTSTAYMELSSLRSEDTAVYYCARGRGRYFEYWGQGTTLTVSS

SEQ ID NO: 46 - protein, 108 aa, synthetic construct
DIQLTQSPSSLSASVGDRVTMTCRASSSVSSSYLHWYQQKPGKAPKLLIYSTSNLASGVPSRFSGSGSGTDYTLTISSVQPEDFATYYCQQYDSSPSTFGQGTKLEIK

SEQ ID NO: 47 - protein, 120 aa, synthetic construct
QVQLVQSGAEVKKPGASVKVSCKASGYTFTDYYMNWVRQAPGQGLEWMGVIYPYSGGIGYAQKFQGRVTMTVDKSTSTAYMELSSLRSEDTAVYYCARGRGDYFGLFDFWGQGTTVTVSS

SEQ ID NO: 48 - protein, 105 aa, synthetic construct
DIQLTQSPSSLSASVGDRVTITCRATSSLSYIHWYQQKPGKAPKPLIYEISKLASGVPSRFSGSGSGTDYTLTISSLQPEDFATYYCQQWNYPFTFGQGTKLEIK

SEQ ID NO: 49 - protein, 122 aa, synthetic construct
QVQLVQSGAEVKKPGSSVKISCKVSGYTFTDHTIHWMRQAPGQGLEWIGYIFPRDDIVVYAQKFQGRATLTADKSTSTAYMELSSLRSEDTAVYYCARPPYYYSRNFYFDYWGQGTTLTVSS

SEQ ID NO: 50 - protein, 108 aa, synthetic construct
DIQMTQSPSSLSASVGDRVTITCRVSSIISSSKLHWYQQKPGKAPKPLIYGTSTLASGVPSRFSGSGSGTDYTLTISSLQPEDFATYYCQQWSNYPFTFGQGTKLEIK

SEQ ID NO: 51 - protein, 765 aa, synthetic construct
QEGKFSGPLKPMTFSIYEGQEPSQIIFQFKANPPAVTFELTGETDNIFVIEREGLLYYNRALDRETRSTHNLQVAALDANGIIVEGPVPITIKVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTVGM

SEQ ID NO: 52 - protein, 788 aa, synthetic construct
QEGKFSGPLKPMTFSIYEGQEPSQIIFQFKANPPAVTFELTGETDNIFVIEREGLLYYNRALDRETRSTHNLQVAALDANGIIVEGPVPITIKVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTVGMGPEQKLISEEDLNSAVDHHHHHH

SEQ ID NO: 53 - protein, 7 aa, synthetic construct
LDANGII

SEQ ID NO: 54 - protein, 5 aa, synthetic construct
DANGI

SEQ ID NO: 55 - protein, 8 aa, synthetic construct
  VARIANT 5: note = N, S, R, Q, A or T
  VARIANT 7: note = H, W, Y or F
GYTFXDXT

SEQ ID NO: 56 - protein, 8 aa, synthetic construct
  VARIANT 5: note = N, S, R, Q, A or T
  VARIANT 7: note = H, W, Y or F
GYTFXSXN

SEQ ID NO: 57 - protein, 8 aa, synthetic construct
  VARIANT 5: note = N, S, R, Q, A or T
  VARIANT 7: note = H, W, Y or F
GYTFXDXY

SEQ ID NO: 58 - protein, 8 aa, synthetic construct
GYTFNDHT

SEQ ID NO: 59 - protein, 8 aa, synthetic construct
GYTFNDWT

SEQ ID NO: 60 - protein, 8 aa, synthetic construct
GYTFNDYT

SEQ ID NO: 61 - protein, 8 aa, synthetic construct
GYTFNDFT

SEQ ID NO: 62 - protein, 8 aa, synthetic construct
GYTFSDHT

SEQ ID NO: 63 - protein, 8 aa, synthetic construct
GYTFSDWT

SEQ ID NO: 64 - protein, 8 aa, synthetic construct
GYTFSDYT

SEQ ID NO: 65 - protein, 8 aa, synthetic construct
GYTFSDFT

SEQ ID NO: 66 - protein, 8 aa, synthetic construct
GYTFRDHT

SEQ ID NO: 67 - protein, 8 aa, synthetic construct
GYTFRDWT

SEQ ID NO: 68 - protein, 8 aa, synthetic construct
GYTFRDYT

SEQ ID NO: 69 - protein, 8 aa, synthetic construct
GYTFRDFT

SEQ ID NO: 70 - protein, 8 aa, synthetic construct
GYTFQDHT

SEQ ID NO: 71 - protein, 8 aa, synthetic construct
GYTFQDWT

SEQ ID NO: 72 - protein, 8 aa, synthetic construct
GYTFQDYT

SEQ ID NO: 73 - protein, 8 aa, synthetic construct
GYTFQDFT

SEQ ID NO: 74 - protein, 8 aa, synthetic construct
GYTFADHT

SEQ ID NO: 75 - protein, 8 aa, synthetic construct
GYTFADWT

SEQ ID NO: 76 - protein, 8 aa, synthetic construct
GYTFADYT

SEQ ID NO: 77 - protein, 8 aa, synthetic construct
GYTFADFT

SEQ ID NO: 78 - protein, 8 aa, synthetic construct
GYTFTDWT

SEQ ID NO: 79 - protein, 8 aa, synthetic construct
GYTFTDYT

SEQ ID NO: 80 - protein, 8 aa, synthetic construct
GYTFTDFT

SEQ ID NO: 81 - protein, 8 aa, synthetic construct
  VARIANT 5: note = N, S, R, Q, A or T
  VARIANT 6: note = D or S
  VARIANT 7: note = H, W, Y or F
  VARIANT 8: note = Y, T or N
GYTFXXXX

SEQ ID NO: 82 - protein, 23 aa, synthetic construct
THNLQVAALDANGIIVEGPVPIT

SEQ ID NO: 83 - protein, 15 aa, synthetic construct
THNLQVAALDANGII

SEQ ID NO: 84 - protein, 15 aa, synthetic construct
QVAALDANGIIVEGP

SEQ ID NO: 85 - protein, 15 aa, synthetic construct
LDANGIIVEGPVPIT

SEQ ID NO: 86 - protein, 471 aa, synthetic construct
MGWSCIILFLVATATGVHSQVQLVQSGAEVKKPGSSVKISCKVSGYTFTDHTIHWMRQAPGQGLEWIGYIFPRDDIVVYAQKFQGRATLTADKSTSTAYMELSSLRSEDTAVYYCARPPYYYSRNFYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 235 aa, synthetic construct
METDTLLLWVLLLWVPGSTGDIQMTQSPSSLSASVGDRVTITCRVSSIISSSKLHWYQQKPGKAPKPLIYGTSTLASGVPSRFSGSGSGTDYTLTISSLQPEDFATYYCQQWSNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 1413 aa, synthetic construct
ATGGGATGGTCATGTATCATCCTTTTTCTGGTAGCAACTGCAACTGGAGTACATAGCCAGGTTCAGCTAGTTCAATCCGGGGCCGAGGTTAAAAAGCCAGGTTCAAGCGTCAAAATCTCCTGCAAAGTCTCCGGATACACATTCACCGATCATACCATCCACTGGATGCGACAGGCTCCTGGACAAGGCCTGGAGTGGATTGGCTACATTTTTCCACGGGACGACATTGTCGTTTACGCACAAAAATTCCAGGGCCGGGCCACACTTACCGCCGACAAAAGCACTTCAACCGCATACATGGAACTCTCTTCTCTGCGTTCCGAGGACACTGCCGTCTACTACTGTGCACGGCCTCCTTATTATTACTCTCGGAATTTCTACTTCGACTACTGGGGCCAAGGCACAACCCTGACGGTGTCCTCCGCTAGCACCAAGGGCCCATCGGTCTTCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAACTCAGGCGCCCTGACCAGCGGCGTGCACACCTTCCCGGCCGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACAAGAAGGTTGAGCCCAAATCTTGTGACAAAACTCACACATGCCCACCGTGCCCAGCACCTGAACTCCTGGGGGGACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAGTACAAGTGCAAGGTCTCCAACAAAGCCCTCCCAGCCCCCATCGAGAAAACCATCTCCAAAGCCAAAGGGCAGCCCCGAGAACCACAGGTGTACACCCTGCCCCCATCCCGGGACGAGCTGACCAAGAACCAGGTCAGCCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCTCCGGGCAAA

SEQ ID NO: 89 - protein, 705 aa, synthetic construct
ATGGAGACAGACACACTCCTGCTATGGGTACTGCTGCTCTGGGTTCCAGGCTCCACCGGCGATATTCAGATGACCCAGAGCCCCTCTAGCCTCTCCGCCTCTGTTGGGGACAGAGTGACCATTACATGCCGAGTATCTAGCATCATCTCTTCCTCCAAACTGCACTGGTACCAGCAAAAGCCTGGCAAAGCCCCTAAGCCTCTGATTTATGGGACTTCTACTTTAGCCTCCGGCGTCCCAAGTCGGTTCTCTGGAAGTGGCTCCGGCACCGACTACACTCTGACTATCTCCAGTCTGCAGCCCGAGGACTTCGCTACATACTACTGCCAACAATGGTCCAACTATCCTTTTACATTCGGACAAGGCACTAAGCTGGAAATCAAACGTACGGTGGCTGCACCATCTGTCTTCATCTTCCCGCCATCTGATGAGCAGTTGAAATCTGGAACTGCCTCTGTTGTGTGCCTGCTGAATAACTTCTATCCCAGAGAGGCCAAAGTACAGTGGAAGGTGGATAACGCCCTCCAATCGGGTAACTCCCAGGAGAGTGTCACAGAGCAGGACAGCAAGGACAGCACCTACAGCCTCAGCAGCACCCTGACGCTGAGCAAAGCAGACTACGAGAAACACAAAGTCTACGCCTGCGAAGTCACCCATCAGGGCCTGAGCTCGCCCGTCACAAAGAGCTTCAACAGGGGAGAGTGT

SEQ ID NO: 90 - protein, 471 aa, synthetic construct
MEFGLSWVFLVALFRGVQCQVQLVQSGAEVKKPGSSVKISCKVSGYTFTDHTIHWMRQAPGQGLEWIGYIFPRDDIVVYAQKFQGRATLTADKSTSTAYMELSSLRSEDTAVYYCARPPYYYSRNFYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 237 aa, synthetic construct
MDMRVPAQLLGLLLLWLSGARCDIQMTQSPSSLSASVGDRVTITCRVSSIISSSKLHWYQQKPGKAPKPLIYGTSTLASGVPSRFSGSGSGTDYTLTISSLQPEDFATYYCQQWSNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - DNA, 1416 bp, synthetic construct
  source 1..1416: mol_type = other DNA
atggagtttgggctgagctgggttttcctcgttgctctttttagaggtgtccagtgtcaagtgcagctggtgcagagcggagccgaggtgaaaaagccaggcagctctgtgaagatcagctgcaaggtgtctggctacaccttcaccgaccacaccatccactggatgcggcaggcccctggccagggcctggaatggatcggctacatctttcctagagatgacatcgtggtctacgcccagaagttccagggcagagccacactgaccgctgataagtctacaagcacagcttacatggaactgagctccctgcggagcgaggacaccgccgtgtactattgtgccagacctccctactactacagcagaaacttctacttcgactactggggccagggaaccaccctgacagtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 93 - DNA, 714 bp, synthetic construct
  source 1..714: mol_type = other DNA
atggacatgagggtccctgctcagctcctggggctcctgctgctctggctctcaggtgccagatgtgacatccagatgacccagagccctagcagcctgagcgccagcgtgggagatagagtcacaatcacctgtagagtgtcctccatcatcagctcttctaagctgcactggtatcagcagaaaccaggcaaggcccctaagcctctgatctacggcacaagcaccctggcttctggcgtgcccagccggttcagcggcagcggatctggcaccgactacaccctgaccattagcagcctgcagcctgaggacttcgccacatactactgccagcaatggtccaactacccctttacattcggccagggcaccaagctggaaatcaagcgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 94 - protein, 452 aa, synthetic construct
QVQLVQSGAEVKKPGSSVKISCKVSGYTFTDHTIHWMRQAPGQGLEWIGYIFPRDDIVVYAQKFQGRATLTADKSTSTAYMELSSLRSEDTAVYYCARPPYYYSRNFYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 215 aa, synthetic construct
DIQMTQSPSSLSASVGDRVTITCRVSSIISSSKLHWYQQKPGKAPKPLIYGTSTLASGVPSRFSGSGSGTDYTLTISSLQPEDFATYYCQQWSNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 240 aa, synthetic construct
DIQLTQSPSSLSASVGDRVTMTCRASSSVSSSYLHWYQQKPGKAPKLLIYSTSNLASGVPSRFSGSGSGTDYTLTISSVQPEDFATYYCQQYDSSPSTFGQGTKLEIKGGGGSGGGGSGGGGSQVQLVQSGAEVKKPGASVKMSCKASGYTFTSYNMHWVRQAPGQGLEWIGAIYPGNGDTSYAQKFQGRATLTVDTSTSTAYMELSSLRSEDTAVYYCARGRGRYFEYWGQGTTLTVSS

SEQ ID NO: 97 - protein, 240 aa, synthetic construct
DIQLTQSPSSLSASVGDRVTITCRATSSLSYIHWYQQKPGKAPKPLIYEISKLASGVPSRFSGSGSGTDYTLTISSLQPEDFATYYCQQWNYPFTFGQGTKLEIKGGGGSGGGGSGGGGSQVQLVQSGAEVKKPGASVKVSCKASGYTFTDYYMNWVRQAPGQGLEWMGVIYPYSGGIGYAQKFQGRVTMTVDKSTSTAYMELSSLRSEDTAVYYCARGRGDYFGLFDFWGQGTTVTVSS

SEQ ID NO: 98 - protein, 245 aa, synthetic construct
DIQMTQSPSSLSASVGDRVTITCRVSSIISSSKLHWYQQKPGKAPKPLIYGTSTLASGVPSRFSGSGSGTDYTLTISSLQPEDFATYYCQQWSNYPFTFGQGTKLEIKGGGGSGGGGSGGGGSQVQLVQSGAEVKKPGSSVKISCKVSGYTFTDHTIHWMRQAPGQGLEWIGYIFPRDDIVVYAQKFQGRATLTADKSTSTAYMELSSLRSEDTAVYYCARPPYYYSRNFYFDYWGQGTTLTVSS

SEQ ID NO: 99 - protein, 488 aa, synthetic construct
DIQLTQSPSSLSASVGDRVTMTCRASSSVSSSYLHWYQQKPGKAPKLLIYSTSNLASGVPSRFSGSGSGTDYTLTISSVQPEDFATYYCQQYDSSPSTFGQGTKLEIKGGGGSGGGGSGGGGSQVQLVQSGAEVKKPGASVKMSCKASGYTFTSYNMHWVRQAPGQGLEWIGAIYPGNGDTSYAQKFQGRATLTVDTSTSTAYMELSSLRSEDTAVYYCARGRGRYFEYWGQGTTLTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 100 - protein, 488 aa, synthetic construct
DIQLTQSPSSLSASVGDRVTITCRATSSLSYIHWYQQKPGKAPKPLIYEISKLASGVPSRFSGSGSGTDYTLTISSLQPEDFATYYCQQWNYPFTFGQGTKLEIKGGGGSGGGGSGGGGSQVQLVQSGAEVKKPGASVKVSCKASGYTFTDYYMNWVRQAPGQGLEWMGVIYPYSGGIGYAQKFQGRVTMTVDKSTSTAYMELSSLRSEDTAVYYCARGRGDYFGLFDFWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 101 - protein, 493 aa, synthetic construct
DIQMTQSPSSLSASVGDRVTITCRVSSIISSSKLHWYQQKPGKAPKPLIYGTSTLASGVPSRFSGSGSGTDYTLTISSLQPEDFATYYCQQWSNYPFTFGQGTKLEIKGGGGSGGGGSGGGGSQVQLVQSGAEVKKPGSSVKISCKVSGYTFTDHTIHWMRQAPGQGLEWIGYIFPRDDIVVYAQKFQGRATLTADKSTSTAYMELSSLRSEDTAVYYCARPPYYYSRNFYFDYWGQGTTLTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 102 - DNA, 720 bp, synthetic construct
  source 1..720: mol_type = other DNA
gatatccagctgacacagagccctagcagcctgagcgccagcgtgggcgaccgggtgaccatgacctgtagagcctctagcagcgtgtcctccagctacctgcactggtatcagcaaaagcccggcaaagcccctaagctcctgatctacagcaccagcaacctggcttctggagtgcccagcagattcagcggatctggcagcggcacagattacaccctgaccatcagctctgtccagcctgaggacttcgccacctactactgccagcagtacgacagctccccatctacatttggccagggcaccaagctggaaatcaagggtggtggtggttctggaggaggaggatctggagggggggggtcccaagtgcagctggtccagagcggcgccgaggtgaaaaagcctggagcttctgtgaagatgagctgcaaggcctctggctacaccttcaccagctacaacatgcactgggtgcggcaggcccctggccagggcctggaatggatcggcgctatctaccccggcaacggcgatacatcttatgcccagaagtttcagggaagagccacactgaccgtggacaccagcacctccaccgcctacatggaactgagcagcctgagaagcgaggacacagccgtgtactactgtgccagaggaagaggccggtacttcgagtactggggccagggcaccaccctgacagtgtccagc

SEQ ID NO: 103 - DNA, 720 bp, synthetic construct
  source 1..720: mol_type = other DNA
gatatccagctgacacagagccctagcagcctctccgccagcgtgggcgaccgggtgaccatcacctgtagagccaccagcagcctgagctacatccactggtatcagcagaaacccggcaaggcccctaagcctctgatctacgagattagcaagctggcttctggagtgccatctagattcagcggcagcggatctggcaccgactacaccctgaccatctcctccctgcagcctgaggacttcgccacatactactgccagcaatggaactaccccttcacctttggccagggcacaaagctggaaatcaagggtggtggtggttctggaggaggaggatctggagggggggggtcccaagtccaactcgtccaatccggtgctgaagtcaagaaacctggtgcatccgtcaaagtctcctgtaaagcaagtggttatacatttactgattattatatgaattgggtccgccaagcacctgggcaagggctcgaatggatgggtgtaatttatccatattctggcggaataggatatgctcaaaagtttcaagggcgagtaacaatgacagttgataaatccacatcaactgcttacatggaattgtcctcactccgaagtgaagatacggctgtttattattgtgcaagagggcgtggggattattttggactctttgatttctggggacaaggaacgactgtaacagtctcttcc

SEQ ID NO: 104 - DNA, 735 bp, synthetic construct
  source 1..735: mol_type = other DNA
gacatccagatgacccagagccctagcagcctgagcgccagcgtgggagatagagtcacaatcacctgtagagtgtcctccatcatcagctcttctaagctgcactggtatcagcagaaaccaggcaaggcccctaagcctctgatctacggcacaagcaccctggcttctggcgtgcccagccggttcagcggcagcggatctggcaccgactacaccctgaccattagcagcctgcagcctgaggacttcgccacatactactgccagcaatggtccaactacccctttacattcggccagggcaccaagctggaaatcaagggtggtggtggttctggaggaggaggatctggagggggggggtcccaagtgcagctggtgcagagcggagccgaggtgaaaaagccaggcagctctgtgaagatcagctgcaaggtgtctggctacaccttcaccgaccacaccatccactggatgcggcaggcccctggccagggcctggaatggatcggctacatctttcctagagatgacatcgtggtctacgcccagaagttccagggcagagccacactgaccgctgataagtctacaagcacagcttacatggaactgagctccctgcggagcgaggacaccgccgtgtactattgtgccagacctccctactactacagcagaaacttctacttcgactactggggccagggaaccaccctgacagtgtccagc

SEQ ID NO: 105 - DNA, 1464 bp, synthetic construct
  source 1..1464: mol_type = other DNA
gatatccagctgacacagagccctagcagcctgagcgccagcgtgggcgaccgggtgaccatgacctgtagagcctctagcagcgtgtcctccagctacctgcactggtatcagcaaaagcccggcaaagcccctaagctcctgatctacagcaccagcaacctggcttctggagtgcccagcagattcagcggatctggcagcggcacagattacaccctgaccatcagctctgtccagcctgaggacttcgccacctactactgccagcagtacgacagctccccatctacatttggccagggcaccaagctggaaatcaagggtggtggtggttctggaggaggaggatctggagggggggggtcccaagtgcagctggtccagagcggcgccgaggtgaaaaagcctggagcttctgtgaagatgagctgcaaggcctctggctacaccttcaccagctacaacatgcactgggtgcggcaggcccctggccagggcctggaatggatcggcgctatctaccccggcaacggcgatacatcttatgcccagaagtttcagggaagagccacactgaccgtggacaccagcacctccaccgcctacatggaactgagcagcctgagaagcgaggacacagccgtgtactactgtgccagaggaagaggccggtacttcgagtactggggccagggcaccaccctgacagtgtccagcggcggcggcggcagcgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaag

SEQ ID NO: 106 - DNA, 1464 bp, synthetic construct
  source 1..1464: mol_type = other DNA
gatatccagctgacacagagccctagcagcctctccgccagcgtgggcgaccgggtgaccatcacctgtagagccaccagcagcctgagctacatccactggtatcagcagaaacccggcaaggcccctaagcctctgatctacgagattagcaagctggcttctggagtgccatctagattcagcggcagcggatctggcaccgactacaccctgaccatctcctccctgcagcctgaggacttcgccacatactactgccagcaatggaactaccccttcacctttggccagggcacaaagctggaaatcaagggtggtggtggttctggaggaggaggatctggagggggggggtcccaagtccaactcgtccaatccggtgctgaagtcaagaaacctggtgcatccgtcaaagtctcctgtaaagcaagtggttatacatttactgattattatatgaattgggtccgccaagcacctgggcaagggctcgaatggatgggtgtaatttatccatattctggcggaataggatatgctcaaaagtttcaagggcgagtaacaatgacagttgataaatccacatcaactgcttacatggaattgtcctcactccgaagtgaagatacggctgtttattattgtgcaagagggcgtggggattattttggactctttgatttctggggacaaggaacgactgtaacagtctcttccggcggcggcggcagcgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaag

SEQ ID NO: 107 - DNA, 1479 bp, synthetic construct
  source 1..1479: mol_type = other DNA
gacatccagatgacccagagccctagcagcctgagcgccagcgtgggagatagagtcacaatcacctgtagagtgtcctccatcatcagctcttctaagctgcactggtatcagcagaaaccaggcaaggcccctaagcctctgatctacggcacaagcaccctggcttctggcgtgcccagccggttcagcggcagcggatctggcaccgactacaccctgaccattagcagcctgcagcctgaggacttcgccacatactactgccagcaatggtccaactacccctttacattcggccagggcaccaagctggaaatcaagggtggtggtggttctggaggaggaggatctggagggggggggtcccaagtgcagctggtgcagagcggagccgaggtgaaaaagccaggcagctctgtgaagatcagctgcaaggtgtctggctacaccttcaccgaccacaccatccactggatgcggcaggcccctggccagggcctggaatggatcggctacatctttcctagagatgacatcgtggtctacgcccagaagttccagggcagagccacactgaccgctgataagtctacaagcacagcttacatggaactgagctccctgcggagcgaggacaccgccgtgtactattgtgccagacctccctactactacagcagaaacttctacttcgactactggggccagggaaccaccctgacagtgtccagcggcggcggcggcagcgacatcaagctgcagcagagcggcgccgagctggccaggcccggcgccagcgtgaagatgagctgcaagaccagcggctacaccttcaccaggtacaccatgcactgggtgaagcagaggcccggccagggcctggagtggatcggctacatcaaccccagcaggggctacaccaactacaaccagaagttcaaggacaaggccaccctgaccaccgacaagagcagcagcaccgcctacatgcagctgagcagcctgaccagcgaggacagcgccgtgtactactgcgccaggtactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagggccagcagcagcgtgagctacatgaactggtaccagcagaagagcggcaccagccccaagaggtggatctacgacaccagcaaggtggccagcggcgtgccctacaggttcagcggcagcggcagcggcaccagctacagcctgaccatcagcagcatggaggccgaggacgccgccacctactactgccagcagtggagcagcaaccccctgaccttcggcgccggcaccaagctggagctgaag

SEQ ID NO: 108 - protein, 832 aa, Macaca fascicularis
MILQAHLHSLCLLMLYLATGYGQEGKFSGPLKPMTFSIYEGQEPSQIIFQFKANPPAVTFELTGETDNIFKIEREGLLYYTKALDRETRSTHNLQVAALDANGAIVEGPVPITIEVKDVNDNRPTFLQSKYEGSVRQNSRPGKPFLYVNATDLDDPATPNGQLSYQIVIQLPMINNVMYFQINNKTGGISLTREGSQELNPAKNPSYNLVISVKDMGGQSENSFSDTTSVDIIVTENIWKAPEPVEMVENSTDPHPIKITQVRWNDPGAQYSLVDKEKLPRFPFSIDQEGDIYVTQPLDREEKDAYVFYAVAKDEYGKPLSYPLEIHVKVQDINDNPPTCPSPVTVFEVQENERLGNSIGTLTAHDSDEENTANSLLNYRIVEQTPKLPMDGLFLIQTYAGMLQLAKQSLKKQDTPQYNLTIEVSDKDFKTLCFVQINVIDINDQIPIFEKSDYGNLTLAEDTNVGSTILTIQATDADEPFTGSSKILYHVIKGDSEGRLGVDTDPHTNTGYVIIKKPLDFETAAISNIVFKAENPEPLVFGVTYNASSFAKFTLFVTDVNEAPEFSQYVFQAKVSEDVAIGTKVGNVTAKDPEGLDISYSLRGDTRGWLKIDHVTGEIFSVAPLDREAGSPYRVQVVATEVGGSSLSSVSQFHLILTDVNDNPPRLAKDYMDLYFCHPLSAPGSLIFEATDDDQHLFRGPHFTFSIASESLQNDWQVSKINGTHARLSTRHTDFEEKEYVVSIRINDGGRPPLESTVSLTVTFCSCGEDGCFRPAGHQPGIPTVGMAVGILLTTLLVIGIILAVVFIRMKTDKGKDNVESAQASEVKPLRS

SEQ ID NO: 109 - protein, 827 aa, Rattus norvegicus
MVSAQLHFLCLLTLYLTGAYGQEGKFSGPLKPMTFSIFEGQEPSQIIFQFKANPPAVTFELTGETDGIFKIEKDGLLYHTRVLDRETRAVHHLQLAALDSQGAIVDGPVPIIIEVKDINDNRPTFLQTKYEGSVRQNSRPGKPFMYVNATDLDDPATPNGQLFYQIVIQLPKINNVMYFQIDNKTGAISLTPEGSQVLDPIKNPYYNLVVSVKDMGGQNENSFSDTTSVDITVRENIWKAPEPVEIRENLTDPHPIKITQVQWNDPGAHYSLINKEKLPQFPFSIDQEGNIYVTQPLDREEKDSHVFFATAKDENGKPLAYPLEIRVKVIDINDNPPTCLSQVTVFEVQENEVLGSSIGIFAAHDMDEANNINSILKYRLVDQTPKVPSDELFLIDEYGGKVQLGKRSLKKQDSPQYNLTVEVSDIDFKTLCSLQVNVIDINDQIPIFERSDYGSKTLSEDTAIGSTILIIQATDDDEPFTGSSKILYKIVQGDTEGRLEVVTDPMTNTGYVKIRKPLDFETEPVTSIVFKAENPEPLVNGIEYNASSFASFELTVTDVNEVPVFPQQIFQANVSEDTAIGTKVGTVTARDPEGLTVSYSLKDNKRGWLKIDSVTGDIFSTAPLDRETESVYRVQVVATEVGGSSLSSTAYFHLVLMDVNDNPPRLAKDYTGLFFCHPLSAPGSLIFEATDDDQQSVRRPKFTFALGRESLQSDWEVSKINGTHARLSTKHTRFEEQVYDIPILINDGGQPPMEGIVSLSVTFCQCVDGSCFRPAGNQVGIPTVGMAVGILLTTFLVIGIILAVVFIRMRKDKVEDPQSPENKPLRS

SEQ ID NO: 110 - protein, 23 aa, synthetic construct
THNLQVAALDANGIIVEGPVPIT

SEQ ID NO: 111 - protein, 15 aa, synthetic construct
THNLQVAALDANGII

SEQ ID NO: 112 - protein, 15 aa, synthetic construct
QVAALDANGIIVEGP

SEQ ID NO: 113 - protein, 15 aa, synthetic construct
LDANGIIVEGPVPIT

SEQ ID NO: 114 - protein, 7 aa, synthetic construct
ADANGII

SEQ ID NO: 115 - protein, 7 aa, synthetic construct
LAANGII

SEQ ID NO: 116 - protein, 7 aa, synthetic construct
LDGNGII

SEQ ID NO: 117 - protein, 7 aa, synthetic construct
LDAAGII

SEQ ID NO: 118 - protein, 7 aa, synthetic construct
LDANAII

SEQ ID NO: 119 - protein, 7 aa, synthetic construct
LDANGAI

SEQ ID NO: 120 - protein, 7 aa, synthetic construct
LDANGIA

SEQ ID NO: 121 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 122 - protein, 330 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 123 - protein, 107 aa, Homo sapiens [sequence omitted]

SEQ ID NO: 124 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 125 - protein, 330 aa, Homo sapiens [sequence omitted]

SEQ ID NO: 126 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 127 - protein, 104 aa, Homo sapiens
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 4 aa, synthetic construct
LEIK

SEQ ID NO: 129 - protein, 4 aa, synthetic construct
GGFG

SEQ ID NO: 130 - protein, 4 aa, synthetic construct
  SITE 1: note = maleimidocaproyl glycine
GGFG

SEQ ID NO: 131 - protein, 6 aa, synthetic construct
HHHHHH

SEQ ID NO: 132 - protein, 25 aa, synthetic construct
QVQLVQSGAEVKKPGASVKMSCKAS

SEQ ID NO: 133 - protein, 8 aa, synthetic construct
GYTFTSYN

SEQ ID NO: 134 - protein, 17 aa, synthetic construct
MHWVRQAPGQGLEWIGA

SEQ ID NO: 135 - protein, 8 aa, synthetic construct
IYPGNGDT

SEQ ID NO: 136 - protein, 38 aa, synthetic construct; identical to SEQ ID NO: 23

SEQ ID NO: 137 - protein, 10 aa, synthetic construct
ARGRGRYFEY

SEQ ID NO: 138 - protein, 11 aa, synthetic construct
WGQGTTLTVSS

SEQ ID NO: 139 - protein, 117 aa, synthetic construct; identical to SEQ ID NO: 45

SEQ ID NO: 140 - protein, 26 aa, synthetic construct
DIQLTQSPSSLSASVGDRVTMTCRAS

SEQ ID NO: 141 - protein, 7 aa, synthetic construct
SSVSSSY

SEQ ID NO: 142 - protein, 17 aa, synthetic construct
LHWYQQKPGKAPKLLIY

SEQ ID NO: 143 - skipped

SEQ ID NO: 144 - protein, 36 aa, synthetic construct; identical to SEQ ID NO: 27

SEQ ID NO: 145 - protein, 9 aa, synthetic construct
QQYDSSPST

SEQ ID NO: 146 - protein, 10 aa, synthetic construct
FGQGTKLEIK

SEQ ID NO: 147 - protein, 108 aa, synthetic construct; identical to SEQ ID NO: 46

SEQ ID NO: 148 - protein, 30 aa, synthetic construct
QVQLVQSGAEVKKPGASVKMSCKASGYTFT

SEQ ID NO: 149 - protein, 5 aa, synthetic construct
SYNMH

SEQ ID NO: 150 - protein, 14 aa, synthetic construct
WVRQAPGQGLEWIG

SEQ ID NO: 151 - protein, 17 aa, synthetic construct
AIYPGNGDTSYAQKFQG

SEQ ID NO: 152 - protein, 32 aa, synthetic construct
RATLTVDTSTSTAYMELSSLRSEDTAVYYCAR

SEQ ID NO: 153 - protein, 8 aa, synthetic construct
GRGRYFEY

SEQ ID NO: 154 - protein, 11 aa, synthetic construct
WGQGTTLTVSS

SEQ ID NO: 155 - protein, 117 aa, synthetic construct; identical to SEQ ID NO: 45

SEQ ID NO: 156 - protein, 23 aa, synthetic construct
DIQLTQSPSSLSASVGDRVTMTC

SEQ ID NO: 157 - protein, 12 aa, synthetic construct
RASSSVSSSYLH

SEQ ID NO: 158 - protein, 15 aa, synthetic construct
WYQQKPGKAPKLLIY

SEQ ID NO: 159 - protein, 7 aa, synthetic construct
STSNLAS

SEQ ID NO: 160 - protein, 32 aa, synthetic construct
GVPSRFSGSGSGTDYTLTISSVQPEDFATYYC

SEQ ID NO: 161 - protein, 9 aa, synthetic construct
QQYDSSPST

SEQ ID NO: 162 - protein, 13 aa, synthetic construct
FGQGTKLEIKRTV

SEQ ID NO: 163 - protein, 111 aa, synthetic construct
DIQLTQSPSSLSASVGDRVTMTCRASSSVSSSYLHWYQQKPGKAPKLLIYSTSNLASGVPSRFSGSGSGTDYTLTISSVQPEDFATYYCQQYDSSPSTFGQGTKLEIKRTV

SEQ ID NO: 164 - protein, 25 aa, synthetic construct
QVQLVQSGAEVKKPGASVKMSCKAS

SEQ ID NO: 165 - protein, 10 aa, synthetic construct
GYTFTSYNMH

SEQ ID NO: 166 - protein, 14 aa, synthetic construct
WVRQAPGQGLEWIG

SEQ ID NO: 167 - protein, 10 aa, synthetic construct
AIYPGNGDTS

SEQ ID NO: 168 - protein, 39 aa, synthetic construct
YAQKFQGRATLTVDTSTSTAYMELSSLRSEDTAVYYCAR

SEQ ID NO: 169 - protein, 8 aa, synthetic construct
GRGRYFEY

SEQ ID NO: 170 - protein, 11 aa, synthetic construct
WGQGTTLTVSS

SEQ ID NO: 171 - protein, 117 aa, synthetic construct; identical to SEQ ID NO: 45

SEQ ID NO: 172 - protein, 23 aa, synthetic construct
DIQLTQSPSSLSASVGDRVTMTC

SEQ ID NO: 173 - protein, 12 aa, synthetic construct
RASSSVSSSYLH

SEQ ID NO: 174 - protein, 15 aa, synthetic construct
WYQQKPGKAPKLLIY

SEQ ID NO: 175 - protein, 7 aa, synthetic construct
STSNLAS

SEQ ID NO: 176 - protein, 32 aa, synthetic construct; identical to SEQ ID NO: 160

SEQ ID NO: 177 - protein, 9 aa, synthetic construct
QQYDSSPST

SEQ ID NO: 178 - protein, 13 aa, synthetic construct
FGQGTKLEIKRTV

SEQ ID NO: 179 - protein, 111 aa, synthetic construct; identical to SEQ ID NO: 163

SEQ ID NO: 180 - protein, 25 aa, synthetic construct [sequence omitted]

SEQ ID NO: 181 - protein, 8 aa, synthetic construct
GYTFTDYY

SEQ ID NO: 182 - protein, 17 aa, synthetic construct
MNWVRQAPGQGLEWMGV

SEQ ID NO: 183 - protein, 8 aa, synthetic construct
IYPYSGGI

SEQ ID NO: 184 - protein, 38 aa, synthetic construct; identical to SEQ ID NO: 31

SEQ ID NO: 185 - protein, 13 aa, synthetic construct
ARGRGDYFGLFDF

SEQ ID NO: 186 - protein, 11 aa, synthetic construct
WGQGTTVTVSS

SEQ ID NO: 187 - protein, 120 aa, synthetic construct; identical to SEQ ID NO: 47

SEQ ID NO: 188 - protein, 26 aa, synthetic construct
DIQLTQSPSSLSASVGDRVTITCRAT

SEQ ID NO: 189 - protein, 5 aa, synthetic construct
SSLSY

SEQ ID NO: 190 - protein, 17 aa, synthetic construct
IHWYQQKPGKAPKPLIY

SEQ ID NO: 191 - skipped

SEQ ID NO: 192 - protein, 36 aa, synthetic construct; identical to SEQ ID NO: 35

SEQ ID NO: 193 - protein, 8 aa, synthetic construct
QQWNYPFT

SEQ ID NO: 194 - protein, 10 aa, synthetic construct
FGQGTKLEIK

SEQ ID NO: 195 - protein, 105 aa, synthetic construct; identical to SEQ ID NO: 48

SEQ ID NO: 196 - protein, 30 aa, synthetic construct
QVQLVQSGAEVKKPGASVKVSCKASGYTFT

SEQ ID NO: 197 - protein, 5 aa, synthetic construct
DYYMN

SEQ ID NO: 198 - protein, 14 aa, synthetic construct
WVRQAPGQGLEWMG

SEQ ID NO: 199 - protein, 17 aa, synthetic construct
VIYPYSGGIGYAQKFQG

SEQ ID NO: 200 - protein, 32 aa, synthetic construct
RVTMTVDKSTSTAYMELSSLRSEDTAVYYCAR

SEQ ID NO: 201 - protein, 11 aa, synthetic construct
GRGDYFGLFDF

SEQ ID NO: 202 - protein, 11 aa, synthetic construct
WGQGTTVTVSS

SEQ ID NO: 203 - protein, 120 aa, synthetic construct; identical to SEQ ID NO: 47

SEQ ID NO: 204 - protein, 23 aa, synthetic construct
DIQLTQSPSSLSASVGDRVTITC

SEQ ID NO: 205 - protein, 10 aa, synthetic construct
RATSSLSYIH

SEQ ID NO: 206 - protein, 15 aa, synthetic construct
WYQQKPGKAPKPLIY

SEQ ID NO: 207 - protein, 7 aa, synthetic construct
EISKLAS

SEQ ID NO: 208 - protein, 32 aa, synthetic construct
GVPSRFSGSGSGTDYTLTISSLQPEDFATYYC

SEQ ID NO: 209 - protein, 8 aa, synthetic construct
QQWNYPFT

SEQ ID NO: 210 - protein, 13 aa, synthetic construct
FGQGTKLEIKRTV

SEQ ID NO: 211 - protein, 108 aa, synthetic construct
DIQLTQSPSSLSASVGDRVTITCRATSSLSYIHWYQQKPGKAPKPLIYEISKLASGVPSRFSGSGSGTDYTLTISSLQPEDFATYYCQQWNYPFTFGQGTKLEIKRTV

SEQ ID NO: 212 - protein, 25 aa, synthetic construct [sequence omitted]

SEQ ID NO: 213 - protein, 10 aa, synthetic construct
GYTFTDYYMN

SEQ ID NO: 214 - protein, 14 aa, synthetic construct
WVRQAPGQGLEWMG

SEQ ID NO: 215 - protein, 10 aa, synthetic construct
VIYPYSGGIG

SEQ ID NO: 216 - protein, 39 aa, synthetic construct
YAQKFQGRVTMTVDKSTSTAYMELSSLRSEDTAVYYCAR

SEQ ID NO: 217 - protein, 11 aa, synthetic construct
GRGDYFGLFDF

SEQ ID NO: 218 - protein, 11 aa, synthetic construct
WGQGTTVTVSS

SEQ ID NO: 219 - protein, 120 aa, synthetic construct; identical to SEQ ID NO: 47

SEQ ID NO: 220 - protein, 23 aa, synthetic construct
DIQLTQSPSSLSASVGDRVTITC

SEQ ID NO: 221 - protein, 10 aa, synthetic construct
RATSSLSYIH

SEQ ID NO: 222 - protein, 15 aa, synthetic construct
WYQQKPGKAPKPLIY

SEQ ID NO: 223 - protein, 7 aa, synthetic construct
EISKLAS

SEQ ID NO: 224 - protein, 32 aa, synthetic construct; identical to SEQ ID NO: 208

SEQ ID NO: 225 - protein, 8 aa, synthetic construct
QQWNYPFT

SEQ ID NO: 226 - protein, 13 aa, synthetic construct
FGQGTKLEIKRTV

SEQ ID NO: 227 - protein, 108 aa, synthetic construct; identical to SEQ ID NO: 211

SEQ ID NO: 228 - protein, 25 aa, synthetic construct
QVQLVQSGAEVKKPGSSVKISCKVS

SEQ ID NO: 229 - protein, 8 aa, synthetic construct
GYTFTDHT

SEQ ID NO: 230 - protein, 17 aa, synthetic construct
IHWMRQAPGQGLEWIGY

SEQ ID NO: 231 - protein, 8 aa, synthetic construct
IFPRDDIV

SEQ ID NO: 232 - protein, 38 aa, synthetic construct; identical to SEQ ID NO: 39

SEQ ID NO: 233 - protein, 15 aa, synthetic construct
ARPPYYYSRNFYFDY

SEQ ID NO: 234 - protein, 11 aa, synthetic construct
WGQGTTLTVSS

SEQ ID NO: 235 - protein, 122 aa, synthetic construct; identical to SEQ ID NO: 49

SEQ ID NO: 236 - protein, 26 aa, synthetic construct
DIQMTQSPSSLSASVGDRVTITCRVS

SEQ ID NO: 237 - protein, 7 aa, synthetic construct
SIISSSK

SEQ ID NO: 238 - protein, 17 aa, synthetic construct
LHWYQQKPGKAPKPLIY

SEQ ID NO: 239 - skipped

SEQ ID NO: 240 - protein, 36 aa, synthetic construct; identical to SEQ ID NO: 43

SEQ ID NO: 241 - protein, 9 aa, synthetic construct
QQWSNYPFT

SEQ ID NO: 242 - protein, 10 aa, synthetic construct
FGQGTKLEIK

SEQ ID NO: 243 - protein, 108 aa, synthetic construct; identical to SEQ ID NO: 50

SEQ ID NO: 244 - protein, 30 aa, synthetic construct
QVQLVQSGAEVKKPGSSVKISCKVSGYTFT

SEQ ID NO: 245 - protein, 5 aa, synthetic construct
DHTIH

SEQ ID NO: 246 - protein, 14 aa, synthetic construct
WMRQAPGQGLEWIG

SEQ ID NO: 247 - protein, 17 aa, synthetic construct
YIFPRDDIVVYAQKFQG

SEQ ID NO: 248 - protein, 32 aa, synthetic construct
RATLTADKSTSTAYMELSSLRSEDTAVYYCAR

SEQ ID NO: 249 - protein, 13 aa, synthetic construct
PPYYYSRNFYFDY

SEQ ID NO: 250 - protein, 11 aa, synthetic construct
WGQGTTLTVSS

SEQ ID NO: 251 - protein, 122 aa, synthetic construct; identical to SEQ ID NO: 49

SEQ ID NO: 252 - protein, 23 aa, synthetic construct
DIQMTQSPSSLSASVGDRVTITC

SEQ ID NO: 253 - protein, 12 aa, synthetic construct
RVSSIISSSKLH

SEQ ID NO: 254 - protein, 15 aa, synthetic construct
WYQQKPGKAPKPLIY

SEQ ID NO: 255 - protein, 7 aa, synthetic construct
GTSTLAS

SEQ ID NO: 256 - protein, 32 aa, synthetic construct; identical to SEQ ID NO: 208

SEQ ID NO: 257 - protein, 9 aa, synthetic construct
QQWSNYPFT

SEQ ID NO: 258 - protein, 13 aa, synthetic construct
FGQGTKLEIKRTV

SEQ ID NO: 259 - protein, 111 aa, synthetic construct
DIQMTQSPSSLSASVGDRVTITCRVSSIISSSKLHWYQQKPGKAPKPLIYGTSTLASGVPSRFSGSGSGTDYTLTISSLQPEDFATYYCQQWSNYPFTFGQGTKLEIKRTV

SEQ ID NO: 260 - protein, 25 aa, synthetic construct
QVQLVQSGAEVKKPGSSVKISCKVS

SEQ ID NO: 261 - protein, 10 aa, synthetic construct
GYTFTDHTIH

SEQ ID NO: 262 - protein, 14 aa, synthetic construct
WMRQAPGQGLEWIG

SEQ ID NO: 263 - protein, 10 aa, synthetic construct
YIFPRDDIVV

SEQ ID NO: 264 - protein, 39 aa, synthetic construct
YAQKFQGRATLTADKSTSTAYMELSSLRSEDTAVYYCAR

SEQ ID NO: 265 - protein, 13 aa, synthetic construct
PPYYYSRNFYFDY

SEQ ID NO: 266 - protein, 11 aa, synthetic construct
WGQGTTLTVSS

SEQ ID NO: 267 - protein, 122 aa, synthetic construct; identical to SEQ ID NO: 49

SEQ ID NO: 268 - protein, 23 aa, synthetic construct
DIQMTQSPSSLSASVGDRVTITC

SEQ ID NO: 269 - protein, 12 aa, synthetic construct
RVSSIISSSKLH

SEQ ID NO: 270 - protein, 15 aa, synthetic construct
WYQQKPGKAPKPLIY

SEQ ID NO: 271 - protein, 7 aa, synthetic construct
GTSTLAS

SEQ ID NO: 272 - protein, 32 aa, synthetic construct; identical to SEQ ID NO: 208

SEQ ID NO: 273 - protein, 9 aa, synthetic construct
QQWSNYPFT

SEQ ID NO: 274 - protein, 13 aa, synthetic construct
FGQGTKLEIKRTV

SEQ ID NO: 275 - protein, 111 aa, synthetic construct; identical to SEQ ID NO: 259

SEQ ID NO: 276 - DNA, 324 bp, synthetic construct
  source 1..324: mol_type = other DNA
gacatccagatgacccagagccctagcagcctgagcgccagcgtgggagatagagtcacaatcacctgtagagtgtcctccatcatcagctcttctaagctgcactggtatcagcagaaaccaggcaaggcccctaagcctctgatctacggcacaagcaccctggcttctggcgtgcccagccggttcagcggcagcggatctggcaccgactacaccctgaccattagcagcctgcagcctgaggacttcgccacatactactgccagcaatggtccaactacccctttacattcggccagggcaccaagctggaaatcaag

SEQ ID NO: 277 - DNA, 366 bp, synthetic construct
  source 1..366: mol_type = other DNA
caagtgcagctggtgcagagcggagccgaggtgaaaaagccaggcagctctgtgaagatcagctgcaaggtgtctggctacaccttcaccgaccacaccatccactggatgcggcaggcccctggccagggcctggaatggatcggctacatctttcctagagatgacatcgtggtctacgcccagaagttccagggcagagccacactgaccgctgataagtctacaagcacagcttacatggaactgagctccctgcggagcgaggacaccgccgtgtactattgtgccagacctccctactactacagcagaaacttctacttcgactactggggccagggaaccaccctgacagtgtccagc

SEQ ID NO: 278 - DNA, 315 bp, synthetic construct
  source 1..315: mol_type = other DNA
gatatccagctgacacagagccctagcagcctctccgccagcgtgggcgaccgggtgaccatcacctgtagagccaccagcagcctgagctacatccactggtatcagcagaaacccggcaaggcccctaagcctctgatctacgagattagcaagctggcttctggagtgccatctagattcagcggcagcggatctggcaccgactacaccctgaccatctcctccctgcagcctgaggacttcgccacatactactgccagcaatggaactaccccttcacctttggccagggcacaaagctggaaatcaag

SEQ ID NO: 279 - DNA, 360 bp, synthetic construct
  source 1..360: mol_type = other DNA
caagtccaactcgtccaatccggtgctgaagtcaagaaacctggtgcatccgtcaaagtctcctgtaaagcaagtggttatacatttactgattattatatgaattgggtccgccaagcacctgggcaagggctcgaatggatgggtgtaatttatccatattctggcggaataggatatgctcaaaagtttcaagggcgagtaacaatgacagttgataaatccacatcaactgcttacatggaattgtcctcactccgaagtgaagatacggctgtttattattgtgcaagagggcgtggggattattttggactctttgatttctggggacaaggaacgactgtaacagtctcttcc

SEQ ID NO: 280 - DNA, 324 bp, synthetic construct
  source 1..324: mol_type = other DNA
gatatccagctgacacagagccctagcagcctgagcgccagcgtgggcgaccgggtgaccatgacctgtagagcctctagcagcgtgtcctccagctacctgcactggtatcagcaaaagcccggcaaagcccctaagctcctgatctacagcaccagcaacctggcttctggagtgcccagcagattcagcggatctggcagcggcacagattacaccctgaccatcagctctgtccagcctgaggacttcgccacctactactgccagcagtacgacagctccccatctacatttggccagggcaccaagctggaaatcaag

SEQ ID NO: 281 - DNA, 351 bp, synthetic construct
  source 1..351: mol_type = other DNA
caagtgcagctggtccagagcggcgccgaggtgaaaaagcctggagcttctgtgaagatgagctgcaaggcctctggctacaccttcaccagctacaacatgcactgggtgcggcaggcccctggccagggcctggaatggatcggcgctatctaccccggcaacggcgatacatcttatgcccagaagtttcagggaagagccacactgaccgtggacaccagcacctccaccgcctacatggaactgagcagcctgagaagcgaggacacagccgtgtactactgtgccagaggaagaggccggtacttcgagtactggggccagggcaccaccctgacagtgtccagc